SEQUENCE LISTING (140 sequences)

SEQ ID NO: 1 - protein, 163 aa, Neospora caninum
MSDWDPVVKEWLVDTGYCCAGGIANAEDGVVFAAAADDDDGWSKLYKEDHEEDTIGEDGNVNGKVTVNEASTIKAAVDDVSAPNGVWIGGQKYKVVRPEKGFEYNDCTFDITMCARSKGGAHLIKTPNGSIVIALYDEEKEQDKGNSRTSALAFAEYLHQSGY

SEQ ID NO: 2 - protein, 170 aa, Sarcosystis neurona
MAEEQAGTEEWDTLCQDWLPGTGYCSAGGLCSAEDGVIYAAASNSHKGWAVLYRDDHEQDELGEDGNPIGKVTINEGSTIKKAMEEGSAPNGVWIGGVKYKVVRPEKNVEYNGIMYDTVMCARPKGGAHLIKTPKGTIIVAVYDEEKEQSAGNSRTCALAFAHHLNFLGC

SEQ ID NO: 3 - protein, 163 aa, Toxoplasma gondii
MSDWDPVVKEWLVDTGYCCAGGIANAEDGVVFAAAADDDDGWSKLYKDDHEEDTIGEDGNACGKVSINEASTIKAAVDDGSAPNGVWIGGQKYKVVRPEKGFEYNDCTFDITMCARSKGGAHLIKTPNGSIVIALYDEEKEQDKGNSRTSALAFAEYLHQSGY

SEQ ID NO: 4 - protein, 171 aa, Plasmodium falciparum
MAEEYSWDSYLNDRLLATNQVSGAGLASEEDGVVYACVAQGEESDPNFDKWSLFYKEDYDIEVEDENGTKTTKTINEGQTILVVFNEGYAPDGVWLGGTKYQFINIERDLEFEGYNFDVATCAKLKGGLHLVKVPGGNILVVLYDEEKEQDRGNSKIAALTFAKELAESSQ

SEQ ID NO: 5 - protein, 170 aa, Eimeria acervulina
MGEEADTQAWDTSVKEWLVDTGKVYAGGIASIADGCRLFGAAIDNGEDAWSQLVKTGYQIEVLQEDGSSTQEDCDEAETLRQAIVDGRAPNGVYIGGIKYKLAEVKRDFTYNDQNYDVAILGKNKGGGFLIKTPNDNVVIALYDEEKEQNKADALTTALAFAEYLYQGGF

SEQ ID NO: 6 - protein, 169 aa, Eimeria tenella
MGEADTQAWDTSVREWLVDTGRVFAGGVASIADGCRLFGAAVEGEGNAWEELVKTNYQIEVPQEDGTSISVDCDEAETLRQAVVDGRAPNGVYIGGTKYKLAEVKRDFTFNDQNYDVAILGKNKGGGFLIKTPNENVVIALYDEEKEQNKADALTTALNFAEYLYQGGF

SEQ ID NO: 7 - DNA, 492 bp, Neospora caninum
atgtcggactgggatcccgttgtcaaggagtggcttgttgacacgggctactgctgcgcaggcggcattgcaaatgccgaggacggtgtggttttcgctgcggcggcggatgacgatgacggatggtcaaagttgtacaaggaggaccacgaggaggacacaatcggagaggacggcaacgtgaacggcaaggtgacggtcaatgaggcctccaccattaaagctgcagttgatgatgtcagcgccccgaatggcgtttggattggcggccaaaagtataaagttgtccgacctgagaaaggattcgagtacaacgactgcaccttcgacatcaccatgtgtgcacgatccaagggtggtgcgcacctgatcaagactccgaatggctctattgtcatcgccctctacgatgaggagaaggagcaggacaaagggaacagcaggacgtcggccttggcatttgccgagtaccttcaccagtctggctattaa

SEQ ID NO: 8 - DNA, 513 bp, Sarcosystis neurona
atggcggaggagcaggcagggactgaggagtgggacacgctgtgccaggactggctgcctggcacaggatactgcagtgccggcgggctttgtagcgcggaagacggagtgatttacgccgcagcttcgaatagtcataaggggtgggcagtgctgtacagagacgaccacgaacaagatgaactaggagaagatggaaatcccattgggaaagtgacaataaacgaaggcagcacgatcaaaaaggcgatggaggaagggagcgctcccaatggcgtgtggataggcggagtgaagtataaggtggtgcgaccggagaaaaacgtggaatataacggcatcatgtacgacacagtaatgtgtgctcgcccgaaaggcggtgcgcatttaatcaaaaccccgaagggcactattattgttgcggtctatgatgaagagaaagagcaatctgctggaaactcccgcacgtgcgcgctggcctttgcgcaccacctgaacttcctgggttgctga

SEQ ID NO: 9 - DNA, 492 bp, Toxoplasma gondii
atgtccgactgggaccctgttgtcaaggagtggcttgttgacacaggctactgctgcgcaggcggcatcgccaacgcggaggacggtgttgtgttcgccgcggcggctgatgatgatgacggatggtccaagctgtacaaggatgatcatgaggaggacactatcggagaggatggcaacgcgtgcggcaaggtgtcgatcaacgaggcctccacgatcaaagctgcagttgacgatggcagtgcccctaacggtgtttggattggcggccagaagtacaaggttgtccgacctgagaaaggattcgagtacaacgactgcaccttcgacatcaccatgtgtgcacggtccaagggtggcgcgcacttgatcaagaccccgaatggctctatcgtcattgccctttacgatgaggagaaggaacaggacaagggaaacagcaggacttcggcattggcctttgccgagtatcttcaccagtctgggtactaa

SEQ ID NO: 10 - DNA, 516 bp, Plasmodium falciparum
atggcagaagaatattcatgggacagttatttaaatgatcgccttttagcaaccaatcaagtttcaggagctggattagcttcggaagaagatggagttgtctatgcttgtgtagctcagggtgaagagagtgacccaaattttgataaatggtcacttttttataaagaagattatgatattgaagttgaagatgaaaatggtactaaaactaccaaaacgataaatgaaggacaaacgatcctggtcgtttttaatgaaggatatgctcctgatggagtttggttaggtggtactaaatatcaatttataaatattgaaagagatttagaatttgaaggttataattttgatgtagctacttgtgctaaattaaaaggtggtcttcacttggtgaaagttccaggaggaaatatattagttgtattatatgatgaagaaaaagagcaagacagaggaaattccaaaatcgctgccttgacttttgcaaaagagctagctgaaagcagtcaatag

SEQ ID NO: 11 - DNA, 513 bp, Eimeria acervulina
atgggtgaagaggctgatactcaggcgtgggatacctcagtgaaggaatggctcgtggatacggggaaggtatacgccggcggcattgctagcattgcagatgggtgccgcctgtttggcgctgcaatagacaatggggaggatgcgtggagtcagttggtgaagacaggatatcagattgaagtgcttcaagaggacggctcttcaactcaagaggactgcgatgaagcggaaaccctgcggcaagcaattgttgacggccgtgccccaaacggtgtttatattggaggaattaaatataaactcgcagaagttaaacgtgatttcacctataacgaccagaactacgacgtggcgattttggggaagaacaagggtggcggtttcctgattaagactccgaacgacaatgtggtgattgctctttatgacgaggagaaagagcagaacaaagcagatgcgctgacaacggcacttgccttcgctgagtacctgtaccagggcggcttctaa

SEQ ID NO: 12 - DNA, 510 bp, Eimeria tenella
atgggagaagcagacacccaggcctgggacacttcggtccgcgagtggctggttgacaccggcagggtcttcgccggcggcgttgctagcatagccgacggctgccggctcttcggagcagcagtggagggcgagggcaacgcctgggaagaactcgtcaagaccaactaccaaattgaagtcccccaggaagacggaacctccatttcagtggattgcgacgaggcggagactctgcggcaggcggtggtggacggccgcgcgcccaacggcgtctacatcggcggcaccaagtacaagctcgccgaagtcaaaagggacttcaccttcaacgaccaaaactatgatgtggcgattctgggaaaaaacaaaggcggagggtttttgattaaaactccaaagcaaaatgttgttatagctttgtatgatgaagaaaaagaacaaaacaaagctgatgctctcacaacagctcttaacttcgcggagtatctgtaccagggaggcttctaa

SEQ ID NO: 13 - protein, 56 aa, Artificial Sequence
  Description of Artificial Sequence Synthetic peptide
  MOD_RES (3)..(4): Variable amino acid
  MOD_RES (8): Variable amino acid
  MOD_RES (10)..(11): Variable amino acid
  MOD_RES (17)..(18): Variable amino acid
  MOD_RES (22)..(23): Variable amino acid
  MOD_RES (26): Variable amino acid
  MOD_RES (31): Variable amino acid
  MOD_RES (33)..(36): Variable amino acid
  MOD_RES (47): Variable amino acid
LYXXDHEXDXXGEDGNXXGKVXXNEXSTIKXAXXXXSAPNGVWIGGXKYKVVRPEK

SEQ ID NO: 14 - protein, 30 aa, Artificial Sequence
  Description of Artificial Sequence Synthetic peptide
  MOD_RES (3)..(4): Variable amino acid
  MOD_RES (8): Variable amino acid
  MOD_RES (10)..(11): Variable amino acid
  MOD_RES (17)..(18): Variable amino acid
  MOD_RES (22)..(23): Variable amino acid
  MOD_RES (26): Variable amino acid
LYXXDHEXDXXGEDGNXXGKVXXNEXSTIK

SEQ ID NO: 15 - protein, 48 aa, Artificial Sequence
  Description of Artificial Sequence Synthetic peptide
  MOD_RES (2)..(3): Variable amino acid or not present
  MOD_RES (5)..(11): Variable amino acid or not present
  MOD_RES (13): Variable amino acid or not present
  MOD_RES (15)..(18): Variable amino acid or not present
  MOD_RES (20)..(22): Variable amino acid or not present
  MOD_RES (25)..(26): Variable amino acid or not present
  MOD_RES (29)..(36): Variable amino acid or not present
  MOD_RES (39): Variable amino acid or not present
  MOD_RES (43): Variable amino acid or not present
  MOD_RES (46): Variable amino acid or not present
YXXDXXXXXXXEXGXXXXKXXXNEXXTIXXXXXXXXAPXGVWXGGXKY

SEQ ID NO: 16 - protein, 133 aa, Betula pendula
MSWQTYVDEHLMCDIDGQASNSLASAIVGHDGSVWAQSSSFPQFKPQEITGIMKDFEEPGHLAPTGLHLGGIKYMVIQGEAGAVIRGKKGSGGITIKKTGQALVFGIYEEPVTPGQCNMVVERLGDYLIDQGL

SEQ ID NO: 17 - protein, 133 aa, Betula pendula; identical to SEQ ID NO: 16

SEQ ID NO: 18 - protein, 197 aa, Eimeria tenella
AAASTSKLKSSFLSSFCFLFQIIFYLVCKMGEADTQWDTSVREWLVDTGRVFAGGVASIADGCRLFGAAVEGEGNAWEELVKTNYQIEVPQEDGTSISVDCDEAETLRQAVVDGRAPNGVYIGGTKYKLAEVKRDFTFNDQNYDVAILGKNKGGGFLIKTPNENVVIALYDEEKEQNKADALTTALNFAEYLYQGGF

SEQ ID NO: 19 - DNA, 853 bp, Eimeria tenella
gcggccgcgtcgaccagcaagcttaaaagctcttttttgtccagtttttcgtttcttttccaaattattttctatttagtttgcaaaatgggagaagcagacacccaggcctgggacacttcggtccgcgagtggctggttgacaccggcagggtcttcgccggcggcgttgctagcatagccgacggctgccggctcttcggagcagcagtggagggcgagggcaacgcctgggaagaactcgtcaagaccaactaccaaattgaagtcccccaggaagacggaacctccatttcagtggattgcgacgaggcggagactctgcggcaggcggtggtggacggccgcgcgcccaacggcgtctacatcggcggcaccaagtacaagctcgccgaagtcaaaagggacttcaccttcaacgaccaaaactatgatgtggcgattctgggaaaaaacaaaggcggagggtttttgattaaaactccaaacgaaaatgttgttatagctttgtatgatgaagaaaaagaacaaaacaaagctgatgctctcacaacagctcttaacttcgcggagtatctgtaccagggaggcttctaagtgctgcagcagcggcagcagcagcagcagcagcagcagcagcaggagcggcgggagcagcagcgaaagtggtggtggtgtggcattatatgatgatgagaaacaagtaaagctgatgcattcactgcagcacttatttcgctgagaatctttatcatgggagcttccatgctgcagcagcggcagcagcagcagcggcagcagcagcagcagcagaagtggtgtgggttgttgctctgtacggtggaaagctgct

SEQ ID NO: 20 - protein, 197 aa, Neospora caninum
GYLLYFLFSQALQGNAVFRVISCFFFGLSPLFSKMSDWDPVVKEWLVDTGYCCAGGIANAEDGVVFAAAADDDDGWSKLYKEDHEEDTIGEDGNVNGKVTVNEASTIKAAVDDVSAPNGVWIGGQKYKVVRPEKGFEYNDCTFDITMCARSKGGAHLIKTPNGSIVIALYDEEKEQKDGNSRTSALAFAEYLHQSGY

SEQ ID NO: 21 - DNA, 837 bp, Neospora caninum
ggttacttgctttattttcttttctcccaagctctccaaggtaacgccgtatttcgggttatttcttgcttttttttcggtttgtctcctttattttccaagatgtcggactgggatcccgttgtcaaggagtggcttgttgacacgggctactgctgcgcaggcggcattgcaaatgccgaggacggtgtggttttcgctgcggcggcggatgacgatgacggatggtcaaagttgtacaaggaggaccacgaggaggacacaatcggagaggacggcaacgtgaacggcaaggtgacggtcaatgaggcctccaccattaaagctgcagttgatgatgtcagcgccccgaatggcgtttggattggcggccaaaagtataaagttgtccgacctgagaaaggattcgagtacaacgactgcaccttcgacatcaccatgtgtgcacgatccaagggtggtgcgcacctgatcaagactccgaatggctctattgtcatcgccctctacgatgaggagaaggagcaggacaaagggaacagcaggacgtcggccttggcatttgccgagtaccttcaccagtctggctattaacttgtctgctttgtttgtaacgaaactggagcatgataatcgttcagggcgagcgcttttgaaggctgtgttgattgtgggtgatgcactatcgaacttcggtagtccttccgtatgttgagcatcttcactgcccggtgtctcaactgtttgcctccaccctgttccgtaaacatgtcggcgctcggaaaaaatttctctaaaaaaaaaaaaaactcgagggggacgccctattatgagtcg

SEQ ID NO: 22 - protein, 178 aa, Plasmodium falciparum
GTKLELHRGGGRSRPFRSPGLQEFGTRVLGAGLASEEDGVVYACVAQGEEDSPNFDKWSLFYKEDYDIEVEDENGTKTTKTINEGQTILVVFNEGYAPDGVWLGGTKYQFINIERDLEFEGYNFDVATCAKLKGGLHLVKVPGGNILVVLYDEEKEQDRGNSKIAALTFAKELAESSQ

SEQ ID NO: 23 - DNA, 1196 bp, Plasmodium falciparum
ggtacttcatacctcctaagggacaaagctggagctccaccgcggtggcggccgctctagaccttttagatcccccgggctgcaggaattcggcacgagggtcttaggagctggattagcttcggaagaagatggagttgtctatgcttgtgtagctcagggtgaagagagtgacccaaattttgataaatggtcacttttttataaagaagattatgatattgaagttgaagatgaaaatggtactaaaactaccaaaacgataaatgaaggacaaacgatcctggtcgtttttaatgaaggatatgctcctgatggagtttggttaggtggtactaaatatcaatttataaatattgaaagagatttagaatttgaaggttataattttgatgtagctacttgtgctaaattaaaaggtggtcttcacttggtgaaagttccaggaggaaatatattagttgtattatatgatgaagaaaaagagcaagacagaggaaattccaaaatcgctgccttgacttttgcaaaagagctagctgaaagcagtcaatagggaaattacaaaatgataaataatagaaaaatatgaacacatcatagaaaggaggaatataaaattttgaatacatttaaaaaaaaaaaaaaaaaaactcgagggggggcccggtacccaattcgccctatagtgagtcgtattacaattcactggccgtcgttttacaacgtcctgactggggaaaaccctggcggtacccaacttaatcgccttgcagcacatccccctttccccgctggggtaataaccaaaaggccccgcacgaatcgcccttcccaaaatttgcgccaactgaatggggaatgggcaaattgtagccttaaaatttttgttaaaaatcgccgttaaattttttgttaaatcaacttctttttttaaccaaaagggccgaaatctggaaaatcctttttaaattaaaaaaataaaccccaaaaaggggtgaaggtttttttccccttttgaaaaaagattccacttttaaaaaaagggggattccacccttaaaagggcgaaaaaacctttttttaggggggagggcccccttccggaaaccatcccccaactaaatttttttggggggcgggggcccgaaaagcactaaattcgaaatcctaagggggcccccccatttaaaact

SEQ ID NO: 24 - protein, 217 aa, Sacrocystis neurona
GRIPSGCECRFCPTSHIHLPLCFIHSAFFYFRDSSSSVLPSHLGVTIMAEEQAGTEEWDTLCQDWLPGTGYCSAGGLCSAEDGVIYAAASNSHKGWAVLYRDDHEQDELGEDGNPIGKVTINEGSTIKKAMEEGSAPNGVWIGGVKYKVVRPEKNVEYNGIMYDTVMCARPKGGAHLIKTPKGTIIVAVYDEEKEQSAGNSRTCALAFAHHLNFLGC

SEQ ID NO: 25 - DNA, 1104 bp, Sacrocystis neurona
ggtcgtatcccttcgggttgtgagtgccgtttctgtcccacctcgcacatccatctccctctgtgtttcatccattccgcgtttttttatttccgtgactcgtcttccagcgtcttaccaagccatctgggcgtcaccatcatggcggaggagcaggcagggactgaggagtgggacacgctgtgccaggactggctgcctggcacaggatactgcagtgccggcgggctttgtagcgcggaagacggagtgatttacgccgcagcttcgaatagtcataaggggtgggcagtgctgtacagagacgaccacgaacaagatgaactaggagaagatggaaatcccattgggaaagtgacaataaacgaaggcagcacgatcaaaaaggcgatggaggaagggagcgctcccaatggcgtgtggataggcggagtgaagtataaggtggtgcgaccggagaaaaacgtggaatataacggcatcatgtacgacacagtaatgtgtgctcgcccgaaaggcggtgcgcatttaatcaaaaccccgaagggcactattattgttgcggtctatgatgaagagaaagagcaatctgctggaaactcccgcacgtgcgcgctggcctttgcgcaccacctgaacttcctgggttgctgaaaagagaggccgttgcttgcacgacacgccacgggaaggggaggtctgcaaaagacagagtggagggaggaaaggaggaggaggaggaaacagaagatgaagagagcgaggaagaatgaaaaccgaggaggagcaggaaagggaagaaagtgtgaagaagaatacgaagcgaaataacgcaaaaggagatatggagagacgccgagggagggctgggcggccggtggaaaagaacaacgcagggggagggaaggggaaggataagatacgtgaagaggggaaaacacgaggaaagggagaggcgtgactgaaaagagcagagaaagtgcataacacttatccggacatctcagacatgccgttgctgtactactatatatttcctgaaagagagaaaggacagaagcaactgtgtaaaaggtatatctcaaaaaaaaaaaagaaaaactc

SEQ ID NO: 26 - protein, 187 aa, Toxoplasma gondii
GTRSATSYRILFRFFRGLFPFFSKMSDWDPVVKEWLVDTGYCCAGGIANAEDGVVFAAAADDDDGWSKLYKDDHEEDTIGEDGNACGKVSINEASTIKAAVDDGSAPNGVWIGGQKYKVVRPEKGFEYNDCTFDITMCARSKGGAHLIKTPNGSIVIALYDEEKEQDKGNSRTSALAFAEYLHQSGY

SEQ ID NO: 27 - DNA, 771 bp, Toxoplasma gondii
ggcacgagatcggctacatcttatagaattctttttcgcttctttcgcggcttgtttccattcttttccaagatgtccgactgggaccctgttgtcaaggagtggcttgttgacacaggctactgctgcgcaggcggcatcgccaacgcggaggacggtgttgtgttcgccgcggcggctgatgatgatgacggatggtccaagctgtacaaggatgatcatgaggaggacactatcggagaggatggcaacgcgtgcggcaaggtgtcgatcaacgaggcctccacgatcaaagctgcagttgacgatggcagtgcccctaacggtgtttggattggcggccagaagtacaaggttgtccgacctgagaaaggattcgagtacaacgactgcaccttcgacatcaccatgtgtgcacggtccaagggtggcgcgcacttgatcaagaccccgaatggctctatcgtcattgccctttacgatgaggagaaggaacaggacaagggaaacagcaggacttcggcattggcctttgccgagtatcttcaccagtctgggtactaacttgtctgctttgtttgtaacggaacttgagcgtggtaatcgtccagaattagcgattttgaagaccgtgtctattgtgggtgacgcactatcgaccttcggaagtgctttcgtatgttgagcatctctttcagcccctggtggctcaactctttgcctgcgacctgttccgtaagcatgtcgttgctcaaaaaaaaaaaaaaaaaa

SEQ ID NO: 28 - protein, 940 aa, Escherichia coli
MDKIEVRGARTHNLKNINLVIPRDKLIVVTGLSGSGKSSLAFDTLYAEGQRRYVESLSAYARQFLSLMEKPDVDHIEGLSPAISIEQKSTSHNPRSTVGTITEIHDYLRLLYARVGEPRCPDHDVPLAAQTVSQMVDNVLSQPEGKRLMLLAPIIKERKGEHTKTLENLASQGYIRARIDGEVCDLSDPPKLELQKKHTIEVVVDRFKVRDDLTQRLAESFETALELSGGTAVVADMDDPKAEELLFSANFACPICGYSMRELEPRLFSFNNPAGACPTCDGLGVQQYFDPDRVIQNPELSLAGGAIRGWDRRNFYYFQMLKSLADHYKFDVEAPWGSLSANVHKVVLYGSGKENIEFKYMNDRGDTSIRRHPFEGVLHNMERRYKETESSAVREELAKFISNRPCASCEGTRLRREARHVYVENTPLPAISDMSIGHAMEFFNNLKLAGQRAKIAEKILKEIGDRLKFLVNVGLNYLTLSRSAETLSGGEAQRIRLASQIGAGLVGVMYVLDEPSIGLHQRDNERLLGTLIHLRDLGNTVIVVEHDEDAIRAADHVIDIGPGAGVHGGEVVAEGPLEAIMAVPESLTGQYMSGKRKIEVPKKRVPANPEKVLKLTGARGNNLKDVTLTLPVGLFTCITGVSGSGKSTLINDTLFPIAQRQLNGATIAEPAPYRDIQGLEHFDKVIDIDQSPIGRTPRSNPATYTGVFTPVRELFAGVPESRARGYTPGRFSFNVRGGRCEACQGDGVIKVEMHFLPDIYVPCDQCKGKRYNRETLEIKYKGKTIHEVLDMTIEEAREFFDAVPALARKLQTLMDVGLTYIRLGQSATTLSGGEAQRVKLARELSKRGTGQTLYILDEPTTGLHFADIQQLLDVLHKLRDQGNTIVVIEHNLDVIKTADWIVDLGPEGGSGGGEILVSGTPETVAECKASHTARFLKPML

SEQ ID NO: 29 - DNA, 2822 bp, Escherichia coli
ttacagcatcggcttgaggaagcgcgccgtatgcgaagctttgcactctgcgacggtttctggcgtaccggagacgaggatttcgccgcccccactgccgccttccggtcccaggtcgacaatccagtcagcggttttgatcacgtcgagattgtgctcaattaccacaatggtattgccctgatcgcgcagtttatgcagtacgtcgagcagttgctgaatatcggcgaagtgcagaccggtggtcggctcgtcgagaatatacagcgtctgcccggtgccgcgttttgacagctcacgcgccagcttcacgcgctgggcttcaccaccagaaagtgtggttgcggactgcccgaggcgaatgtacgtcaggccaacatccatcagcgtttgcagcttacgcgccagagctggcaccgcatcaaagaactcacgcgcctcttcgatggtcatatccagcacttcgtggatggttttgcctttgtacttaatctccagcgtttcacggttatagcgtttgcctttgcactggtcgcacggtacgtaaatgtccggcaggaagtgcatctccactttgatcacgccgtcgcctgacaggcctcgcagcgtccgccacggacgttaaagctgaaacgtcctggcgtatagccgcgcgcacgggattccggtacgcccgcaaacagttcgcgcacaggtgtaaacacgccggtataggtcgccgggttagaacgcggagtacgaccaattgggctttggtcgatatcgatcactttatcgaaatgctccagtccctgaatatcgcgatacggtgccggttcggcgatggtcgcaccattcaactggcgttgggcaatcgggaacagtgtgtcgttaatcagtgtcgatttaccggaacctgaaacccctgtgatgcaggtaaacagaccgactggcagcgttagcgtcacgtctttcaggttattaccgcgtgcacccgtcagcttcagcactttttccggattcgccggaacgcgtttcttcggcacttcaatcttgcgtttaccgctcatgtactgtccggtcaacgactccggcaccgccataattgcttccagtggaccttccgcgaccacttcaccgccgtgaacacccgcacccggaccgatatcgatcacatgatcagcggcgcgaatcgcgtcttcgtcgtgctccaccacaatcacggtattaccgagatcgcgcagatggataagcgtacccagcaggcgctcgttatcgcgctggtgcaggccgatagacggctcatccagcacgtacatcacgccaaccaggcctgcaccaatctggctcgccagacgaatacgctgggcttcaccgccggaaagtgtctctgctgagcgggaaagtgtcaggtaattcaggccgacgttaacgaggaatttcaggcgatcgccaatctccttaagaattttttccgcaatcttcgctcgttgacctgcgagtttgagattgttgaagaattccatcgcatgaccgatgctcatgtcggagatagcaggcagcggcgtattctcgacatatacatggcgcgcttcccgacgcagacgcgttccttcgcagctggcgcacgggcgattgctgataaacttggctaattcttcacgtaccgcactggattccgtctctttataacggcgctccatattgtgcagcacgccttcgaacggatgacgacgaatggaggtatcgccacgatcgttcatgtatttgaattcaatattttctttgccagaaccgtacaacaccactttatgcacgttcgcgctcaggctgccccacggcgcttcgacgtcgaacttatagtgatctgccagcgatttcagcatctggaaataatagaagttgcggcgatcccagccacggatcgcaccgccagccagcgataattccggattctggatcacgcggtcaggatcgaaatattgctgtacgccaagaccgtcacaggtcgggcaggctcccgccgggttgttaaatgaaaacaggcgaggttccagctcgcgcatactgtaaccgcaaatcgggcaggcaaagttggcggagaacagcagctcttccgctttcgggtcgtccatatccgccactaccgcggtaccaccggaaagctccagcgcggtttcaaacgactcggcaagacgttgggtaagatcgtcacgcactttgaagcgatcaaccaccacttcaatggtatgtttcttttgcagttccagtttcggcggatcggaaagatcgcagacttcgccatcaatacgagcacggatgtaaccctggcttgccaggttctccagcgttttggtgtgttcgcctttgcgctctttaatgattggcgcgagcagcatcagacgcttgccttccggctgcgaaagcacgttatccaccatctggctgacggtttgcgccgccagcggaacatcgtggtccgggcagcgcggctcaccaacgcgagcgtacagcagacgcaaatagtcgtggatttcggtgattgtccccaccgtagagcgcgggttatgagacgtcgatttctgctcaattgagatggcaggagaaagcccctcaatatggtcgacgtccggcttctccatcagtgacagaaactgccgcgcgtaggcggaaagggattcaacgtaacggcgctgcccttcggcatataaggtgtcgaaagcgagcgaggatttgccagaacccgaaagcccggtcacgacaatgagcttgtcgcgggggataacgaggttgatgtttttgagattatgggtgcgggcgccccgaacttcgatcttatccat

SEQ ID NO: 30 - protein, 673 aa, Escherichia coli
MSKPFKLNSAFKPSGDQPEAIRRLEEGLEDGLAHQTLLGVTGSGKTFTIANVIADLQRPTMVLAPNKTLAAQLYGEMKEFFPENAVEYFVSYYDYYQPEAYVPSSDTFIEKDASVNEHIEQMRLSATKAMLERRDVVVVASVSAIYGLGDPDLYLKMMLHLTVGMIIDQRAILRRLAELQYARNDQAFQRGTFRVRGEVIDIFPAESDDIALRVELFDEEVERLSLFDPLTGQIVSTIPRFTIYPKTHYVTPRERIVQAMEEIKEELAARRKVLLENNKLLEEQRLTQRTQFDLEMMNELGYCSGIENYSRFLSGRGPGEPPPTLFDYLPADGLLVVDESHVTIPQIGGMYRGDRARKETLVEYGFRLPSALDNRPLKFEEFEALAPQTIYVSATPGNYELEKSGGDVVDQVVRPTGLLDPIIEVRPVATQVDDLLSEIRQRAAINERVLVTTLTKRMAEDLTEYLEEHGERVRYLRSDIDTVERMEIIRDLRLGEFDVLVGINLLREGLDMPEVSLVAILDADKEGFLRSERSLIQTIGRAARNVNGKAILYGDKITPSMAKAIGETERRREKQQKYNEEHGITPQGLNKKVVDILALGQNIAKTKAKGRGKSRPIVEPDNVPMDMSPKALQQKIHELEGLMMQHAQNLEFEEAAQIRDQLHQLRELFIAAS

SEQ ID NO: 31 - DNA, 2022 bp, Escherichia coli
atgagtaaaccgttcaaactgaattccgcttttaaaccttctggcgatcagccagaggcgattcgacgtctcgaagaggggctggaagatggcctggcgcaccagacgttacttggcgtgactggctcagggaaaaccttcaccattgccaatgtcattgctgaccttcagcgcccaaccatggtacttgcgcccaacaaaacgctggcggcccagctgtatggcgaaatgaaagagttcttcccggaaaacgcggtggaatatttcgtttcctactacgactactatcagccggaagcctatgtaccgagttccgacactttcattgagaaagatgcctcggttaacgaacatattgagcagatgcgtttgtccgccaccaaagcgatgctggagcggcgtgatgtggttgtggtggcgtctgtttccgcgatttatggtctgggcgatcctgatttatatctcaagatgatgctccatctcacggtcggtatgattatcgatcagcgcgcgattctgcgccgactggcggagctgcaatacgctcgtaatgatcaagcattccagcgtggtactttccgcgttcgtggcgaggtgatagatatcttcccggcagaatcggatgacattgcacttcgcgtggaactgtttgacgaggaagtggaacgattgtcgttatttgacccgctgaccgggcagattgtttccactattccacgttttaccatctacccgaaaacgcactacgtcacaccgcgcgagcgcatcgtacaggcgatggaggagatcaaagaagagctggccgccagacgcaaagtgctgttggaaaacaacaaactgctggaagagcagcggctgacccagcgtacccagtttgatctggagatgatgaacgagctgggctactgttcggggattgaaaactactcgcgcttcctctccggtcgtggaccgggtgagccaccgccgacgctgtttgattacctgcctgccgatgggctgctggtcgtcgatgaatctcacgtcaccattccacaaattggcggcatgtatcgcggtgaccgggcgcgtaaagagacactggtggagtacggcttccgcctgccatcagcgctggataaccgtccgcttaagtttgaagagttcgaagcattagcgccgcaaaccatctatgtttcggcgacgccgggtaattacgagctggaaaaatccggcggcgatgtggtggatcaggtggtgcgtccaaccggattgcttgacccgattatcgaagtgcggccggtggcgacacaggttgatgatcttctttcggagattcgtcagcgagcggcaattaacgaacgcgtactggtcaccacactgaccaagcggatggcggaagatcttaccgaatatctcgaagaacatggcgagcgcgtgcgttatcttcgctcagatatcgacaccgtcgaacgtatggagattatccgcgacttgcgtctgggtgagttcgacgtgctggtagggatcaacttactgcgcgaaggtctggatatgccggaagtgtcgctggtggcgatcctcgacgctgacaaagaaggcttcctgcgttccgaacgttcgttgatccagaccattggtcgtgcggcacgtaacgttaacggtaaagcgattctctacggcgataagatcaccccatcaatggcgaaagcgattggcgaaaccgaacgtcgccgtgagaaacagcagaagtacaacgaggaacacggaattacgccgcaaggcttgaacaagaaagtggtcgatatcctggcgctggggcagaacattgccaaaaccaaagcgaagggcagaggaaaatcgcgcccgattgttgagccggataatgtgccgatggatatgtcgcctaaagcgttgcagcagaaaatccatgagctggaagggttgatgatgcaacacgcgcagaatctggagttcgaagaagcggcgcaaattcgtgaccagttgcatcagctgcgtgagctgtttatcgcggcatcgtaa

SEQ ID NO: 32 - protein, 610 aa, Escherichia coli
MSDQFDAKAFLKTVTSQPGVYRMYDAGGTVIYVGKAKDLKKRLSSYFRSNLASRKTEALVAQIQQIDVTVTHTETEALLLEHNYIKLYQPRYNVLLRDDKSYPFIFLSGDTHPRLAMHRGAKHAKGEYFGPFPNGYAVRETLALLQKIFPIRQCENSVYRNRSRPCLQYQIGRCLGPCVEGLVSEEEYAQQVEYVRLFLSGKDDQVLTQLISRMETASQNLEFEEAARIRDQIQAVRRVTEKQFVSNTGDDLDVIGVAFDAGMACVHVLFIRQGKVLGSRSYFPKVPGGTELSEVVETFVGQFYLQGSQMRTLPGEILLDFNLSDKTLLADSLSELAGRKINVQTKPRGDRARYLKLARTNAATALTSKLSQQSTVHQRLTALASVLKLPEVKRMECFDISHTMGEQTVASCVVFDANGPLRAEYRRYNITGITPGDDYAAMNQVLRRRYGKAIDDSKIPDVILIDGGKGQLAQAKNVFAELDVSWDKNHPLLLGVAKGADRKAGLETLFFEPEGEGFSLPPDSPALHVIQHIRDESHDHAIGGHRKKRAKVKNTSSLETIEGVGPKRRQMLLKYMGGLQGLRNASVEEIAKVPGISQGLAEKIFWSLKH

SEQ ID NO: 33 - protein, 905 aa, Mus musculus
MGRYWLLPGLLLSLPLVTGWSTSNCLVTEGSRLPLVSRYFTFCRHSKLSFLAACLSVSNLTQTLEVVPRTVEGLCLGGTVSTLLPDAFSAFPGLKVLALSLHLTQLLPGALRGLGQLQSLSFFDSPLRRSLFLPPDAFSDLISLQRLHISGPCLDKKAGIRLPPGLQWLGVTLSCIQDVGELAGMFPDLVQGSSSRVSWTLQKLDLSSNWKLKMASPGSLQGLQVEILDLTRTPLDAVWLKGLGLQKLDVLYAQTATAELAAEAVAHFELQGLIVKESKIGSISQEALASCHSLKTLGLSSTGLTKLPPGFLTAMPRLQRLELSGNQLQSAVLCMNETGDVSGLTTLDLSGNRLRILPPAAFSCLPHLRELLLRYNQLLSLEGYLFQELQQLETLKLDGNPLLHLGKNWLAALPALTTLSLLDTQIRMSPEPGFWGAKNLHTLSLKLPALPAPAVLFLPMYLTSLELHIASGTTEHWTLSPAIFPSLETLTISGGGLKLKLGSQNASGVFPALQKLSLLKNSLDAFCSQGTSNLFLWQLPKLQSLRVWGAGNSSRPCLITGLPSLRELKLASLQSITQPRSVQLEELVGDLPQLQALVLSSTGLKSLSAAAFQRLHSLQVLVLEYEKDLMLQDSLREYSPQMPHYIYILESNLACHCANAWMEPWVKRSTKTYIYIRDNRLCPGQDRLSARGSLPSFLWDHCPQTLELKLFLASSALVFMLIALPLLQEARNSWIPYLQALFRVWLQGLRGKGDKGKRFLFDVFVSHCRQDQGWVIEELLPALEGFLPAGLGLRLCLPERDFEPGKDVVDNVVDSMLSSRTTLCVLSGQALCNPRCRLELRLATSLLLAAPSPPVLLLVFLEPISRHQLPGYHRLARLLRRGDYCLWPEEEERKSGFWTWLRSRL

SEQ ID NO: 34 - protein, 904 aa, Rattus norvegicus
MGRSFLLPGLLLSLPLVTGWTTPKCLVTEGSQLPLVSRYFTLCHYSKLSFLAACFPVSNLTQTLEAVPRNVEGLCLSGSVSTLLPDAFSAFPGLKFLGLNLHLTRLLPGALRGLGQLRNLSFVDQPSGKNSLFLPPDAFGDLISLQRLHFCGPCLNKKAGVRLPSSLQWLSVTISCLQDSGELAGIFPDLVQNSSSRASWTLKKLDLSLNQKLKMATPGSLQGLQVEILDLRKTQLDAGAVKGLGLQKLNVLYAPTATAELAAETAAHFELQGLNVDRSKIGNISQEALASCHSLETLSLSDTGLTKLPPGFLAAMPRLRRLNLAGNQLQSTMLCMNETGDVSGLSTLDLSGNGLRILPPATFSCLPHLRELLLQDNQLLSLEGHPFQDLQQLETLKLDRNPLLNLGKNCLAALPALTTLSLLDTQILHSPDAGFWGARSLHTLHLSLPPLSAPAVLSLPMYLTSLELHVTPGHLHWTLSPNIFPFLETLTINGRGLKLGVQNASEVFPALQHLFLLQNSLDAFCSQDASSIFLWQLPKLQSLKVWGAGSNSRPCLITGLPSLQELKLESLQSITQPRSVQLEELVGDLPQLQALQLSSTGLKSLSAAAFRRLHSLQALVLDSEKDLVLQDSLREYSPQMPRYVYILQSKLACQCANAWMELWVKQSTKTYVHIRDGHLCPGEVRVPARDSLISFLWDHCPQTLELKLFLASSALVLLLIVLPLLQGARNTWIPYLRALFRIWLQGLRGQGNAGKRFLFDVFVSHCRQDQGWVLEELLPALEGFLPAGLGLRLCLPERDFEPGKDVVDNVVDSMVSSRVTLCVLSGPALCNPRCCLELRLATSLLLAAPSPPVLLLVFLEPISRHQLPSYHRLARLLRRGDYCLWPEEEERKGGFWTWLRSRLG

SEQ ID NO: 35 - protein, 1007 aa, Gallus gallus
MELGCGGQHWSRWAVLGYTGMVSLSHPWHSGLCAPALDLSISPRPSTRMCPSHCCPLWLLLLVTVTLMPMVHPYGFRNCIEDVKAPLYFRCIQRFLQSPALAVSDLPPHAIALNLSYNKMRCLQPSAFAHLTQLHTLDLTYNLLETLSPGAFNGLGVLVVLDLSHNKLTTLAEGVFNSLGNLSSLQVQHNPLSTVSPSALLPLVNLRRLSLRGGRLNGLGAVAAAVQGLAQLELLDLCENNLTTLGPGPPLPASLLTLQLCNNSLRELAGGSPDMLWHVKILDLSYNSISQAEVFTQLHLRNISLLHLIGNPLDVFHLLDISDIQPRSLDFSGLVLGAQGLDKVCLRLQGPQALRRLQLQRNGLKVLHCNALQLCPVLRELDLSWNRLQHVGCAGRLLGKKQREKLEVLTVEHNLLKKLPSCLGAQVLPRLYNISFRFNRILTVGPQAFAYAPALQVLWLNINSLVWLDRQALWRLHNLTELRLDNNLLTDLYHNSFIDLHRLRTLNLRNNRVSVLFSGVFQGLAELQTLDLGGNNLRHLTAQSLQGLPKLRRLYLDRNRLLEVSSTVFAPVQATLGVLDLRANNLQYISQWLRKPPPFRNLSSLYDLKLQAQQPYGLKMLPHYFFQGLVRLQQLSLSQNMLRSIPPDVFEDLGQLRSLALADSSNGLHDLPDGIFRNLGNLRFLDLENAGLHSLTLEVFGNLSRLQVLHLARNELKTFNDSVASRLSSLRYLDLRKCPLSCTCDNMWLQGWLNNSRVQVVYPYNYTCGSQHNAYIHSFDTHVCFLDLGLYLFAGTAPAVLLLLVVPVVYHRAYWRLKYHWYLLRCWVNQRWRREEKCYLYDSFVSYNSADESWVLQKLVPELEHGAFRLCLHHRDFQPGRSIIDNIVDAVYNSRKTVCVVSRSYLRSEWCSLEVQLASYRLLDERRDILVLVLLEDVGDAELSAYHRMRRVLLRRTYLRWPLDPAAQPLFWARLKRALRWVEGGEEEEEEGLGGGTGRPREGDKQM

SEQ ID NO: 36 - protein, 12 aa, Artificial Sequence
  Description of Artificial Sequence Synthetic peptide
  MOD_RES (1)..(3): Ile, Leu or Val
  MOD_RES (5): Ile, Leu or Val
XXXAXYDEEKEQ

SEQ ID NO: 37 - protein, 6 aa, Artificial Sequence
  Description of Artificial Sequence Synthetic peptide
DEEKEQ

SEQ ID NO: 38 - protein, 10 aa, Artificial Sequence
  Description of Artificial Sequence Synthetic peptide
  MOD_RES (6): His or Tyr
  MOD_RES (8): Ser or Gly
FAEYLXQXGY

SEQ ID NO: 39 - DNA, 2103 bp, Mus musculus
atgctgaaacaatatattcctcttgcttttgcaccaattcctggctgcccatggctcactcagaggattcccagtagaatcgatgccaaggatggaaagacatcagttctgctctgttctcctcattctgatactattgactctggaatagaaaaagtgcctgcaagtttgactggctactctgagcttcgtgcacttgaccttgggaaaaaccaaatccaaaacatcttggaaaatggagaaatcccaggttataaagccctggaattccttagccttcatgataaccatctgcaaacacttcctaccaggtttctacatactctaccccagcttcagaagctcaacctatctatgaataagcttggaccaatcttggagcttccagaaggactctttagcacaaacttaaaagtgctagatctatcccataatcaactctgtgatgtaccccatggggctttctcccttttgtcacagctccaggagctctggttgagtggcaataacatctccagtttatccaatgaaagcctgcagggactgaggcagctgaggacactagacttaagttggaatcaaattaaagtactcaaaccaggctggctctctcatcttcctgctctgactaccttgaaccttctgggcacctacttagaaaatatcttaggcatacaacttcagggtcccaagatgctaaggcatctacaactgggttcttatccaatgctggacatatatcctccctggcccccaacactccttagcttagaaatacaagcagaatcatgtattcagtttatgattcacagtggacagccattcttattcttagagaaccttaccttagagacttccattctattactaaaaccagacaacatcacaattcattttccctccctgcgtcgcctcaccttgcgtggctacagcttcatcttctcaaccagtcaacttcagagattcttcccacaacagcttcctcttctggagcacttctttatctggtgtgaaaacagctatgcagtagacctctatctatttgggatgcccaggctacgtgtgctagagctggggtaccttaactttttctatgagtcaagtactatgaagctagagatgctattgaaggaggtacctcagttacaggtactggcattgagccacctgaatctcaggaacctctctgtgtccagctttaagagcttgcaggacctcaaactgctgctcttcaactctgaaagggcgctggagatgaacagcaacctccaggagtttattcctcagatgcctcagtacgtttacttctctgatgtcacctttacttgccagtgtgaagcttcctggctggagtcttgggctacacgggccccaaacacatttgtttatgggctggaaaaatccatttgcatagctaatgcctcagactactccaaaactctactattctctttccttgctactaattgtccacacggtactgagttttggggctttctcaccagtttcattctgctgcttctgttgattatccttcctctgattagctgtcctaaatggtcctggcttcatcacctctggacactctttcatacttgttggtggaaattatgtggacatagactcagaggccaattcaactatgatgtctttatatcctattgtgaggaggatcaagcttgggtgctggaagaactggttccagttctggagaaagcccctcctgaaggtgaaggcttgaggttgtgcctgcctgccagggactttgggattggaaatgacaggatggaatccatgattgccagcatgggcaaaagcagagccaccctctgtgtgctcacaggacaggccttagcaagtccctggtgcaatctagagttacgactggccacttaccacttggtagccaggcctgggaccactcatctcctgctgttgtttctggagccccttgataggcagaggctccatagttaccatcgcctatcccgttggctccagaaggaggactattttgatttgtcccaagggaaagtggagtggaactctttctgtgagcaactgaagagacggctcagcaaagctggacaagaaagagattaa

SEQ ID NO: 40 - protein, 700 aa, Mus musculus
MLKQYIPLAFAPIPGCPWLTQRIPSRIDAKDGKTSVLLCSPHSDTIDSGIEKVPASLTGYSELRALDLGKNQIQNILENGEIPGYKALEFLSLHDNHLQTLPTRFLHTLPQLQKLNLSMNKLGPILELPEGLFSTNLKVLDLSHNQLCDVPHGAFSLLSQLQELWLSGNNISSLSNESLQGLRQLRTLDLSWNQIKVLKPGWLSHLPALTTLNLLGTYLENILGIQLQGPKMLRHLQLGSYPMLDIYPPWPPTLLSLEIQAESCIQFMIHSGQPFLFLENLTLETSILLLKPDNITIHFPSLRRLTLRGYSFIFSTSQLQRFFPQQLPLLEHFFIWCENSYAVDLYLFGMPRLRVLELGYLNFFYESSTMKLEMLLKEVPQLQVLALSHLNLRNLSVSSFKSLQDLKLLLFNSERALLMNSNLQEFIPQMPQYVYFSDVTFTCQCEASWLESWATRAPNTFVYGLEKSICIANASDYSKTLLFSFLATNCPHGTEFWGFLTSFILLLLLIILPLISCPKWSWLHHLWTLFHTCWWKLCGHRLRGQFNYDVFISYCEEDQAWVLEELVPVLEKAPPEGEGLRLCLPARDFGIGNDRMESMIASMGKSRATLCVLTGQALASPWCNLELRLATYHLVARPGTTHLLLLFLEPLDRQRLHSYHRLSRWLQKEDYFDLSQGKVEWNSFCEQLKRRLSKAGQERD

SEQ ID NO: 41 - DNA, 2870 bp, Mus musculus
ggaccttgcaggtactctgaggtggatgagagtattggtaacccggaggcataggagtctaaagtcctctcagctctgattcctctggtgtagagatgggcaggtactggctgctgccaggtctcctcctttccctgcctctggtaactgggtggagcacttccaactgcctggtgaccgaaggctcccgactgcccctggtctcccgctatttcacattctgccgccactccaagctatcctttcttgctgcatgcctctccgtgagcaacctgacacagaccttggaagttgtacctcggactgtggaggggctctgcctcggtggtactgtgtctactctgcttccagatgctttctctgcttttcctggtctcaaggtcctggcactgagtctgcaccttacccaacttctgccaggagctctccggggtctgggacagttgcagagcctctctttttttgactctcctcttaggagatctctctttctacctcctgatgccttcagtgacctgatttccctccagagactccatatctctggcccttgcctggataagaaggcaggcatccgcctgcctcccggtctgcaatggctgggtgtcacgctcagttgcattcaggacgtgggagagctggctggtatgttcccagatctggtgcaaggttcctcctccagggtttcgtggaccctgcagaagttggatctgtcatccaaccggaagctgaagatggctagtcctgggtccctccagggtctccaggtggagattctggacctgacaagaacaccactggatgctgtgtggctgaagggcctgggacttcagaaactcgatgtcttgtatgcacagactgccacggccgagctggctgctgaggctgttgcccactttgagctgcagggcttgattgtgaaagaaagcaagataggatctatatctcaggaggctctggcttcctgccacagcctgaagaccttgggtctttcaagcactggcctaaccaagcttccaccagccttcctgactgccatgcctaggcttcagcgactggagctgtccggaaaccaactgcagagcgccgtgctgtgcatgaatgagacgggagatgtgtcaggactcacaactctggatctgtcaggcaacaggttgcgcatcctgcctccagccgccttctcctgcttaccccacttgcgagagctgctgcttcggtacaaccagctgctttccctggagggatacctattccaggagctccaggaactagagaccttgaagctggatggaaaccccctgcttcacctgggtaagaactggttggcggctctgcctgcattgaccacccttagcttgctagatacccaaatacggatgagcccagagcctggcttctggggagcaaagaatctgcataccttgagcctgaagcttcccgctctccctgctccggcagtattgttcctgcccatgtatctgaccagcttagagcttcatatagcctcaggcacgacggagcactggacgctgtccccagagatctttccttccttggagaccttgactataagcggcgggggactgaagctgaagctggggtcccagaatgcttctggggtcttccctgctctccagaagctctccctgcttaagaacagcttggatgccttctgctcccagggtacctccaaccttttcctctggcagctccccaaacttcagtccttgagggtatggggtgctggaaacagctccagaccctgccttatcactgggctgcccagcctacgggagctgaagctggcgtcgcttcagtccataacccagccccgttcggtgcagctggaggagctggtgggtgaccttccacagctccaggccttagtgctatccagcacaggcctcaagtcactgtcggccgctgctttccagcgcctgcacagtctccaggtcttagtgctagaatacgagaaggacttgatgctgcaggacagtctgagggagtacagccctcagatgccccactatatatacattctggagtcaaacctggcctgccactgtgccaatgcgtggatggagccatgggttaagcggtccactaaaacgtacatatacataagagacaatcgcttatgtccaggacaagacaggctctctgctaggggttcccttccctcctttctctgggaccactgcccccagacgttggagctgaaactctttttggctagttctgccttggtgttcatgctaattgccttgcctctcctccaagaagccaggaactcttggatcccctacctgcaggccttgttcagggtttggctccagggtctgaggggtaagggagacaaggggaagaggttccttttcgatgtattcgtgtcccactgcaggcaagaccagggctgggtgatagaggaacttctgcctgctctggagggcttccttccagctggcctgggcctgcgcctctgtctccccgagcgtgagtttgagcctggtaaggatgtagttgataatgtggtagatagcatgttgagcagccgtaccacactctgcgtgttgagtgggcaggccctgtgtaacccccgatgccgcctggagctccgcttggccacctctctcctcctggctgccccgtcccccccagtgttgctgctagtcttcttggaacccatttgtcggcaccagcttccgggttaccacagactggctcggctgcttcgaagaggagactactgtctgtggcccgaggaagaggagagaaagagtgggttctggacttggctgaggagcaggctagggtagccatagccagcactggtgtggggtggtgcatgtgaattttggggtggggttggg

SEQ ID NO: 42 - protein, 906 aa, Mus musculus
MGRYWLLPGLLLSLPLVTGWSTSNCLVTEGSRLPLVSRYFTFCRHSKLSFLAACLSVSNLTQTLEVVPRTVEGLCLGGTVSTLLPDAFSAFPGLKVLALSLHLTQLLPGALRGLGQLQSLSFFDSPLRRSLFLPPDAFSDLISLQRLHISGPCLDKKAGIRLPPGLQWLGVTLSCIQDVGELAGMFPDLVQGSSSRVSWTLQKLDLSSNRKLKMASPGSLQGLQVEILDLTRTPLDAVWLKGLGLQKLDVLYAQTATAELAAEAVAHFELQGLIVKESKIGSISQEALASCHSLKTLGLSSTGLTKLPPGFLTAMPRLQRLELSGNQLQSAVLCMNETGDVSGLTTLDLSGNRLRILPPAAFSCLPHLRELLLRYNQLLSLEGYLFQELQQLETLKLDGNPLLHLGKNWLAALPALTTLSLLDTQIRMSPEPGFWGAKNLHTLSLKLPALPAPAVLFLPMYLTSLELHIASGTTEHWTLSPEIFPSLETLTISGGGLKLKLGSQNASGVPPALQKLSLLKNSLDAFCSQGTSNLFLWQLPKLQSLRVWGAGNSSRPCLITGLPSLRELKLASLQSITQPRSVQLEELVGDLPQLQALVLSSTGLKSLSAAAFQRLHSLQVLVLEYEKDLMLQDSLREYSPQMPHYIYILESNLACHCANAWMEPWVKRSTKTYIYIRDNRLCPGQDRLSARGSLPSFLWDHCPQTLELKLFLASSALVFMLIALPLLQBARNSWIFYLQALFRVWLQGLRGKGDKGKRFLFDVFVSHCRQDQGWVIEELLPALEGFLPAGLGLRLCLPEREFEPGKDVVDNVVDSMLSSRTTLCVLSGQALCNPRCRLELRLATSLLLAAPSPPVLLLVFLEPISRHQLPGYHRLARLLRRGDYCLWPEEEERKSQFWTWLRSRLG

SEQ ID NO: 43 - DNA, 4290 bp, Mus musculus
ttgaaatctcacagcccggttggttgcagtgacccacttcgttgaacatattcttcctaatcctagtactttcaatttgctctattccctggtgtctatgcatttaaatcgactatggggccattcttccttgaaccaccacagaagacattagctctctgggatccttgttaattttttctcctcttacatagcacctacgcttggaacatatgccagacacatctgtgagacaccccttgccgctgcagctcatggatggatgctgagttcccccacgcaccacacttcagcaggtgggtgtatttctgcttcacattatactcccacacggccatgcatgtcaggcatggagcaggctcataacccacttaattaaggtgatcatatcagatcctttatcaagatgcatatagtgctcagtgcctgtactatgatctcggatctttgggagatgggctagatagagtctgggacagaatacagcagagaaaccgatatgtttattgtccgatcatcagctaagcttctgggagctaggaatggggctccttggatgaacagaagtaaaaatgcctcgtctttatgactttcaacttccctcagcaggtctggaatgggtgaacaaacactgcctgcgtgggtgataaatagcctctttttgctgcttgtttgctgcttttatggttctgggagggaacctagaacctagcacatgctagacaagtcctctagcactgagctatctccccagcttggatgaaatatctgtaaagtactggtgcccgtgtgtaaaatatgcaccattaagtgttcaagaagaaaagactgggcatttctgttccaccaagacaagaagaatctgccagcagaatgtttgcgcagtcatttgagcaaaggggtccaagggacagtaccctccagtgctggggacccatgtgccgagcctcaggctgtgatgtggtgttgtttttaattctctcttttcccataggatcatggcatgtcaacttgacttgctcataggtgtgatcttcatggccagccccgtgttggtaatatctccctgttcttcagacggcaggatagcctttttccgaggctgtaacctcacccagattccctggatcctcaatactaccactgagaggctcctgctcagcttcaactatatcagtatggtggttgccacatcatttccactcctggagcggctccagttgctggagctggggacccagtatgctaacttgaccattggtccaggggctttcagaaacctgcccaatcttaggatcttggacttgggccaaagccagctcgaagtcttgaatcgagatgcctttcaaggtctgccccatctcttggaacttcggctgttttcctgtggactctccagtgctgtgttaagtgacggttacttcagaaatctatattcattagctcgcttagacctatctggcaaccagattcacagcctccgcctccattcttcattccgggaactgaattccttaagcgacgtaaattttgctttcaaccaaatattcactatatgtgaagatgaactcgagcctctgcagggcaaaacactgtctttctttggcctcaaattaactaagctgttcagcagagtctctgtgggctgggagacatgcaggaaccccttcagaggcgtgaggctagaaactctagatctttctgaaaatggctggacggtggacatcacaaggaacttcagcaacatcatccagggaagccagatttcctctttgattcttaaacaccacatcatgggtcctggctttggcttccagaacatcagagatcctgaccagagcacatttgccagcctggccagaagttcggtgctgcaactggacctttcgcacggctttatcttctccttgaatcctcgactgtttgggacactgaaggatttgaagatgctgaaccttgccttcaacaagataaacaagattggagagaatgccttttatgggcttgacagcctccaggttctcaatctatcctataatcttttgggggaactctataattccaacttctatgggcttcctagagtagcctacgttgaccttcaaaggaaccacattgggatcattcaagaccaaacattcagattattaaaaacgttacaaaccttagatctccgtgacaatgctcttaaggccattggttttattccaagcatatagatggtcctcctgggaggcaataagctggtccatttgccacacatccacacatccactttactgccaacttcctagagttatctgaaaacaggctagaaaacctgtccgacctctacttcctcctgcgagtcccccagctccagtttctcatcttgaatcagaatcgcctttcgtcatgcaaggcagcccacactccctcggagaacccaagcttagaacagcttttccttacagagaatatgctgcagctggcctgggagaccggcctctgttgggatgtttttcaaggccttcccgcctccagattctttacctgagtaataactaccttaatttccttccacctgggatatttaacgacctggttgcattacggatgcttagtcttagtgctaacaagctgaccgtgctctctccgggcagtttacctgctaatttagagattctcgacatatctagaaatcagcttttgtgtcctgaccctgctttgttttcttcgcttcgtgttttggacataactcataacgagttcgtctgcaactgtgaacttagcacttttatctcctggctcaaccaaaccaacgtcaccctgttcggctctcctgcagacgtgtattgcatgtaccctaactcactgctagggggctccctctacaacatatccaccgaagactgcgatgaagaggaagccatgcggtccctaaagttttcccttttcatcctgtgcacggtcactttgactctattcctcgtcatcacccttgtagtcataaagttccggggaatctgtttcctgtgctataagaccatccagaagctggtgttcaaggacaaggtctggagtttggaacctggtgcatatagatatgatgcctacttctgcttcagcagcaaagactttgaatgggcacagaatgctttgctcaaacacctggatgctcactacagttcccgaaacaggctcaggctatgctttgaagaaagagacttcattccgggggaaaaccatatctccaacatccaggcggctgtctggggcagcaggaagacggtgtgtctagtgagcagacacttcctgaaggatggttggtgcctggaggccttcaggtatgcccagagccggagtctgtctgacctcaagagcattctcatcgtggtggtggtgggatcgctgtcccagtatcagctgatgagacatgagaccatcagagggttctgcaaaagcaacagtacttgaggtggcctgaagacctccaggatgttggctggtttctcgataaactctccggatgcattctaaaggaagaaaaaggaaagaaaagaagcagttccatccagttgcgaaccatagcaaccatttcctagcaggagcgcctcctagcagaagtgcaagcatcgtagataactctccacgctttatccgcacagccgctgggggtccttccctggagtcatttttctgacaatgaaaacaacaccaatctcttgatttttcatgtcaacagggagctttgtcttcactgttttccaaatggaagtaagaggtccagaaagctgcctctaagggctctcacctgccattgatgtcctttcaggcccaatgacatggtttccctccatcctattgcgtactgtctgctacccaggtggcaagagcaccttgggagaagttacaggcagcttcatgctttctgtgctgttcagttcaaaagcaggtgccttgagaatcctgaattcaagcactctgtagaacatggacagacaagatgggtccttctctggccataggcatgagggccagttgctgaggactgctctcactacacctaagtgcacaagtgataagaagttggacagatagacagatagcagcagtcccattgctgtagccagaatgcacttatttcctgttctgaccctgcaggcccagcttttggggaccacagccatgttctgcacgggacctctcaacctggcattcatgccctttcacgacttagcaccggcctgcccttctttcttccccacaactatacaagagctgttgcaaccactgaaaaaaaaaaaaaaa

SEQ ID NO: 44 - protein, 857 aa, Mus musculus
MACQLDLLIGVIFMASPVLVISPCSSDGRIAFFRGCNLTQIPWILNTTTERLLLSFNYISMVVATSFPLLERLQLLELGTQYANLTIGPGAFRNLPNLRILDLGQSQIEVLNRDAFQGLPHLLELRLFSCGLSSAVLSDGYFRNLYSLARLDLSGNQIHSLRLHSSFRELNSLSDVNFAFNQIFTICEDELEPLQGKTLSFFGLKLTKLFSRVSVGWETCRNPFRGVRLETDLSENGWTVDITRNFSNIIQGSQISSLILKHHIMGPGFGFQNIRDPDQSTFASLARSSVLQLDLSHGFIFSLNPRLFGTLKDLKMLNLAFNKINKIGENAFYGLDSLQVLNLSYNLLGELYNSNFYGLPRVAYVDLQRNHIGIIQDQTFRLLKTLQTLDLRDNALKAIGFIPSIQMVLLGGNKLVHLPHIHFTANFLELSENRLENLSDYFLLRVPQLQFLILNQNRLSSCKAAHTPSENPSLEQLFLTENMLQLAWETGLCWDVFQGLSRLQILYLSNNYLNFLPPGIFNDLVALRMLSLSANKLTVLSPGSLPANLEILDISRNQLLCPDPALFSSLRVLDITHNEFVCNCELSTFISWLNQTNVTLFGSPADVYCMYPNSLLGGSLYNISTEDCDEEEAMRSLKFSLFILCTVTLTLFLVITLVVIKFRGICFLCYKTIQKLVFKDKVWSLEPGAYRYDAYFCFSSKDFEWAQNALLKHLDAHYSSRNRLRLCFEERDFIPGENHISNIQAAVWGSRKTVCLVSRHFLKDGWCLEAFRYAQSRSLSDLKSILIVVVVGSLSQYQLMRHETIRGFLQKQQYLRWPEDLQDVGWFLDKLSGCILKEEKGKKRSSSIQLRTIATIS

SEQ ID NO: 45 - DNA, 3431 bp, Homo sapiens
ggcttatagggctcgagcggccgcccgggcaggtatagaattcagcggccgctgaattctagggttttcaggagcccgagcgagggcgccgcttttgcgtccgggaggagccaaccgtggcgcaggcggcgcggggaggcgtcccagagtctcactctgccgcccaggctggactgcagtgacacaatctcggctgactgcaaccactgcctccagggttcaagcgattctcttgcctcagcctcccaagtagctgggattacagattgatgttcatgttcctggcactactacaagattcatactcctgatgctactgacaacgtggcttctccacagtcaccaaaccagggatgctatactggacttccctactctcatctgctccagccccctgaccttatagttgcccagctttcctggcaattgactttgcccatcaatacacaggatttagcatccagggaagatgtcggagcctcagatgttaattttctaattgagaatgttggcgctgtccgaacctggagacagaaaaacaaaaagtcctttctcctgattcaccaaaaaataaaatactgactaccatcactgtgatgagattcctatagtctcaggaactgaagtctttaaacaaccagggaccctctgcccctagaataagaacatactagaagtcccttctgctaggacaacgaggatcatgggagaccacctggaccttctcctaggagtggtgctcatggccggtcctgtgtttggaattccttcctgctcctttgatggccgaatagccttttatcgtttctgcaacctcacccaggtcccccaggtcctcaacaccactgagaggctcctgctgagcttcaactatatcaggacagtcactgcttcatccttcccctttctggaacagctgcagctgctggagctcgggagccagtatacccccttgactattgacaaggaggccttcagaaacctgcccaaccttagaatcttggacctgggaagtagtaagatatacttcttgcatccagatgcttttcagggactgttccatctgtttgaacttagactgtatttctgtggtctctctgatgctgtattgaaagatggttatttcagaaatttaaaggctttaactcgcttggatctatccaaaaatcagattcgtagcctttaccttcatccttcatttgggaagttgaattccttaaagtccatagatttttcctccaaccaaatattccttgtatgtgaacatgagctcgagcccctacaagggaaaacgctctccttttttagcctcgcagctaatagcttgtatagcagagtctcagtggactggggaaaatgtatgaacccattcagaaacatggtgctggagatagtagatgtttctggaaatggctggacagtggacatcacaggaaactttagcaatgccatcagcaaaagccaggccttctctttgattcttgcccaccacatcatgggtgccgggtttggcttccataacatcaaagatcctgaccagaacacatttgctggcctggccagaagttcagtgagacacctggacctttcacatgggtttgtcttctccctgaactcacgagtctttgagacactcaaggatttgaaggttctgaaccttgcctacaacaagataaataagattgcagatgaagcattttacggacttgacaacctccaagttctcaatttgtcatataaccttctgggggaactttgcagttcgaatttctatggactacctaaggtagcctacattgatttgcaaaagaatcacattgcaataattcaagaccaaacattcaaattcctggaaaaattacagaccttggatctccgagacaatgctcttacaaccattcattttattccaagcatacccgatatcttcttgagtggcaataaactagtgactttgccaaagatcaaccttacagcgaacctcatccacttatcagaaaacaggctagaaaatctagatattctctactttctcctacgggtacctcatctccagattctcattttaaatcaaaatcgcttctcctcctgtagtggagatcaaaccccttcagagaatcccagcttagaacagcttttccttggagaaaatatgttgcaacttgcctgggaaactgagctctgttgggatgtttttgagggactttctcatcttcaagttctgtatttgaatcataactatcttaattcccttccaccaggagtatttagccatctgactgcattaaggggactaagcctcaactccaacaggctgacagttctttctcacaatgatttacctgctaatttagagatcctggacatatccaggaaccagctcctagctcctaatcctgatgtatttgtatcacttagtgtcttggatataactcataacaagttcatttgtgaatgtgaacttagcacttttatcaattggcttaatcacaccaatgtcactatagctgggcctcctgcagacatatattgtgtgtaccctgactcgctctctggggtttccctcttctctctttccacggaaggttgtgatgaagaggaagtcttaaagtccctaaagttctcccttttcattgtatgcactgtcactctgactctgttcctcatgaccatcctcacagtcacaaagttccggggcttctgttttatctgttataagacagcccagagactggtgttcaaggaccatccccagggcacagaacctgatatgtacaaatatgatgcctatttgtgcttcagcagcaaagacttcacatgggtgcagaatgctttgctcaaacacctggacactcaatacagtgaccaaaacagattcaacctgtgctttgaagaaagagactttgtcccaggagaaaaccgcattgccaatatccaggatgccatctggaacagtagaaagatcgtttgtcttgtgagcagacacttccttagagatggctggtgccttgaagccttcagttatgcccagggcaggtgcttatctgaccttaacagtgctctcatcatggtggtggttgggtccttgtcccagtaccagttgatgaaacatcaatccatcagaggctttgtacagaaacagcagtatttgaggtggcctgaggatctccaggatgttggctggtttcttcataaactctctcaacagatactaaagaaagaaaaagaaaagaagaaagacaataacattccgttgcaaactgtagcaaccatctcctaatcaaaggagcaatttccaacttatctcaagccacaaataactcttcactttgtatttgcaccaagttatcattttggggtcctctctggaggttttttttttctttttgctactatgaaaacaacataaatctctcaattttcgtatcaaa

SEQ ID NO: 46 - protein, 858 aa, Homo sapiens
MGDHLDLLLGVVLMAGPVFGIPSCSFDGRIAFYRFCNLTQVPQVLNTTERLLLSFNYIRTVTASSFPFLEQLQLLELGSQYTPLTIDKEAFRNLPNLRILDLGSSKIYFLHPDAFQGLFHLFELRLYFCGLSDAVLKDGYFRNLKALTRLDLSKNQIRSLYLHPSFGKLNSLKSIDFSSNQIFLVCEHELEPLQGKTLSFFSLAANSLYSRVSVDWGKCMNPFRNMVLEIVDVSGNGWTVDITGNFSNAISKSQAFSLILAHHIMGAGFGFHNIKDPDQNTFAGLARSSVRHLDLSHGFVFSLNSRVFETLKDLKVLNLAYNKINKLADEAFYGLDNLQVLNLSYNLLGELCSSNFYGLPKVAYIDLQKNHIAIIQDQTFKFLEKLQTLDLRDNALTTIHFIPSIPDIFLSGNKLVTLPKINLTANLIHLSENRLENLDILYFLLRVPHLQILILNQNRFSSCSGDQTPSENPSLEQLFLGENMLQLAWETELCWDVFEGLSHLQVLYLNHNYLNSLPPGVFSHLTALRGLSLNSNRLTVLSHNDLPANLEILDISRNQLLAPNPDVFVSLSVLDITHNKFICECELSTFINWLNHTNVTIAGPPADIYCVYPDSLSGVSLFSLSTEGCDEEEVLKSLKFSLFIVCTVTLTLFLMTILTVTKFRGFCFICYKTAQRLVFKDHPQGTEPDMYKYDAYLCFSSKDFTWVQNALLKHLDTQYSDQNRFNLCFEERDFVPGENRIANIQDAIWNSRKIVCLVSRHFLRDGWCLEAFSYAQGRCLSDLNSALIMVVVGSLSQYQLMKNQSIRGFVQKQQYLRWPEDLQDVGWFLHKLSQQILKKEKEKKKDNNIPLQTVATIS

SEQ ID NO: 47 - protein, 35 aa, Artificial Sequence
  Description of Artificial Sequence Synthetic peptide
MVLAPNKTLAAQLYGEMKEFFPENAVEYFVSYYDY

SEQ ID NO: 48 - protein, 49 aa, Artificial Sequence
  Description of Artificial Sequence Synthetic peptide
KAIDDSKIPDVILIDGGKGQLAQAKNVFAELDVSWDKNHPLLLGVAKGA

SEQ ID NO: 49 - protein, 170 aa, Eimeria acervulina
MGEEADTQAWDTSVKEWLVDTGKVYAGGIASIADGCRLFGAAIDNGEDAWSQLVKTGYQIEVLQEDGSSTQEDCDEAETLRQAIVDGRAPNGVYTRGVKYKLAEVKRDFTYNDQNYDVAILGKNKGGGFLIKTPNDNVVIALYDEEKEQNKADALTTALAFAEYLYQGGF

SEQ ID NO: 50 - protein, 170 aa, Eimeria tenella
MGEEADTQAWDTSVKEWLVDTGKVYAGGIASIADGCRLFGAAIDNGEDAWSQLVKTGYQIEVLQEDGSSTQEDCDEAETLRQAIVDGRAPNGVYIGGVKYKLAEVKRDFTYNDQNYDVAILGKNKGGGFLIKTPNDNVVIALYDEEKEQNKADALTTALAFAEYLYQGGF

SEQ ID NO: 51 - protein, 169 aa, Eimeria tenella
MGEADTQAWDTSVREWLVDTGRVFAGGVASIADGCRLFGASVEGEGNAWEELVKTNYQIEVPQEDGTSISVDCDEAETLRQAVVDGRAPNGVYIGGTKYKLAEVKRDFTFNDQNYDVAILGKNKGGGFLIKTPNENVVIALYDEEKEQNKADALTTALNFAEYLYQGGF

SEQ ID NO: 52 - protein, 169 aa, Eimeria tenella; identical to SEQ ID NO: 6

SEQ ID NO: 53 - protein, 169 aa, Eimeria tenella
MGEADTQAWDTSVREWLVDTGRVFAGGVASIADGCRLFGAAVEGEGNAWEELVKTNYQIEVPQEDGTSISVDCDEAETLRQAVVDGRAPNGVYIGGTKYKLAEVKRDFTFNDQNYDVAILGKNKGGGFLIKTPNENVVIALYDEEKEQNKADALTTALNFAEYLHQSGF

SEQ ID NO: 54 - protein, 158 aa, Eimeria sp.
  MOD_RES (1): Variable amino acid
  MOD_RES (22): Variable amino acid
  MOD_RES (60)..(69): Variable amino acid
  MOD_RES (74)..(76): Variable amino acid
  MOD_RES (88)..(113): Variable amino acid
  MOD_RES (156)..(158): Variable amino acid
XEWLVDTGKVFAGGVASIADGXRMFGASTDSGGDPNAELVQYNAGYQIESVQEDNGTVQXXXXXXXXXXQAIVXXXAPDGVYIGGVKXXXXXXXXXXXXXXXXXXXXXXXXXXGGGFLIKTPNENIAIALYDEEKEQNKADALTTALNFADFLYQXXX

SEQ ID NO: 55 - protein, 163 aa, Toxoplasma gondii; identical to SEQ ID NO: 3

SEQ ID NO: 56 - protein, 163 aa, Neospora caninum
MSDWDPVVKEWLVDTGYCCAGGIANAEDGVVFAAAADDDDGWSKLYKEDHEEDTIGEDGNVNGKVTVNEASTIKAAVDDGSAPNGVWIGGQKYKVVRPEKGFEYNDCTFDITHCARSKGGAHLIKTPNGSIVIALYDEEKEQDKGNSRTSALAFAEYLHQSGY

SEQ ID NO: 57 - protein, 170 aa, Sarcocystis neurona; identical to SEQ ID NO: 2

SEQ ID NO: 58 - protein, 174 aa, Plasmodium berghei
MEEYSWENFLNDKLLATNQVSAAGLASEEDGVVYECVATPDENNPDFDKWSLFYKEDYDIEIEDENGSKTTKTITEGQSILTMFNEGYASDGIWLGGTKYQFINMDKGLEYEGHSFDVATCAKSKGGMHIIKVGGGHILIVLYDEEKEQDRGNSKNAALAFSKELIESTDTGAA

SEQ ID NO: 59 - protein, 171 aa, Plasmodium falciparum; identical to SEQ ID NO: 4

SEQ ID NO: 60 - protein, 170 aa, Plasmodium yoelii
MEESWENFLNDKLLATNQVSAAGASEEDGVVYECVATPDENNPDFDKWSLFYKEDYIEIEDENGNKTTKTITEGQTILTMFNEGYAPDGIWLGGTKYQFINMEKGLEYEGYSFDVATCAKLKGGMHIIKVGGGILIVLYDEEKEQDRGNSKNAALAFSKELARSTDAGAA

SEQ ID NO: 61 - protein, 164 aa, Babesia bovis
MADWVPTIKQLALADNACYGCGIANAEDGELFSAADIDHDDLCWDSVYRDPYEFEATDENGQPIKHQITEKATIMEVFEKRRSSIGIFIGGNKYTFANYDDDCPVGDYTFKCVSAAKNKGGAHLVKTPGGYIVICVFDENRGQNKTSSRMAAFALAEYMAANGY

SEQ ID NO: 62 - protein, 164 aa, Theleria parva
MAEWVPVLKETALSNNSCYGAGIANGEDGELFVASDLDHEGLHWDSVYKDPYVYETFDDAGNPLKINVDEKFTIREVFEKKMSSEGIFLGGEKYTFASYDPDMESGSFKFECVCGAKNKGGCHLIKTPGNYIVVVVYDETRGQDKTVSRMAAFNLAEYLATNGY

SEQ ID NO: 63 - protein, 162 aa, Cryptosporidium hominis
MSEWDDMVKEWLIDTGSVCAGGLCSIDGAFYAASADQGDAWKTLVREDHEENVIQSDGVSEAAELINDQTTLCQAISEGKAPNGVWVGGNKYKIIRVEKDFQQNDAIVNVTFCNKPQGGCFLVDTQNGTVVVAVYDESKDQSSGNCKKVALQLAEYLVSQGY

SEQ ID NO: 64 - protein, 162 aa, Cryptosporidium parvum
MSEWDDMVKEWLIDTGSVCAGGLCSIDGAFYAASADQGDAWKTLVREDHEENVIQSDGVSEAAELINDQTTLCQAISEGKAPNGVWVGGNKYKIIRVEKDFQQNDATVHVTFCNRPQGGCFLVDTQNGTVVVAVYDESKDQSSGNCKKVALQLAEYLVSQGY

SEQ ID NO: 65 - protein, 164 aa, Theleria annulata
MAEWVPVLKETALANNSCYGAGIANGEDGELFVASDVDHEGLHWDSVYKEGYEYETMDDAGNPLKVKVDEKFTIREVFEKKMSSEGIFLGGEKYTFASYDPDMESGSFKFECVCGAKNKGGCHLIKTPGNYIVVVVYDETRGQDKTVSRMAAFNLAEYLASNGY

SEQ ID NO: 66 - protein, 148 aa, Alexandrium tamarense
RLLLRWSLAQLEDGALYGAAPVKDEVGWSYVFKDDHEEDITQDDDSVKKMSINEPACIQSVINTGKAPDGGLWLGGLKYNVVQYDPNFERSDATLVVCSAARPKKGVHLVRTDTQVVAGFYDEEKNQTAGNAKKATLAFAEYLKGLGY

SEQ ID NO: 67 - protein, 172 aa, Perkinsus marinus
  MOD_RES (85): Variable amino acid
MSWNDYISHYLLSEGKCYAGALGNSSDYKIFAAQGDHAGEGWAATFSEDFITEVLVDEVTERTEQVMINEATTLREAMETGSWLXIWPLLGQKYRIVKYETDFDCAGQECVINTGNRIASLVGRRVLAGTMLVMGMYDEELGQTGGNCKSACAAFAEYQARAAFEVKMVSCF

SEQ ID NO: 68 - protein, 169 aa, Lingulodinium polyedrum
MAEEEGSWDQTIEEWLISEGHCYAAALANAADGGMYAAAPQANEEGWGFVFKDAHEETVTQDDMTEKKNTIDETVCLFTAVSEKTKKTATGGFWLGGKKYTITQYQTEAIGDKDCDTIFANRPKEGVHIVKTPGDQVILGFFSEEKGKLQGNAKKCVVAFAEHLVGLGY

SEQ ID NO: 69 - protein, 149 aa, Trypanosoma cruzi
MSWQAYIDDSLIGSGHMHSAAIVGLSDGSYWGYGGNYIPQPDEVAHILKCLGNFSLVQSSGVTIYGVKFGLQSGKEGEMKYIFFKKGAAGGCIYTSKQTAIIAVYGNPGTSSSLQQDLEKKEGAEIAVNPADCNSTVKRIAEYLISLDY

SEQ ID NO: 70 - protein, 153 aa, Tetrahymena pyriformis
MSGWDQYVQYLTANQQVEYGLILGKTDGTIWASNVGLTTLYNNYQIDVEGQKANVNETANLLAAMNNNGVPTDPLCGIRIMNQKYYTVKYDADSQVWYLKKDHGGACIAITNQALVIGTFDITKKQQNGVAQNPGQVNKVVESLAATLKQAGY

SEQ ID NO: 71 - protein, 133 aa, Betula pendula
MSWQTYVDEHLMCDIDGQGEELAASAIVGHDGSVWAQSSSFPQFKPQEITGIMKDFEEPGHLAPTGLHLGGIKYMVIQGEAGAVIKGKKGSGGITIKKTGQALVFGIYEEPVTPGQCNMVVERLGDYLIDQGL

SEQ ID NO: 72 - protein, 166 aa, Eimeria acervulina
MGEETQAWDTSVKEWLVDTGKVYAGGASIADGCRLFGAAIDNGEDAWSQLVKTGYQIEVLQEDGSSTQEDCDEAETLRQAIVDGRAPNGVYIGGIKYKLAEVKRDFTYNDQNYDVAILGKNKGGFLIKTPNDNVVIALYDEEKEQNKADALTTALAFAEYLYQGGF

SEQ ID NO: 73 - protein, 169 aa, Eimeria tenella; identical to SEQ ID NO: 51

SEQ ID NO: 74 - protein, 168 aa, Eimeria tenella
MGEADTQAWDTSVREWLVDTGRVFAGGVASIADGCRLFGAAVEGEGNAWEELVKTNYQIEVPQEDGTSISVDCDEAETLRQAVVDGRAPNGVYIGGTKYKLAEVKRDFTFNDQNYDVAILGKNKGGGFLIKTPNENVVIALYDEEKEQNKADALTTALNFAEYLYQGG

SEQ ID NO: 75 - protein, 118 aa, Eimeria sp.
  MOD_RES (1): Variable amino acid
  MOD_RES (22): Variable amino acid
  MOD_RES (38): Variable amino acid
  MOD_RES (58): Variable amino acid
  MOD_RES (63): Variable amino acid
  MOD_RES (75): Variable amino acid
  MOD_RES (118): Variable amino acid
XEWLVDTGKVFAGGVASIADGXRMFGASTDSGDPNAEXLVKAGYQIESVQEDNGTVQXQAIVXAPDGVYIGGVKXGGGFLIKTPNENIAIALYDEEKEQNKADALTTALNFADFLYQX

SEQ ID NO: 76 - protein, 161 aa, Neospora caninum
MSDWDPVVKEWLVDTGYCCAGGIANAEDGVVFAAAADDDDGWSKLYKEDHEEDTIGEDGNVNGKVTVNESTIKAAVDDVSAPNGVWIGGQKYKVVRPEKGFEYNDCTFDITMCARSKGGAHLIKTPNGSIVILYDEEKEQDKGNSRTSALAFAEYLHQSGY

SEQ ID NO: 77 - protein, 162 aa, Toxoplasma gondii
MSDWDPVVKEWLVDTGYCCAGGIANAEDGVVFAAAADDDDGWSKLYKDDHEEDTIGEDGNACGKVSINEASTIKAAVDDGAPNGVWIGGQKYKVVRPEKGFEYNDCTFDITMCARSKGGAHLIKTPNGSIVIALYDEEKEQDKGNSRTSALAFAEYLHQSGY

SEQ ID NO: 78 - protein, 170 aa, Sarcocystis neurona
MAEEQAGTEEWDTLCQDWLPGTGYCSAGGLCSAEDGVIYAAASNSHKGWAVLYRDDHEQDELGEDGNPIGKVTINEGSTIKKAMEEGSAPNGVWIGGVKYKVVRPEKNVEYNGIMYDTVMCARPKSGAHLIKTPKGTIIVAVYDEEKEQSAGNSRTCALAFAHHLNFLGC

SEQ ID NO: 79 - protein, 162 aa, Cryptosporidium hominis; identical to SEQ ID NO: 63

SEQ ID NO: 80 - protein, 162 aa, Cryptosporidium parvum; identical to SEQ ID NO: 64

SEQ ID NO: 81 - protein, 174 aa, Plasmodium berghei; identical to SEQ ID NO: 58

SEQ ID NO: 82 - protein, 170 aa, Plasmodium falciparum
MAEESWDSYLNDRLLATNQVSGAGLASEEDGVVYACVAQGEESDPNFDKWSLFYKEDYDIEVEDENGTKTTKTINEGQTILVVFNEGYAPDGVWLGGTKYQFINIERDLEFEGYNFDVATCAKLKGGLHLVKVPGGNILVVLYDEEKEQDRGNSKIAALTFAKELAESSQ

SEQ ID NO: 83 - protein, 171 aa, Plasmodium yoelii
MEESWENFLNDKLLATNQVSAAGASEEDGVVYECVATPDENNPDFDKWSLFYKEDYDIEIEDENGNKTTKTITEGQTILTMFNEGYAPDGIWLGGTKYQFINMEKGLEYEGYSFDVATCAKLKGGMHIIKVGGGILIVLYDEEKEQDRGNSKNAALAFSKELARSTDAGAA

SEQ ID NO: 84 - protein, 164 aa, Babesia bovis; identical to SEQ ID NO: 61

SEQ ID NO: 85 - protein, 164 aa, Theleria annulata
MAEWVPVLKETALANNSCYGAGIANGEDGELFVASDVDHEGLHWDSVYKEGYEYETMDDAGNPLKVKVDEKFTIREVFEKKMSSEGIFLGGEKYTFASYDPDMESGSFKFECVCGAKNKGGCHLIKTPGNYIVVVVYDETRGQDKTVSRMAAFNLAAYLASNGY

SEQ ID NO: 86 - protein, 164 aa, Theleria parva; identical to SEQ ID NO: 62

SEQ ID NO: 87 - protein, 148 aa, Alexandrium tamarense; identical to SEQ ID NO: 66

SEQ ID NO: 88 - protein, 169 aa, Lingulodinium polyedrum
MAEEEGSWDQTIEEWLISEGHCYAAALANAADGGMYAAAPQANEEGWGFVFKDAHEETVTQDDMTEKKMTIDETVCLFTAVSEKTKKTATGGFWLGGKKYTITQYQTEAIGDKDCDTIFANRPKEGVHIVKTPGDQVILGFFSEEKGKLQGNAKKCVVAFAEHLVGLGY

SEQ ID NO: 89 - protein, 126 aa, Dictyostelium discoideum
MTWQAYIDTNLIGSGFISAQILSSADGSSWANSNGFSVSATEAQHILSCFKDSNKASAMGITINNVKNFVLKADDKSIYAKKDAGGVVLVKTNQTILVAVYNSNLQPGAAANACEALGDYLREQGF

SEQ ID NO: 90 - protein, 150 aa, Leishmania major
MSWQAYVDDSLIGSGNMHSAAIIGAADGSYWAYGGSYVPQPEEVQHIQKCLSDFSFVQSSGVNIYGVKFFGLQCGTDGDCKYIFFKKGAAGGCIYTTKQAFIVAVYGNPGDTSSLQQDLEKNTAHAVTVNPADCNTTVKRIADYLIKLGY

SEQ ID NO: 91 - protein, 150 aa, Trypanosoma cruzi
MSWQAYIDDSLIGSGHMHSAAIVGLSDGSYWGYGGNYIPQPDEVAHILKCLGNFSLVQSSGVTIYGVKFFGLQSGEEGEMKYIFFKKGAAGGCIYTSKQTAIIAVYGNPGTSSSLQQDLEKKEGAEIAVNPADCNSTVKRIAEYLISLDY

SEQ ID NO: 92 - protein, 132 aa, Betula pendula
MSWQTYVDEHLMCDDGQGEELAASAIVGHDGSVWAQSSSFPQFKPQEITGIMKDFEEPGHLAPTGLHLGGIKYMVIQGEAGAVIRGKKGSGGITIKKTGQALVFGIYEEPVTPGQCNMVVERLGDYLIDQGL

SEQ ID NO: 93 - protein, 49 aa, Escherichia coli; identical to SEQ ID NO: 48

SEQ ID NO: 94 - protein, 906 aa, Mus musculus
MGRYWLLPGLLLSLPLVTGWSTSNCLVTEGSRLPLVSRYFTFCRHSKLSFLAACLSVSNLTQTLEVVPRTVEGLCLGGTVSTLLPDAFSAFPGLKVLALSLHLTQLLPGALRGLGQLQSLSFFDSPLRRSLFLPPDAFSDLISLQRLHISGPCLDKKAGIRLPPGLQWLGVTLSCIQDVGELAGMFPDLVQGSSSRVSWTLQKLDLSSNWKLKMASPGSLQGLQVEILDLTRTPLDAVWLKGLGLQKLDVLYAQTATAELAAEAVAHFELQGLIVKESKIGSISQEALASCHSLKTLGLSSTGLTKLPPGFLTAMPRLQRLELSGNQLQSAVLCMNETGDVSGLTTLDLSGNRLRILPPAAFSCLPHLRELLLRYNQLLSLEGYLFQELQQLETLKLDGNPLLHLGKNWLAALPALTTLSLLDTQIRMSPEPGFWGAKNLHTLSLKLPALPAPAVLFLPMYLTSLELHIASGTTEHWTLSPAIFPSLETLTISGGGLKLKLGSQNASGVFPALQKLSLLKNSLDAFCSQGTSNLFLWQLPKLQSLRVWGAGNSSRPCLITGLPSLRELKLASLQSITQPRSVQLEELVGDLPQLQALVLSSTGLKSLSAAAFQRLHSLQVLVLEYEKDLMLQDSLREYSPQMPHYIYILESNLACHCANAWMEPWVKRSTKTYIYIRDNRLCPGQDRLSARGSLPSFLWDHCPQTLELKLFLASSALVFMLIALPLLQEARNSWIPYLQALFRVWLQGLRGKGDKGKRFLFDVFVSHCRQDQGWVIEELLPALEGFLPAGLGLRLCLPERDFEPGKDVVDNVVDSMLSSRTTLCVLSGQALCNPRCRLELRLATSLLLAAPSPPVLLLVFLEPISRHQLPGYHRLARLLRRGDYCLWPEEEERKSGFWTWLRSRLG

SEQ ID NO: 95 - protein, 904 aa, Rattus norvegicus
MGRSFLLPGLLLSLPLVTGWTTPKCLVTEGSQLPLVSRYFTLCHYSKLSFLAACFPVSNLTQTLEAVPRNVEGLCLSGSVSTLLPDAFSAFPGLKFLGLNLHLTRLLPGALRGLGQLRNLSFVDQPSGKNSLFLPPDAFGDLISLQRLHFCGPCLNKKAGVRLPSSLQWLSVTISCLQDSGELAGIFPDLVQNSSSRASWTLKKLDLSLNQKLKMATPGSLQGLQVEILDLRKTQLDAGAVKGLGLQKLNVLYAPTATAELAAETAAHFELQGLNVDRSKIGNISQEALASCHSLETLSLSDTGLTKLPPGFLAAMPRLRRLNLAGNQLQSTMLCMNETGDVSGLSTLDLSGNGLRILPPATFSCLPHLRELLLQDNQLLSLEGHPFQDLQQLETLKLDRNPLLNLGKNCLAALPALTTLSLLDTQILHSPDAGFWGARSLHTLHLSLPPLSAPAVLSLPMYLTSLELHVTPGLKHWTLSPNIFPFLETLTINGRGLKLGVQNASEVFPALQHLFLLQNSLDAFCSQDASSIFLWQLPKLQSLKVWGAGSNSRPCLITGLPSLQELKLESLQSITQPRSVQLEELVGDLPQLQALQLSSTGLKSLSAAAFRRLHSLQALVLDSEKDLVLQDSLREYSPQMPRYVYILQSKLACQCANAWMELWVKQSTKTYVHIRDGHLCPGEVRVPARDSLISFLWDHCPQTLELKLFLASSALVLLLIVLPLLQGARNTWIPYLRALFRIWLQGLRGQGNAGKRFLFDVFVSHCRQDQGWVLEELLPALEGFLPAGLGLRLCLPERDFEPGKDVVDNVVDSMVSSRVTLCVLSGPALCNPRCCLELRLATSLLLAAPSPPVLLLVFLEPISRHQLPSYHRLARLLRRGDYCLWPEEEERKGGRWTWLRSRLG

SEQ ID NO: 96 - protein, 166 aa, Homo sapiens
MDRHLLLPGLLLSLPLTAGWTISNSLVTEGSRLSIVSRFFLICLLDSSLPFLTTCLSVINLVRALETVLQNVEGLCQSGSTSALPQDAFSRFPGLKVLGLNLHLTQLLPGALWGLGQLHYVFHSSHRGSINLPTADAFGDLRSLQDLAFLGSCLDGSLGVRLPPSL

SEQ ID NO: 97 - protein, 166 aa, Pan troglodytes [sequence omitted]

SEQ ID NO: 98 - DNA, 2727 bp, Homo sapiens
atggacaggcacttgctgttgcctggtctgctcctgtcccttcctctgaccgcaggctggaccatctccaatagtttagtgactgaaggctcccggctgtctatggtctcccgcttcttcctgatttgcctcttggactccagcctgcctttcctcaccacatgcctctcagtgatcaacttggtgcgggccttggaaactgtgctgcagaacgtggagggtctctgtcaatctggttccacttctgctctgcctcaggatgccttctcccgctttcctgggctcaaggtcctggggctgaatctgcatctcacccagctcctgccaggagctctctgggggttggggcagctgcattatgtctttcatagctcccaccgtgggagcattaatcttcctaccgctgatgcctttggtgacctgagatccctccaggaccttgctttcttgggttcctgcctggatgggagcttgggtgtccggttgcctcccagtctgtgatggctgtcgatcaggtgtaatttccttcagaatgtgggggtgctggctgatatcttcccagatctggtgcatggcccctcctctggggatgcctgggccttggacatgttggacctgtcattcaatagtaggctgaagctggccagtcctggagccttccaggtcctcaagctggggactctgaatctggaccacacaaagatgaaggcagatgcactggtgggacgggggctgcagagattggatgccctgtgacactcactgacatggctgagctgcctgccaggatggttgcccattttgagcttcaggagctgaatttggggattaatcggacaaggcacatagccctggaaggcctggcttcctgtcacagcctgaagagctcgggtcttcggagcaatggcctgattgagttaccacgaggtttcctggctgccatgcccaggcttcagagactgaacctggccaacaaccaactgaggagcgccatgttgtgtatgaatgagacagggtttgtgtcaggattgtgggccctggatctgtccaagaataggctgtgtaccctgtccccagtcatcttctcctgtttgccccacctgcgggagctgctacttcaagggaaccaactggtttgcttgaaagaccaggtattccagggcctacagaggctacagaccttgaacttgggcaataatccactggtaaccctgggtgagggctggctggctcctctgcctacactgaccacccaaaacctggtaggtactcacatggtgctgagcccaacctggggcttccggggcccagaaagtctgcacagcttgagaatacagtttccctttggccctgcgggagtagcattttccctgctcacaagactgactagcttggagctccacgcagtttcaggcatgaagcattggaggttgtctcctaatgtctttccagtcttgcagatcctgactttaaagggctggggactgcagctagagacccagaatatctccaagatcttccctgcccttcatcaactctccctgcttggcagtaggttggagcccctctgttcccaggacacctccagcttcttcctctggcagctcccgaagctcaagtccttgaaggatggggaaacaggcatagccctaggccctactgcatcacgggactgcccagtctacaggagctgaagctgcaggcactgcagtctcaagcatgcccctgcccagtgcggcttgaggagctggtgggtgagttgcccaggcttgatatgctgcagctgtcccaaacagggttggagacactgtctgctgctgcttttgggggcctcggcagtctccaggtcttagtactagacagggagaaagacttcatgctggatgacagcctccaggagcacagtcctcggatgccccagtacatctatattctgacctcatccttggcctgccagtgtgccaatgcctgcgtggggccctggctttagcagtcccccagaacatacatgcacatagtatcacagcagctgtgccattcagaagctgggggccactcaaagaatctctttttcccttttctctggagccactgccccaagactttggggttggagctcttttttgcgcagctctgccctgctgcttctgctggtctccttgcccttcctaaaggaagccaggaattcctggatcctctaactcaaggccttgctcagggtttggttccagagtctgaggagtcagaagggtaaaggcaagaggttcctctatgacgtgtttgtgtcccactgcaggcaagaccagggctggatggtgcaggagctgctgcctgctctagaggactgccctccagctggccgggggctgccactctgcctccatgagtgggattttgagccaggcaaggatgtggctgacaatgcagcagacagcatggtgggcagctgggtcacgctctgtgtgctgagtcaccaggccctgcacaccccctgctgatgcctggagctcctcctggccacctcctttctgctggctgtcccccaccccccaagggctactgctggtcttcctggagcccatcttacgccactagctcccttgctgccacagattggcctggttgctccgctgaagagactattgcatgtggcccaaggaagaggaaagaaagaatgacttctgggcttggttagggagcaggctggagcaccctggggtagggtag

SEQ ID NO: 99 - DNA, 2728 bp, Homo sapiens
atggacaggcacttgctgttgcctggtctgctcctgtcccttcctctgaccgcaggctggaccatctccaatagtttagtgactgaaggctcccggctgtctatggtctcccgcttcttcctgatttgcctcttggactccagcctgcctttcctcaccacatgcctctcagtgatcaacttggtgcgggccttggaaactgtgctgcagaacgtggagggtctctgtcaatctggttccacttctgctctgcctcaggatgccttctcccgctttcctgggctcaaggtcctggggctgaatctgcatctcacccagctcctgccaggagctctctgggggttggggcagctgcattatgtctttcatagctcccaccgtgggagcattaatcttcctaccgctgatgcctttggtgacctgagatccctccaggaccttgctttcttgggttcctgcctggatgggagcttgggtgtccggttgcctcccagtctgcaatggctgtcgatcaggtgtaatttccttcagaatgtgggggtgctggctgatatcttcccagatctggtgcatggcccctcctctggggatgcctgggccttggacatgttggacctgtcattcaatagtaggctgaagctggccagtcctggagccttccaggtcctcaagctggggactctgaatctggaccacacaaagatgaaggcagatgcactggtgggacgggggctgcagagattggatgccctgtatgcactcactgacatggctgagctgcctgccaggatggttgcccattttgagcttcaggagctgaatttggggattaatcggacaaggcacatagccctggaaggcctggcttcctgtcacagcctgaagagctcgggtcttcggagcaatggcctgattgagttaccacgaggtttcctggctgccatgcccaggcttcagagactgaacctggccaacaaccaactgaggagcgccatgttgtgtatgaatgagacagggtttgtgtcaggattgtgggccctggatctgtccaagaataggctgtgtaccctgtccccagtcatcttctcctgtttgccccacctgcgggagctgctacttcaagggaaccaactggtttgcttgaaagaccaggtattccagggcctacagaggctacagaccttgaacttgggcaataatccactggtaaccctgggtgagggctggctggctcctctgcctacactgaccacccaaaacctggtaggtactcacatggtgctgagcccaacctggggcttccggggcccagaaagtctgcacagcttgagaatacagtttccctttggccctgcgggagtagcattttccctgctcacaagactgactagcttggagctccacgcagtttcaggcatgaagcattggaggttgtctcctaatgtctttccagtcttgcagatcctgactttaaagggctggggactgcagctagagacccagaatatctccaagatcttccctgcccttcatcaactctccctgcttggcagtaggttggagcccctctgttcccaggacacctccagcttcttcctctggcagctcccgaagctcaagtccttgaaggtatggggaaacaggcatagccctaggccctactgcatcacgggactgcccagtctacaggagctgaagctgcaggcactgcagtctcaagcatgcccctgcccagtgcggcttgaggagctggtgggtgagttgcccaggcttgatatgctgcagctgtcccaaacagggttggagacactgtctgctgctgcttttgggggcctcggcagtctccaggtcttagtactagacagggagaaagacttcatgctggatgacagcctccaggagcacagtcctcggatgccccagtacatctatattctgacctcatccttggcctgccagtgtgccaatgcctgcgtggggccctggcttaagcagtcccccagaacatacatgcacatagtatcacagcagctgtgccattcagaagctgggggccactcaaagaatctctttttcccttttctctggagccactgccccaagactttggggttggagctcttttttgccagctctgccctgctgcttctgctggtctccttgcccttcctaaaggaagccaggaattcctggatcctctacctcaaggccttgctcagggtttggttccagagtctgaggagtcagaagggtaaaggcaagaggttcctctatgacgtgtttgtgtcccactgcaggcaagaccagggctggatggtgcaggagctgctgcctgctctagaggactgccctccagctggccgggggctgccactctgcctccatgagtgggattttgagccaggcaaggatgtggctgacaatgcagcagacagcatggtgggcagctgggtcacgctctgtgtgctgagtcaccaggccctgcacaccccctgctgctgcctggagctcctcctggccacctcctttctgctggctgtcccccaccccccaaggactactgctggtcttcctggagcccatcttacgccaccagctcccttgctgccacagattggcctggttgctccgccgaagagactattgcatgtggcccaaggaagaggaaagaaagaatgacttctgggcttggttagggagcaggctggagcaccctggggtagggtag

SEQ ID NO: 100 - protein, 908 aa, Homo sapiens
MDRHLLLPGLLLSLPLTAGWTISNSLVTEGSRLSMVSRFFLICLLDSSLPFLTTCLSVINLVRALETVLQNVEGLCQSGSTSALPQDAFSRFPGLKVLGLNLHLTQLLPGALWGLGQLHYVFHSSHRGSINLPTADAFGDLRSLQDLAFLGSCLDGSLGVRLPPSLQWLSIRCNFLQNVGVLADIFPDLVHGPSSGDAWALDMLDLSFNSRLKLASPGAFQVLKLGTLNLDHTKMKADALVGRGLQRLDALYALTDMAELPARMVAHFELQELNLGINRTRHIALEGLASCHSLKSSGLRSNGLIELPRGFLAAMPRLQRLNLANNQLRSAMLCMNETGFVSGLWALDLSKNRLCTLSPVIFSCLPHLRELLLQGNQLVCLKDQVFQGLQRLQTLNLGNNPLVTLGEGWLAPLPTLTTQNLVGTHMVLSPTWGFRGPESLHSLRIQFPFGPAGVAFSLLTRLTSLELHAVSGMKHWRLSPNVFPVLQILTLKGWGLQLETQNISKIFPALHQLSLLGSRLEPLCSQDTSSFFLWQLPKLKSLKVWGNRHSPRPYCITGLPSLQELKLQALQSQACPCPVRLEELVGELPRLDMLQLSQTGLETLSAAAFGGLGSLQVLVLDREKDFMLDDSLQEHSPRMPQYIYILTSSLACQCANACVGPWLKQSPRTYMHIVSQQLCHSEAGGHSKNLFFPFLWSHCPKTLGLELFFASSALLLLLVSLPFLKEARNSWILYLKALLRVWFQSLRSQKGKGKRFLYDVFVSHCRQDQGWMVQELLPALEDCPPAGRGLPLCLHEWDFEPGKDVADNAADSMVGSWVTLCVLSHQALHTPCCCLELLLATSFLLAVPHPPRLLLVFLEPILRHQLPCCHRLAWLLRRRDYCMWPKEEERKNDFWAWLGSRLEHPGVG

SEQ ID NO: 101 - DNA, 2721 bp, Mus musculus
  CDS (1)..(2718)
atgggcaggtactggctgctgccaggtctcctcctttccctgcctctggtaactgggtggagcacttccaactgcctggtgaccgaaggctcccgactgcccctggtctcccgctatttcacattctgccgccattccaagctatcctttcttgctgcatgcctctccgtgagcaacctgacacagaccttggaagttgtacctcggactgtggaggggctctgcctcggtggtactgtgtctactctgcttccagatgctttctctgcttttcctggtctcaaggtcctggcactgagtctgcaccttacccaacttctgccaggagctctccggggtctgggacagttgcagagcctctctttttttgactctcctcttaggagatctctctttctacctcctgatgccttcagtgacctgatttccctccagagactccatatctctggcccttgcctggataagaaggcaggcatccgcctgcctcccggtctgcaatggctgggtgtcacgctcagttgcattcaggacgtgggagagctggctggtatgttcccagatctggtgcaaggttcctcctccagggtttcgtggaccctgcagaagttggatctgtcatccaactggaagctgaagatggctagtcctgggtccctccagggtctccaggtggagattctggacctgacaagaacaccactggatgccgtgtggctgaagggcctgggacttcagaaactcgatgtcttgtatgcacagactgccacggccgagctggctgctgaggctgttgcccactttgagctgcagggcttgattgtgaaagaaagcaagataggatctatatctcaggaggctctggcttcctgccacagcctgaagaccttgggtctttcaagcactggcctaaccaagcttccaccaggcttcctgactgccatgcctaggcttcagcgactggagctgtccggaaaccaactgcagagcgccgtgctgtgcatgaatgagacgggagatgtgtcaggactcacgactctggatctgtcaggcaacaggttgcgcatcctgcctccagccgccttctcctgcttaccccacttgcgagagctgctgcttcggtacaaccagctgctttccctggagggatacctattccaggagctccagcaactagagaccttgaagctggatggaaaccccctgcttcacctgggtaagaactggttggcggctctgcctgcattgaccacccttagcttgctagatacccaaatacggatgagcccagagcctggcttctggggagcaaagaatctgcataccttgagcctgaagcttcccgctctccctgctccggcagtattgttcctgcccatgtatctgaccagcttagagcttcatatagcctcaggcacgacggagcactggacgctgtccccagcgatctttccttccttggagaccttgactataagcggcgggggactgaagctgaagctggggtcccagaatgcttctggggtcttccctgctctccagaagctctccctgcttaagaacagcttggatgccttctgctcccagggtacctccaaccttttcctctggcagctccccaaacttcagtccttgagggtatggggtgctggaaacagctccagaccctgccttatcactgggctgcccagcctacgggagctgaagctggcgtcgcttcagtccataacccagccccgttcggtgcagctggaggagctggtgggtgaccttccacagctccaggccttagtgctatccagcacaggcctcaagtcactgtcggctgctgctttccagcgcctgcacagtctccaggtcttagtgctagaatacgagaaggacttgatgctgcaggacagtctgagggagtacagccctcagatgccccactatatatacattctggagtcaaacctggcctgccactgtgccaatgcgtggatggagccatgggttaagcggtccactaaaacgtacatatacataagagacaatcgcttatgtccaggacaagacaggctctctgctaggggttcccttccctcctttctctgggaccactgcccccagacgttggagctgaaactctttttggctagttctgccttggtgttcatgctaattgccttgcctctcctccaagaagccaggaactcttggatcccctacctgcaggccttgttcagggtttggctccagggtctgaggggtaagggagacaaggggaagaggttcctttttgatgtattcgtgtcccactgcaggcaagaccagggctgggtgatagaggaacttctgcctgctctggagggcttccttccagctggcctgggcctgcgcctctgtctccccgagcgtgactttgagcctggtaaggatgtagttgataatgtggtagatagcatgttgagcagccgtaccacactctgcgtgttgagtgggcaggccctgtgtaacccccgatgccgcctggagctccgcttggccacctctctcctcctggctgccccgtcccctccagtgttgctgctagtcttcttggaacccatttctcggcaccagcttccgggttaccacagactggctcggctgcttcgaagaggagactactgtctgtggcccgaggaagaggagagaaagagtgggttctggacttggctgaggagcaggctagggtag

SEQ ID NO: 102 - protein, 906 aa, Mus musculus; identical to SEQ ID NO: 94

SEQ ID NO: 103 - DNA, 2717 bp, Rattus norvegicus
atgggcaggtccttcctgctgcccggtctcctcctttcactgcctctggtaacagggtggagcactcccaaatgcctggtgactgaaggctcccaactgcccctggtctcccgctatttcacactctgccactactccaagctgtcctttcttgctgcatgcttccctgtgagcaacctgacgcagaccttggaagctgtgcctcggaatgtggaggggctctgcctcagtggttctgtgtctactctgcttccggatgccttctctgcttttccctggactcaagttcctgggactgaatctgcaccttacccgactcctgccaggagctctccggggcctgggacagttacggaacctctcttttgttgaccaaccttctggaaagaactctctctttctacctcctgatgcctttggtgacctgatttccctccagagactccatttctgtggcccctgtctgaataagaaggcaggtgtccgtctaccttccagtctgcaatggctctctgtcacaatcagttgccttcaggactcaggagagctggctggtatattcccagatctggtgcaaaattcctcctccagggcttcgtggaccctgaagaagttggatctgtcattaaaccagaagctgaagatggctactcctgggtccctacagggtctccaggtggagattctggacctgaggaaaacacaactggatgctggggcagtgaagggcctgggacttcagaaactcaatgttttgtatgcaccgactgccacggccgagctggctgctgagactgctgcccactttgagctgcagggcctgaatgtggacagaagcaagataggaaatatatctcaggaggccctggcttcctgccatagcctggagaccttgagtctgtcagacactggactgactaagctgccaccaggctttctggctgccatgcctaggcttcggagactgaacctggccggaaaccaactacagagcaccatgctgtgtatgaatgagacaggagacgtgtcaggactctcaactctggatctgtcaggcaatgggttgcgcatcctgcctccagccaccttctcctgtttaccacacttgcgagagctgctgcttcaggacaaccagctgctttccctggaggggcacccgttccaagatctacagcaactagagaccttgaagctggataggaaccccctgcttaacctggggaagaactgtttggctgctctgcctgcattgactacccttagcctgctagatacccaaatattgcacagcccagatgctggcttctggggagcaaggagtctgcataccttacacctgagcttgccccctctctctgctccggcagtattgtccctgcccatgtatctgaccagtttagagcttcatgtaaccccaggcttgaagcattggacactgtccccaaatatctttcctttcttggagaccttgaccataaacggcaggggactgaagctgggggtccaaaatgcctctgaggtcttccctgctctccagcatctctttctgctccagaatagcttggatgccttctgctcccaggacgcctccagcatttttctctggcagctccccaaacttcagtccttgaaggtatggggtgctggaagtaattccagaccctgcctcatcactgggctgcccagcctgcaggagctaaagctggagtctctacagtccatcacccagccccgctcagtacagctggaggagctggtgggtgaccttccacaactccaggcgttacagctgtccagcacagggctcaaatccctgtcggctgctgctttccggcgcctgcacagtctccaggccttagtgctggacagtgagaaagacttggtgctgcaggacagtctgagggagtacagccctcagatgccccgctatgtatacattctgcagtcaaagttggcctgccagtgtgctaatgcgtggatggagctgtgggttaagcagtccaccaaaacgtacgtacacataagagacggtcacctgtgccccaggagaagtcagggtccccgccagggattcccttatctcctttctctgggaccactgcccccagactttggagctgaaactctttttggccagttctgccttggtgttattgctaattgtcttgcctctcctccaaggagccaggaacacttggatcccctacctgcgggccttgttcaggatttggctccagggtctgaggggtcaggggaatgcggggaagaggttcctttttgatgtattcgtgtcccactgcaggcaagaccagggctgggtgctagaggaacttctgcctgctctggagggcttccttccagctggcctcggcctacgcctctgtctccccgagcgtgactttgagcctggtaaggatgtagttgataatgtggtagacagcatggtgagcagccgagtcacgctctgcgtgctgagtggcccggccttgtgtaacccccgatgctgcctggagctccgcttggccacttctcttctcctggctgccccgtcccctccagtgttgctgctggtcttcctggaacccatttcccgacaccagctccccagttaccacagactggctcggttgcttcgacgaggagactactgcctgtggcctgaggaggaggaaagaaagggtgggttctggacgtggctgaggagcaggctagggtag

SEQ ID NO: 104 - DNA, 2727 bp, Homo sapiens; identical to SEQ ID NO: 98

SEQ ID NO: 105 - DNA, 2727 bp, Pan troglodytes
atggacaggcacttgctgttgcctggtctgctcctgtcccttcctctgaccgcaggctggaccatctccaatagtttagtgactgaaggctcccggctgtctattgtctcccgcttcttcctgatttgcctcttggactccagcctgcccttcctcaccacatgcctctcagtgatcaacttggtgcgggccttggaaactgtgctgcagaacgtggagggtctctgtcaatctggttccacttctgctctgcctcaggatgccttctcccgctttcctgggctcaaggtcctggggctgaatctgcatctcacccagctcctgccaggagctctctgggggttggggcagctgcattatgtctttcatagctcccaccgtgggagcattaatcttcctaccgctgatgcctttggtgacctgagatccctccaggaccttgctttcttgggttcctgcctggatgggagcttgggtgtccggttgcctcccagtctgtgatggctgtcgatcaggtgtaatttccttcagaatgtgggggtgctggctgatatcttcccagatctggtgcatggcccctcctctggggatgcctgggccttggacatgttggacctgtcattcaatagtaggctgaagctggccagtcctggagccttccagatcctcaagctggggactctgaatctggaccacacaaagatgaaggcagatgcactggtgggactggggctgcagagattggatgccctgtgacactcactgacatggctgagctgcctgccaggatggttgcccattttgagcttcaggagctgaatttggggattaatcggacaaggcacatagccctggaaggcctggcttcctgtcacagcctgaagagctcgggtcttcagagcaatggcctgattgagttaccacgaggtttcctggctgccatggccaggcttcagagactgaacctggccaacaaccaactgaggagcaccatgttgtgtatgaatgagacagggtttgtgtcaggattgtggggccctggatctgtccaagaataggctgtgtaccctgtccccagtcatcttctcctgtttgccccacctgcgggagctgctacttcaagggaaccaactggtttgcttgaaaggccaggtattcagggcctacagaggctacagaccttgaacttgggcagtaatccactggtaaccctgggtgagggctggctggctcctctgcctacactgaccacccaaaacctggtaggtactcacatggtactgagcccaacctggggcttccggggcccagaaagtctgcacagcttgagaatacagtttccctttggccctgcgggagtagcattttccctgctcacaagactgactagcttggagctccacgcagtttcaggcatgaagcattggaggttgtctcctaatgtctttccagtcttgcagatcctgactttaaagggctggggactgcagctagagacccagaatatctccaagatcttccctgcccttcatcaactctccctgcttggcagtaggttggagcccctctgttcccaggacacctccagcttcttcctctggcagctcccgaagctcaagtccttgaaggatggggaaacaggcatagccctaggccctactgcatcacgggactgcccagcctacaggagctgaagctgcaggcactgcagtctcaagcacgcccctgcccagtgcggcttgaggagctggggggtgagttgcccaggcttgatatgctgcagctgtcccaaacagggttggagacactgtctgctgctgcttttgggggcctcggcagtctccaggtcttagtactagacagggagaaagaccttatgctggatgacagcctccaggagcacagtcctcggatgccccagtacatctatattctgacttcatccttggcctgccagtgtgccaatgcctgcgtggggccctggctttagcagtcccccagaacatacatgcacatagtatcacagcagctgtgccattcagaagctgggggccactcaaagaatctctttttcccttttctctggagccactgccccaagactttggggttggagctcttttttgcgcagctctgccctgctgcttctgctggtctccttgcccttcctaaaggaagccaggaattcctggatcctctaactcaaggccttgctcagggtttggttccagagtccgaggagtcagaagggtaaaggcaagaggttcctctatgacgtgtttgtgtcccactgcaggcaagaccagggctggatggtgcaggagctgctgcctgctctagaggactgccctccagctggccgggggctgccactctgcctccatgagtgggattttgagccaggcaaggatgtggctgacaatgcagcagagagcatggtgggcagctgggtcacgctctgtgtgctgagtcaccaggccctgcacaccccctgctgatgcctggagctcctcctggccacctcctttctgctggctgccccccaccccccaagggctactgctggtcttcctggagcccatcttacgccactagctcccttgctgccacagattggcctggttgctccgctgaagagactattgcatgtggcccaaggaagaggaaagaaagaatgacttctgggcttggttagggagcaggctggagcagcctggggtagggtag

SEQ ID NO: 106 - protein, 171 aa, Plasmodium falciparum; identical to SEQ ID NO: 4

SEQ ID NO: 107 - protein, 170 aa, Sarcocystis neurona; identical to SEQ ID NO: 2

SEQ ID NO: 108 - protein, 163 aa, Neospora caninum; identical to SEQ ID NO: 1

SEQ ID NO: 109 - protein, 163 aa, Toxoplasma gondii
MSDWDPVVKEWLVDTGYCCAGGIANAEDGVVFAAAADDDDGWSKLYKDDHEEDTIGEDGNACGKVSINEASTIKAAVDDGSAPNGVWIGGQKYKVVRPEKCFEYNDCTFDITMCARSKGGAHLIKTPNGSIVIALYDEEKEQDKGNSRTSALAFAEYLHQSGY

SEQ ID NO: 110 - protein, 169 aa, Eimeria tenella
MGEADTQAWDTSVREWLVDTGRVFAGGVASIADGCRLFGAAVEGEGNAWEELVKTNYQIEVPQEDGTSISVDCDEAETLRQAVVDGRAPNGVYIGGTKYKLAEVKRDFTFNDQNYDVAILGKNKGGGPLIKTPNENVVIALYDEEKEQNKADALTTALNFAEYLYQGGF

SEQ ID NO: 111 - DNA, 513 bp, Eimeria acervulina; identical to SEQ ID NO: 11

SEQ ID NO: 112 - DNA, 510 bp, Eimeria tenella
atgggagaagcagacacccaggcctgggacacttcggtccgcgagtggctggttgacaccggcagggtcttcgccggcggcgttgctagcatagccgacggctgccggctcttcggagcagcagtggagggcgagggcaacgcctgggaagaactcgtcaagaccaactaccaaattgaagtcccccaggaagacggaacctccatttcagtggattgcgacgaggcggagactctgcggcaggcggtggtggacggccgcgcgcccaacggcgtctacatcggcggcaccaagtacaagctcgccgaagtcaaaagggacttcaccttcaacgaccaaaactatgatgtggcgattctgggaaaaaacaaaggcggagggtttttgattaaaactccaaacgaaaatgttgttatagctttgtatgatgaagaaaaagaacaaaacaaagctgatgctctcacaacagctcttaacttcgcggagtatctgtaccagggaggcttctaa

SEQ ID NO: 113 - DNA, 492 bp, Neospora caninum; identical to SEQ ID NO: 7

SEQ ID NO: 114 - DNA, 492 bp, Toxoplasma gondii; identical to SEQ ID NO: 9

SEQ ID NO: 115 - DNA, 513 bp, Sarcocystis neurona; identical to SEQ ID NO: 8

SEQ ID NO: 116 - DNA, 516 bp, Plasmodium falciparum; identical to SEQ ID NO: 10

SEQ ID NO: 117 - protein, 940 aa, Escherichia coli; identical to SEQ ID NO: 28

SEQ ID NO: 118 - DNA, 2823 bp, Escherichia coli
ttacagcatcggcttgaggaagcgcgccgtatgcgaagctttgcactctgcgacggtttctggcgtaccggagacgaggatttcgccgcccccactgccgccttccggtcccaggtcgacaatccagtcagcggttttgatcacgtcgagattgtgctcaattaccacaatggtattgccctgatcgcgcagtttatgcagtacgtcgagcagttgctgaatatcggcgaagtgcagaccggtggtcggctcgtcgagaatatacagcgtctgcccggtgccgcgttttgacagctcacgcgccagcttcacgcgctgggcttcaccaccagaaagtgtggttgcggactgcccgaggcgaatgtacgtcaggccaacatccatcagcgtttgcagcttacgcgccagagctggcaccgcatcaaagaactcacgcgcctcttcgatggtcatatccagcacttcgtggatggttttgcctttgtacttaatctccagcgtttcacggttatagcgtttgcctttgcactggtcgcacggtacgtaaatgtccggcaggaagtgcatctccactttgatcacgccgtcgccctgacaggcctcgcagcgtccgccacggacgttaaagctgaaacgtcctggcgtatagccgcgcgcacgggattccggtacgcccgcaaacagttcgcgcacaggtgtaaacacgccggtataggtcgccgggttagaacgcggagtacgaccaattgggctttggtcgatatcgatcactttatcgaaatgctccagtccctgaatatcgcgatacggtgccggttcggcgatggtcgcaccattcaactggcgttgggcaatcgggaacagtgtgtcgttaatcagtgtcgatttaccggaacctgaaacccctgtgatgcaggtaaacagaccgactggcagcgttagcgtcacgtctttcaggttattaccgcgtgcacccgtcagcttcagcactttttccggattcgccggaacgcgtttcttcggcacttcaatcttgcgtttaccgctcatgtactgtccggtcaacgactccggcaccgccataattgcttccagtggaccttccgcgaccacttcaccgccgtgaacacccgcacccggaccgatatcgatcacatgatcagcggcgcgaatcgcgtcttcgtcgtgctccaccacaatcacggtattaccgagatcgcgcagatggataagcgtacccagcaggcgctcgttatcgcgctggtgcaggccgatagacggctcatccagcacgtacatcacgccaaccaggcctgcaccaatctggctcgccagacgaatacgctgggcttcaccgccggaaagtgtctctgctgagcgggaaagtgtcaggtaattcaggccgacgttaacgaggaatttcaggcgatcgccaatctccttaagaattttttccgcaatcttcgctcgttgacctgcgagtttgagattgttgaagaattccatcgcatgaccgatgctcatgtcggagatagcaggcagcggcgtattctcgacatatacatggcgcgcttcccgacgcagacgcgttccttcgcagctggcgcacgggcgattgctgataaacttggctaattcttcacgtaccgcactggattccgtctctttataacggcgctccatattgtgcagcacgccttcgaacggatgacgacgaatggaggtatcgccacgatcgttcatgtatttgaattcaatattttctttgccagaaccgtacaacaccactttatgcacgttcgcgctcaggctgccccacggcgcttcgacgtcgaacttatagtgatctgccagcgatttcagcatctggaaataatagaagttgcggcgatcccagccacggatcgcaccgccagccagcgataattccggattctggatcacgcggtcaggatcgaaatattgctgtacgccaagaccgtcacaggtcgggcaggctcccgccgggttgttaaatgaaaacaggcgaggttccagctcgcgcatactgtaaccgcaaatcgggcaggcaaagttggcggagaacagcagctcttccgctttcgggtcgtccatatccgccactaccgcggtaccaccggaaagctccagcgcggtttcaaacgactcggcaagacgttgggtaagatcgtcacgcactttgaagcgatcaaccaccacttcaatggtatgtttcttttgcagttccagtttcggcggatcggaaagatcgcagacttcgccatcaatacgagcacggatgtaaccctggcttgccaggttctccagcgttttggtgtgttcgcctttgcgctctttaatgattggcgcgagcagcatcagacgcttgccttccggctgcgaaagcacgttatccaccatctggctgacggtttgcgccgccagcggaacatcgtggtccgggcagcgcggctcaccaacgcgagcgtacagcagacgcaaatagtcgtggatttcggtgattgtccccaccgtagagcgcgggttatgagacgtcgatttctgctcaattgagatggcaggagaaagcccctcaatatggtcgacgtccggcttctccatcagtgacagaaactgccgcgcgtaggcggaaagggattcaacgtaacggcgctgcccttcggcatataaggtgtcgaaagcgagcgaggatttgccagaacccgaaagcccggtcacgacaatgagcttgtcgcgggggataacgaggttgatgtttttgagattatgggtgcgggcgccccgaacttcgatcttatccat

SEQ ID NO: 119 - protein, 673 aa, Escherichia coli
  MOD_RES (215): Variable amino acid
MSKPFKLNSAFKPSGDQPEAIRRLEEGLEDGLAHQTLLGVTGSGKTFTIANVIADLQRPTMVLAPNKTLAAQLYGEMKEFFPENAVEYFVSYYDYYQPEAYVPSSDTFIEKDASVNEHIEQMRLSATKAMLERRDVVVVASVSAIYGLGDPDLYLKMMLHLTVGMIIDQRAILRRLAELQYARNDQAFQRGTFRVRGEVIDIFPAESDDIALRVXLFDEEVERLSLFDPLTGQIVSTIPRFTIYPKTHYVTPRERIVQAMEEIKEELAARRKVLLENNKLMEEQRLTQRTQFDLEMMNELGYCSGIENYSRFLSGRGPGEPPPTLFDYLPADGLLVVDESHVTIPQIGGMYRGDRARKETLVEYGFRLPSALDNRPLKFEEFEALAPQTIYVSATPGNYELEKSGGDVVDQVVRPTGLLDPIIEVRPVATQVDDLLSEIRQRAAINERVLVTTLTKRMAEDLTEYLEEHGERVRYLHSDIDTVERMEIIRDLRLGEFDVLVGINLLREGLDMPEVSLVAILDADKEGFLRSERSLIQTIGRAARNVNGKAILYGDKITPSMAKAIGETERRREKQQKYNEEHGITPQGLNKKVVDILALGQNIAKTKAKGRGKSRPIVEPDNVPMDMSPKALQQKIHELEGLMMQHAQNLEFEEAAQIRDQLHQLRELFIAAS

SEQ ID NO: 120 - DNA, 2022 bp, Escherichia coli
atgagtaaaccgttcaaactgaattccgcttttaaaccttctggcgatcagccggaggcgattcgacgtctcgaagaggggctggaagatggcctggcgcaccagacgttacttggcgtgactggctccgggaaaaccttcaccattgccaatgtcattgctgaccttcagcgcccaaccatggtacttgcgcccaacaaaacgctggcggcccagctgtatggcgaaatgaaagaattctttccggaaaacgcggtggaatatttcgtctcctactacgactactatcagccggaggcctatgtaccgagttccgacaccttcattgagaaagatgcctcggtaaacgaacatatcgaacagatgcgtttgtccgccaccaaagcgatgctggagcggcgtgatgtggttgtggtggcgtctgtttccgcgatttatggtctgggcgatcctgatttatatctcaagatgatgctccatctcacggtcggtatgattatcgatcagcgcgcgatcctgcgccgactggcggagctgcaatatgctcgtaatgatcaagcattccagcgtggtactttccgtgttcgtggcgaggtgattgatatcttcccggcagaatcggatgacattgcacttcgcgtgragctgtttgacgargaagtggaacgattgtcgttatttgacccgctgaccgggcagattgtttccactattccacgttttaccatctacccgaaaacgcactacgtcacgccacgcgagcgcatcgtccaggcgatggaggagatcaaagaagagctggccgccagacgtaaagtgctattggaaaacaacaaactgatggaagagcagcggctgacccagcgtacccagtttgatctggagatgatgaacgagctaggctactgttcggggattgagaactactcgcgcttcctctccggtcgtggaccgggtgagccaccgccgacgctgtttgattacctgcctgctgatgggctgctggtggtcgatgaatctcacgtcaccattccacaaattggcggcatgtatcgcggtgaccgggcgcgtaaagagacactggtggagtacggcttccgcctgccatcagcgctggataaccgtccgctgaaatttgaagagttcgaagcattagcgccgcaaaccatctatgtttcggcgacgccgggtaattacgagctggaaaaatccggtggcgatgtggtggatcaggtggtgcgtccaacaggcttactcgacccgattatcgaagtgcggccagtggcaacacaggtggatgatcttctttcggagattcgtcagcgagcggcaattaacgaacgcgtactggttacaactctgaccaagcggatggcggaagatctcactgaatatctcgaagaacacggtgagcgcgtgcgttatcttcactcagatatcgacaccgtcgaacgtatggagattatccgcgacttgcgtctgggtgagttcgacgtattggtagggatcaacttactgcgcgaaggtctggatatgccggaagtttcgctggtggcgatcctcgacgctgacaaagaaggcttcctgcgttccgaacgttcgttgatccagaccattggtcgtgcggcacgtaacgttaacggtaaagcgattctctacggcgataagatcaccccatcaatggcgaaagcgattggcgaaaccgaacgtcgccgcgagaaacagcagaagtacaacgaagaacacggcattacgccgcaaggcttgaacaagaaagtggtcgatatcctggcgctggggcagaacattgccaaaaccaaagcgaagggcagaggaaaatcgcgcccgattgttgaaccggataatgtgccgatggatatgtcgcctaaagcgttacagcagaagatccatgaactggaagggttgatgatgcaacacgcgcagaatctggagttcgaagaagcggcgcaaattcgtgaccagttgcatcagttgcgtgagctgtttattgccgcgtcgtga

SEQ ID NO: 121 - protein, 610 aa, Escherichia coli; identical to SEQ ID NO: 32

SEQ ID NO: 122 - protein, 218 aa, Escherichia coli
MINVLLVDDHELVRAGIRRILEDIKGIKVVGEASCGEDAVKWCRANAVDVVLMDMSMPGIGGLEATRKIARSTADVKIIMLTVHTENPLPAKVMQAGAAGYLSKGAAPQEVVSAIRSVYSGQRYIASDIAQQMALSQIEPEKTESPFASLSERELQIMLMITKGQKVNEISEQLNLSPKTVNSYRYRMFSKLNIHGDVELTHLAIRHGLCNAETLSSQ

SEQ ID NO: 123 - DNA, 1833 bp, Escherichia coli
tcaatgtttcaacgaccagaagatcttttctgccagaccttgcgaaatacccggcacttttgcaatttcctcgacgctggcgttacgtaaaccttgcaaaccgcccatatatttcaacaacatttgccgacgttttggcccgacgccttcaatggtttccagggaactggtatttttgaccttcgcccgttttttacggtgcccgccaatcgcgtgatcgtgtgattcatcgcgaatatgctggataacatgcagcgcgggagaatctggcggcaaactaaatccctcaccttccggctcaaagaacagcgtttccagcccagccttacgatctgctcctttggcaacaccaagtagcagcggatgatttttatcccatgagacatccagttcggcgaagacatttttcgcctgcgcaagctggcctttgccgccgtcgataagaatcacatccgggatcttactgtcgtcaatggctttaccataacgccgacgcagcacctgattcatcgccgcataatcatcgcccggcgtgatgccagtaatgttatagcgccgatactccgcacgcagcgggccgttagcatcaaacaccacacaggaagcgacggtttgttcacccatggtatggctgatgtcaaagcactccatccgcttcacttccggcaatttcaacacgctggcaagcgcggtcagccgctggtgaacggtagattgctgcgaaagtttgctggttaaggccgtcgccgcattggtgcgcgcgagtttcagataacgcgctctatcgccacgaggtttggtttgaacattaatcttgcgtcccgccagttctgaaagggaatcggcgagcagcgttttatcgctaagattaaaatcgagcaggatctcacccggtaaggtgcgcatctggctgccttgtaaatagaactgacctacgaaggtttccaccacctcgctcagttccgtaccgccaggcactttcgggaaatagctgcggctgccgagcactttgccctgacgaatgaacaatacgtggacacaagccatacccgcatcgaacgccacaccaataacgtcaaggtcgtcgccggtattggaaacaaactgtttttcggtgacacgtcgcaccgcctgaatttggtcgcgaatccgcgccgcttcttcaaactccagattctggctggcagtttccatacgactaatgagttgcgtaagcacctgatcatctttgccagacaaaaacaggcgcacatactcgacctgctgagcgtattcttcttcactcaccagtccttcaacgcacggccccagacagcgccctatctgatattgcagacacggacgcgagcggttgcgataaacactgttttcgcactggcgaatggggaaaatcttttgcagtagcgccagtgtttcacgtacggcatagccattcgggaacgggccgaaatattcacctttggcatgcttcgctccacgatgcatcgccagacgcgggtgggtatcgccactcaggaagataaaaggatatgatttatcatcgcgtagcaaaacgttgtaacgcggctgataaagtttgatgtagttgtgttccagtaacagcgcttcggtttctgtatgagtaaccgttacatcaatttgctggatctgggcgaccagcgcttcggttttgcgcgaagcgaggttgctacggaaatagctggaaagccgttttttcaggtctttcgctttgccgacatagataaccgtaccaccagcatcgtacatgcgataaacgcctggctggctggttacggtttttaaaaacgcttttgcgtcaaactgatcactcac

SEQ ID NO: 124 - protein, 132 aa, Pinus pinaster
MSWQTYVDDHLMCEIGRGNRLTAAAIIGQDGSVWAQSDSFPAIKPEEVTAIVNDFAEPGSLAPTGLYIGGTKYMVIQGEPGAVIRGKKGSAGVTIKKTTCALIFGLYDEPVTPGECNMIVERLGDYLIDQGI

SEQ ID NO: 125 - DNA, 399 bp, Pinus pinaster
atgtcgtggcagacttacgtcgatgaccatctcatgtgcgagatcgggcggggaaatcggctcacagcggcggccattattgggcaggatggcagcgtttgggctcagagcgatagcttcccagcgatcaagcctgaggaagtgacggccatagtgaatgatttcgcggagcctggatcccttgcaccaactggcctgtatattggaggaacaaagtatatggtcatccaaggtgaacctggagctgtcatcagagggaagaaaggatctgcaggtgtcaccattaagaagacaacgtgtgcacttatctttggcctttatgatgaaccggtgactccaggagaatgtaatatgattgtggagaggcttggtgactatcttattgaccagggcatctaa

SEQ ID NO: 126 - protein, 133 aa, Betula verrucosa; identical to SEQ ID NO: 16

SEQ ID NO: 127 - DNA, 402 bp, Betula verrucosa
atgtcgtggcaaacgtacgtggatgaacatttgatgtgcgatatcgacgggcaagccagcaactcgctggcatctgcgatcgtcggtcacgatggctctgtgtgggcccagagctcttccttcccacagtttaagcctcaggaaatcactggtatcatgaaggactttgaggagccgggtcatcttgctccgacgggcttacaccttgggggcataaaatacatggtcatccagggagaggctggtgctgtcatccgtggaaagaagggatctggaggtattactataaagaagactggtcaagctctcgtttttggcatctatgaagagcctgtgacaccaggacagtgcaacatggttgttgagaggttgggggattaccttattgaccagggcctgtag

SEQ ID NO: 128 - protein, 35 aa, Escherichia coli; identical to SEQ ID NO: 47

SEQ ID NO: 129 - protein, 495 aa, Pan troglodytes
WLSIRCNFLQNVGVLADIFPDLVHGPSSGDAWALDMLDLSFNSRLKLASPGAFQILKLGTLNLDHTKMKADALVGLGLQRLDALTLTDMAELPARMVAHFELQELNLGINRTRHIALEGLASCHSLKSSGLQSNGLIELPRGFLAAMARLQRLNLANNQLRSTMLCMNETGFVSGLWALDLSKNRLCTLSPVIFSCLPHLRELLLQGNQLVCLKGQVFQGLQRLQTLNLGSNPLVTLGEGWLAPLPTLTTQNLVGTHMVLSPTWGFRGPESLHSLRIQFPFGPAGVAFSLLTRLTSLELHAVSGMKHWRLSPNVFPVLQILTLKGWGLQLETQNISKIFPALHQLSLLGSRLEPLCSQDTSSFFLWQLPKLKSLKGWGNRHSPRPYCITGLPSLQELKLQALQSQARPCPVRLEELGGELPRLDMLQLSQTGLETLSAAAFGGLGSLQVLVLDREKDLMLDDSLQEHSPRMPQYIYILTSSLACQCANACVGPWL

SEQ ID NO: 130 - protein, 69 aa, Pan troglodytes
QSPRTYMHIVSQQLCHSEAGGHSKNLFFPFLWSHCPKTLGLELFLRSSALLLLLVSLPFLKEARNSWIL

SEQ ID NO: 131 - protein, 98 aa, Pan troglodytes
LKALLRVWFQSPRSQKGKGKRFLYDVFVSHCRQDQGWMVQELLPALEDCPPAGRGLPLCLHEWDFEPGKDVADNAAESMVGSWVTLCVLSHQALHTPC

SEQ ID NO: 132 - protein, 31 aa, Pan troglodytes
CLELLLATSFLLAAPHPPGLLLVFLEPILRH

SEQ ID NO: 133 - protein, 12 aa, Pan troglodytes
LPCCHRLAWLLR

SEQ ID NO: 134 - protein, 30 aa, Pan troglodytes
RDYCMWPKEEERKNDFWAWLGSRLEQPGVG

SEQ ID NO: 135 - protein, 495 aa, Homo sapiens
WLSIRCNFLQNVGVLADIFPDLVHGPSSGDAWALDMLDLSFNSRLKLASPGAFQVLKLGTLNLDHTKMKADALVGRGLQRLDALTLTDMAELPARMVAHFELQELNLGINRTRHIALEGLASCHSLKSSGLRSNGLIELPRGFLAAMPRLQRLNLANNQLRSAMLCMNETGFVSGLWALDLSKNRLCTLSPVIFSCLPHLRELLLQGNQLVCLKDQVFQGLQRLQTLNLGNNPLVTLGEGWLAPLPTLTTQNLVGTHMVLSPTWGFRGPESLHSLRIQFPFGPAGVAFSLLTRLTSLELHAVSGMKHWRLSPNVFPVLQILTLKGWGLQLETQNISKIFPALHQLSLLGSRLEPLCSQDTSSFFLWQLPKLKSLKGWGNRHSPRPYCITGLPSLQELKLQALQSQACPCPVRLEELVGELPRLDMLQLSQTGLETLSAAAFGGLGSLQVLVLDREKDFMLDDSLQEHSPRMPQYIYILTSSLACQCANACVGPWL

SEQ ID NO: 136 - protein, 69 aa, Homo sapiens; identical to SEQ ID NO: 130

SEQ ID NO: 137 - protein, 98 aa, Homo sapiens
LKALLRVWFQSLRSQKGKGKRFLYDVFVSHCRQDQGWMVQELLPALEDCPPAGRGLPLCLHEWDFEPGKDVADNAADSMVGSWVTLCVLSHQALHTPC

SEQ ID NO: 138 - protein, 31 aa, Homo sapiens
CLELLLATSFLLAVPHPPGLLLVFLEPILRH

SEQ ID NO: 139 - protein, 12 aa, Homo sapiens
LPCCHRLAWLLR

SEQ ID NO: 140 - protein, 30 aa, Homo sapiens
RDYCMWPKEEERKNDFWAWLGSRLEHPGVG

(19) United States
(12) Patent Application Publication
(10) Pub. No.: US 2008/0038250 A1
(43) Pub. Date: Feb. 14, 2008

(54) Profilin and related immunomodulatory ligands

(75) Inventors: Igor Zlatkin, Lansing, MI (US); J. Justin McCormick, Port Austin, MI (US)

Correspondence Address: WILMERHALE/BOSTON, 60 STATE STREET, BOSTON, MA, 02109, US

(73) Assignee: Michigan State University, East Lansing, MI (US)

(21) Appl. No.: 11/641,084

(22) Filed: Dec. 18, 2006

Related U.S. Application Data
(60) Provisional application No. 60/751,195, filed on Dec. 16, 2005.
(60) Provisional application No. 60/801,036, filed on May 17, 2006.

(51) Int. Cl. A61K 31/70; A61K 39/00; A61P 37/00; C07K 16/00; C12Q 1/68

(52) U.S. Cl. 424/130.1; 424/141.1; 424/191.1; 435/6; 514/44; 514/9; 530/350

(57) ABSTRACT

The invention provides profilin-related immunomodulatory polypeptides and toll-like receptor agonists, as well as related pharmaceutical compositions and methods of treatment, useful for treating cancer and infectious disease.

### CROSS REFERENCE TO RELATED APPLICATIONS

This application claims the benefit of priority to U.S. Provisional Application No. 60/801,036, filed May 17, 2006, and to U.S. Provisional Application No. 60/751,195, filed Dec. 16, 2005.

### FIELD OF THE INVENTION

The invention is in the fields of medical science and immunology. More specifically, the invention relates to the treatment of cancer and infectious disease using immunomodulatory proteins derived from bacteria, protozoa, plants and other organisms, as well as synthetic immunomodulatory ligands that mimic the effects of these proteins.

### 1. BACKGROUND OF THE INVENTION

The effective treatment of cancer and infectious disease presents a continuing challenge to medical science. Traditional therapies for these diseases are not always successful and are severely limited in their applicability and/or effectiveness. Furthermore, despite the development of many effective new drug treatments for both cancer and infectious disease, drug-resistant varieties of these diseases develop and confound effective treatment.

For example, while surgical procedures have been developed and used to treat patients whose tumors are confined to particular anatomical sites, only about 25% of patients have tumors that are truly confined and amenable to surgical treatment alone at the time of diagnosis (Slapak et al. (1994) in Harrison's Principles of Internal Medicine, Isselbacher et al., eds. McGraw-Hill, Inc., NY pp. 1826-1850). Similarly, radiation therapy is also not always successful. Radiation therapy is a localized treatment strategy, and its usefulness in the treatment of cancer depends to a large extent on the inherent radiosensitivity of the tumor and adjacent normal tissues. Furthermore, radiation therapy is associated with both acute toxicity and long-term aftereffects and complications. Indeed, radiation therapy is known to be mutagenic, carcinogenic, and teratogenic (Slapak et al., ibid.). Chemotherapy is still another type of cancer therapy. Systemic chemotherapy alone or in combination with surgery and/or radiation therapy is a primary treatment available for disseminated malignancies. Most chemotherapeutic agents are designed to treat cancer by specifically targeting rapidly dividing cells (e.g., by blocking DNA replication), however this strategy causes unwanted side effects in many normal cell types. This lack of specificity of chemotherapeutic agents for neoplastic cells accounts for their systemic toxicity. Accordingly, there is a need for better strategies for treating cancer.

Similarly, there is a need for additional methods to treat infectious diseases in humans and other animals caused by numerous organisms, including bacteria, viruses, and protozoa. Current therapies for infectious diseases, particularly infectious diseases caused by bacteria, include the use of one or more antibiotics. However, effective antibiotics are not available for all types of infectious bacteria, and continued use of antibiotics can lead to the development of antibiotic-resistant infections. Furthermore, safe and effective chemotherapeutic agents targeting infectious viruses and protozoa are particularly difficult to identify and develop. Accordingly, there is further a need for new strategies for treating infectious diseases

### 2. SUMMARY OF THE INVENTION

Aspects of the invention provide novel immunomodulatory compositions for use in the treatment of cancer and infectious disease. The invention is based, in part, upon the discovery of a class of immunomodulatory proteins that are structurally related to the profilin-like Eimeria tenella Apicomplexa-related protein (ARP) described in WO2005/010040 and US 2005/169935 A1, the contents of both of which are incorporated herein by reference in their entirety. The novel immunomodulatory proteins of the invention include new protozoan profilin-related proteins, as well as profilin, profilin-related immunomodulatory polypeptides (PRIPs) and profilin-like immunomodulatory proteins (PLIPs), from bacteria, plants and other organisms. Further aspects of the invention provide synthetic immunomodulatory ligands, such as antibodies, aptamers, small molecules, and peptidomimetics that target toll-like receptors responsive to PRIPs (e.g., TLR11/TLR12 and/or TLR5).

Accordingly, aspects of the invention are based, in part, upon the discovery of important structural features identifying numerous previously-unrecognized immunomodulatory PRIPs, as well as the recognition of a cellular target of these polypeptides and a class of target agonists with profilin-like immunomodulatory activity. It has been discovered that there are additional amino acid and nucleic acid sequences related to the Eimeria tenella profilin-related proteins, and that compositions and preparations containing these sequences can be used to treat cancer and/or infectious diseases in humans and other animals.

Various aspects of the instant invention provide chemically unique therapeutic compositions, including new members of a class of structurally-related polypeptides as well as unique TLR11/TLR12 and/or TLR5-targeting compositions.

In certain aspects, the invention provides an isolated immunomodulatory polypeptide encoded by a nucleic acid that hybridizes under stringent conditions to a nucleic acid encoding a protozoan profilin-related immunomodulatory polypeptide. In some embodiments, this protozoan nucleic acid is SEQ ID NO: 7 (from N. caninum), SEQ ID NO: 8 (from S. neurona), SEQ ID NO: 9 (from T. gondii) or SEQ ID NO: 10 (from P. falciparum). In some embodiments, the isolated immunomodulatory polypeptide has a toll-like receptor agonist activity. In particular embodiments, the toll-like receptor to which it has agonist activity is TLR11, TLR12, or TLR5. In other embodiments, the immunomodulatory polypeptide causes an increase in the level of IL-12 when administered to a subject (e.g., a mammalian subject generally, including non-human animals, as well as human subjects in particular). In still other embodiments, the immunomodulatory polypeptide stimulates Interleukin-12 (IL-12) synthesis in dendritic cells (DCs). In certain embodiments, the isolated immunomodulatory polypeptide is encoded by a nucleic acid that hybridizes under stringent conditions that include a hybridization occurring at 65° C. in 4×SSC. In other useful embodiments, the isolated immunomodulatory polypeptide is encoded by a nucleic acid that hybridizes under stringent conditions that further include a washing step at 65° C. in 1×SSC.

In another aspect, the invention provides an isolated profilin-related immunomodulatory polypeptide encoded by a nucleic acid that hybridizes under stringent conditions to a nucleic acid encoding a polypeptide having an amino acid sequence corresponding to any of SEQ ID NOS: 1-4 (corresponding to profilin-related immunomodulatory polypeptides from N. caninum, S. neurona, T. gondii and P. falciparum). In some embodiments, the isolated profilin-related immunomodulatory polypeptide encoded by a nucleic acid that hybridizes to the encoding nucleic acid sequences under stringent conditions that include hybridization at 65° C. in 4×SSC. In further embodiments, the stringent hybridization conditions further include washing at 65° C. in 1×SSC.

In a further aspect, the invention provides an isolated immunomodulatory polypeptide corresponding to any of SEQ ID NOS: 1-4 (corresponding to profilin-related immunomodulatory polypeptides from N. caninum, S. neurona, T. gondii and P. falciparum, respectively). In particular embodiments, the isolated immunomodulatory polypeptide, when transgenically expressed in a human HT1080 fibrosarcoma cell line, causes a delay and/or reduced tumor growth in an implanted athymic mouse.

In still another aspect, the invention provides an isolated profilin-related immunomodulatory polypeptide encoded by a nucleic acid that hybridizes under stringent conditions to a plant profilin-encoding nucleic acid. In some embodiments the plant profilin-encoding nucleic acid is B. nigra or P. banksiana nucleic acid. In particular embodiments, the isolated profilin-related immunomodulatory polypeptide is encoded by a nucleic acid that hybridizes to the encoding nucleic acid sequences under stringent conditions that include hybridization at 65° C. in 4×SSC. In further embodiments, the stringent hybridization conditions further include washing at 65° C. in 1×SSC.

In still further aspects, the invention provides an isolated immunomodulatory polypeptide from B. nigra or from P. banksiana.

In yet further aspects, the invention provides an isolated immunomodulatory polypeptide from a bacteria. In some embodiments the bacterial immunomodulatory polypeptides are an isolated profilin-related immunomodulatory UvrBC polypeptide complex comprising a UvrB polypeptide and a UvrC polypeptide. In certain embodiments, the isolated profilin-related immunomodulatory UvrBC polypeptide complex includes a UvrB polypeptide having the contiguous sequence MVLAPNKTLAAQLYGEMKEFFPENAVEYFV-SYYDY (SEQ ID NO: ______) and/or a UvrC polypeptide having the contiguous sequence KAIDDSKIPDVILIDGG-KGQLAQAKNVAELDVSWDKNHPLLLGVAKGA (SEQ ID NO: ______)-. In other embodiments, the isolated profilin-related immunomodulatory UvrBC polypeptide complex includes a UvrB polypeptide having the sequence of SEQ ID NO: 30 (E. coli UvrB subunit in FIG. 12) and/or a UvrC polypeptide having the sequence of SEQ ID NO: 32 (E. coli UvrC subunit in FIG. 12).

In further aspects, the invention provides an isolated immunomodulatory fusion polypeptide encoded by a nucleic acid that hybridizes under stringent conditions to a nucleic acid encoding a protozoan profilin-related, PA19-like immunomodulatory polypeptide. IN some embodiments the nucleic acid encodes the fusion polypeptide such as SEQ ID NO: 7 (from N. caninum), SEQ ID NO: 8 (from S. neurona), SEQ ID NO: 9 (from T. gondii) or SEQ ID NO: 10 (from P. falciparum), and that is further fused to an heterologous polypeptide sequence. In certain embodiments, the heterologous polypeptide sequence includes the sequence pre-pro-trypsin. In other embodiments, the heterologous polypeptide sequence includes an affinity tag. In one embodiment, the affinity tag is a FLAG tag.

In yet another aspect, the invention provides synthetic immunostimulatory TLR11/TLR12 agonists. In some embodiments the agonists are antibodies, aptamers, polypeptides, peptidomimetics, small molecules or circular polypeptides. In certain embodiments, the TLR11/TLR12 agonist is a high affinity ligand of TLR11/TLR12. In other embodiments, the immunostimulatory TLR11/TLR12 agonist causes an increase in the level of IL-12 when administered to a subject (e.g., a mammalian subject (e.g., a mouse)). In still other embodiments, the immunostimulatory TLR11/TLR12 agonist stimulates Interleukin-12 (IL-12) synthesis in dendritic cells (DCs). In particular embodiments, the immunostimulatory TLR11/TLR12 agonist is an antibody. In certain embodiments the antibody is a monoclonal antibody. In some embodiments, the antibody causes an increase in the level of IL-12 when administered to a subject. In further embodiments, the immunostimulatory TLR11/TLR12 agonist is an aptamer. In other embodiments, the immunostimulatory TLR11/TLR12 agonist is a small molecule. Instill other embodiments, the immunostimulatory TLR11/TLR12 agonist is a circular polypeptide. In further embodiments, the immunostimulatory TLR11/TLR12 agonist is a peptidomimetic.

In another aspect, the invention provides pharmaceutical formulations which include a pharmaceutically acceptable carrier in combination with an immunomodulatory polypeptide encoded by a nucleic acid that hybridizes under stringent conditions to a nucleic acid encoding a protozoan profilin-related PA19-like immunomodulatory polypeptide. In some embodiments, the nucleic acid encoding the protozoa profilin has SEQ ID NO: 7 (from N. caninum), SEQ ID NO: 8 (from S. neurona), SEQ ID NO: 9 (from T. gondii), or SEQ ID NO: 10 (from P. falciparum). In certain embodiments, the pharmaceutical formulation includes a profilin-related immunomodulatory polypeptide encoded by a nucleic acid that hybridizes under stringent conditions to a nucleic acid encoding a polypeptide having an amino acid sequence corresponding to any of SEQ ID NOS: 1-4 (corresponding to profilin-related immunomodulatory polypeptides from N. caninum, S. neurona, T. gondii and P. falciparum, respectively). In further embodiments, the pharmaceutical formulation includes an immunomodulatory polypeptide which, when transgenically expressed, causes a delay and/or reduced tumor growth in an implanted athymic mouse. In some embodiments, the transgenic expression is in a human HT1080 fibrosarcoma cell line. In still further embodiments, the pharmaceutical formulation includes a profilin-related immunomodulatory polypeptide encoded by a nucleic acid that hybridizes under stringent conditions to a plant profilin-encoding nucleic acid. In some embodiments, the plant profilin-encoding nucleic acid is from B. nigra or one from P. banksiana. In yet further embodiments, the pharmaceutical formulation includes an immunomodulatory polypeptide from B. nigra or from P. banksiana. In still further embodiments, the pharmaceutical formulation includes an immunomodulatory polypeptide from a bacteria. In some embodiments the bacterial polypeptide is an isolated profilin-related immunomodulatory UvrBC polypeptide complex comprising a UvrB polypeptide and a UvrC polypeptide. In particularly useful embodiments, the pharmaceutical formulations of the invention include a pharmaceutically acceptable carrier and a synthetic immunostimulatory TLR11/TLR12 agonist. In certain embodiments, the agonist is an antibody, an aptamer, a small molecular or a peptide or peptidomimetic.

In yet another aspect, the invention provides a method of activating TLR11/TLR12 and/or increasing the level of IL-12 in a subject, by administering to the subject an effective amount of a composition which includes a polypeptide having an amino acid sequence corresponding to any of SEQ ID NOS: 1-4 (corresponding to profilin-related immunomodulatory polypeptides from N. caninum, S. neurona, T. gondii and P. falciparum).

In another aspect, the invention provides a method of activating TLR11/TLR12 and/or increasing the level of IL-12 in a subject. In this method the subject is administered an effective amount of a composition which includes a profilin-related immunomodulatory polypeptide sequence from B. nigra or from P. banksiana.

In yet another aspect, the invention provides a method of activating TLR11/TLR12 and/or increasing the level of IL-12 in a subject, by administering to the subject an effective amount of a composition which includes a profilin-related immunostimulatory polypeptide that is encoded by a nucleic acid that hybridizes under stringent conditions to a nucleic acid corresponding to any of SEQ ID NOS:7-10 (corresponding to profilin-related immunomodulatory polypeptides from N. caninum, S. neurona, T. gondii and P. falciparum, respectively).

In still another aspect, the invention provides a method of activating TLR11/TLR12 and/or increasing the level of IL-12 in a subject, by administering to the subject an effective amount of a composition which includes a profilin-related immunostimulatory polypeptide that is encoded by a nucleic acid that hybridizes under stringent conditions to a plant nucleic acid from B. nigra or from P. banksiana. In particular embodiments, the subject is a mammal. In certain useful embodiments, the subject is a human.

In still further aspects, the invention provides a method of activating TLR11/TLR12 and/or increasing the level of IL-12 in a subject, by administering to the subject an effective amount of a composition that includes a synthetic immunostimulatory TLR11/TLR12 agonist. In some embodiments the agonist is an antibody, an aptamer, a small molecule, or a polypeptide or peptidomimetic. In certain embodiments the agonist is a circular polypeptide or peptidomimetic) agonist. In particular embodiments of the method, the subject is in need of treatment for a cancer. In further embodiments, the subject is in need of treatment for an infectious disease.

In a further aspect, the invention provides a method of treating an infectious disease in a subject, by administering to the subject an effective amount of a pharmaceutical formulation which includes a protozoan polypeptide having an amino acid sequence corresponding to any of SEQ ID NO: 1 (from N. caninum), SEQ ID NO: 2 (from S. neurona), or SEQ ID NO: 3 (from T. gondii).

In yet a further aspect, the invention provides a method of treating an infectious disease in a subject, by administering to the subject an effective amount of a composition which includes a plant polypeptide having an amino acid sequence from B. nigra or from P. banksiana.

In still another aspect, the invention provides a method of treating an infectious disease in a subject, by administering to the subject an effective amount of a profilin-related immunostimulatory polypeptide, wherein the profilin-related immunostimulatory polypeptide is encoded by a nucleic acid that hybridizes under stringent conditions to a nucleic acid corresponding to any of SEQ ID NOS:7-10.

In yet another aspect, the invention provides a method of treating an infectious disease in a subject, by administering to the subject an effective amount of a profilin-related immunostimulatory polypeptide, wherein the profilin-related immunostimulatory polypeptide is encoded by a nucleic acid that hybridizes under stringent conditions to a nucleic acid from B. nigra or from P. banksiana.

In particular embodiments of the above-described methods of the invention, the infectious disease treated is one that is caused by a virus, a bacterium, or a protozoa.

In further embodiments, the subject treated is a non-human animal. In other embodiments, the subject treated is a mammal. In a particular embodiment the mammal is a human.

In still further aspects, the invention provides a method of treating a cancer in a subject, by administering to the subject an effective amount of a pharmaceutical formulation which includes a protozoan polypeptide having an amino acid sequence corresponding to any of SEQ ID NO: 1 (from N. caninum), SEQ ID NO: 2 (from S. neurona), or SEQ ID NO: 3 (from T. gondii). In certain embodiments, the subject is a mammal. In particular embodiments, the subject is a human. In further embodiments, the cancer is a sarcoma. In particular embodiments, the sarcoma is a fibrosarcoma, such as a human fibrosarcoma. In other embodiments, the cancer is a carcinoma. In particular embodiments, the carcinoma is an ovarian carcinoma, such as a human ovarian carcinoma.

In yet a further aspect, the invention provides a method of treating a cancer in a subject, by administering to the subject an effective amount of a composition which includes a plant polypeptide having an amino acid sequence from B. nigra or from P. banksiana. In certain embodiments, the subject is a mammal. In particular embodiments, the subject is a human. In further embodiments, the cancer is a sarcoma. In particular embodiments, the sarcoma is a fibrosarcoma, such as a human fibrosarcoma. In other embodiments, the cancer is a carcinoma. In particular embodiments, the carcinoma is an ovarian carcinoma, such as a human ovarian carcinoma.

In still another aspect, the invention provides a method of treating a cancer in a subject by administering to the subject an effective amount of a profilin-related immunostimulatory fragment, wherein the profilin-related immunostimulatory polypeptide is encoded by a nucleic acid that hybridizes under stringent conditions to a nucleic acid corresponding to any of SEQ ID NOS: 7-10. In certain embodiments, the subject is a mammal. In particular embodiments, the subject is a human. In further embodiments, the cancer is a sarcoma. In particular embodiments, the sarcoma is a fibrosarcoma, such as a human fibrosarcoma. In other embodiments, the cancer is a carcinoma. In particular embodiments, the carcinoma is an ovarian carcinoma, such as a human ovarian carcinoma.

In yet another aspect, the invention provides a method of treating a cancer in a subject, by administering to the subject an effective amount of a profilin-related immunostimulatory fragment, wherein the profilin-related immunostimulatory fragment is encoded by a nucleic acid that hybridizes under stringent conditions to a nucleic acid from B. nigra or from P. banksiana. In certain embodiments, the subject is a mammal. In particular embodiments, the subject is a human. In further embodiments, the cancer is a sarcoma. In particular embodiments, the sarcoma is a fibrosarcoma, such as a human fibrosarcoma. In other embodiments, the cancer is a carcinoma. In particular embodiments, the carcinoma is an ovarian carcinoma, such as a human ovarian carcinoma.

In still another aspect, the invention provides a method of identifying a candidate subject for treatment with a profilin-related immunomodulatory polypeptide, by obtaining a cellular sample from the subject and detecting the presence of a TLR11/TLR12 polypeptide or a TLR11/TLR12-encoding nucleic acid sequence in the subject sample. By this method, the presence of the TLR11/TLR12 polypeptide or TLR11/TLR12-encoding nucleic acid sequence in the subject sample indicates that the subject is a candidate for treatment with a profilin-related immunomodulatory polypeptide. In certain embodiments, the method includes the step of detecting the presence of a TLR12 polymorphism in the subject. In some embodiments the subject is a mammal. In particular embodiments, the subject is a human.

### 3. BRIEF DESCRIPTION OF THE DRAWINGS

The present invention and the various features thereof may be more fully understood from the following description, when read together with the accompanying drawings in which:

FIG. 1A is a schematic representation of the polypeptide sequence of a N. caninum profilin-related, PA19-like polypeptide (SEQ ID NO: 1).

FIG. 1B is a schematic representation of the nucleotide sequence of a N. caninum profilin-encoding nucleic acid sequence (SEQ ID NO: 7). The initiation and termination codons of the profilin protein open reading frame (ORF) are underlined.

FIG. 2A is a schematic representation of the polypeptide sequence of a S. neurona profilin-related, PA19-like polypeptide (SEQ ID NO: 2).

FIG. 2B is a schematic representation of the nucleotide sequence of a S. neurona profilin-related polypeptide encoding nucleic acid sequence (SEQ ID NO: 8). The initiation and termination codons of the profilin-related polypeptide open reading frame are underlined.

FIG. 3A is a schematic representation of the polypeptide sequence of a T. gondii profilin-related polypeptide (SEQ ID NO: 3).

FIG. 3B is a schematic representation of the nucleotide sequence of a T. gondii profilin-related polypeptide encoding nucleic acid sequence (SEQ ID NO: 9). The initiation and termination codons of the profilin-related polypeptide open reading frame are underlined.

FIG. 4A is a schematic representation of the polypeptide sequence of a P. falciparum profilin-related polypeptide (SEQ ID NO: 4).

FIG. 4B is a schematic representation of the nucleotide sequence of a P. falciparum profilin-related polypeptide encoding nucleic acid sequence (SEQ ID NO: 10). The initiation and termination codons of the profilin-related polypeptide open reading frame are underlined.

FIG. 5A is a schematic representation of the polypeptide sequence of an Eimeria acervulina profilin-related polypeptide (SEQ ID NO: 5).

FIG. 5B is a schematic representation of the nucleotide sequence of an Eimeria acervulina profilin-related polypeptide encoding nucleic acid sequence (SEQ ID NO: 11). The initiation and termination codons of the profilin-related protein open reading frame are underlined.

FIG. 6A is a schematic representation of the polypeptide sequence of an Eimeria tenella profilin-related polypeptide (SEQ ID NO: 6).

FIG. 6B is a schematic representation of the nucleotide sequence of an Eimeria tenella profilin-encoding nucleic acid sequence (SEQ ID NO: 12). The initiation and termination codons of the profilin-related protein open reading frame are underlined.

FIG. 7A is a schematic representation of an alignment of the profilin-related polypeptide sequences of E. tenella (SEQ ID NO: 6) (at lines 3, 10 and 17) compared to the profilin-related polypeptide sequences of N. caninum (SEQ ID NO: 1) (at lines 4, 11 and 18), S. neurona (SEQ ID NO: 2) (at lines 5, 12 and 19), and T. gondii (SEQ ID NO: 3) (at lines 6, 13 and 20).

FIG. 7B is a schematic representation of a conserved profilin-related polypeptide subsequence (SEQ ID NO: 13) of N. caninum, S. neurona and T. gondii.

FIG. 7C is a schematic representation of a further conserved profilin-related polypeptide subsequence (SEQ ID NO: 14) of N. caninum, S. neurona and T. gondii.

FIG. 7D is a schematic representation of an alignment of the profilin-related polypeptide sequences of N. caninum (SEQ ID NO: 1), S. neurona (SEQ ID NO: 2), T. gondii (SEQ ID NO: 3), and P. falciparum (SEQ ID NO: 4).

FIG. 7E is a schematic representation of a conserved profilin-related polypeptide subsequence (SEQ ID NO: 1) of N. caninum, S. neurona (SEQ ID NO: 2), T. gondii (SEQ ID NO: 3), and P. falciparum (SEQ ID NO: 4).

FIG. 7F is a representation of an alignment (produce by the BioEdit program) of profilin-related polypeptides from different organisms.

FIG. 7G is a schematic representation of an alignment (produced by the BioEdit program) of profilin-related polypeptides from different organisms.

FIG. 7H is a schematic representation of the designations of the abbreviations used in FIG. 7F and FIG. 7G.

FIG. 8A is a schematic representation of the taxonomic relations between profilin-related sequences from E. tenella and other representative organisms.

FIG. 8B is a schematic representation of the taxonomic relations between profilin-related sequences from E. tenella and other representative organisms.

FIG. 9A is a diagrammatic representation of the mammalian expression vector pIRESpuro3 used for cloning the gene for the profilin-related polypeptide PA19.

FIG. 9B is a diagrammatic representation of a comparison of the structures of vector constructs used to express the profilin-related PA19 protein in HT1080 human sarcoma cells.

FIG. 9C is a graphical representation of the DCA activity of the serum collected from mice injected with HT1080 cell lines expressing, or not expressing the secreted PA19 protein.

FIG. 9D is a graphical representation of a DEAE chromatography separation profile of the medium conditioned in vitro by HT108 cell line expressing and secreting the PA19 protein.

FIG. 9E is a graphical representation of the in vivo growth of HT1080 cells transfected with vector (open figures) or vector with the gene for PA19 protein in native form (closed figures).

FIG. 9F is a graphical representation of tumor growth in athymic mice as a function of time following administration of an HT1080 cell line expressing the PA19 protein in native form.

FIG. 9G is a graphical representation of the in vivo growth of HT1080 cells transfected with vector (open symbols) or vector with the gene for PA19 in secreted form (closed symbols).

FIG. 9H is a graphical representation of an example of tumor growth in athymic mice for an HT1080 cell line expressing the PA19 protein in secreted form.

FIG. 10A is a schematic representation of the polypeptide sequence of B. pendula (European white birch) profiling (SEQ ID NO: X(16)).

FIG. 10B is a schematic representation of the polypeptide sequence of B. pendula (European white birch) profiling (SEQ ID NO: X(4)).

FIG. 11A is a schematic representation of a profilin-related polypeptide sequence from Eimeria tenella (SEQ ID NO: X(18)) showing a presequence (underlined) not shown in FIG. 6A.

FIG. 11B is a schematic representation of the nucleotide sequence (SEQ ID NO: X(19)) of a Eimeria tenella profilin-related polypeptide.

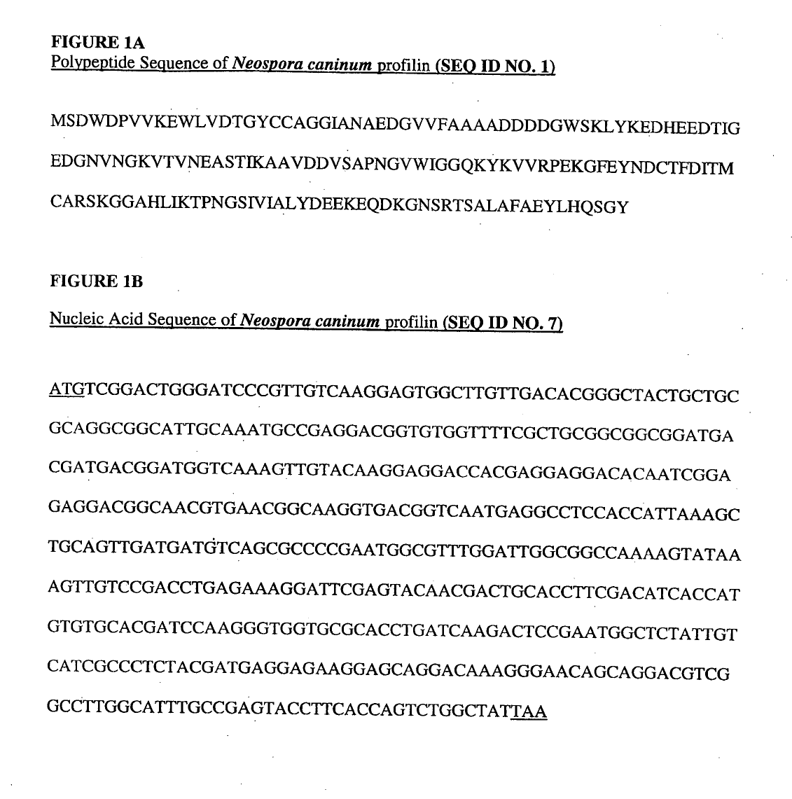

FIG. 1 IC is a schematic representation of a profilin-related polypeptide sequence from N. caninum (SEQ ID NO: X(20)) showing a presequence (underlined) not shown in FIG. 1A.

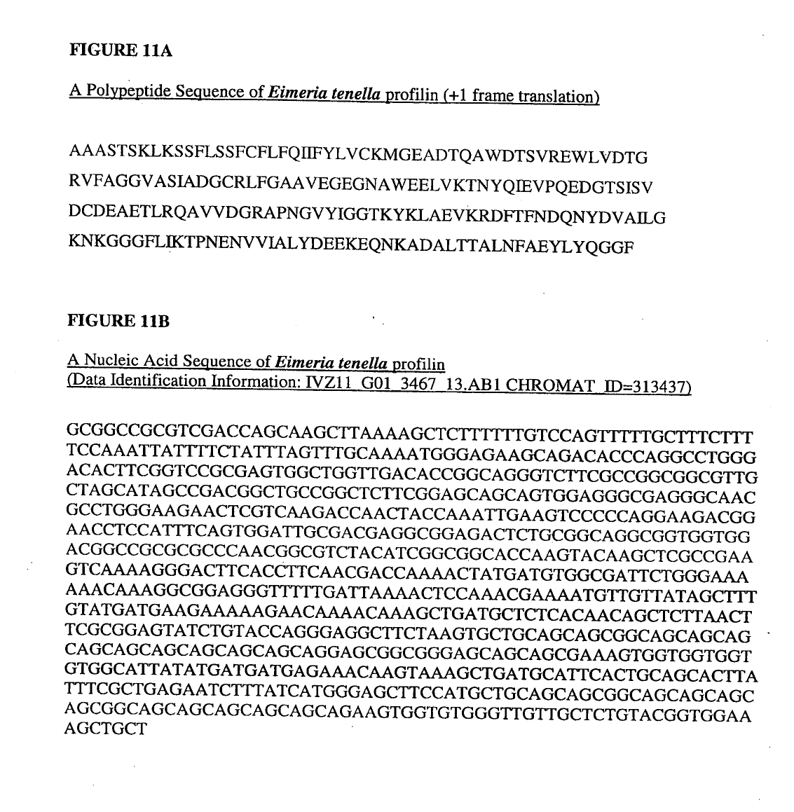

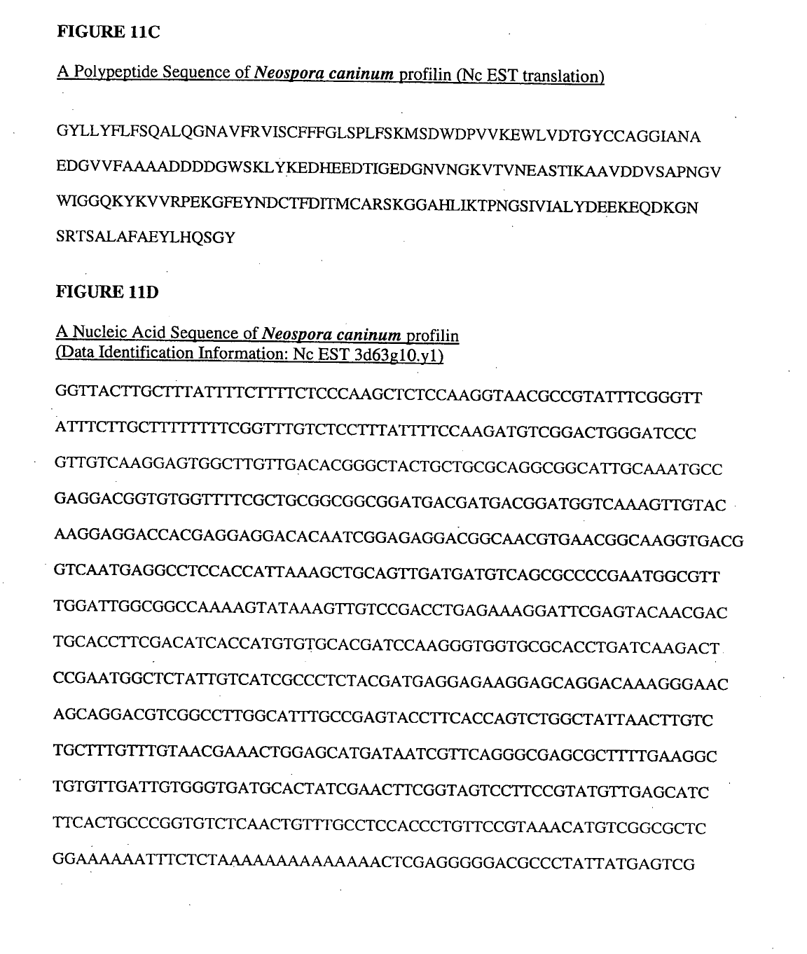

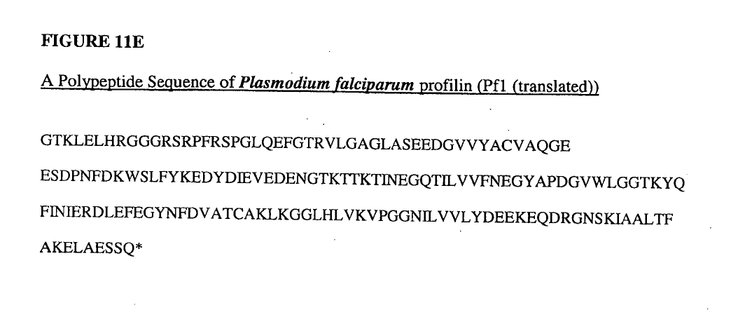

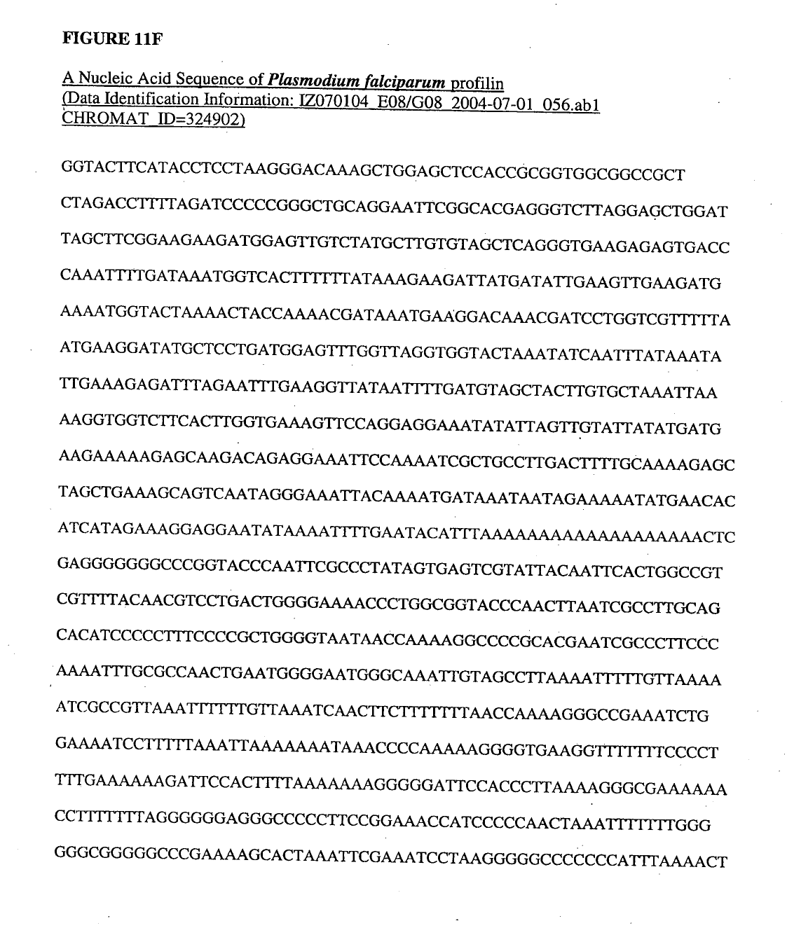

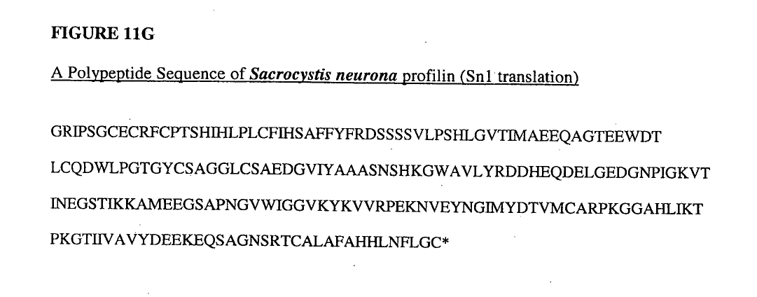

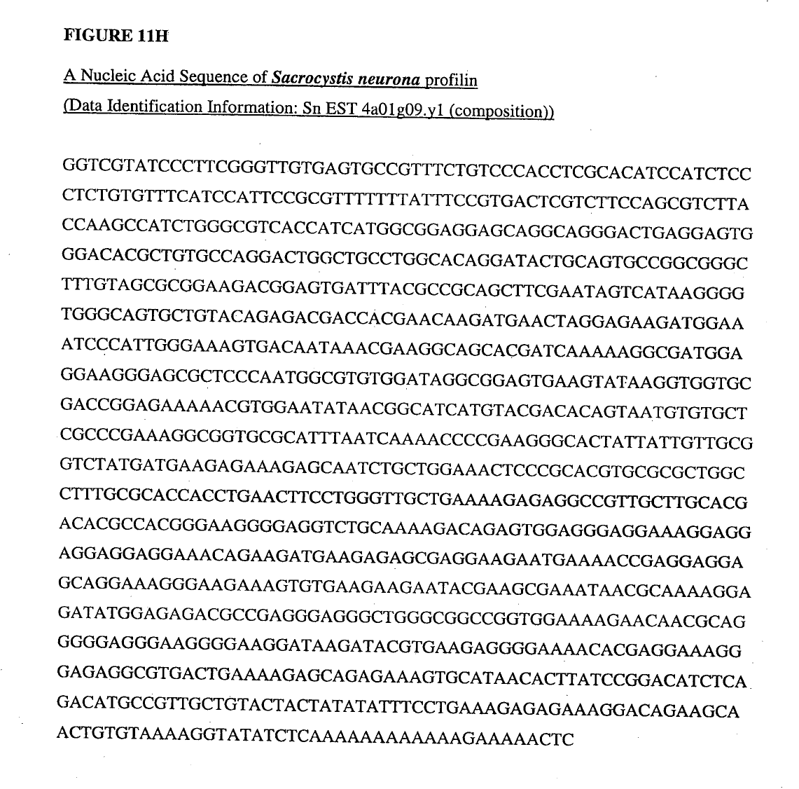

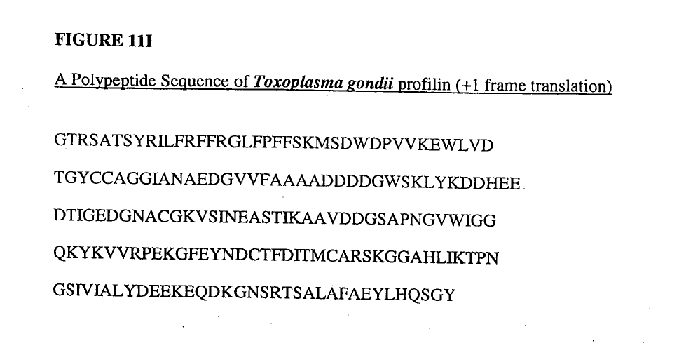

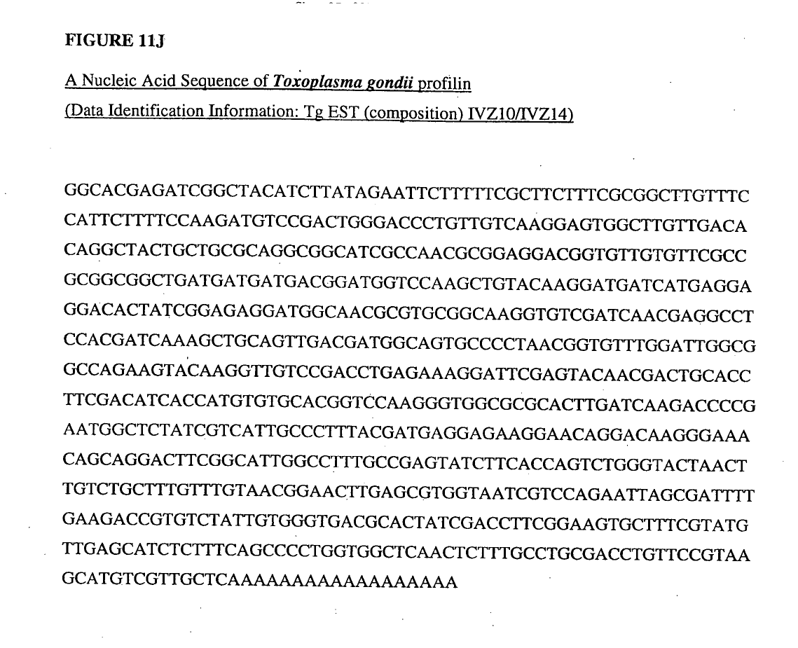

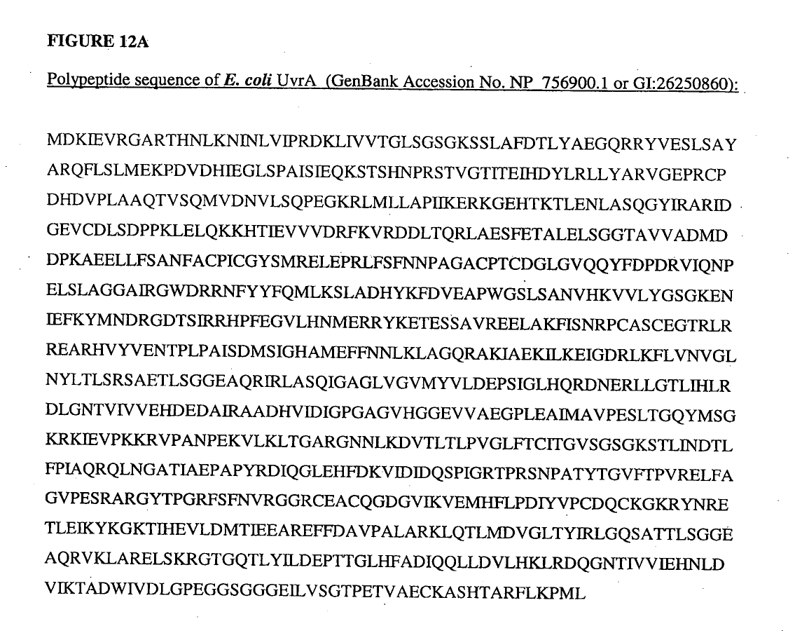

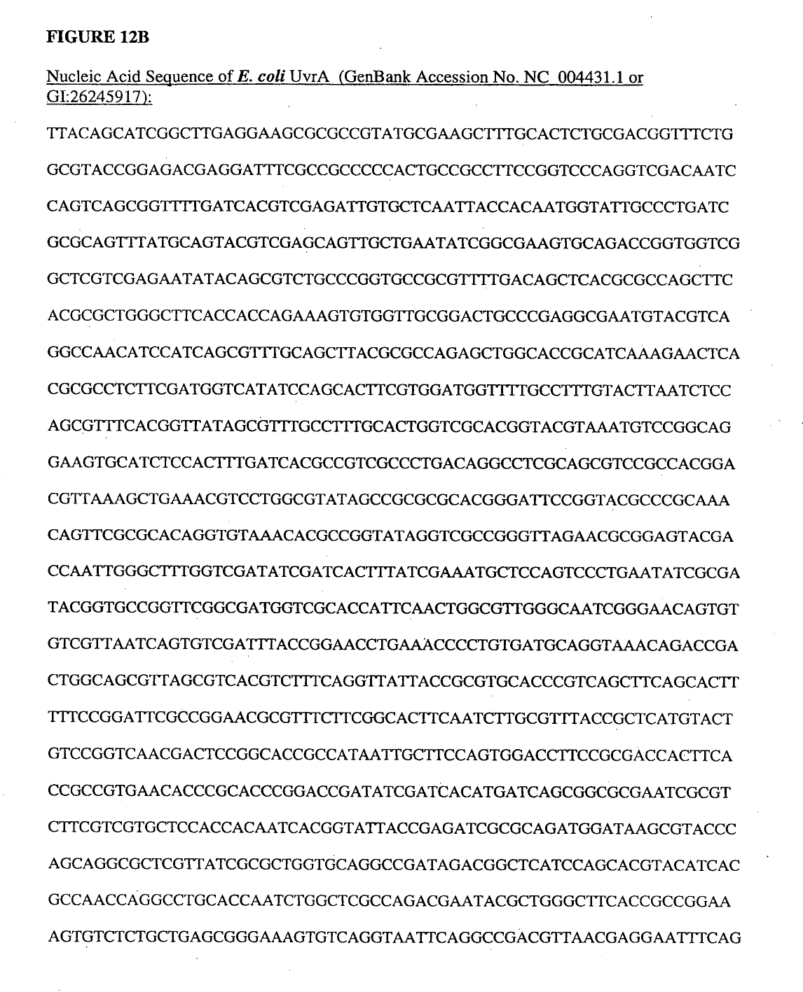

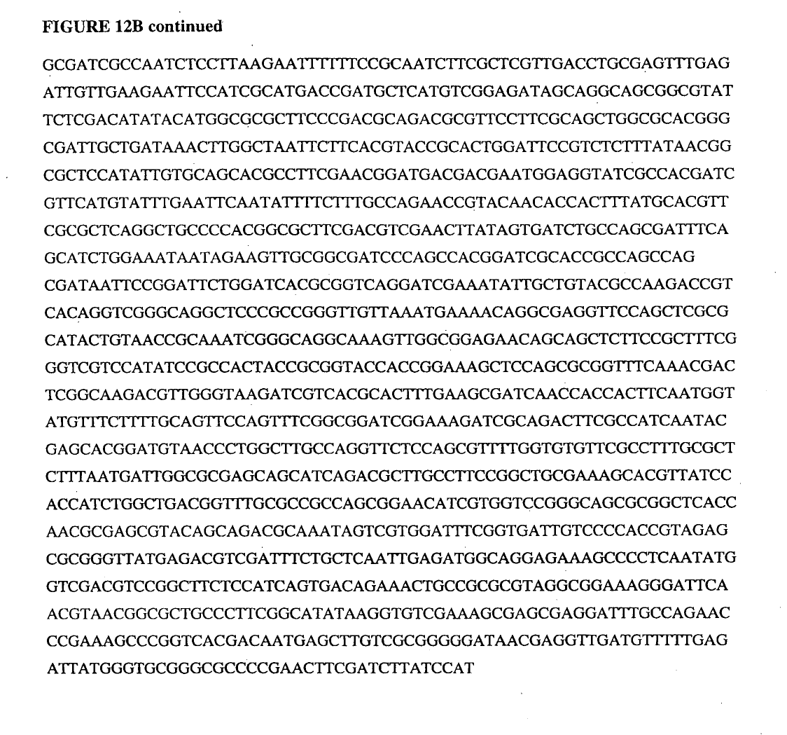

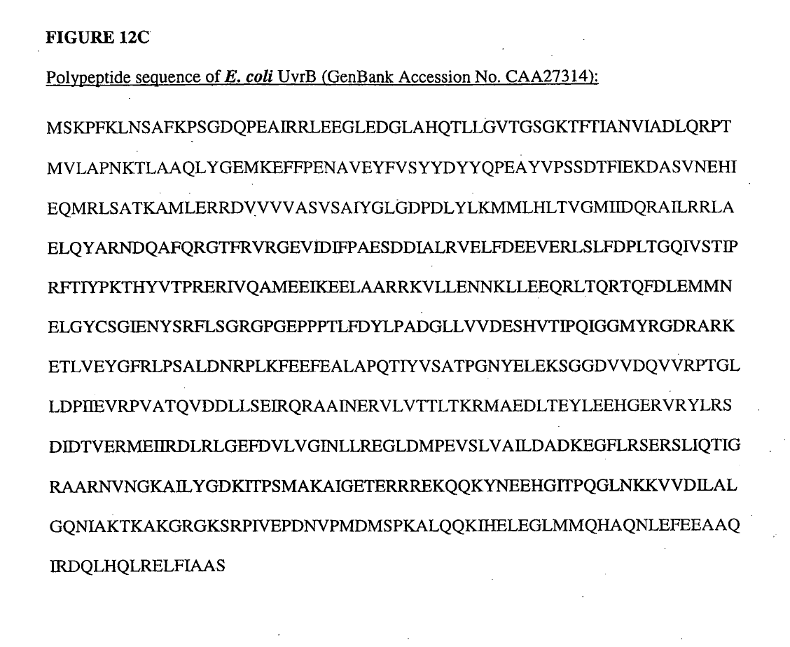

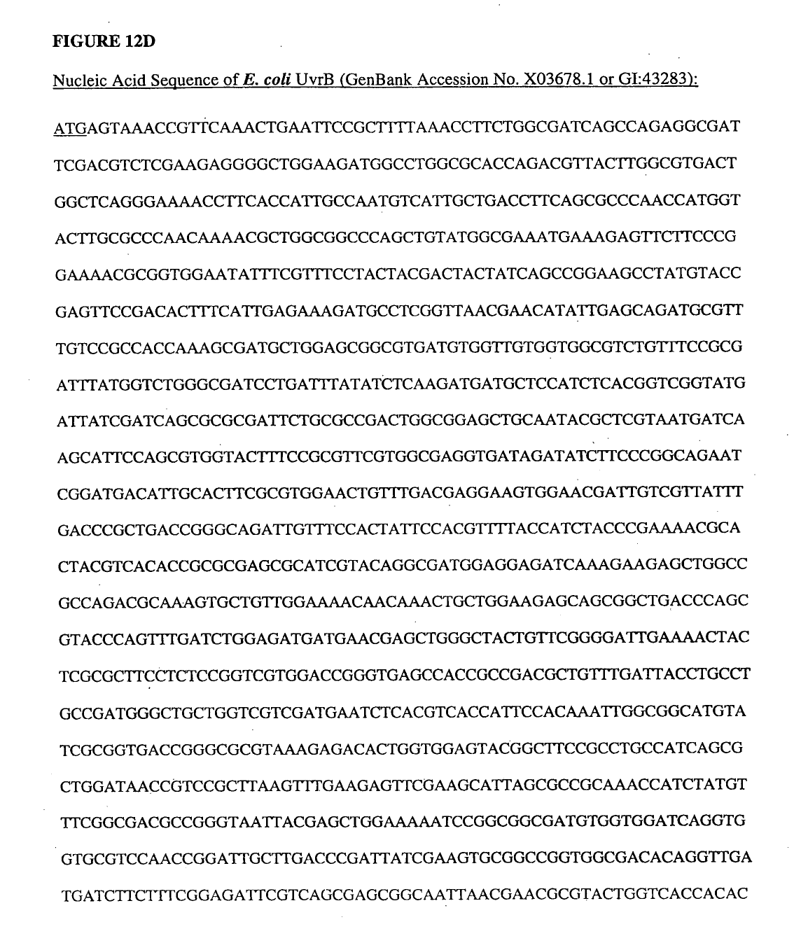

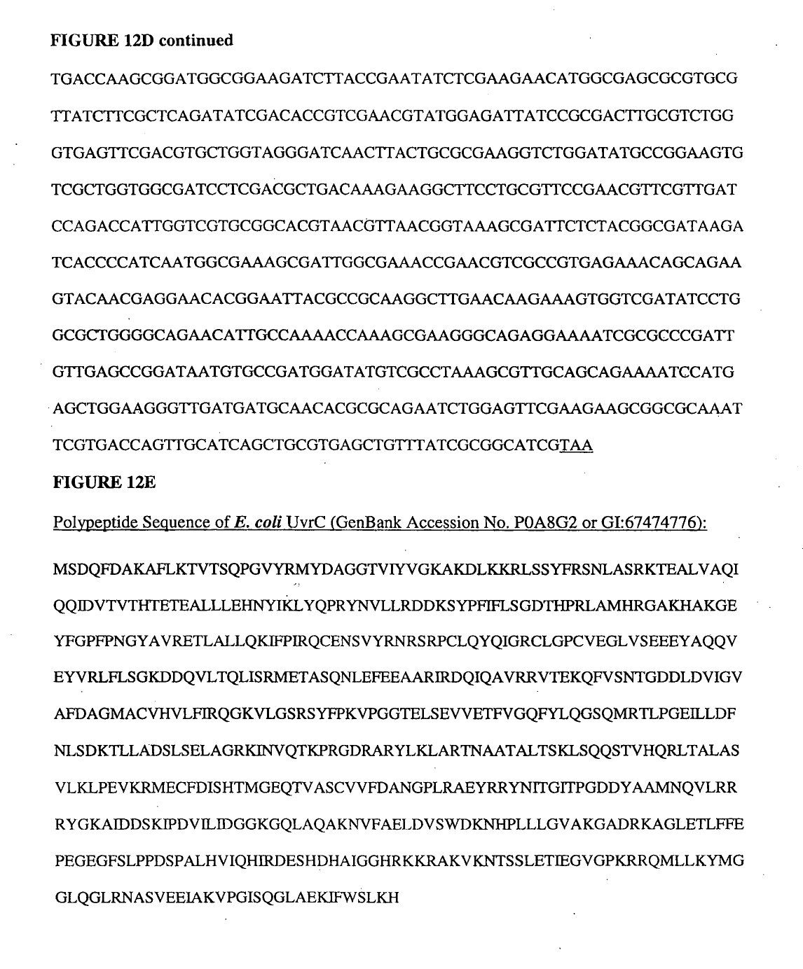

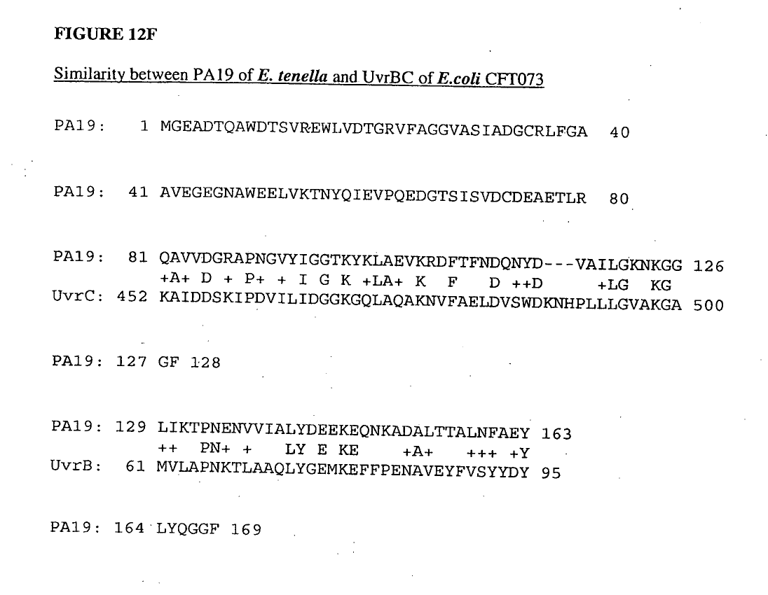

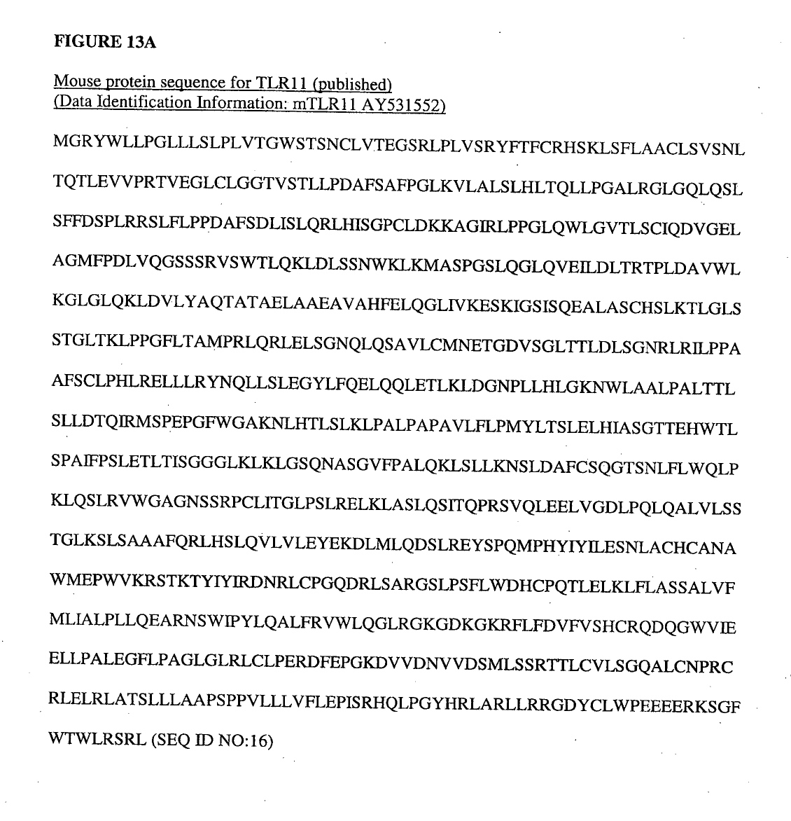

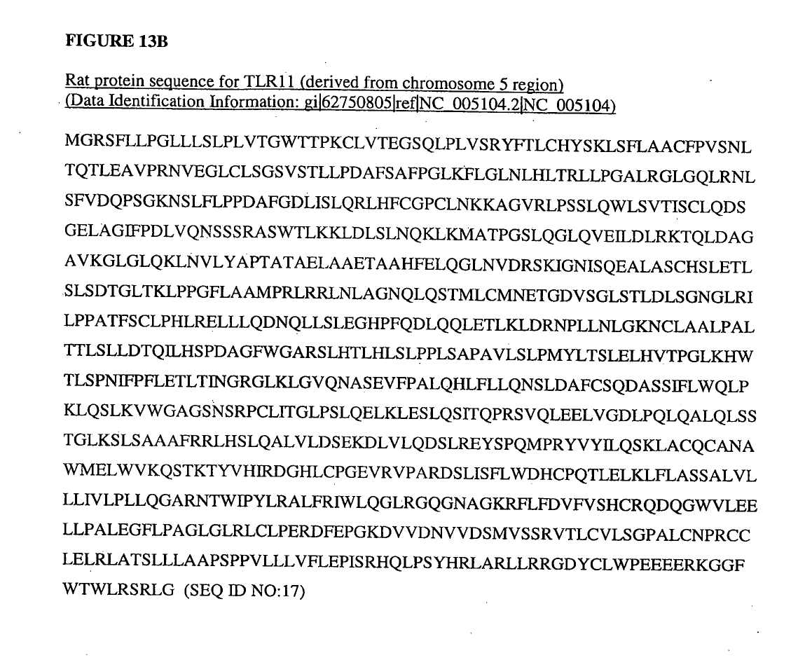

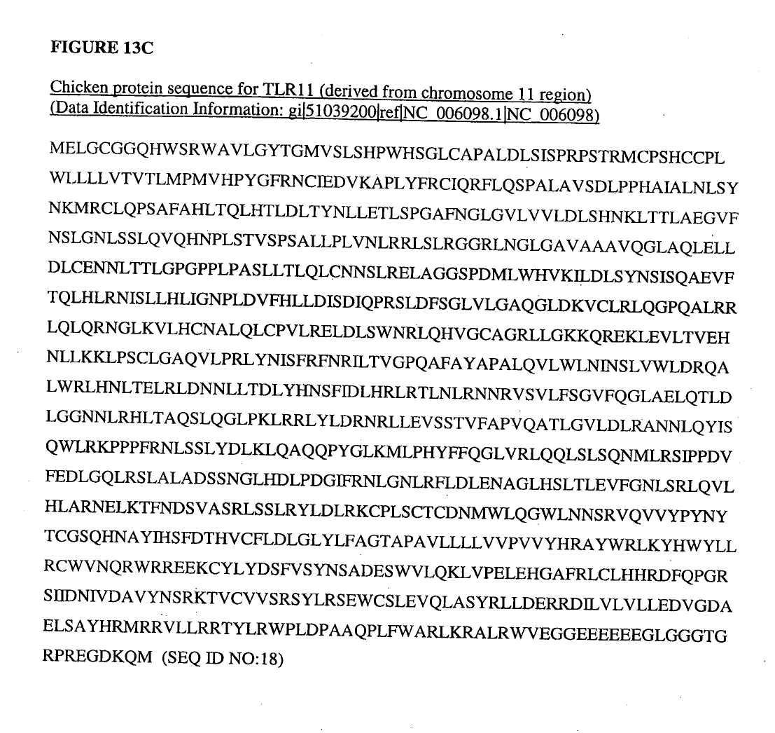

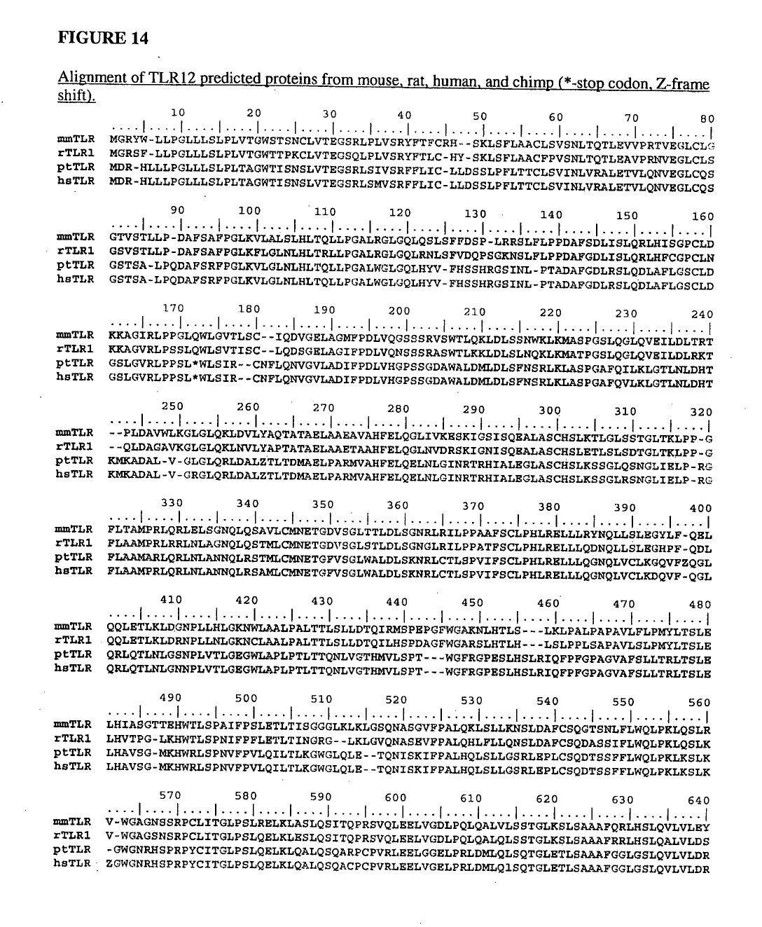

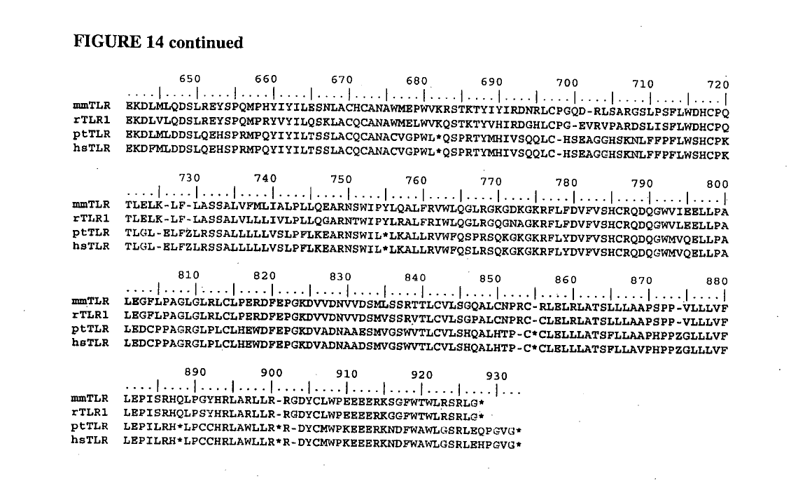

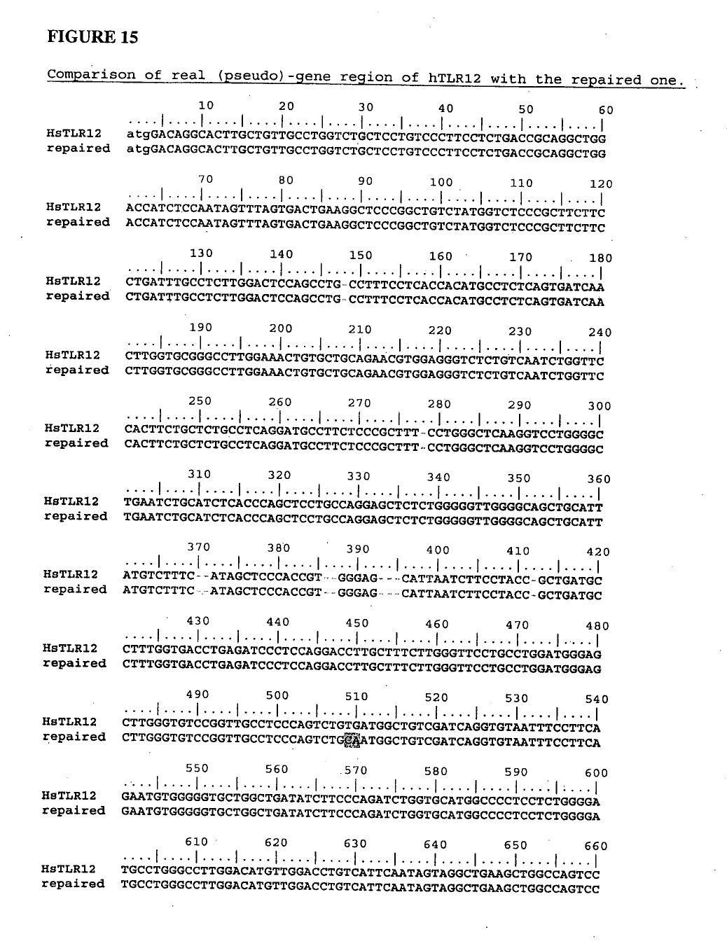

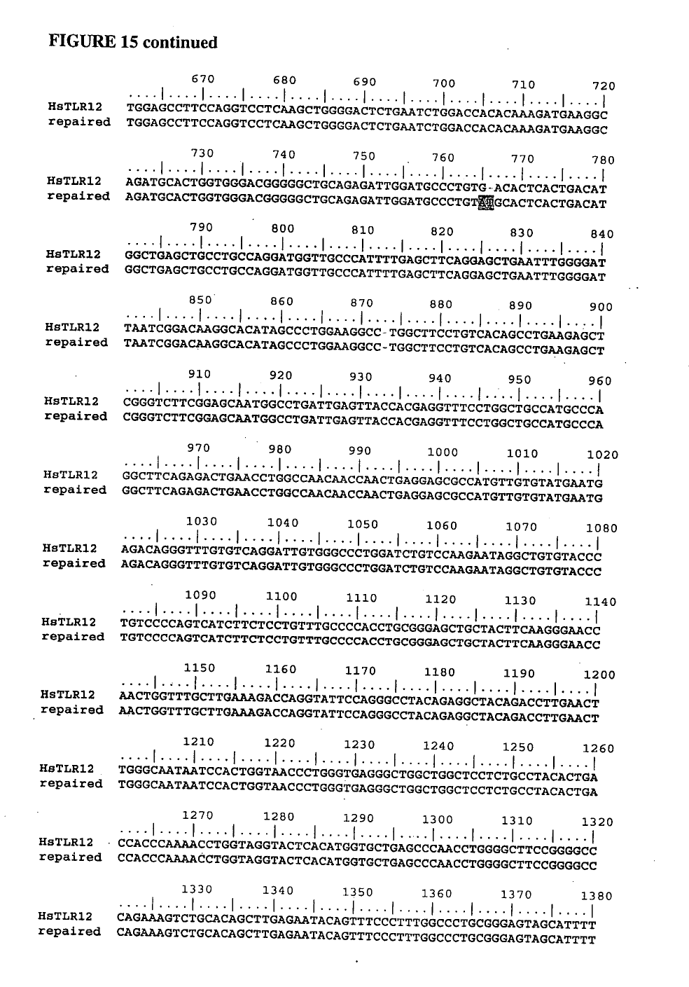

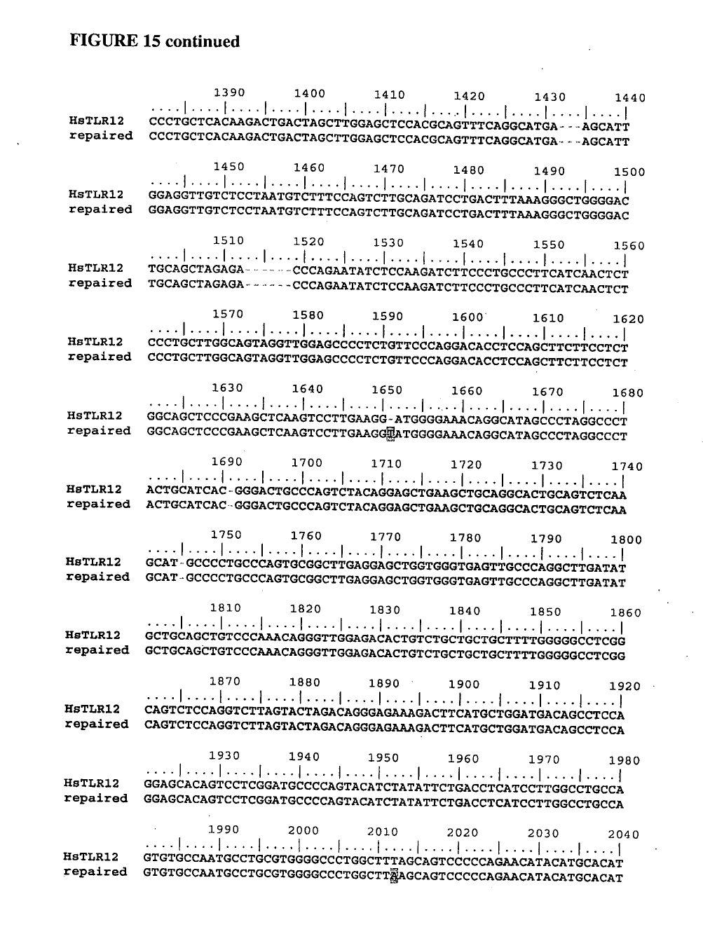

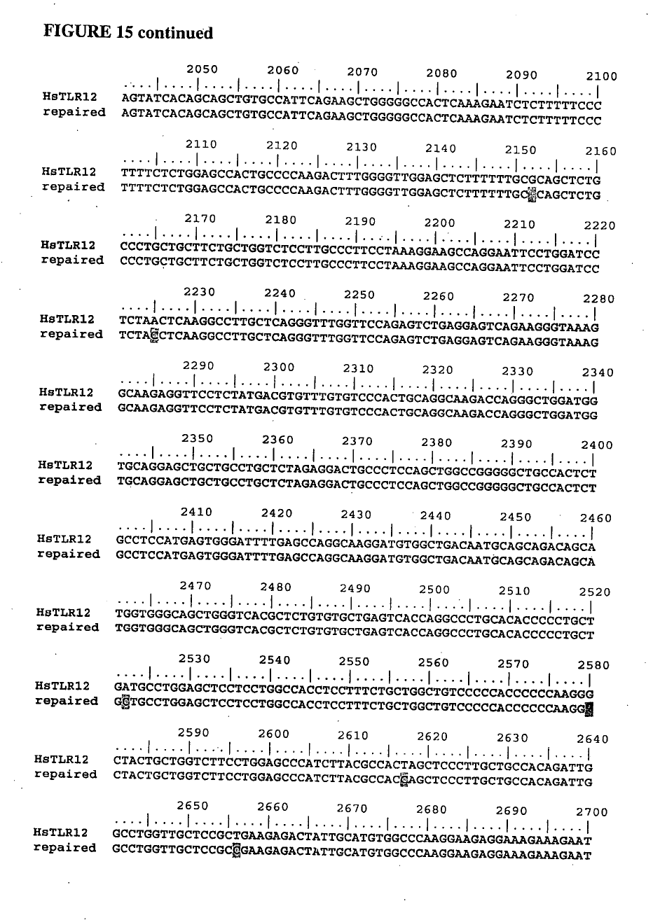

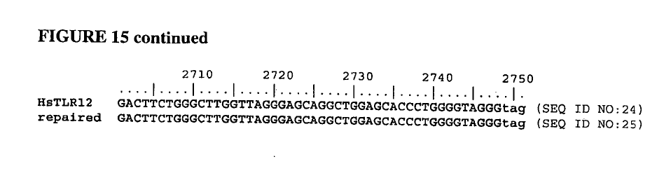

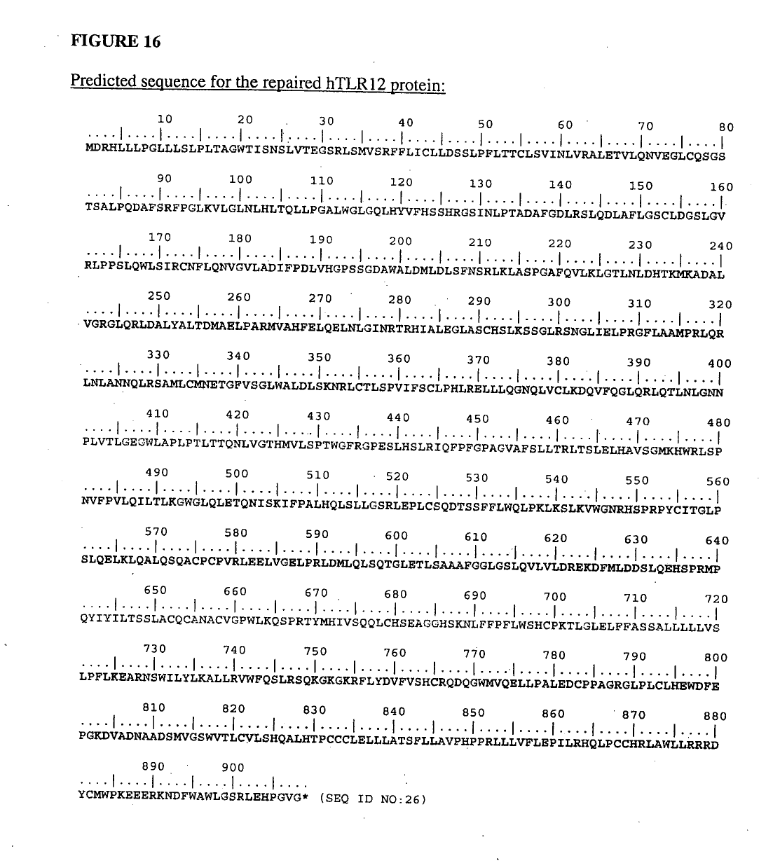

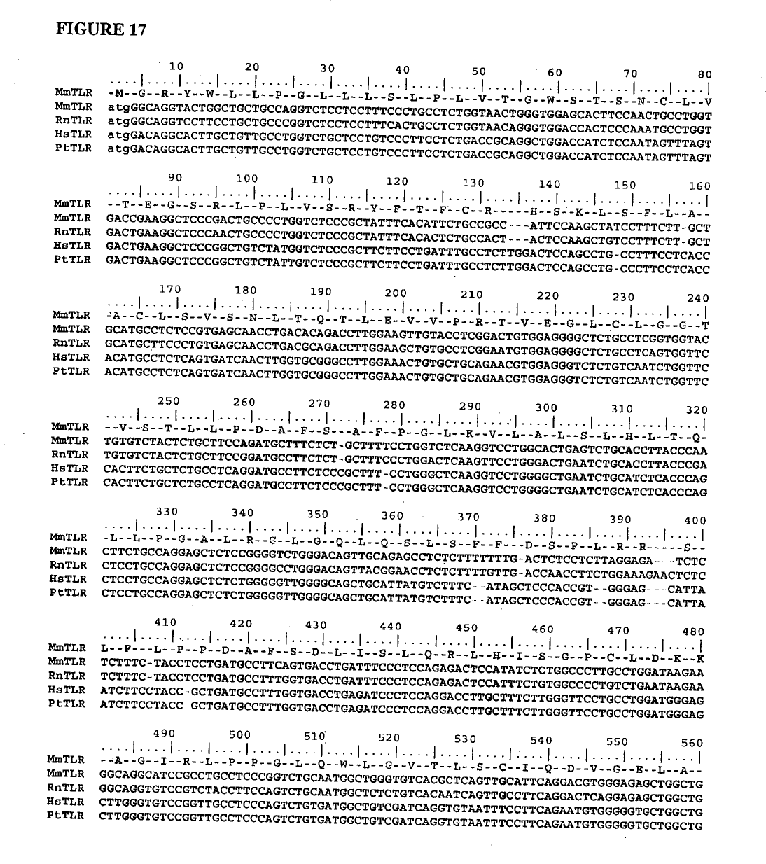

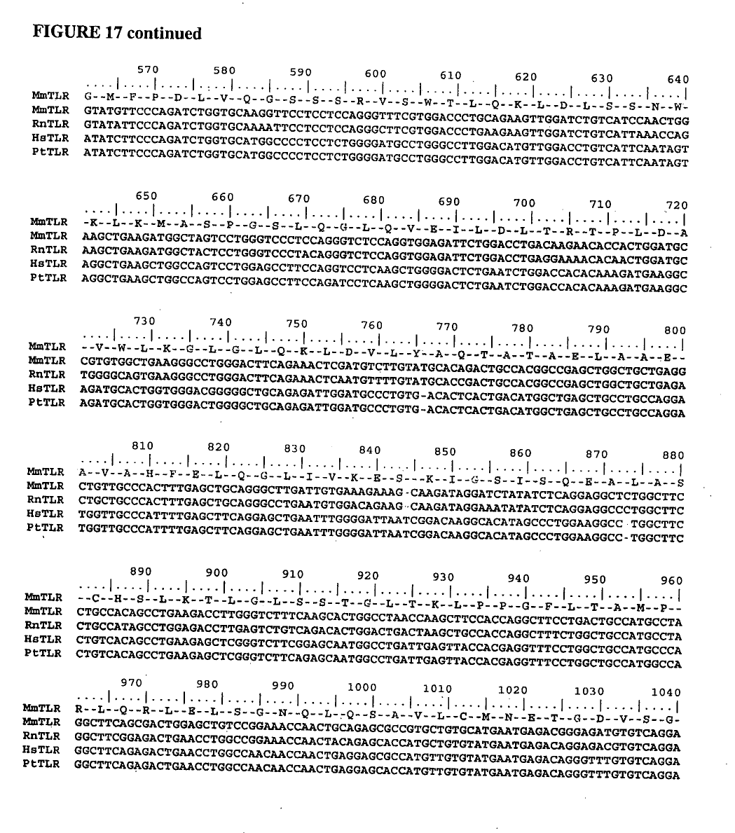

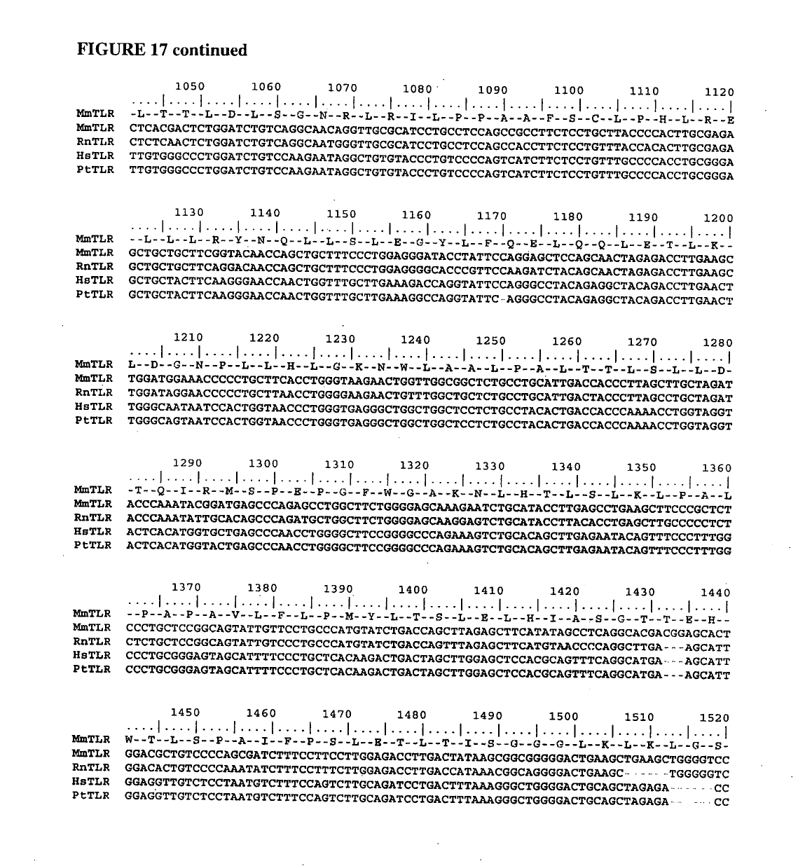

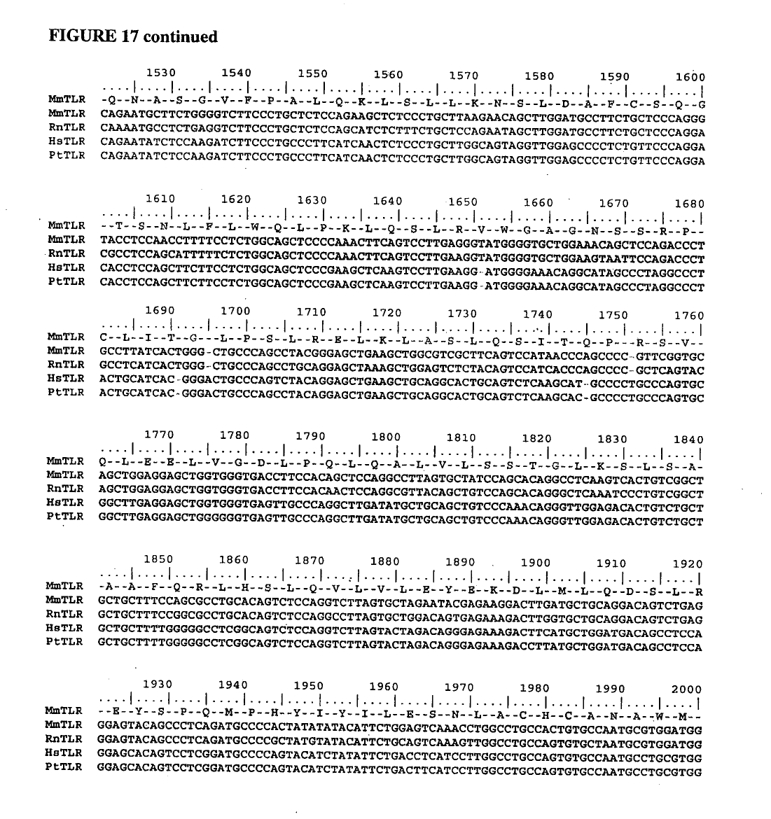

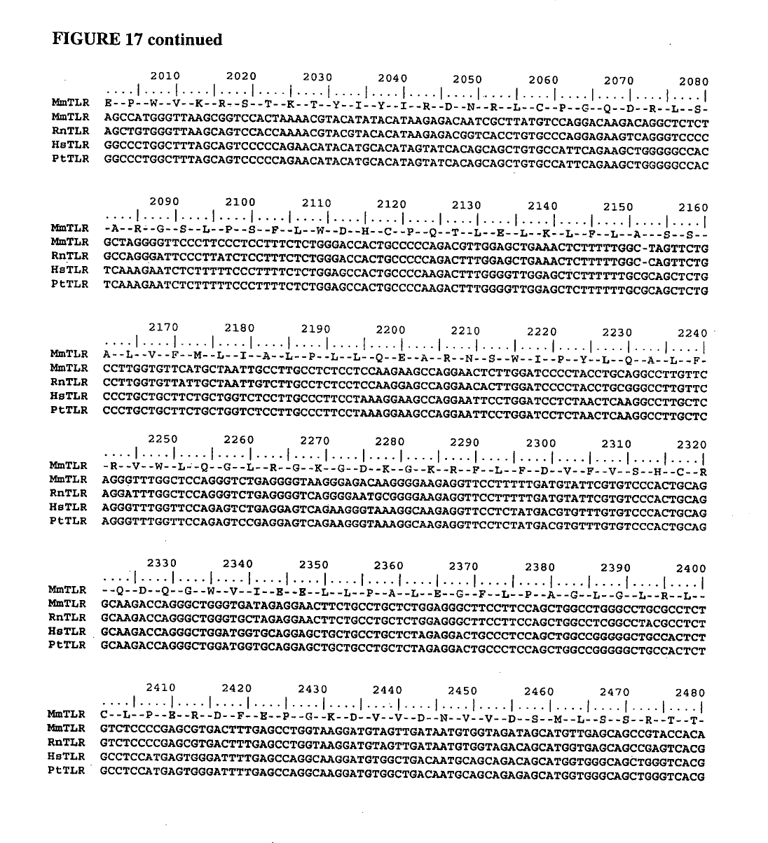

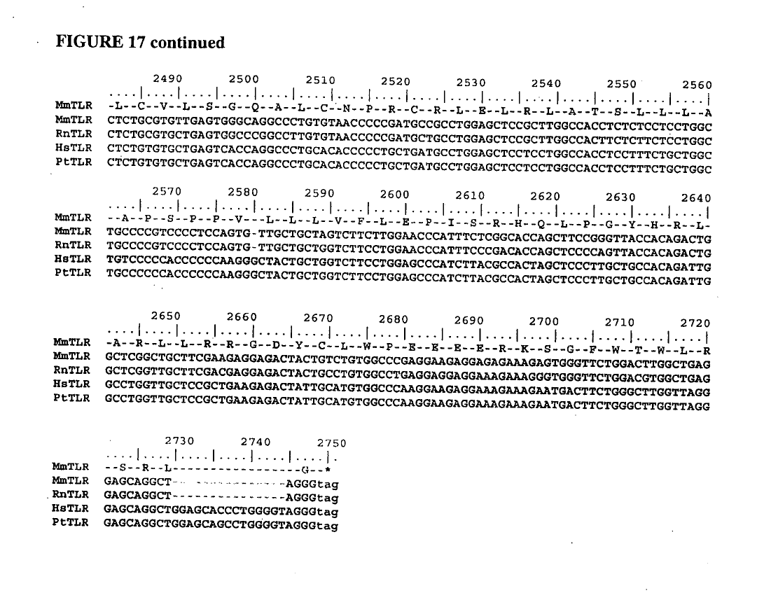

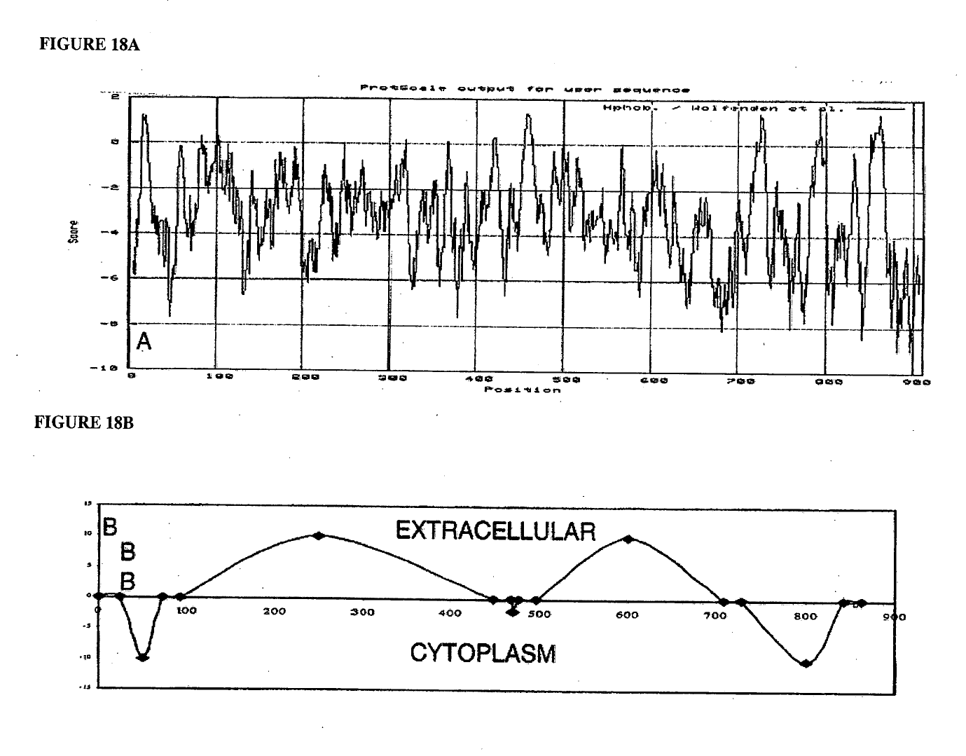

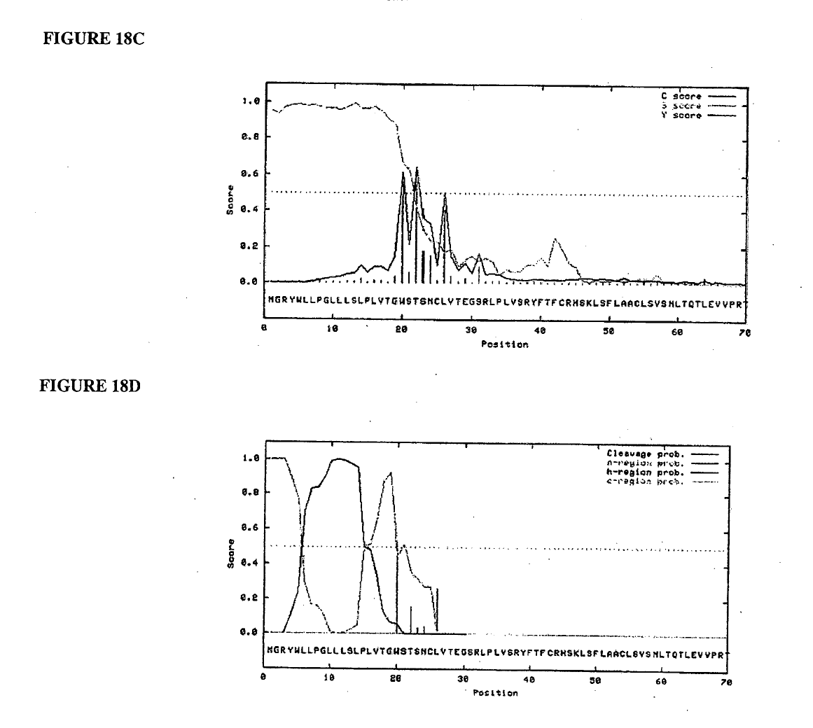

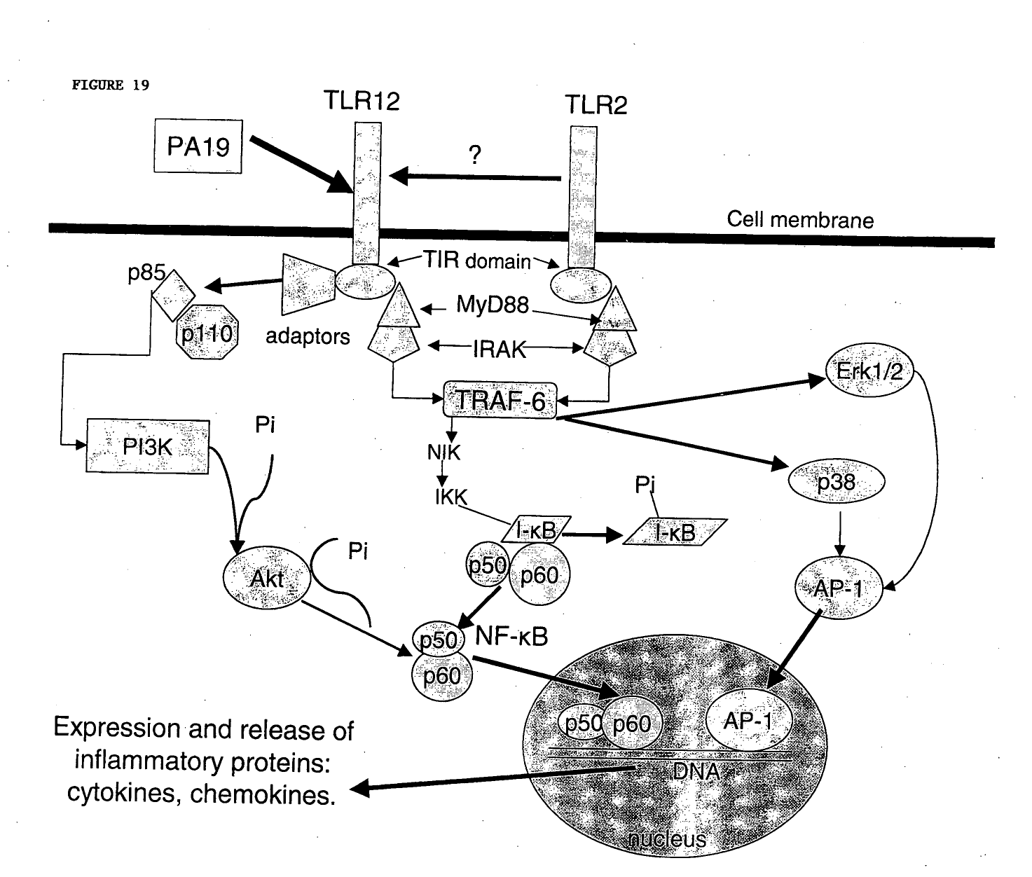

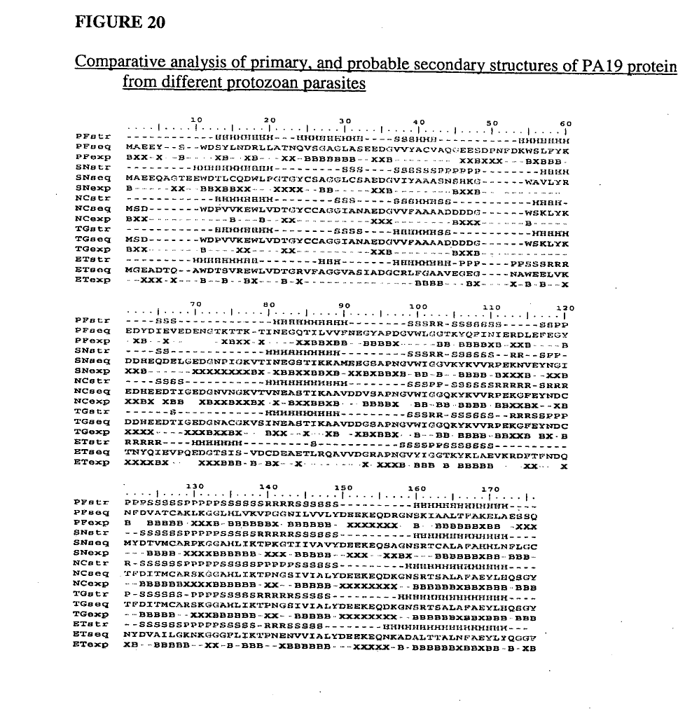

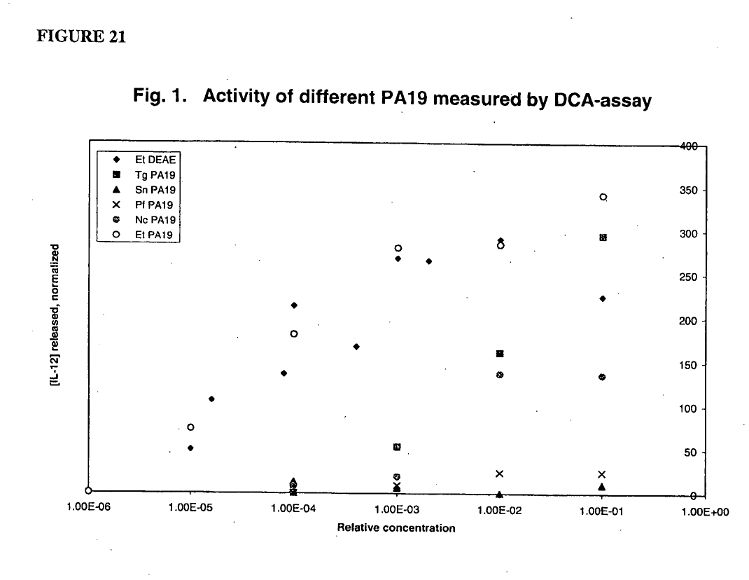

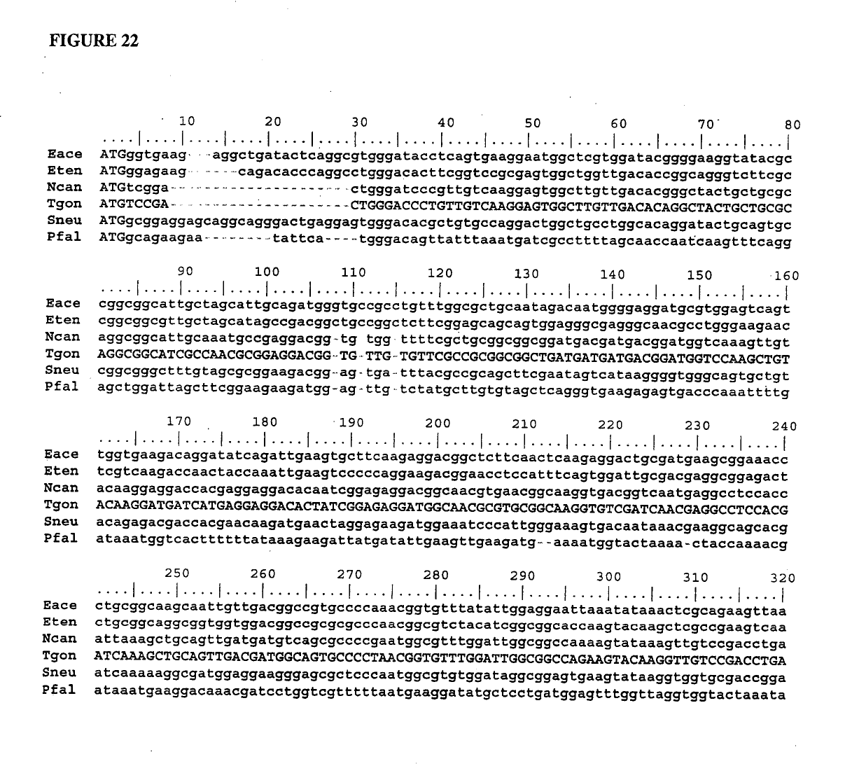

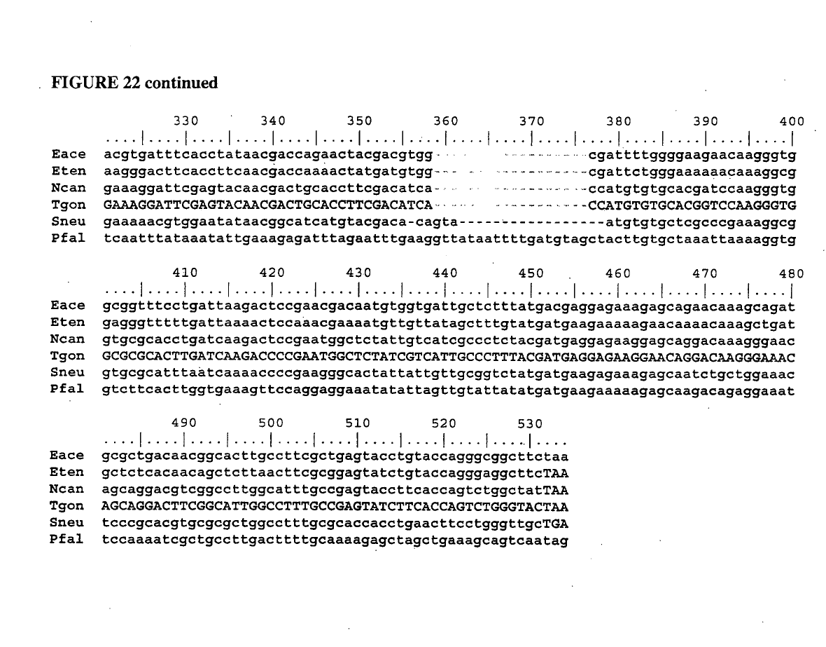

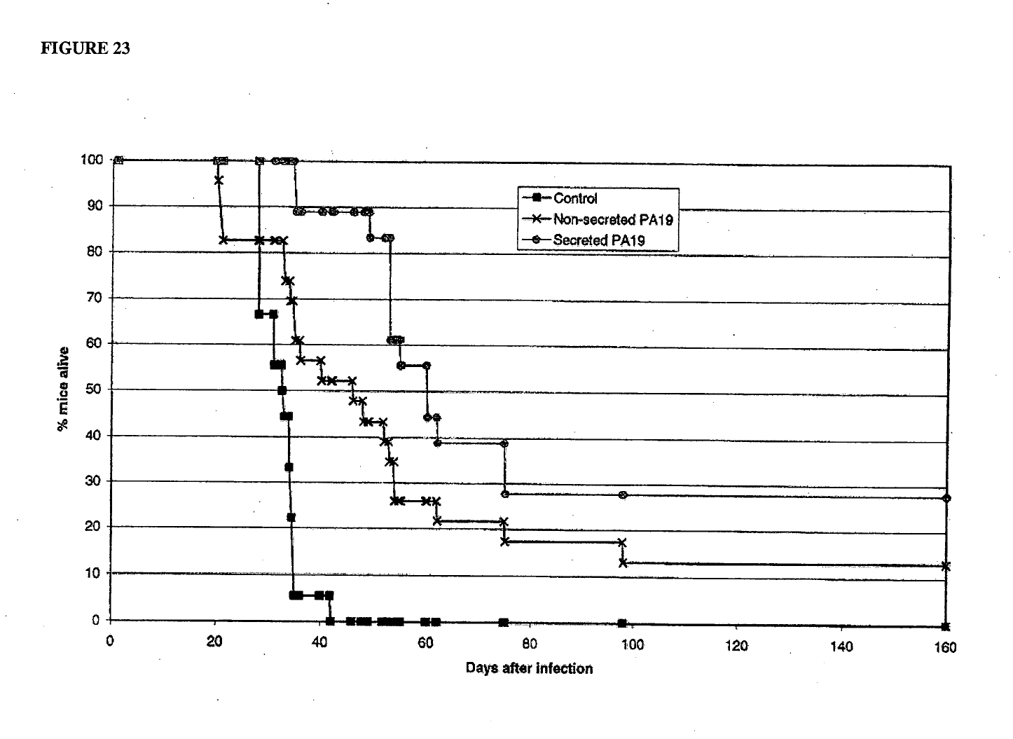

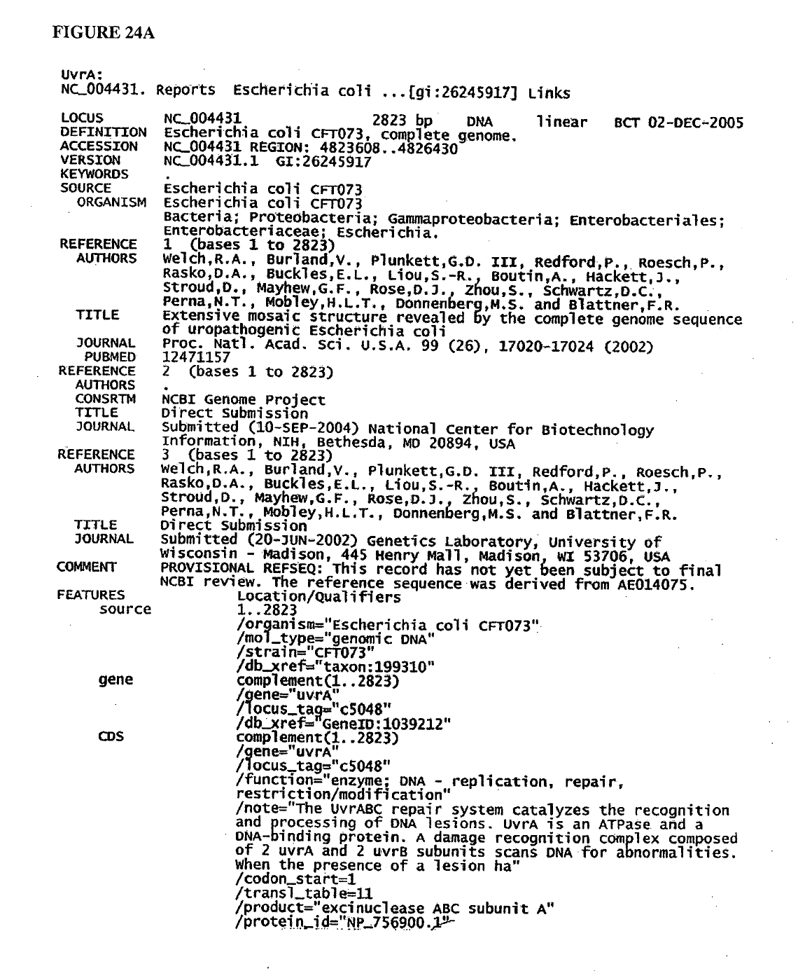

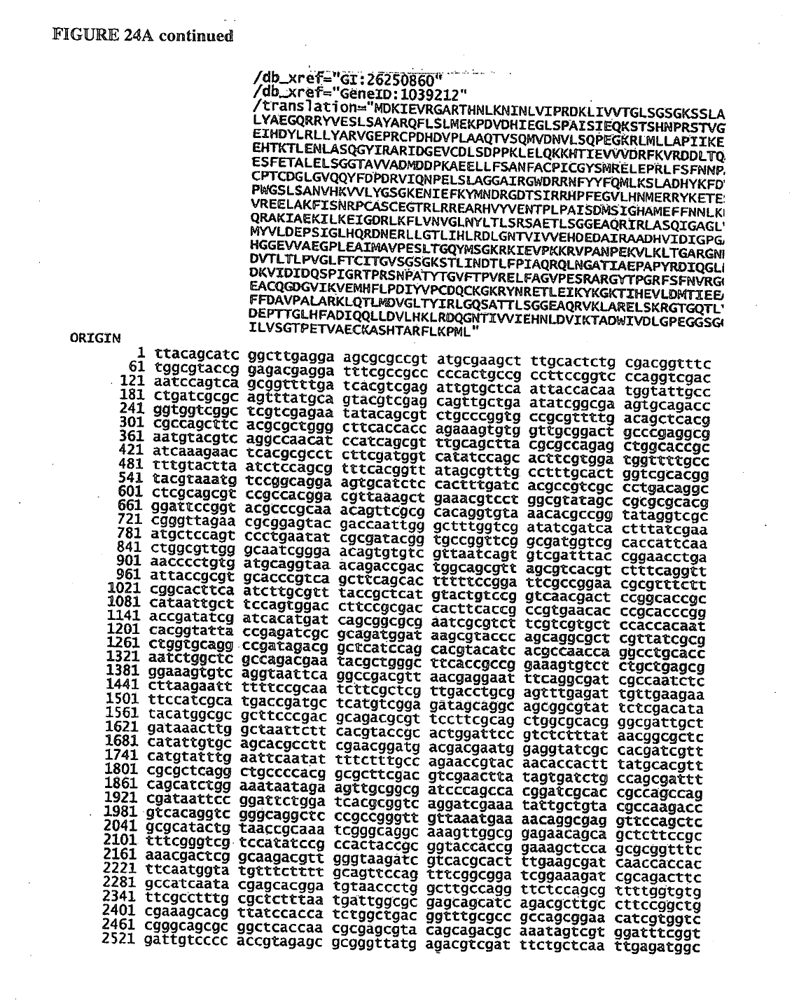

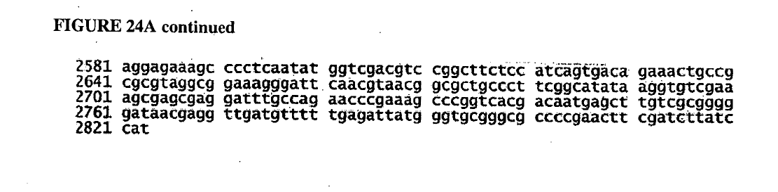

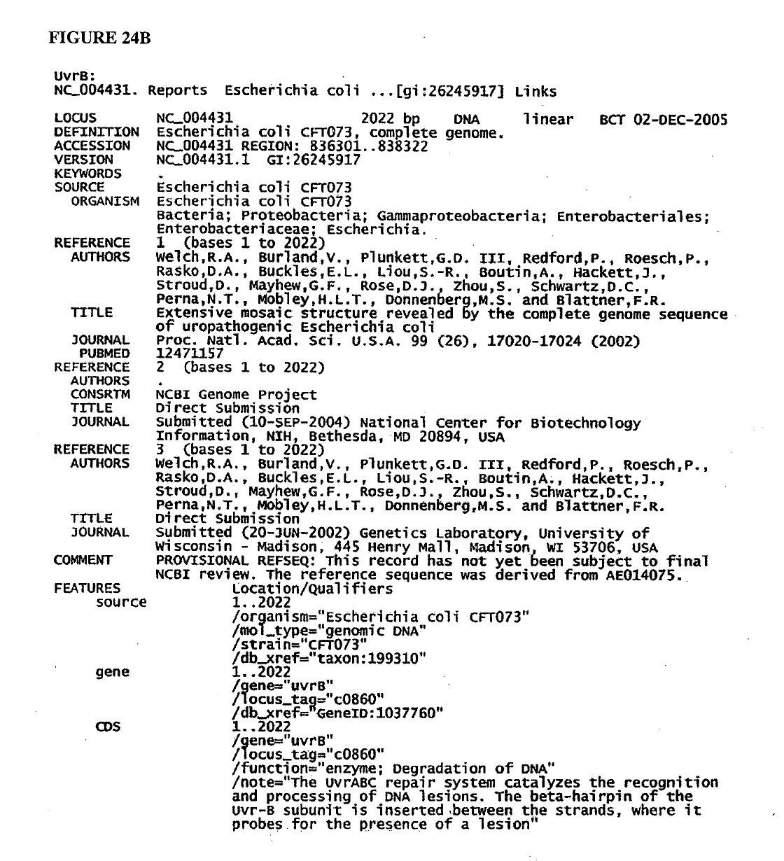

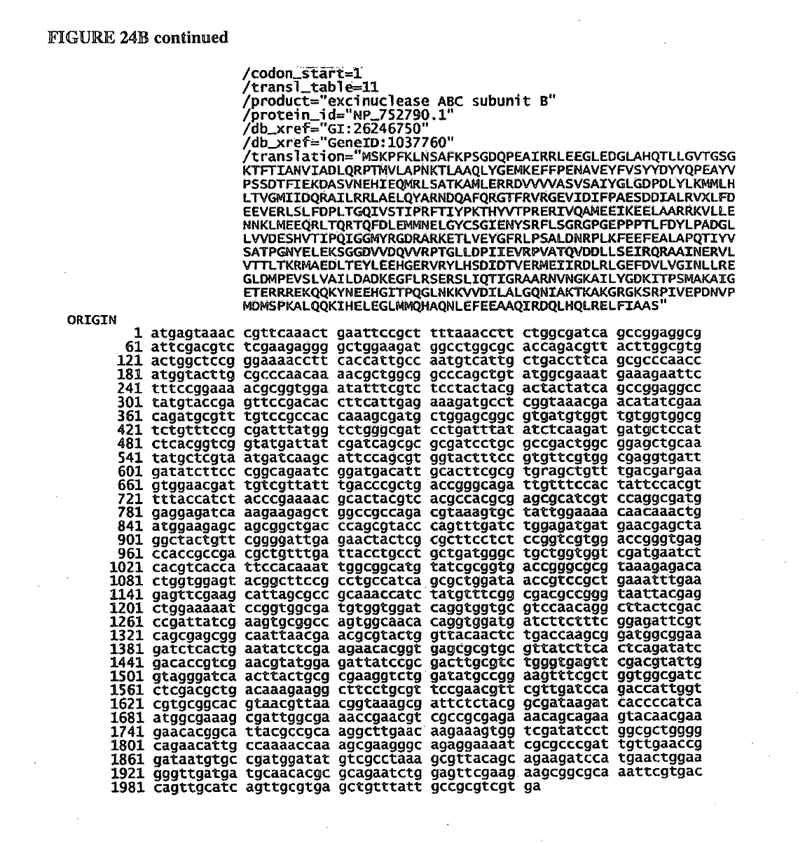

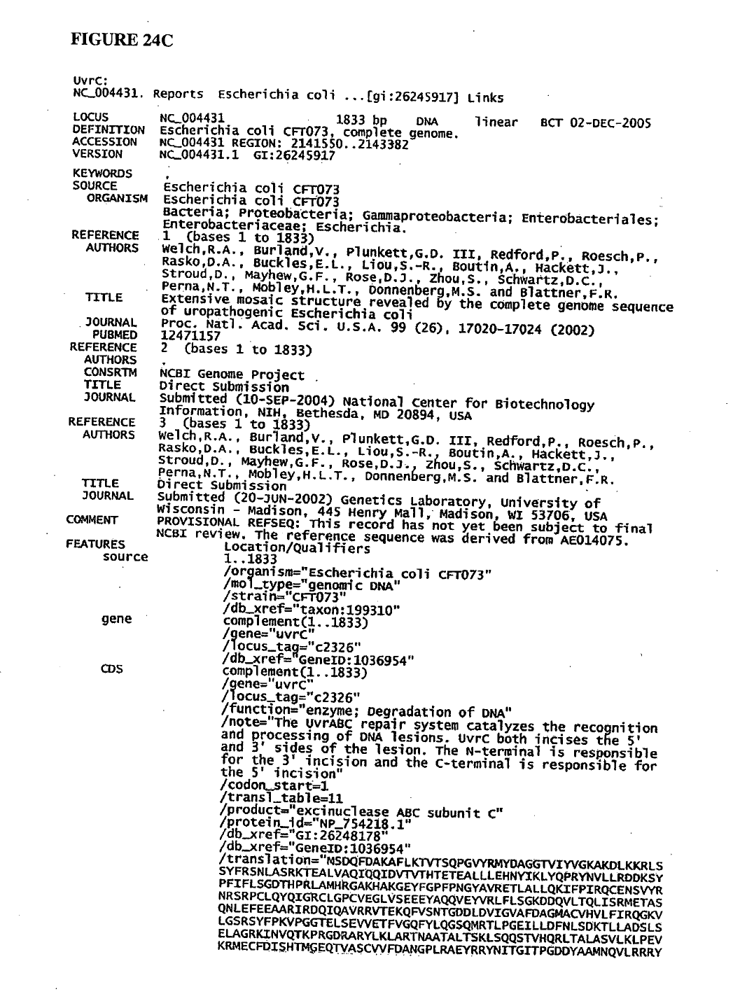

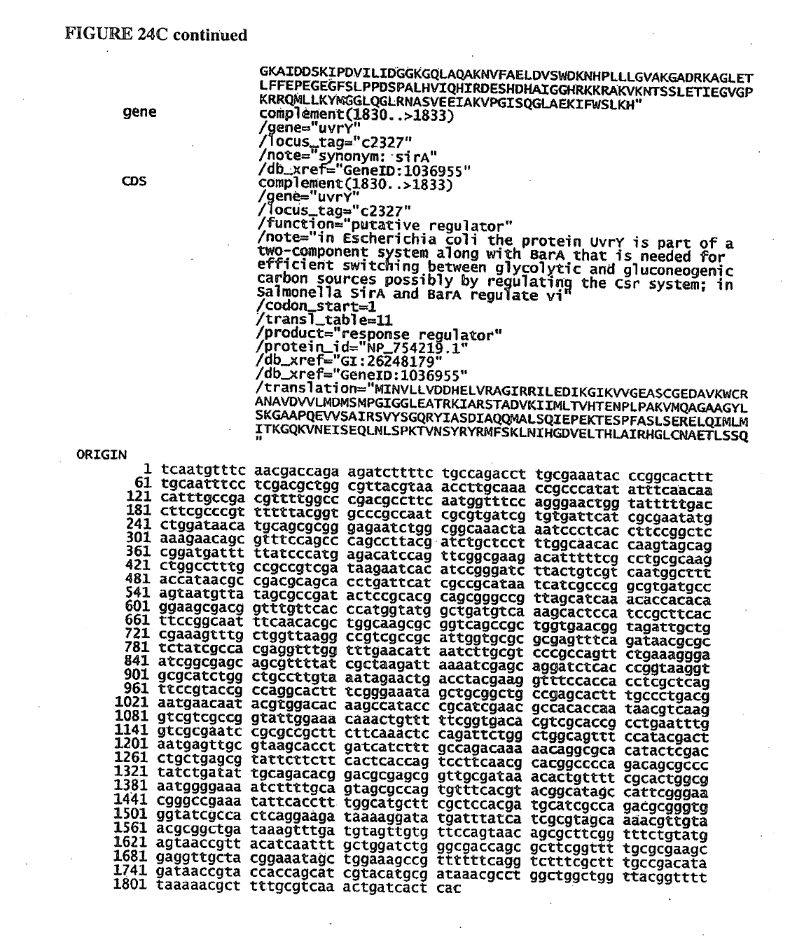

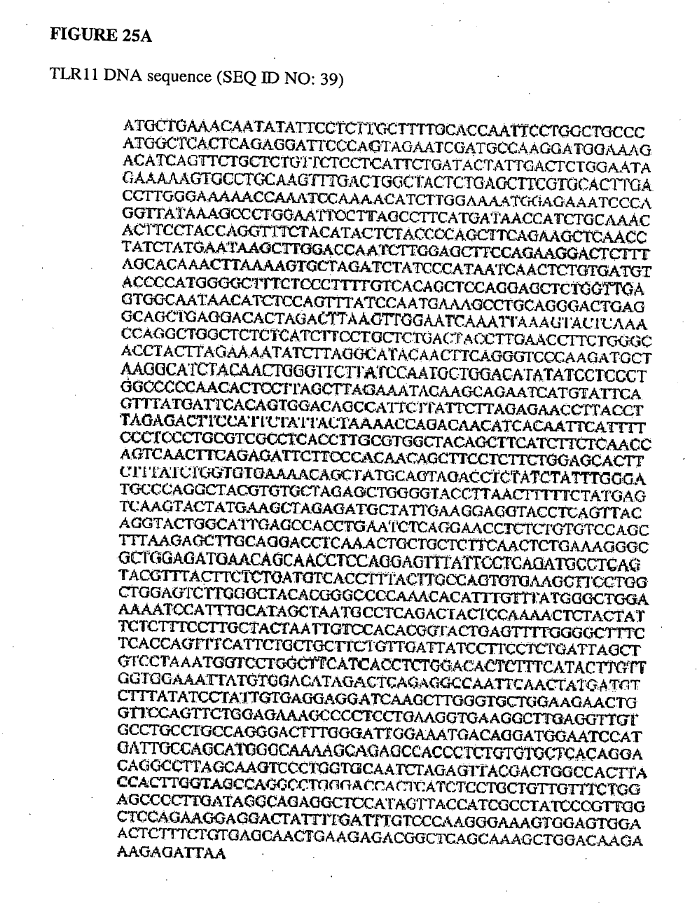

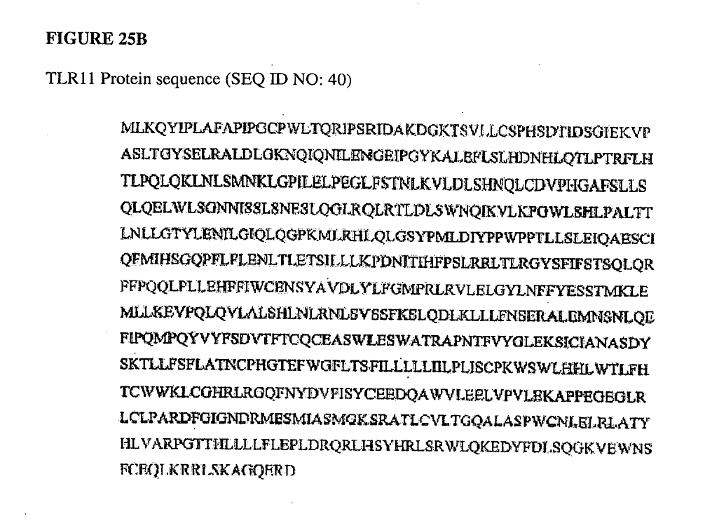

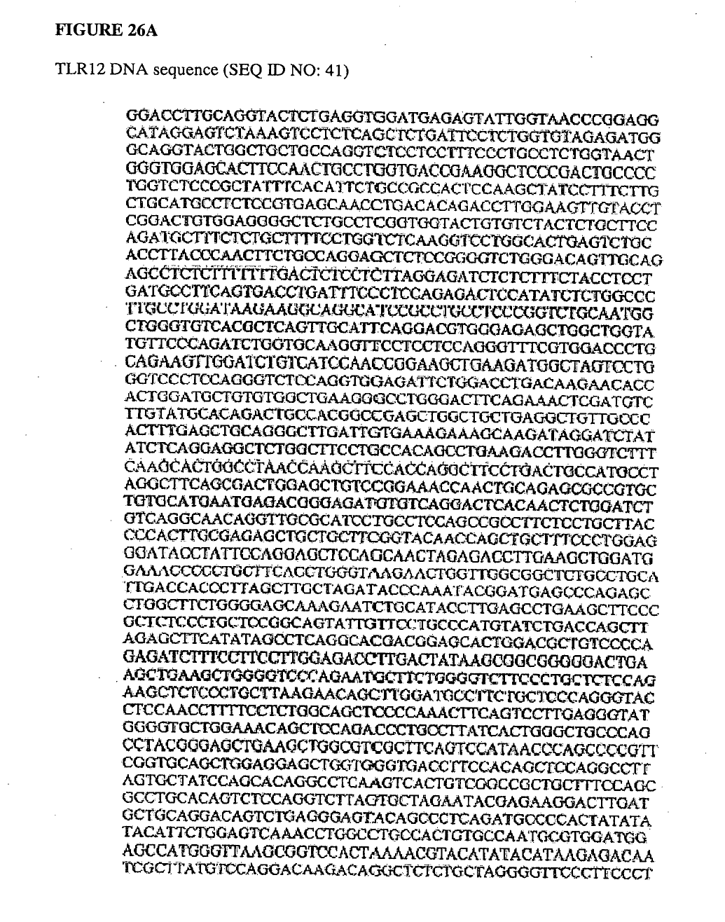

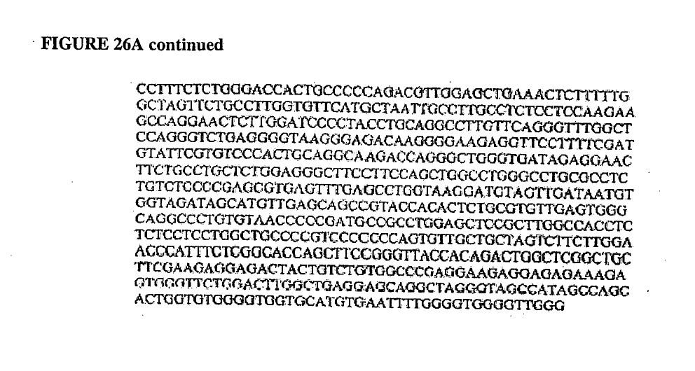

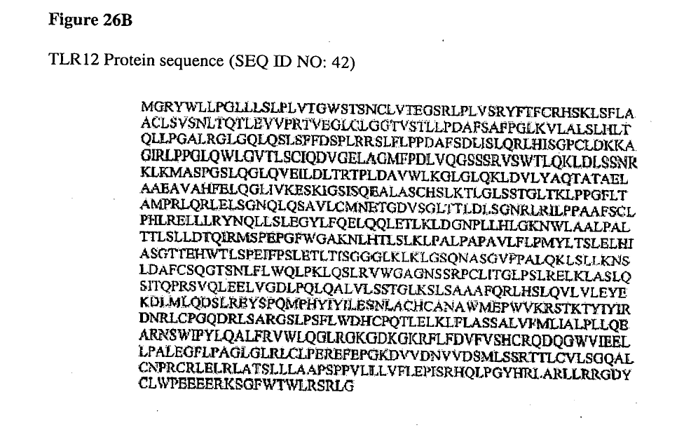

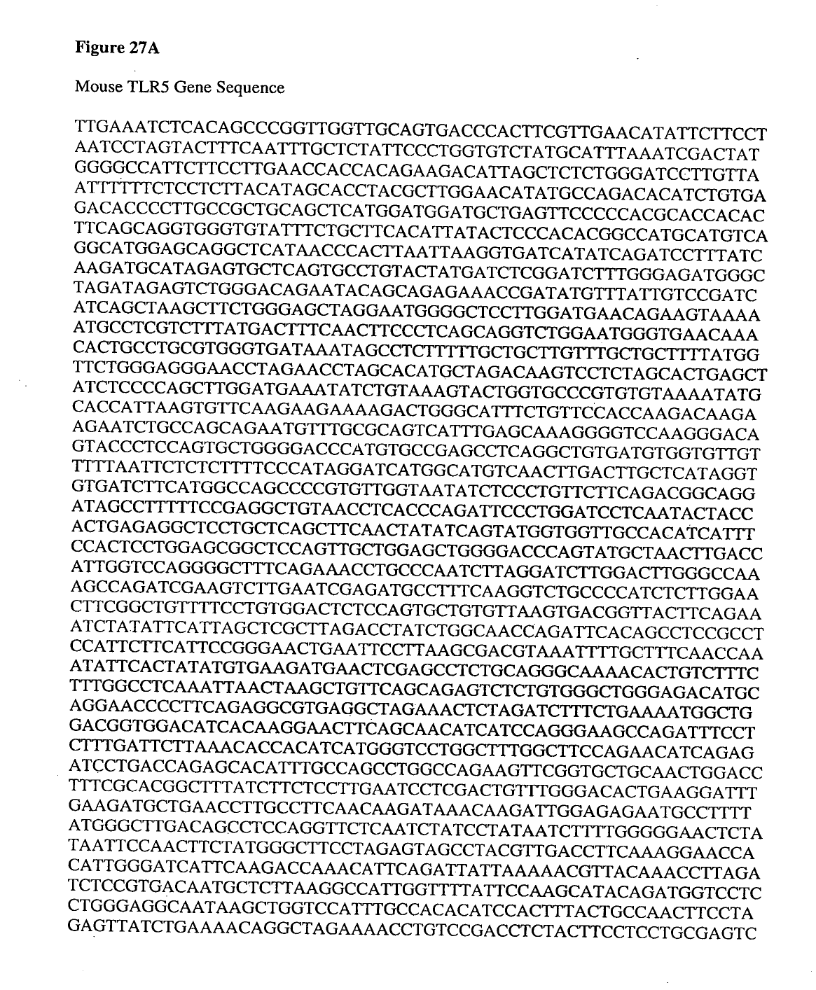

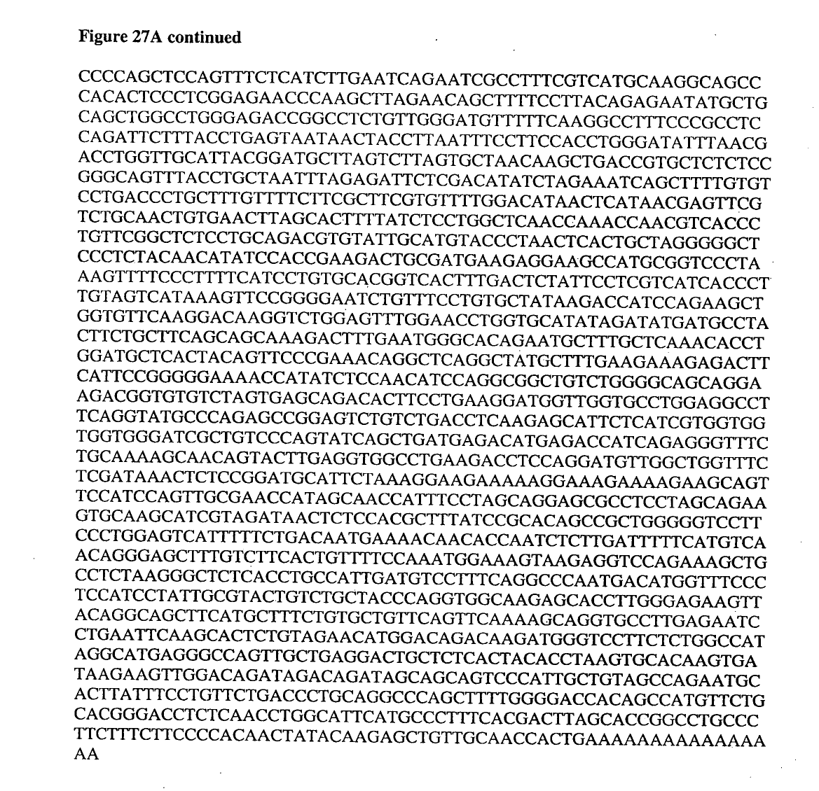

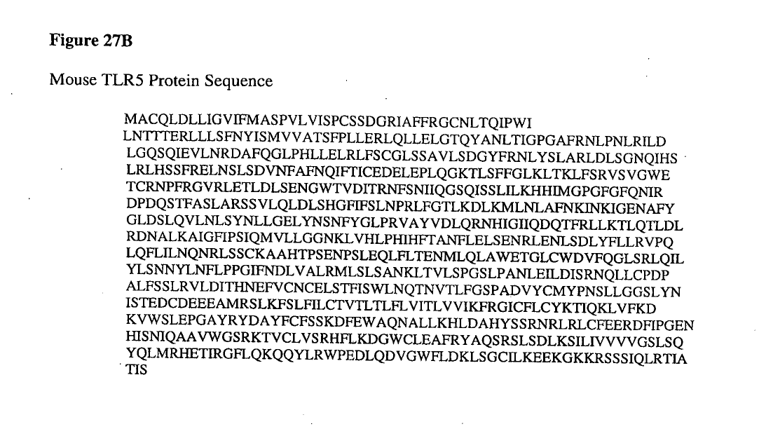

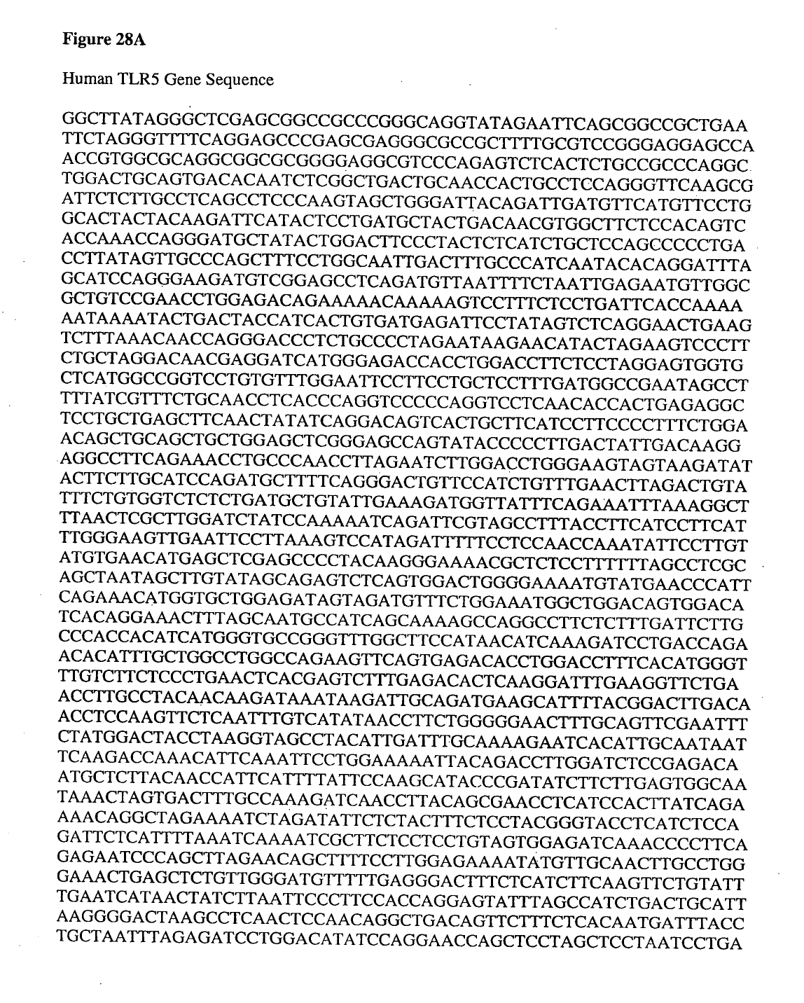

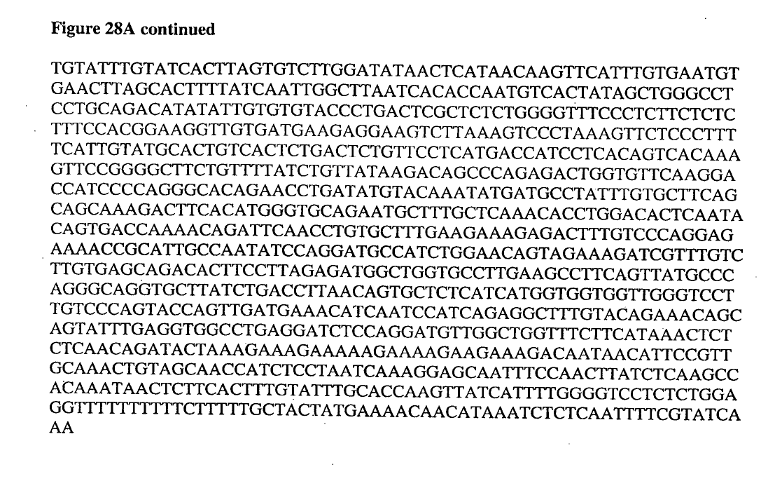

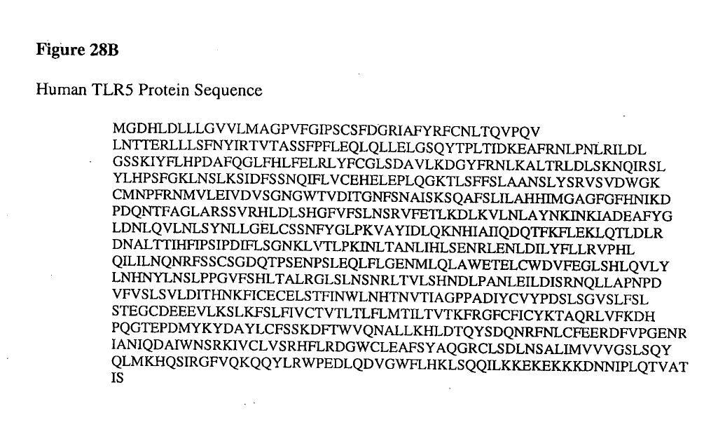

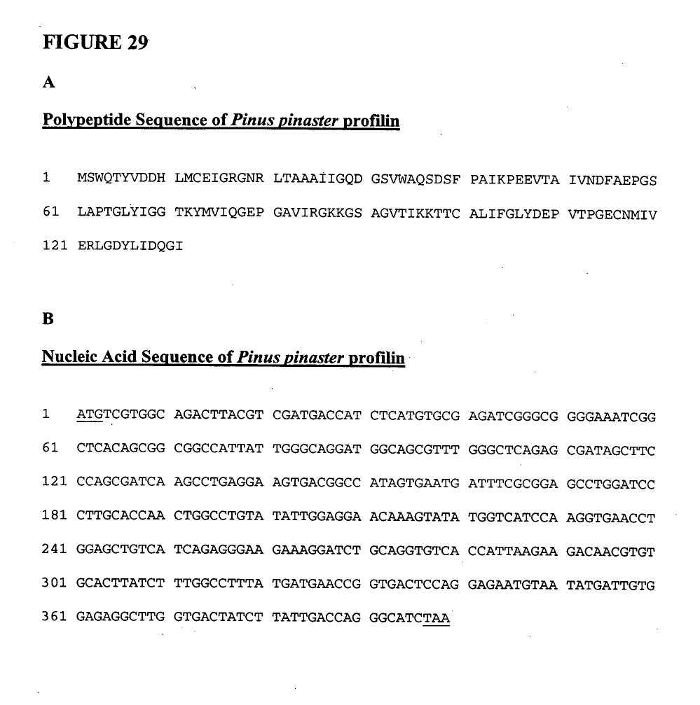

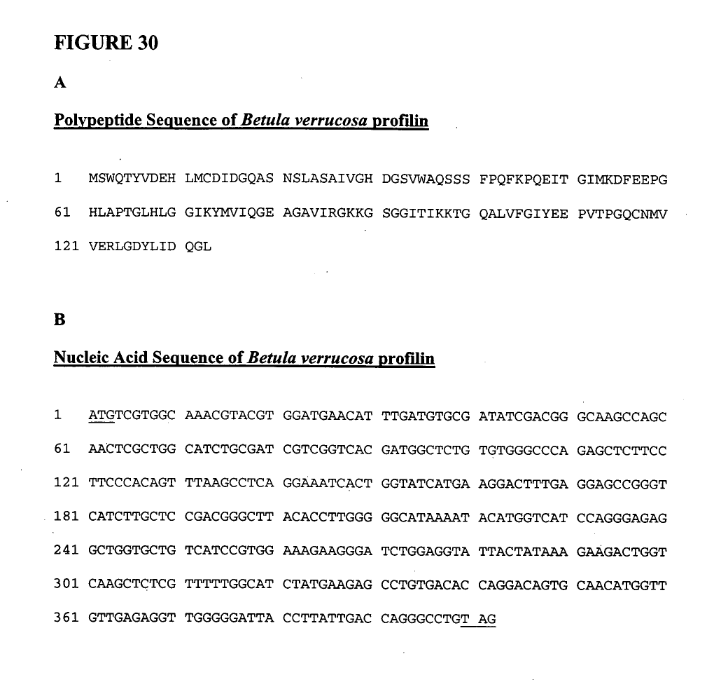

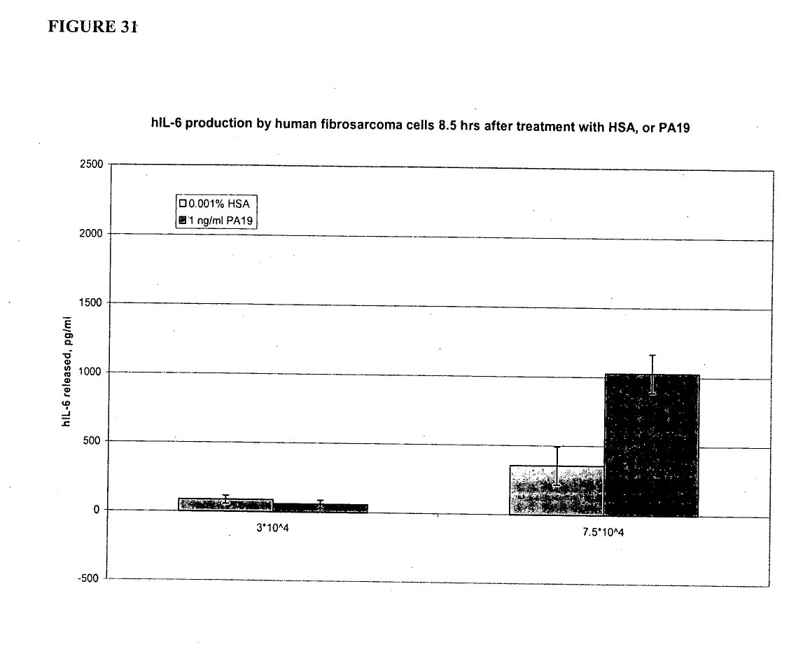

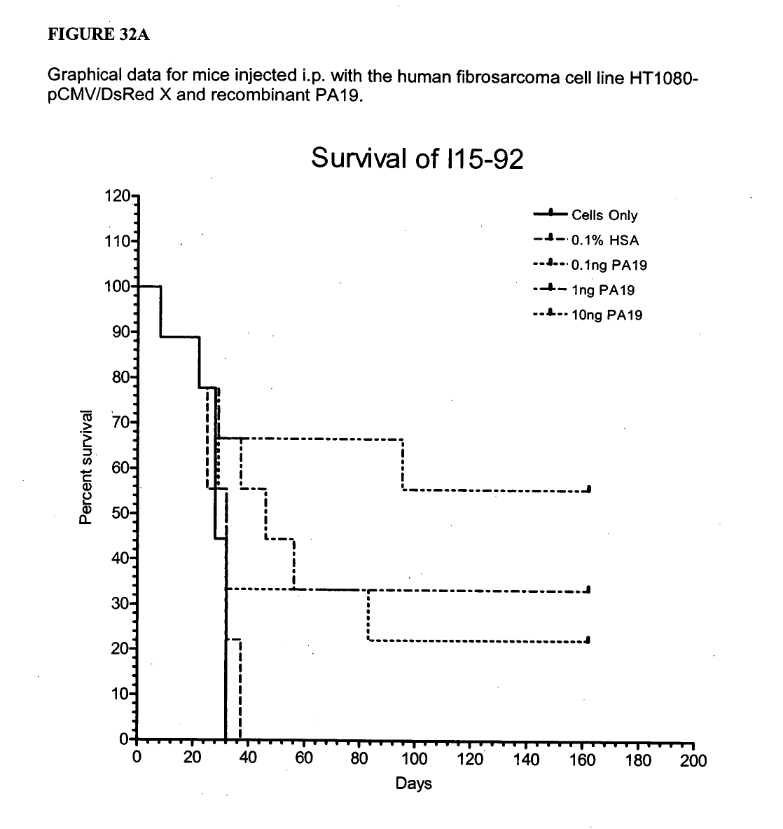

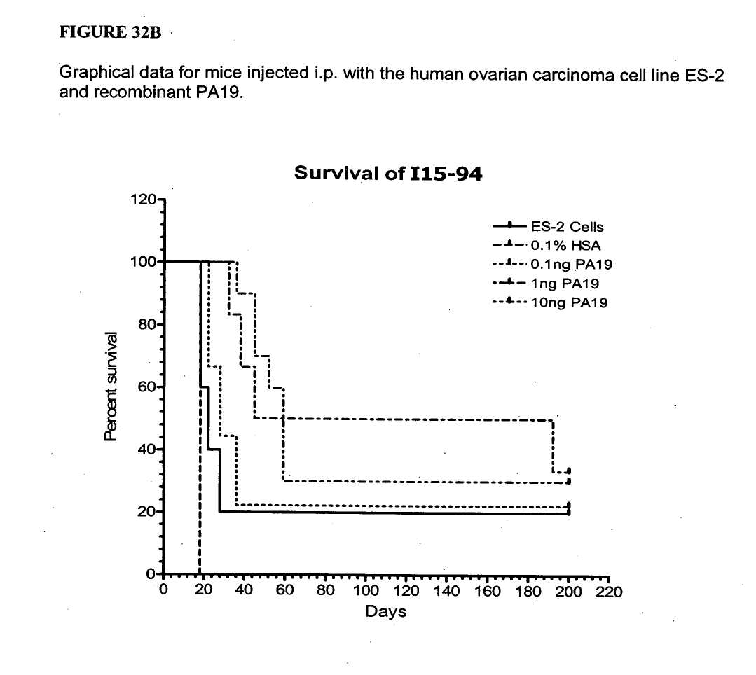

FIG. 11D is a schematic representation of the nucleotide sequence (SEQ ID NO: X(21?)) of a N. caninum profilin-related polypeptide.

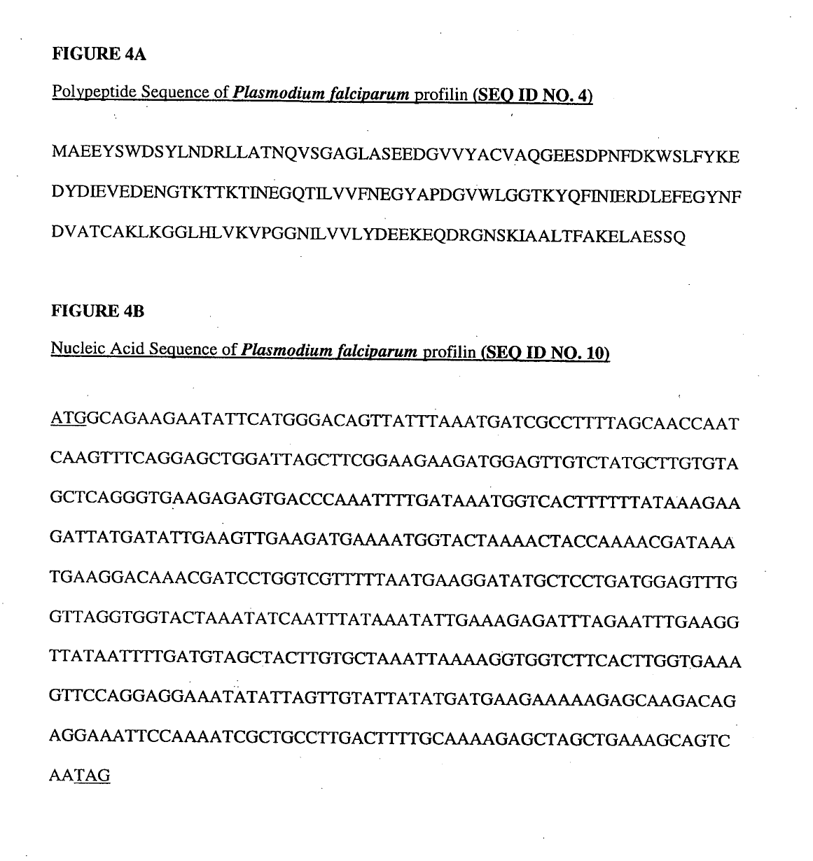

FIG. 11E is a schematic representation of a profilin-related polypeptide sequence from P. falciparum (SEQ ID NO: X(22)) showing a presequence (underlined) not shown in FIG. 4A.

FIG. 11F is a schematic representation of the nucleotide sequence (SEQ ID NO: X(23?)) of a P. falciparum profilin-related polypeptide.

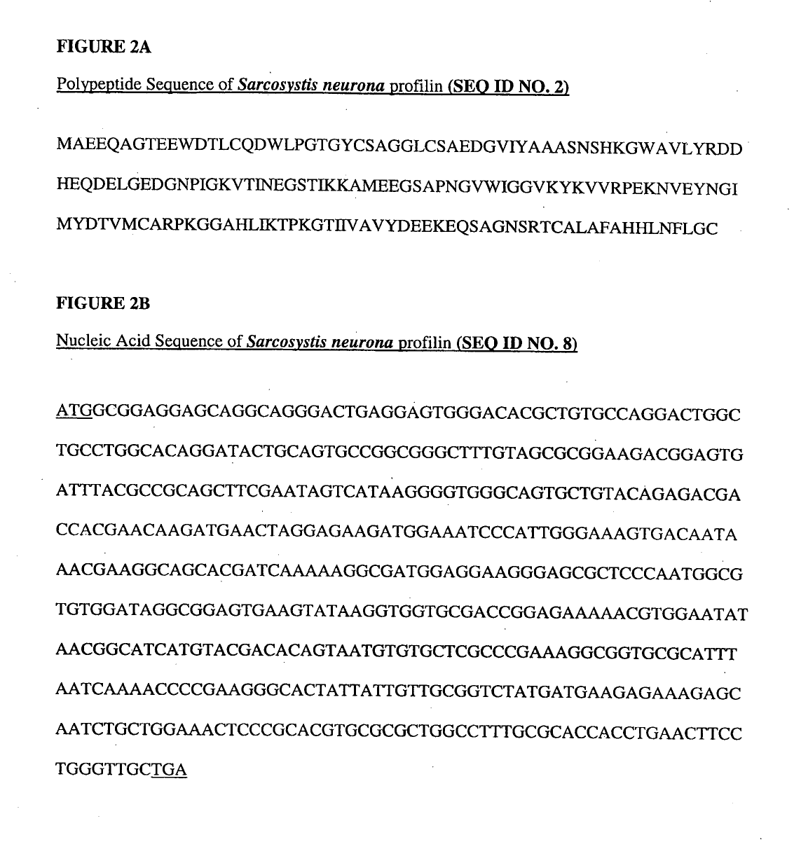

FIG. 11G is a schematic representation of a profilin-related polypeptide sequence from S. neurona (SEQ ID NO: X(24)) showing a presequence (underlined) not shown in FIG. 2A.

FIG. 11H is a schematic representation of the nucleotide sequence (SEQ ID NO: X(25)) of a S. neurona profilin-related polypeptide.

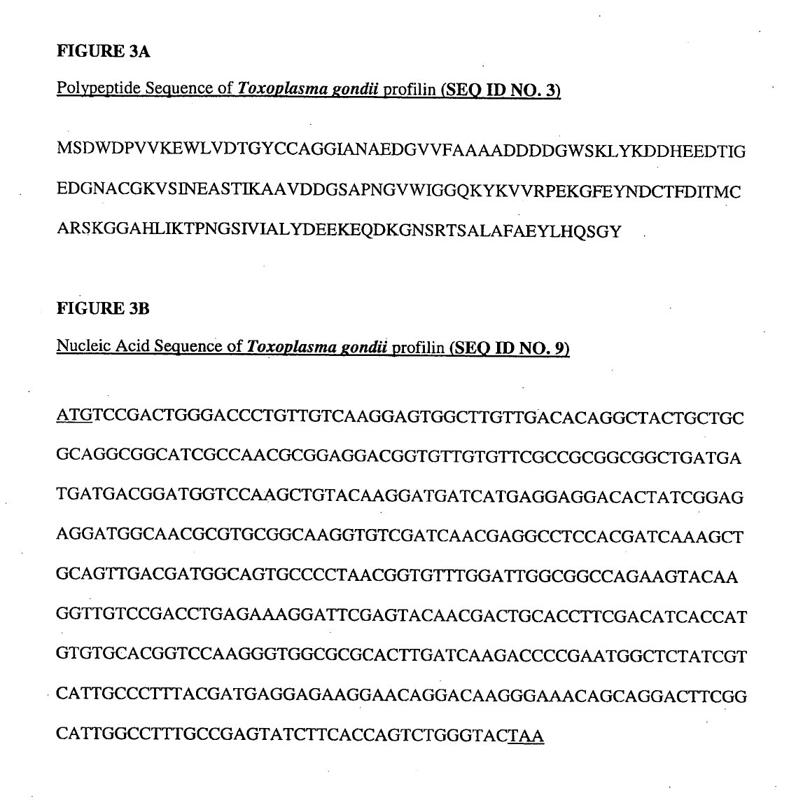

FIG. 11I is a schematic representation of a profilin-related polypeptide sequence from T. gondii (SEQ ID NO: X(26)) showing a presequence (underlined) not shown in FIG. 3A.

FIG. 11J is a schematic representation of the nucleotide sequence (SEQ ID NO: X(27?)) of a T. gondii profilin-related polypeptide.

FIG. 12A is a schematic representation of a polypeptide sequence of E. coli UvrA (SEQ ID NO: X(28)).

FIG. 12B is a schematic representation of the nucleotide sequence of E. coli UvrA (SEQ ID NO: X(29)).

FIG. 12C is a schematic representation of a polypeptide sequence of E. coli UvrB (SEQ ID NO: X(30)).

FIG. 12D is a schematic representation of the nucleotide sequence of E. coli UvrB (SEQ ID NO: X(31)).

FIG. 12E is a schematic representation of a polypeptide sequence of E. coli UvrC (SEQ ID NO: X(32)).

FIG. 12F is a schematic representation of the similarity between E. tenella profilin-related polypeptide and homologous regions of the UvrB and UvrC subunits of E. coli CFT073 UvrBC complex.

FIG. 13A is a schematic representation of the polypeptide sequence of a murine TLR11/TLR12 (SEQ ID NO: X(33)).

FIG. 13B is a schematic representation of the polypeptide sequence of a rat TLR11 (SEQ ID NO: X(34)).

FIG. 13C is a schematic representation of the polypeptide sequence of a chicken TLR11/TLR12 (SEQ ID NO: X(35)).

FIG. 14 is a schematic representation of an alignment of TLR11/TLR12 predicted proteins from mouse, rat, human, and chimp. In this figure (*) signifies a stop codon (−) is a gap in the alignment, and (Z) signifies a frameshift.

FIG. 15 is a schematic representation of a comparison of the gene region of hTLR12 with a corresponding repaired gene sequence.

FIG. 16 is a schematic representation of a predicted sequence for the repaired hTLR12 protein shown in FIG. 15.

FIG. 17 is a schematic representation of an alignment of TLR12 genes including human TLR12 and the murine TLR11/12 protein.

FIG. 18A is a graphical representation of the possible topology of mTLR12 showing hydrophobicity according to the Wolfenden algorithm.

FIG. 18B is a graphical representation of the possible topology of mTLR12 showing exposure on cell surface (inwards or outwards).

FIG. 18C is a graphical representation of a SignalP-NN prediction (eukaryote model) of mTLR11, which predicts eukaryotic secretory signal sequences.

FIG. 18D is a graphical representation of a SignalP-HMM prediction (eukaryote model) of mTLR11, which indicates the presence of an amino-terminal secretion signal sequence in the full-length receptor polypeptide.

FIG. 19 is a schematic representation of a proposed pathway for PA19 signaling through TLR11/TLR12 and/or TLR5.

FIG. 20 is a schematic representation of a comparative analysis of primary and probable secondary structures of PA19 protein from different protozoan parasites

FIG. 21 is a graphic representation of the activity of different PA19 proteins measured by DCA assay.

FIG. 22 is a schematic representation of a preliminary alignment of PA19 sequences from various organisms.

FIG. 23 is a graphical representation of the effect of PA19 expression by HT1080 fibrosarcoma cell lines on its tumorigenicity in athymic mice.

FIG. 24A is a schematic representation of polypeptide and nucleic acid sequence information for UvrA.

FIG. 24B is a schematic representation of polypeptide and nucleic acid sequence information for UvrB.

FIG. 24C is a schematic representation of polypeptide and nucleic acid sequence information for UvrC.

FIG. 25A is a schematic representation of the nucleic acid sequence encoding mouse TLR11.

FIG. 25B is a schematic representation of the amino acid sequence of mouse TLR11.

FIG. 26A is a schematic representation of the nucleic acid sequence encoding mouse TLR12.

FIG. 26B is a schematic representation of the amino acid sequence of mouse TLR12.

FIG. 27A is a schematic representation of the nucleic acid sequence encoding mouse TLR2.

FIG. 27B is a schematic representation of the amino acid sequence of mouse TLR5.

FIG. 28A is a schematic representation of the nucleic acid sequence encoding human TLR5.

FIG. 28B is a schematic representation of the amino acid sequence of human TLR5.

FIG. 29A is a schematic representation of the amino acid sequence of Pinus pinaster prolilin.

FIG. 29B is a schematic representation of the nucleic acid sequence encoding Pinus pinaster prolilin. The initiation and termination codons of the profilin protein open reading frame (ORF) are underlined.

FIG. 30A is a schematic representation of the amino acid sequence of Betula verrusoca prolilin.

FIG. 30B is a schematic representation of the nucleic acid sequence encoding Betula verrusoca prolilin. The initiation and termination codons of the profilin protein open reading ORF) are underlined.

FIG. 31 is a graphical representation of the effect of PA19 on hIL-6 production by human fibrosarcoma cells in vitro.

FIG. 32A is a graphical representation of the protective effect of purified recombinant PA19 on survival of mice injected intraperoneously with a human fibrosarcoma.

FIG. 32B is a graphical representation of the protective effect of purified recombinant PA19 on survival of mice injected intraperoneously with a human ovarian carcinoma.

### 4. DETAILED DESCRIPTION OF THE INVENTION

The patent and scientific literature referred to herein establishes the knowledge that is available to those with skill in the art. The issued U.S. patents, applications, published foreign applications, and references cited herein are hereby incorporated by reference in their entirety.

### 4.1 General

In general, aspects of the invention provide immunomodulatory profilins, profilin-related polypeptides and profilin-like proteins, as well as cognate nucleic acid sequences which encode them and pharmaceutical formulations that contain them. In addition, aspects of the invention relate to compositions for, and methods of, activating an immune response in a subject, including immunomodulatory and/or immunostimulatory TLR11/TLR12 and/or TLR5 agonists and associated methods of increasing the level of immune cytokines in a subject, including, without limitation, IL-12. Historically, the TLR11/TLR12 protein was named TLR11 and TLR12 and references cited herein generally refer to the protein and its gene as TLR11/TLR12. Further aspects of the invention provide methods of identifying a candidate subject for treatment with a profilin-related immunostimulatory polypeptide as well as methods of treating an infectious disease or cancer in a subject.

### 4.2 Definitions

As used herein, the following terms and phrases shall have the meanings set forth below. Unless defined otherwise, all technical and scientific terms used herein have the same meaning as commonly understood to one of ordinary skill in the art to which this invention belongs.

The term “profilin” as used herein refers to a class of proteins that binds to monomeric actin and prevents the polymerization of actin. Nonlimiting examples include human profilin 1 (GenBank Accession NO: NP_{—}005013) and human profilin 2 (GenBank Accession NO: NP_{—}444152).

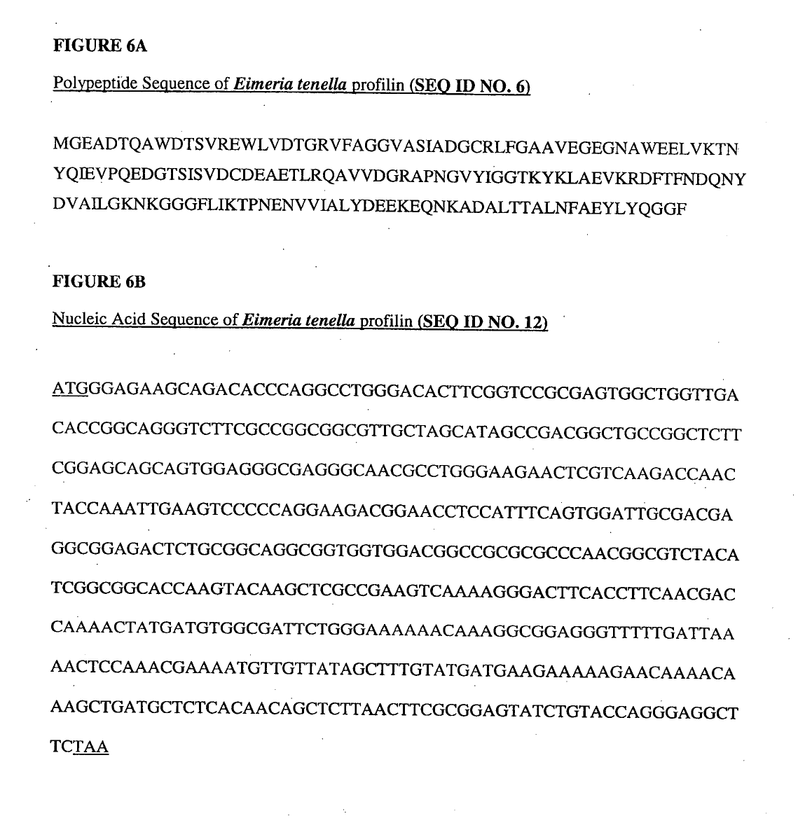

The terms “profilin-like immunomodulatory protein (PLIP)” and “profilin-related immunomodulatory protein (PRIP)” refer to polypeptides with one or more properties of a profilin protein, including primary, secondary, and/or tertiary structural similarities, and which further possess immunomodulatory activity (e.g., IL-12 stimulation). Nonlimiting examples include the Eimeria tenella profilin-related immunomodulatory protein (PRIP) shown in FIG. 6A.

The term “about” means an acceptable degree of error for the quantity measured given the nature or precision of the measurements. Typically, exemplary degrees of error are within 20%. Numerical quantities given herein are approximate unless stated otherwise, meaning that the term “about” can be inferred when not expressly stated.

The term “antibody” as used herein is intended to include whole antibodies, e.g., of any isotype (IgG, IgA, IgM, IgE, etc), and includes fragments thereof which are also specifically reactive with a vertebrate, e.g., mammalian, protein. Antibodies can be fragmented using conventional techniques and the fragments screened for utility in the same manner as described above for whole antibodies. Thus, the term includes segments of proteolytically cleaved or recombinantly-prepared portions of an antibody molecule that are capable of selectively reacting with a certain protein. Nonlimiting examples of such proteolytic and/or recombinant fragments include Fab, F(ab′)2, Fab′, Fv, and single chain antibodies (scFv) containing a V[L] and/or V[H] domain joined by a peptide linker. The scFv's may be covalently or noncovalently linked to form antibodies having two or more binding sites. The subject invention includes polyclonal, monoclonal, or other purified preparations of antibodies and recombinant antibodies.

An “antigenic function” means possession of an epitope or antigenic site that is capable of cross-reacting with antibodies raised against native sequence profilin or a PRIP. The principal antigenic function of a PRIP polypeptide is that it binds with an affinity of at least about 10⁶ L/mole (binding affinity constant, i.e., Kₐ) to an antibody raised against PRIP. Ordinarily the polypeptide binds with an affinity of at least about 10⁷ L/mole. The binding affinity of the subject PRIP antibodies may also be measured in terms of a binding dissociation constant (K_{d}), which refers to the concentration of a binding protein (i.e., the antibody) at which 50% of the antigen protein (i.e., profilin) is occupied. In general, particularly useful profilin antibodies of the invention have a K_{d} value in the range of 0.1 to 3 nM (corresponding to a Kₐ of approximately 3×10⁸ L/mole to 1×10¹⁰ L/mole).

“Antigenically active” profilin is defined as a polypeptide that possesses an antigenic function of profilin, and that may (but need not) in addition possess a biological activity of profilin.

“Biological property” when used in conjunction with PRIP means having any of the activities associated with a native profilin.

The term “biological sample”, as used herein, refers to a sample obtained from an organism or from components (e.g., cells) of an organism. The sample may be of any biological tissue or fluid. Frequently the sample will be a “clinical sample” which is a sample derived from a patient. Such samples include, but are not limited to, tumors, sputum, blood, blood cells (e.g., white cells), tissue or fine needle biopsy samples, urine, lacrinal fluid, seminal fluid, vaginal secretions, peritoneal fluid, and pleural fluid, or cells there from. Biological samples may also include sections of tissues such as frozen sections taken for histological purposes. “Cells”, “host cells” or “recombinant host cells” are terms used interchangeably herein. It is understood that such terms refer not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein.

A “chimeric polypeptide” or “fusion polypeptide” is a fusion of a first amino acid sequence encoding one of the subject polypeptides with a second amino acid sequence defining a domain (e.g. polypeptide portion) foreign to and not substantially homologous with any domain of the subject polypeptide. A chimeric polypeptide may present a foreign domain which is found (albeit in a different polypeptide) in an organism which also expresses the first polypeptide, or it may be an “interspecies”, “intergenic”, etc. fusion of polypeptide structures expressed by different kinds of organisms. In general, a fusion polypeptide can be represented by the general formula X-polypeptide-Y, wherein polypeptide represents a first or subject protein or polypeptide, and X and Y are independently absent or represent amino acid sequences which are not related to the first sequence in an organism, including naturally occurring mutants. Nonlimiting examples of a chimeric polypeptide include a PRIP-fusion protein.

A “chimeric PRIP polypeptide” is a polypeptide comprising full-length PRIP or one or more fragments thereof fused or bonded to a second protein or one or more fragments thereof.

As used herein, “conservatively modified variations” of a particular nucleic acid sequence refer to those nucleic acids which encode identical or essentially identical amino acid sequences, or where the nucleic acid does not encode an amino acid sequence, to essentially identical sequences. Because of the degeneracy of the genetic code, a large number of functionally identical nucleic acids encode any given polypeptide. For instance, the codons CGU, CGC, CGA, COG, AGA, and AGG all encode the amino acid arginine. Thus, at every position where an arginine is specified by a codon, the codon can be altered to any of the corresponding codons described without altering the encoded polypeptide. Such nucleic acid variations are “silent variations,” which are one species of “conservatively modified variations.” Every nucleic acid sequence herein which encodes a polypeptide also describes every possible silent variation. One of skill will recognize that each codon in a nucleic acid (except AUG, which is ordinarily the only codon for methionine) can be modified to yield a functionally identical molecule by standard techniques. Accordingly, each “silent variation” of a nucleic acid which encodes a polypeptide is implicit in each described sequence. Furthermore, one of skill will recognize that individual substitutions, deletions or additions which alter, add or delete a single amino acid or a small percentage of amino acids (typically less than 5%, more typically less than 1%) in an encoded sequence are “conservatively modified variations” where the alterations result in the substitution of an amino acid with a chemically similar amino acid. Conservative substitution tables providing functionally similar amino acids are well known in the art. The following six groups each contain amino acids that are conservative substitutions for one another:

1) Alanine (A), Serine (S), Threonine (T);

2) Aspartic acid (D), Glutamic acid (E);

3) Asparagine (N), Glutamine (Q);

4) Arginine (R), Lysine (K);

5) Isoleucine (I), Leucine (L), Methionine (M), Valine (V); and

6) Phenylalanine (F), Tyrosine (Y), Tryptophan (W).

As described herein, sequences may be optimized for expression in a particular host cell used to produce the protein (e.g., a plant cell such as a tomato, or a cloning and expression system such as a yeast cell). Similarly, “conservative amino acid substitutions,” in one or a few amino acids in an amino acid sequence are substituted with different amino acids with highly similar properties (see, the definitions section, supra), are also readily identified as being highly similar to a particular amino acid sequence, or to a particular nucleic acid sequence which encodes an amino acid. Such conservatively substituted variations of any particular sequence are a feature of the present invention.

A “delivery complex” refers to a targeting means (e.g., a molecule that results in higher affinity binding of a gene, protein, polypeptide or peptide to a target cell surface and/or increased cellular or nuclear uptake by a target cell). Examples of targeting means include: sterols (e.g., cholesterol), lipids (e.g., a cationic lipid, virosome or liposome), viruses (e.g., tobacco mosaic virus) or target cell specific binding agents (e.g., ligands recognized by target cell specific receptors). Useful complexes are sufficiently stable in vivo to prevent significant uncoupling prior to internalization by the target cell. However, the complex is cleavable under appropriate conditions within the cell so that the gene, protein, polypeptide or peptide is released in a functional form.

The term “epitope” refers to portion of a molecule that is specifically recognized by an immunoglobulin product. It is also referred to as the determinant or antigenic determinant.

The term “epitope tagged,” when used herein, refers to a chimeric polypeptide comprising an entire profilin sequence, or a portion thereof, fused to a “tag polypeptide”. The tag polypeptide has enough residues to provide an epitope against which an antibody there against can be made, yet is short enough such that it does not interfere with activity of the profilin. The tag polypeptide may be fairly unique so that the antibody there against does not substantially cross-react with other epitopes. Suitable tag polypeptides generally have at least 6 amino acid residues and usually between about 8 to about 50 amino acid residues or between about 9 and about 30 residues.

The term “evolutionarily related to”, with respect to amino acid sequences of profilin proteins, refers to both polypeptides having amino acid sequences which have arisen naturally, and also to mutational variants of human PRIPs which are derived, for example, by combinatorial mutagenesis.

As used herein, an “immunoglobulin” is a multimeric protein containing the immunologically active portions of an immunoglobulin heavy chain and immunoglobulin light chain covalently coupled together and capable of specifically combining with antigen.

As used herein, “Fab fragment” is a multimeric protein consisting of the portion of an immunoglobulin molecule containing the immunologically active portions of an immunoglobulin heavy chain and an immunoglobulin light chain covalently coupled together and capable of specifically combining with antigen. Fab fragments are typically prepared by proteolytic digestion of substantially intact immunoglobulin molecules with papain using methods that are well known in the art. However, a Fab fragment may also be prepared by expressing in a suitable host cell the desired portions of immunoglobulin heavy chain and immunoglobulin light chain using methods well known in the art.

As used herein, an “Fv fragment” refers to a multimeric protein consisting of the immunologically active portions of an immunoglobulin heavy chain variable region and an immunoglobulin light chain variable region covalently coupled together and capable of specifically combining with antigen. Fv fragments are typically prepared by expressing in suitable host cell the desired portions of immunoglobulin heavy chain variable region and immunoglobulin light chain variable region using methods well known in the art.

As used herein, the term “gene” or “recombinant gene” refers to a nucleic acid comprising an open reading frame encoding a polypeptide of the present invention, including both exon and (optionally) intron sequences. A “recombinant gene” refers to nucleic acid encoding such regulatory polypeptides, which may optionally include intron sequences which are either derived from a chromosomal DNA. Exemplary recombinant genes include those which encode a profilin-related polypeptide activity.

As used herein, “heterologous DNA” or “heterologous nucleic acid” include DNA that does not occur naturally as part of the genome in which it is present or which is found in a location or locations in the genome that differs from that in which it occurs in nature. Heterologous DNA is not endogenous to the cell into which it is introduced, but has been obtained from another cell. Generally, although not necessarily, such DNA encodes RNA and proteins that are not normally produced by the cell in which it is expressed. Heterologous DNA may also be referred to as foreign DNA, Any DNA that one of skill in the art would recognize or consider as heterologous or foreign to the cell in which is expressed is herein encompassed by heterologous DNA. Examples of heterologous DNA include, but are not limited to, isolated DNA that encodes a sulfotransferase protein.

“Homology” or “identity” or “similarity” refers to sequence similarity between two peptides or between two nucleic acid molecules. Homology can be determined by comparing a position in each sequence which may be aligned for purposes of comparison. When a position in the compared sequence is occupied by the same base or amino acid, then the molecules are identical at that position. A degree of homology or similarity or identity between nucleic acid sequences is a function of the number of identical or matching nucleotides at positions shared by the nucleic acid sequences. A degree of identity of amino acid sequences is a function of the number of identical amino acids at positions shared by the amino acid sequences. A degree of homology or similarity of amino acid sequences is a function of the number of amino acids, i.e., structurally related, at positions shared by the amino acid sequences. In certain instances, the “homology” or “identity” or “similarity” of two or more peptides or nucleic acids is defined by a “percent identity” determined using an algorithm such as BLAST, as described in further detail below.

The comparison of sequences and determination of percent identity between two sequences can be accomplished using a mathematical algorithm. The percent identity between two amino acid sequences can be determined using the Needleman and Wunsch algorithm ((1970) J. Mol. Biol. 48:444-453) which has been incorporated into the GAP program in the GCG software package (available at http://www.gcg.com), using either a Blossum 62 matrix or a PAM250 matrix, and a gap weight of 16, 14, 12, 10, 8, 6, or 4 and a length weight of 1, 2, 3, 4, 5, or 6. The percent identity between two nucleotide sequences is determined using the GAP program in the GCG software package (available at http://www.gcg.com), using a NWSgapdna.CMP matrix and a gap weight of 40, 50, 60, 70, or 80 and a length weight of 1, 2, 3, 4, 5, or 6. A particularly useful set of parameters (and the one that should be used if the practitioner is uncertain about what parameters should be applied to determine if a molecule is within a sequence identity or homology limitation of the invention) are a Blossum 62 scoring matrix with a gap penalty of 12, a gap extend penalty of 4, and a frameshift gap penalty of 5.

The percent identity between two amino acid or nucleotide sequences can be determined using the algorithm of Meyers and Miller ((1989) CABIOS, 4:11-17) which has been incorporated into the ALIGN program (version 2.0), using a PAM120 weight residue table, a gap length penalty of 12 and a gap penalty of 4.

The nucleic acid and protein sequences described herein can be used as a “query sequence” to perform a search against public databases to, for example, identify other family members or related sequences. Such searches can be performed using the NBLAST and XBLAST programs (version 2.0) of Altschul et al. (1990) J. Mol. Biol. 215:403-10. BLAST nucleotide searches can be performed with the NBLAST program, score=100, wordlength=12 to obtain nucleotide sequences homologous to nucleic acid molecules described herein. BLAST protein searches can be performed with the XBLAST program, score=50, wordlength=3 to obtain amino acid sequences homologous to TLR11/TLR12 or TLR5 protein molecules of the invention. To obtain gapped alignments for comparison purposes, Gapped BLAST can be utilized as described in Altschul et al., (1997) Nucleic Acids Res. 25:3389-3402. When utilizing BLAST and Gapped BLAST programs, the default parameters of the respective programs (e.g., XBLAST and NBLAST) can be used. See http://www.ncbi.nlm.nih.gov.

The term “humanized” forms of non-human (e.g., murine) antibodies as used herein means specific chimeric immunoglobulins, immunoglobulin chains or fragments thereof (such as Fv, Fab, Fab′, F(ab)₂ or other antigen-binding subsequences of antibodies) which contain minimal sequence derived from non-human immunoglobulin. For the most part, humanized antibodies are human immunoglobulins (recipient antibody) in which residues from the complementary determining regions (CDRs) of the recipient antibody are replaced by residues from the CDRs of a non-human species (donor antibody) such as mouse, rat or rabbit having the desired specificity, affinity and capacity. In some instances, Fv framework region (FR) residues of the human immunoglobulin are replaced by corresponding non-human FR residues. Furthermore, the humanized antibody may comprise residues that are found neither in the recipient antibody nor in the imported CDR or FR sequences. These modifications are made to further refine and optimize antibody performance. In general, the humanized antibody will comprise substantially all of at least one, and typically two, variable domains, in which all or substantially all of the CDR regions correspond to those of a non-human immunoglobulin and all or substantially all of the FR residues are those of a human immunoglobulin consensus sequence. The humanized antibody optimally also will comprise at least a portion of an immunoglobulin constant region (Fc), typically that of a human immunoglobulin.

An “immunomodulatory molecule” is a molecule that alters an immune response. An immunomodulatory molecule can be, for example, a compound, such as an organic chemical; a polypeptide, such as an antibody or cytokine; a nucleic acid, such as a DNA or RNA molecule; or any other type of molecule that alters an immune response. An immunomodulatory molecule can alter an immune response by directly or indirectly altering an activity of a cell that mediates an immune response. An immunomodulatory molecule can act directly on an immune system cell, for example, by binding to a cell surface receptor and stimulating or inhibiting proliferation, differentiation, or expression, secretion or receptor binding of immune system regulatory molecules such as co-stimulatory receptors and ligands, cytokines, and chemokines. Examples of naturally occurring molecules that act directly on immune system cells to alter an immune response include PAMPs, cytokines, chemokines and growth factors. Other examples of molecules that act directly on immune system cells to alter an immune response include molecules that alter receptor functions, such as antibodies to receptors, soluble cytokine receptors, receptor agonists and antagonists, molecules that alter the production of immunomodulatory molecules, such as inhibitors of converting enzymes and molecules involved in the intracellular transport and secretion of immunomodulatory molecules.

An immunomodulatory molecule can indirectly alter the activity of a particular immune system cell by altering the amount or activity of a molecule that regulates a cellular activity of the cell. For example, a cytokine, chemokine, or growth factor produced by an immune system cell, such as a macrophage, can stimulate or inhibit various cellular activities of B and T lymphocytes. Immune cell functions that can be stimulated or inhibited by an immunomodulatory molecule include, for example, immune cell activation, co-activation, proliferation, production of cytokines, cellular interactions and migration. An immunomodulatory molecule can therefore act on a variety of immune cell types and can alter a variety of cellular functions. Immunomodulatory profilin peptides, polypeptides and modifications thereof, used in the methods of the invention, are examples of immunomodulatory molecules useful for inducing an immune response by, for example, binding to TLR5 and inducing a TLR5-mediated increase in macrophage production of TNF-α, IL-1 and IL-6. The profilin polypeptides, peptides and modifications thereof, are also useful for indirectly inducing an immune response because immunomodulatory molecules produced by a TLR5-expressing cell in response to profilin will alter the activities of immune system cells that respond to the particular immunomodulatory molecules produced. An immunomodulatory molecule can mediate an immune response that is nonspecific or augment a specific response.

A specific immunomodulatory molecule alters an immune response to a particular target antigen. Nonlimiting examples of specific immunomodulatory molecules include monoclonal antibodies, including naked monoclonal antibodies, drug-, toxin- or radioactive compound-conjugated monoclonal antibodies, and ADCC targeting molecules. Such immunomodulatory molecules stimulate an immune response by binding to antigens and targeting cells for destruction. An immunomodulatory molecule can be used to suppress an immune response to an antigen. For example, a tolerogenizing molecule can be used to suppress an immune response to a self-antigen.

The term “isolated” as also used herein with respect to nucleic acids, such as DNA or RNA, refers to molecules separated from other DNAs, or RNAs, respectively, that are present in the source of the macromolecule. For example, isolated nucleic acids encoding the subject polypeptides may include no more than 10 kilobases (kb) of nucleic acid sequence which naturally immediately flanks that gene in genomic DNA, and typically no more than 5 kb of such naturally occurring flanking sequences, and most often less than 1.5 kb of such naturally occurring flanking sequence. The term isolated as used herein also refers to a nucleic acid or polypeptide that is substantially free of cellular material, viral material, or culture medium when produced by recombinant DNA techniques, or chemical precursors or other chemicals when chemically synthesized. Moreover, an “isolated nucleic acid” is meant to include nucleic acid fragments which are not naturally occurring as fragments and would not be found in the natural state. The term “isolated” is also used herein to refer to polypeptides which are isolated from other cellular proteins and is meant to encompass both purified and recombinant polypeptides.

“Isolated PRIP”, “highly purified PRIP” and “substantially homogeneous PRIP” are used interchangeably and mean a PRIP that has been purified from a PRIP source or has been prepared by recombinant or synthetic methods and is sufficiently free of other peptides or proteins. “Homogeneous” here means less than about 10 and more usefully less than about 5% contamination with other source proteins.

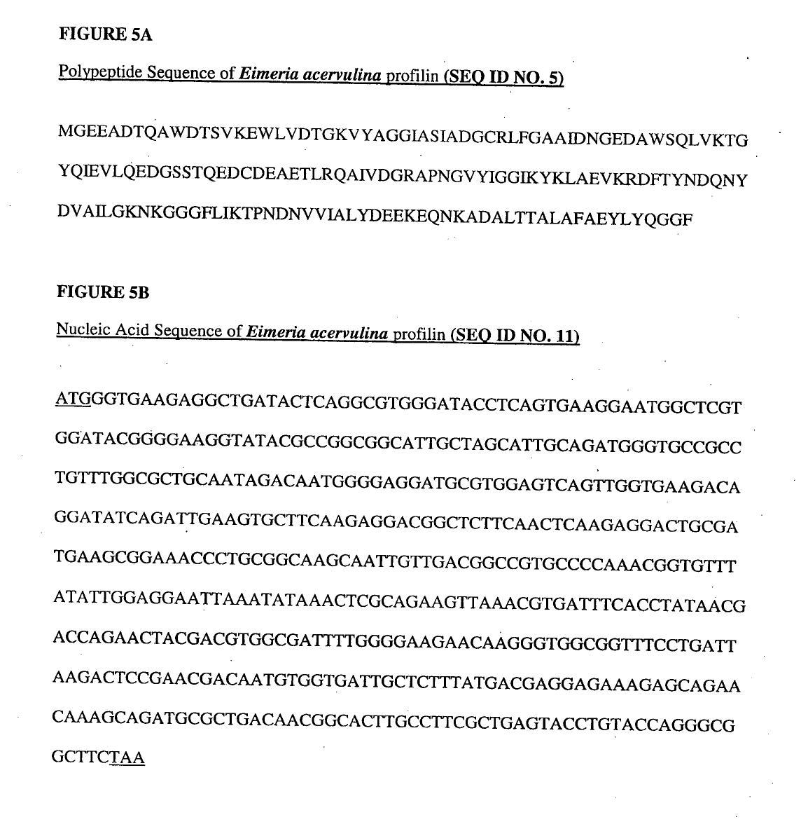

“Isolated PRIP nucleic acid” is RNA or DNA containing greater than 16, and usefully 20 or more, sequential nucleotide bases that encodes biologically active profilin or a fragment thereof, is complementary to the RNA or DNA, or hybridizes to the RNA or DNA and remains stably bound under moderate to stringent conditions. This RNA or DNA is free from at least one contaminating source nucleic acid with which it is normally associated in the natural source and usefully substantially free of any other mammalian RNA or DNA. The phrase “free from at least one contaminating source nucleic acid with which it is normally associated” includes the case where the nucleic acid is present in the source or natural cell but is in a different chromosomal location or is otherwise flanked by nucleic acid sequences not normally found in the source cell. An example of isolated PRIP nucleic acid is RNA or DNA that encodes a biologically active PRIP sharing at least 75%, at least 80%, at least 85%, at least 90%, and at least 95% sequence identity with the PRIPS shown in FIGS. 1B, 2B, 3B, 4B, 5B, 6B, and 7B (SEQ ID NOS: 1, 2, 3, 4, 5, 6, or 7, respectively).

The expression “labeled” when used herein refers to a molecule (e.g., PRIP or anti-PRIP antibody) that has been conjugated, directly or indirectly, with a detectable compound or composition. The label may be detectable by itself (e.g., radioisotope labels or fluorescent labels) or, in the case of an enzymatic label, may catalyze a chemical alteration of a substrate compound or composition, which is detectable. A useful label is an enzymatic one which catalyzes a color change of a non-radioactive color reagent.

“Mammal” for purposes of treatment refers to any animal classified as a mammal, and which bears its young live, including, but not limited to, humans, domestic and farm animals, and zoo, sports, or pet animals, such as sheep, dogs, horses, cats, cows, etc.

The term “marker” or “marker sequence” or similar phrase means any gene that produces a selectable genotype or a selectable phenotype. Nonlimiting representative markers are the neo gene, green fluorescent protein (GFP) gene, TK gene, β-galactosidase gene, etc. The marker sequence may be any sequence known to those skilled in the art that serves these purposes, although typically the marker sequence will be a sequence encoding a protein that confers a selectable trait, such as an antibiotic resistance gene, or an enzyme that can be detected and that is not typically found in the cell. The marker sequence may also include regulatory regions such as a promoter or enhancer that regulates the expression of that protein. However, it is also possible to transcribe the marker using endogenous regulatory sequences. The marker facilitates separation of transfected from untransfected cells by fluorescence activated cell sorting, for example by the use of a fluorescently labeled antibody or the expression of a fluorescent protein such as GFP. Other DNA sequences that facilitate expression of marker genes may also be incorporated into the DNA constructs of the present invention. These sequences include, but are not limited to transcription initiation and termination signals, translation signals, post-translational modification signals, intron splicing junctions, ribosome binding sites, and polyadenylation signals, to name a few. The marker sequence may also be used to append sequence to the target gene. For example, it may be used to add a stop codon to truncate IL-1RN translation. The use of selectable markers is well known in the art and need not be detailed herein. The term “modulation” as used herein refers to both upregulation (i.e., activation or stimulation (e.g., by agonizing or potentiating)) and downregulation (i.e., inhibition or suppression (e.g., by antagonizing, decreasing or inhibiting)).

The term “monoclonal antibody” as used herein refers to an antibody obtained from a population of substantially homogeneous antibodies, i.e., the individual antibodies comprising the population are identical except for possible naturally occurring mutations that may be present in minor-amounts. Monoclonal antibodies are highly specific, being directed against a single antigenic site. Furthermore, in contrast to conventional (polyclonal) antibody preparations that typically include different antibodies directed against different determinants (epitopes), each monoclonal antibody is directed against a single determinant on the antigen. Novel monoclonal antibodies or fragments thereof include in principle all immunoglobulin classes such as IgM, IgG, IgD, IgE, IgA or their subclasses such as the IgG subclasses or mixtures thereof. IgG and its subclasses, such as IgG₁, IgG₂, IgG₂ₐ, IgG_{2b}, IgG₃ or IgG_{M} are useful. The IgG subtypes IgG_{1/kappa} and IgG_{2b/kapp} are also useful.

The monoclonal antibodies herein include hybrid and recombinant antibodies produced by splicing a variable (including hypervariable) domain of an anti-profilin antibody with a constant domain (e.g., “humanized” antibodies), or a light chain with a heavy chain, or a chain from one species with a chain from another species, or fusions with heterologous proteins, regardless of species of origin or immunoglobulin class or subclass designation, as well as antibody fragments (e.g., Fab, F(ab)₂, and Fv), so long as they exhibit the desired biological activity. (See, e.g., U.S. Pat. No. 4,816,567 and Mage & Lamoyi, in Monoclonal Antibody Production Techniques and Applications, pp. 79-97 (Marcel Dekker, Inc.), New York (1987)). Thus, the modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by the hybridoma method first described by Kohler & Milstein, Nature 256:495 (1975), or may be made by recombinant DNA methods (U.S. Pat. No. 4,816,567). The “monoclonal antibodies” may also be isolated from phage libraries generated using the techniques described in McCafferty et al., Nature 348:552-554 (1990), for example.

A “mutated gene” or “mutation” refers to an allelic form of a gene (e.g., a PRIP), which is capable of altering the biological activity of that gene relative to the nonmutated or “cord type” form of that gene.

As used herein, the term “nucleic acid” refers to polynucleotides such as deoxyribonucleic acid (DNA), ribonucleic acid (RNA), and RNA/DNA hybrids. The term should be understood to include either single- or double-stranded forms of nucleic acid, and, as equivalents, analogs of either RNA and/or DNA. Such nucleic acid analogs may be composed of nucleotide analogs, and, as applicable to the embodiment being described, may be single-stranded (such as sense or antisense) or double-stranded polynucleotides.

By “neutralizing antibody” is meant an antibody molecule as herein defined which is able to block or significantly reduce an effector function of e.g., native sequence profilin. Such a “neutralizing antibody” includes an antibody molecule that is able to block or significantly reduce a biological activity of native sequence profilin. For example, a neutralizing antibody may inhibit or reduce the ability of profilin to modulate and/or activate TLR11/TLR12 and/or TLR5.

The phrase “nucleotide sequence complementary to the nucleotide sequence set forth in SEQ ID NO: x” refers to the nucleotide sequence of the complementary strand of a nucleic acid strand having SEQ ID NO: x. The term “complementary strand” is used herein interchangeably with the term “complement”. The complement of a nucleic acid strand can be the complement of a coding strand or the complement of a non-coding strand. When referring to double-stranded nucleic acids, the complement of a nucleic acid having SEQ ID NO: x refers to the complementary strand of the strand having SEQ ID NO: x or to any nucleic acid having the nucleotide sequence of the complementary strand of SEQ ID NO: x. When referring to a single-stranded nucleic acid having the nucleotide sequence SEQ ID NO: x, the complement of this nucleic acid is a nucleic acid having a nucleotide sequence which is complementary to that of SEQ ID NO: x. The nucleotide sequences and complementary sequences thereof are always given in the 5′ to 3′ direction, unless indicated otherwise.

“Operably linked” when referring to nucleic acids means that the nucleic acids are placed in a functional relationship with another nucleic acid sequence. For example, DNA for a presequence or secretory leader is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are contiguous and, in the case of a secretory leader, contiguous and in reading phase. However, enhancers do not have to be contiguous. Linking is accomplished by ligation at convenient restriction sites. If such sites do not exist, the synthetic oligonucleotide adaptors or linkers are used in accord with conventional practice.

The term “percent identical” refers to sequence identity between two amino acid sequences or between two nucleotide sequences. Identity can each be determined by comparing a position in each sequence which may be aligned for purposes of comparison. When an equivalent position in the compared sequences is occupied by the same base or amino acid, then the molecules are identical at that position; when the equivalent site occupied by the same or a similar amino acid residue (e.g., similar in steric and/or electronic nature), then the molecules can be referred to as homologous (similar) at that position. Expression as a percentage of homology/similarity or identity refers to a function of the number of identical or similar amino acids at positions shared by the compared sequences. Various alignment algorithms and/or programs may be used, including FASTA, BLAST or ENTREZ. FASTA and BLAST are available as a part of the GCG sequence analysis package (University of Wisconsin, Madison, Wis.), and can be used with, e.g., default settings. ENTREZ is available through the National Center for Biotechnology Information, National Library of Medicine, National Institutes of Health, Bethesda, Md. The percent identity of two sequences can be determined by the GCG program with a, gap weight of 1, e.g., each amino acid gap is weighted as if it were a single amino acid or nucleotide mismatch between the two sequences.

A “PRIP fragment” is a portion of a naturally occurring full-length profiling-related immunomodulatory protein sequence having one or more amino acid residues deleted. The deleted amino acid residue(s) may occur anywhere in the polypeptide, including at either the N-terminal or C-terminal end or internally. Accordingly, a “PRIP fragment” of the invention may or may not possess one or more biological activities of a profiling-related immunomodulatory protein. “PRIP fragments” typically, will have a consecutive sequence of at least 20, 30, or 40 amino acid residues of a PRIP polypeptide (e.g., human PRIPs shown in FIGS. 2A and 2B (SEQ ID NOS: 1 and 2)). Nonlimiting representative PRIP fragments have about 30-150 residues, which are identical to the sequence of a profiling-related immunomodulatory polypeptide. Other useful PRIP fragments include those produced as a result of chemical or enzymatic hydrolysis or digestion of the purified PRIP polypeptides.

The terms “PRIP variants” or “sequence variants”, as used herein, means biologically active (i.e., immunomodulatory) PRIPs having less than 100% sequence identity with a native PRIPs as described herein.

A “recombinant nucleic acid” comprises or is encoded by one or more nucleic acid which is derived from a nucleic acid which was artificially constructed. For example, the nucleic acid can comprise or be encoded by a cloned nucleic acid formed by joining heterologous nucleic acids (see, e.g., Berger and Kimmel, Guide to Molecular Cloning Techniques, in Meth. Enzymol. Vol. 152 Academic Press, Inc., San Diego, Calif., and in Sambrook et al. Molecular Cloning—A Laboratory Manual (2nd ed.) Vol. 1-3 (1989) (Sambrook) and in Current Protocols in Molecular Biology, Ausubel, F. M., et al., eds., Greene Publishing Associates, Inc. and John Wiley & Sons, Inc., (1996 Supplement). Alternatively, the nucleic acid can be synthesized chemically.

As used herein, a “reporter gene construct” is a nucleic acid that includes a “reporter gene” operably linked to a transcriptional regulatory sequences. Transcription of the reporter gene is controlled by these sequences. The transcriptional regulatory sequences include the promoter and other regulatory regions, such as enhancer sequences, that modulate the activity of the promoter, or regulatory sequences that modulate the activity or efficiency of the RNA polymerase that recognizes the promoter, or regulatory sequences are recognized by effector molecules.

As used herein, the term “promoter” means a DNA sequence that regulates expression of a selected DNA sequence operably linked to the promoter, and which effects expression of the selected DNA sequence in cells. The term encompasses “tissue specific” promoters, i.e., promoters, which effect expression of the selected DNA sequence only in specific cells (e.g., cells of a specific tissue). The term also covers so-called “leaky” promoters, which regulate expression of a selected DNA primarily in one tissue, but cause expression in other tissues as well, The term also encompasses non-tissue specific promoters and promoters that constitutively express or that are inducible (i.e., expression levels can be controlled).

The term “recombinant protein” refers to a polypeptide of the present invention which is produced by recombinant DNA, techniques, wherein generally, DNA encoding a specific polypeptide is inserted into a suitable expression vector which is in turn used to transform a host cell to produce the heterologous protein. Moreover, the phrase “derived from”, with respect to a recombinant target gene, is meant to include within the meaning of “recombinant protein” those proteins having an amino acid sequence of a native target polypeptide, or an amino acid sequence similar thereto which is generated by mutations including substitutions and deletions (including truncation) of a naturally occurring form of the polypeptide.

As used herein, “recombinant cells” include any cells that have been modified by the introduction of heterologous DNA. Control cells include cells that are substantially identical to the recombinant cells, but do not express one or more of the proteins encoded by the heterologous DNA, e.g., do not include or express a recombinant sulfotransferase gene.

The word “sample” refers to body fluid, excretion, tissue or a cell from a patient. Normally, the sample is removed from the patient, but in vivo diagnosis is also contemplated. Patient samples include urine, serum, blood, sputum, cell extracts, lymph, spinal fluid, synovial fluid, feces, lacrinal secretions, seminal fluid, vaginal secretions, and the like, are also included within the meaning of the term.

The term “substantially free of other cellular proteins” (also referred to herein as “contaminating proteins”) or “substantially pure or purified preparations” are defined as encompassing preparations of PRIPs having less than about 20% (by dry weight) contaminating protein, and usefully having less than about 5% contaminating protein.

“Small molecule” as used herein, is meant to refer to a composition, which has a molecular weight of less than about 5 kD and most typically less than about 4 kD. Small molecules can be nucleic acids, peptides, polypeptides, peptidomimetics, carbohydrates, lipids or other organic (carbon containing) or inorganic molecules. Many pharmaceutical companies have extensive libraries of chemical and/or biological mixtures, often fungal, bacterial, or algal extracts, which can be screened with any of the assays of the invention to identify compounds that modulate a target bioactivity.

As used herein, the term “specifically hybridizes” or “specifically detects” refers to the ability of a nucleic acid molecule of the invention to bind via hydrogen bonds or van der Waals forces to at least approximately 6, 12, 20, 30, 50, 100, 150, 200, 300, 350, 400 or 425 consecutive nucleotides of a gene, e.g., a profilin-related immunomodulatory protein (PRIP)-encoding gene.

The term “substantially homologous”, when used in connection with amino acid sequences, refers to sequences which are substantially identical to or similar in sequence, giving rise to a homology in conformation and thus to similar biological activity. The term is not intended to imply a common evolution of the sequences.

As used herein, the term “transfection” means the introduction of a nucleic acid, e.g., via an expression vector or by force using, e.g., a gene gun, into a recipient cell by nucleic acid-mediated gene transfer. Methods for transformation which are known in the art include any electrical, magnetic, physical, biological or chemical means. As used herein, “transfection” includes such specific techniques as electroporation, magnetoporation, Ca⁺⁺ treatment, injection, bombardment, retroviral infection and lipofection, among others. “Transformation” as used herein, refers to a process in which a cell's genotype is changed as a result of the cellular uptake of exogenous nucleic acid, and, for example, the transformed cell expresses a recombinant form of a target polypeptide or, in the case of anti-sense expression from the transferred gene, the expression of a naturally-occurring form of the target polypeptide is disrupted.

As used herein, the term “transgene” means a nucleic acid sequence (encoding, e.g., a PRIP) which has been introduced into a cell. A transgene could be partly or entirely heterologous, i.e., foreign, to the transgenic animal or cell into which it is introduced, or, is homologous to an endogenous gene of the transgenic animal or cell into which it is introduced, but which is designed to be inserted, or is inserted, into the animal's genome in such a way as to alter the genome of the cell into which it is inserted (e.g., it is inserted at a location which differs from that of the natural gene or its insertion results in a knockout). A transgene can also be present in a cell in the form of an episome. A transgene can include one or more transcriptional regulatory sequences and any other nucleic acid, such as introns, that may be necessary for optimal expression of a selected nucleic acid.

“Treatment” refers to both therapeutic treatment and prophylactic or preventative measures. Those in need of treatment include those harboring the disease, disorder (e.g., cancer or an infectious disease), as well as those prone to have the disorder or those in which the disorder is to be prevented.

The term “vector” refers to a nucleic acid molecule capable of transporting another nucleic acid to which it has been linked. One nonlimiting type of vector is an episome, i.e., a nucleic acid capable of extra-chromosomal replication. Other useful vectors are those capable of autonomous replication and/or expression of nucleic acids to which they are linked. Vectors capable of directing the expression of genes to which they are operatively linked are referred to herein as “expression vectors”. In general, expression vectors of utility in recombinant DNA techniques are often in the form of “plasmids” which refer generally to circular double-stranded DNA loops which, in their vector form are not bound to the chromosome. The terms “plasmid” and “vector” are used herein interchangeably. However, the invention is intended to include such other forms of expression vectors which serve equivalent functions and which become known in the art subsequently hereto.

The term “wild-type allelle” refers to an allele of a gene which, when present in two copies in a subject results in a wild-type phenotype. There can be several different wild-type alleles of a specific gene, since certain nucleotide changes in a gene may not affect the phenotype of a subject having two copies of the gene with the nucleotide changes.

“Percent amino acid sequence identity” with respect to the profilin sequence is defined herein as the percentage of amino acid residues in the candidate sequence that are identical with the residues in a PRIP sequence, after aligning the sequences and introducing gaps, if necessary, to achieve the maximum percent sequence identity, and not considering any conservative substitutions as part of the sequence identity. None of N-terminal, C-terminal, or internal extensions, deletions, or insertions into the profilin sequence shall be construed as affecting sequence identity or homology. Percent amino acid sequence identity may be conveniently determined using an appropriate algorithm (e.g., the BLAST algorithm available through NCBI at www.ncbi.nlm.nih.gov/).

### 4.3 PRIPs and PLIPs and Cognate Nucleic Acids

4.3.1 Profilins

The invention provides, in part, profilin, profilin-related and profilin-like immunomodulatory polypeptides and cognate isolated natural and synthetic nucleic acids that encode them.

The invention includes profilins possessing immunomodulatory activity, as well as structural features which are shared by the immunomodulatory proteins of the invention. Profilin is a multi-functional protein. In particular, profilin has multiple binding sites (i.e., for actin, Arp2/3 complex, proline-rich peptides and proteins, poly-L-Pro, and phosphatidylinositol (PIP2) phosphate); and possesses tumor suppressor activity (over-expression of profilin by human cancer cells makes them less tumorigenic). In addition, a plant profilin has been shown to trigger both T-cell and B-cell responses, which may be responsible for observed allergic effects in humans.

Profilin itself is a low molecular weight (12-16 kD) ubiquitous protein expressed in all eukaryotes which binds to actin in muscle and non-muscle cells, controlling actin polymerization. Profilin has two isoforms, profilin type-1 and profilin type-2. It is also known that many plant profilins are allergens. Profilin can inhibit actin polymerization into F-actin by binding to monomeric actin (G-actin) and terminal F-actin subunits, but, as a regulator of the cytoskeleton, it may also promote actin polymerization. It plays a role in the assembly of branched actin filament networks, by activating the Wiskott-Aldrich Syndrome protein (WASP) via binding to the proline-rich domain of WASP. Profilin may link the cytoskeleton with major signaling pathways by interacting with components of the phosphatidylinositol cycle and Ras pathway. While human profilin type-1 is inactive in dendritic cell activation (“DCA”) assays and does not appear to have immunomodulatory and/or TLR11/TLR12 or TLR5 agonist activity, other proteins structurally related to profilin do possess one or more of these activities.

4.3.2 Profilin-Related-Immunomodulatory Proteins (PRIPs)

The present invention makes available PRIPs which are isolated from, or otherwise substantially free of, other cellular proteins, such as other signal transduction factors and/or transcription factors which may normally be associated with the PRIP. Functional forms of a PRIP can be prepared, as purified preparations by using a cloned gene as described herein. Full length proteins or fragments corresponding to one or more particular motifs and/or domains or to arbitrary sizes, for example, at least about 5, at least about 10, at least about 25, at least about 50, at least about 75, or at least about 100, amino acids in length are within the scope of the present invention.

Alternatively, the PRIP fragment includes the core domain of profilin and comprises at least 5 contiguous amino acid residues, at least 20 contiguous amino acid residues, or at least 50 contiguous amino acid residues of SEQ ID NOS: 1-6.

Isolated PRIPs can be encoded by all or a portion of a nucleic acid sequence shown in any of SEQ ID NOS: 7-12. Isolated peptidyl portions of PRIPs can be obtained by any known method, including by screening peptides recombinantly produced from the corresponding fragment of the nucleic acid encoding such peptides. In addition, fragments can be chemically synthesized using techniques known in the art, such as conventional Merrifield solid phase f-Moc or t-Boc chemistry. For example, a PRIP of the present invention may be arbitrarily divided into fragments of desired length with no overlap of the fragments, or usefully divided into overlapping fragments of a desired length. The fragments can be produced (recombinantly or by chemical synthesis) and tested to identify those peptidyl fragments which can function as either agonists or antagonists of a wild-type profilin protein.

Another aspect of the present invention includes recombinant forms of the PRIPs. In addition to native profilin proteins, which are encoded by a nucleic acid that is at least 60%, at least 80%, at least 85%, at least 90%, or at least 95% identical to an amino acid sequence represented by SEQ ID NOS: 1-6 or encoded by SEQ ID NOS: 7-12. Polypeptides which are encoded by a nucleic acid that is at least about 98-99% identical to the sequence of SEQ ID NOS: 7-12 or which are 98-99% identical with the amino acid sequence set forth in SEQ ID NOS: 1-6, are also within the scope of the invention.

A PRIP of the present invention can be a mammalian PRIP such as a human PRIP. The PRIP can have an amino acid sequence as set forth in SEQ ID NOS: 1-6. In some cases, the PRIP retains profilin bioactivity.

Recombinant PRIPs are capable of functioning in one of either role of an agonist or antagonist with at least one biological activity of a wild-type profilin protein, as set forth in the appended Sequence Listing.

In general, polypeptides referred to herein as having an activity of a PRIP are defined as polypeptides which include an amino acid sequence encoded by all or a portion of the nucleic acid sequences shown in one of SEQ ID NOS: 7-12 and which mimic or antagonize all or a portion of the biological/biochemical activities of a naturally occurring profilin protein. Biological activities of the subject PRIPs include activity as a tumor suppressor, functions in cell cycle control of various developmental processes, apoptosis, gene expression, modulation of proliferation and differentiation, and tumorigenesis. Assays for determining whether a compound, e.g., a protein, such as a PRIP or variant thereof, has one or more of the above immunomodulatory biological activities are well known in the art, some of which are described herein.

Profilin-related genes similar to profilin genes are found in 1-5 copies in every living being, including some viruses. Profilins in higher plants represent up to 5% of the total weight of plant pollen, are highly antigenic, and are responsible for a significant percentage of cases of human allergy to peach, birch pollen, and natural rubber.

A protein that is structurally similar to profilin is PA19. PA19 is a 19 kD protozoan sporozite antigen originally isolated from Eimeria acervulina, and later shown to be conserved in 3 Eimeria species (see Jenkins et al. (1988) Exp. Parasitol. 66:96-107; and Laurent et al. (1994) Mol. Biochem. Parasitol. 63:79-86). PA19 in Eimeria is a low-abundance surface protein and the nucleotide sequence of the first PA19 clone has been submitted to GenBank under the name of 19 kD sporozite antigen (GenBank Accession Z26584). Analysis of the primary sequence of PA19 protein from Eimeria has revealed structural similarity to the actin-binding protein profilin.

The invention further includes novel non-protozoan immunomodulatory polypeptides, such as profilin-related plant polypeptides that include an amino acid sequence from B. nigra (river birch tree) and from P. banksiana (ack pine tree). These polypeptides are both non-PA19, in that they are non-Eimeria in origin, and non-protozoan in that they are derived from non-protozoan organisms (e.g., plant, animal or fungi).

In most protozoan parasites the PA19 gene homolog is interrupted by two long introns. The promoter region typically contains several SPI-like signals in it. For example, in P. falciparum, the single copy gene is located on chromosome 9, and it has been newly discovered that PA19 protein is clearly expressed in the schizont phase of development, with the levels of mRNA at a minimum at 15 hrs, and at a maximum at 36-40 hrs.

A search for the profiles most similar to profilin in the PlasmoDB transcriptome database identified: actin (with 0.991 correlation); membrane protein ag-1 (0.984); cAMP-dependent protein kinase (0.982); Leu/Phe-tRNA protein transferase (0.976); and several hypothetical proteins (with correlations from 0.986 till 0.973). The functional relation of the PA19 protein to profilin indicates that the PA19 from E. acervulina can interact to some extent with rabbit muscle actin and poly-L-Proline (Fetterer, R. H., et al., J. Parasitol. 2004. 90(6): 1321-8)

PA19 from different parasites are not as homologous. These sequences would not be predicted based on the previously known sequences. The newly discovered sequences sometimes share only 70-80% protein similarity and even less similarity at the nucleotide level. Table 1 below shows a comparison of PA19 protein sequences from different organisms (BLASTP).

TABLE 1

| | E. tenella | | E. acervulina | | N. canium | | T. gondii | | S. neurona | | P. falciparum | | C. parvum | |
|---|---|---|---|---|---|---|---|---|---|---|---|---|---|---|
| E. acervulina | 84% | Iden.¹ | | | | | | | | | | | | |
| | 92% | Posit. | | | | | | | | | | | | |
| | 0.1% | Gaps | | | | | | | | | | | | |
| N. canium | 45% | Iden. | 50% | Iden. | | | | | | | | | | |
| | 64% | Posit. | 69% | Posit. | | | | | | | | | | |
| | 1% | Gaps | 1% | Gaps | | | | | | | | | | |
| T. gondii | 48% | Iden. | 51% | Iden. | 90% | Iden. | | | | | | | | |
| | 68% | Posit. | 71% | Posit. | 91% | Posit. | | | | | | | | |
| | 1% | Gaps | 1.5% | Gaps | 0.1% | Gaps | | | | | | | | |
| S. neurona | 39% | Iden. | 40% | Iden. | 63% | Iden. | 64% | Iden. | | | | | | |
| | 59% | Posit. | 60% | Posit. | 79% | Posit. | 79% | Posit. | | | | | | |
| | 1% | Gaps | 3% | Gaps | 0.1% | Gaps | 0% | Gaps | | | | | | |
| P. falciparum | 37% | Iden. | 38% | Iden. | 42% | Iden. | 42% | Iden. | 42% | Iden. | | | | |
| | 57% | Posit. | 57% | Posit. | 60% | Posit. | 61% | Posit. | 57% | Posit. | | | | |
| | 2% | Gaps | 2% | Gaps | 4% | Gaps | 4% | Gaps | 4% | Gaps | | | | |
| C. parvum | 43% | Iden. | 46% | Iden. | 49% | Iden. | 46% | Iden. | 44% | Iden. | 35% | Iden. | | |
| | 62% | Posit. | 62% | Posit. | 66% | Posit. | 63% | Posit. | 61% | Posit. | 51% | Posit. | | |
| | 2% | Gaps | 2% | Gaps | 1% | Gaps | 0.1% | Gaps | 0.1% | Gaps | 6% | Gaps | | |
| B. bovis | 27% | Iden. | 31% | Iden. | 37% | Iden. | 40% | Iden. | 34% | Iden. | 34% | Iden. | 25% | Iden. |
| | 45% | Posit. | 47% | Posit. | 55% | Posit. | 56% | Posit. | 52% | Posit. | 53% | Posit. | 47% | Posit. |
| | 0% | Gaps | 0% | Gaps | 1.5% | Gaps | 1.5% | Gaps | 6% | Gaps | 6% | Gaps | 2% | Gaps |

¹“Iden.” and “Posit.” are the abbreviations for the terms “identity” and “position,” respectively.

This table shows a calculated percent identity and similarity (called “positives” by the BLASTP program) for PA19 from selected parasites, including E. acervulina, a close relative of E. tenella, for which the protein sequence has been published, as well as C. parvum and B. bovis, for which the published protein sequences are not available but were newly derived from the public databases.

The homology level even for closely related species (E. tenella and E. acervulina) is only 77% starting from residue 213 (in E. tenella) to the stop codon. The BLASTN program does not see any reasonable similarity between E. tenella and E. acervulina mRNAs in the region before residue 213. A preliminary alignment is provided in FIG. 22.

PA19 from N. canium (NC) and T. gondii (TG) share the highest homology (they have only 6 differences out of 163 amino acids with 4 of these differences being conservative changes E to D (pos. 48), V to A (pos. 61), T to S (pos. 66), and V to I (pos. 67); and 2 are non-conservative changes: N to C (pos. 62), and V to G (pos. 80). In both of these positions (pos. 62 and 80) the more active version of PA19 (TG) more closely resembles the most active molecule of PA19 E. Tenella (ET): I to C (pos. 62), and G in both (pos. 80). Therefore, one of these positions might be important for activity of the protein. Five of the PA19-related proteins have been tested for activity in DC assays: PA 19 related proteins from P. falciparum, S. neurona, E. tenella, T. gondii, and N. caninum have shown activity.

These polypeptides can be synthesized or isolated from a natural source, by using any methods known in the art. Such methods would be within the routine skill of one of sill in the art once the sequence is known.

Representative profilin-related immunomodulatory polypeptides (PRIPs) include an amino acid sequence corresponding to SEQ ID NO:1 (N. caninum), SEQ ID NO:2 (S. neurona), and SEQ ID NO: 3 (T. gondii). These new profilin-related protozoan polypeptides are novel PA19-like non-Eimeria protozoan immunomodulatory proteins.

Protozoan profilin-like proteins are ligands for TLR11 (Yarovinsky, F., et al., Science, 308, 1626-1629, 2005) and proteins from uropathogenic strains of E. coli may also serve as ligands for TLR11. While not wishing to be limited by theory, these ligands from microbial pathogens may interact with the same TLR11/TLR12 and/or TLR5 receptors. If so, the protein in the uropathogenic strain of E. coli may interact with the receptor through a region that is homologous to the corresponding region on PA19 protein. To find this region, an extended homology search (expectation parameter set for 20000) of the complete genome of E. coli CFT073 (the only uropathogenic strain available so far through GenBank) was performed using as queries PA19 from three protozoan parasites that have been shown to activate dendritic cells (DCs). This search reveals numerous entries with low similarity. Two of these entries were common for all three queries. One belonged to UvrB protein, and the other to UvrC protein. FIG. 12 shows the similarity between PA19 of E. tenella and UvrBC of E. coli CFT073 (25% identities, 53% positives for UvrB and 33% identities, 53% positives for UvrC as assigned by BLAST2 program).

Accordingly, the invention includes UvrABC complexes and UvrB and UvrC polypeptides and polypeptide fragments having some homology to PA19 from E. tenella/T. gondii/N. caninum or other PRIPs of the invention. One region of PA19 was found to be homologous to UvrB (residues 123-163 in PA19, residues 61-101 in UvrB, 24%-25% identities, about 45%-53% positives), and one region of PA19 was found homologous to UvrC (residues 81-125 in PA19, residues 453-499 in UvrC, 28%-38% identities, 44%-53% positives, 6% gaps). Thus, the region close to the C-terminal end of the protein (residues 81-163) of PA19 has some similarity to UvrBC complex. This region aligns with a region from profilin that participates in binding actin. Analysis of the literature shows that there are some “folding-conservative” residues in the region, and the region additionally has a similarity to a conserved domain, COG720, of 6-pyrovoyl-tetrahydropterin synthase (residues 103-167 of PA19, score 27.6 bits).

An equimolar mixture of UvrB and UvrC proteins prepared from E. coli effectively interfered with the ability of PA19 to activate DCs, suggesting a structural relatedness between UvrB and UvrC and PA19.

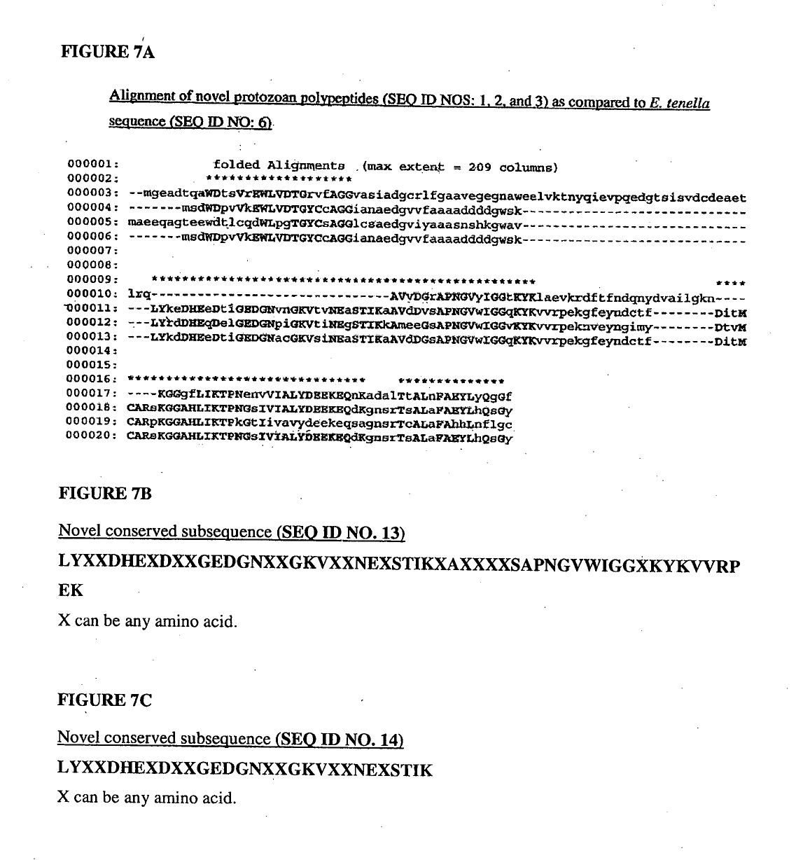

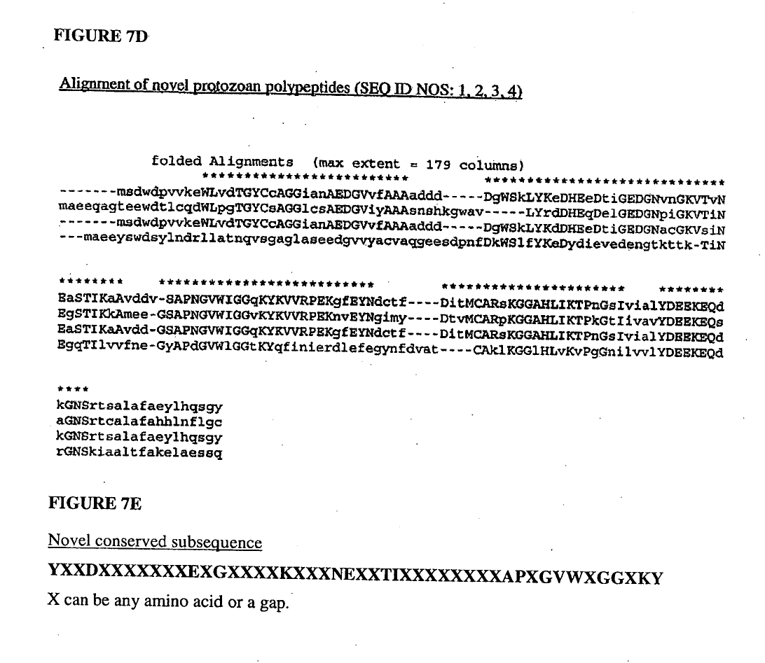

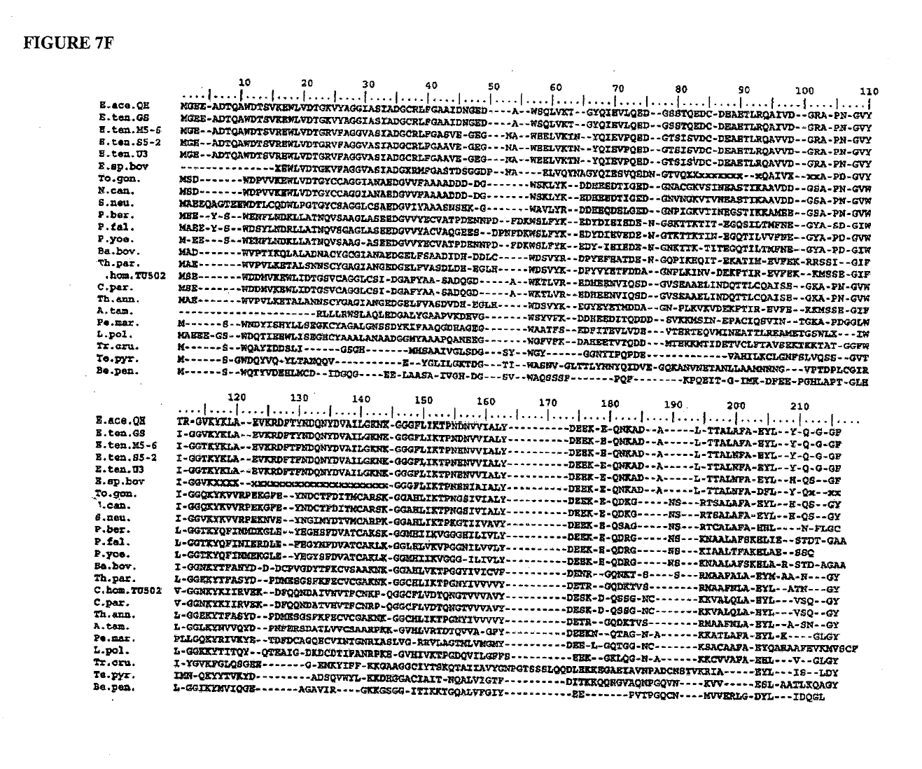

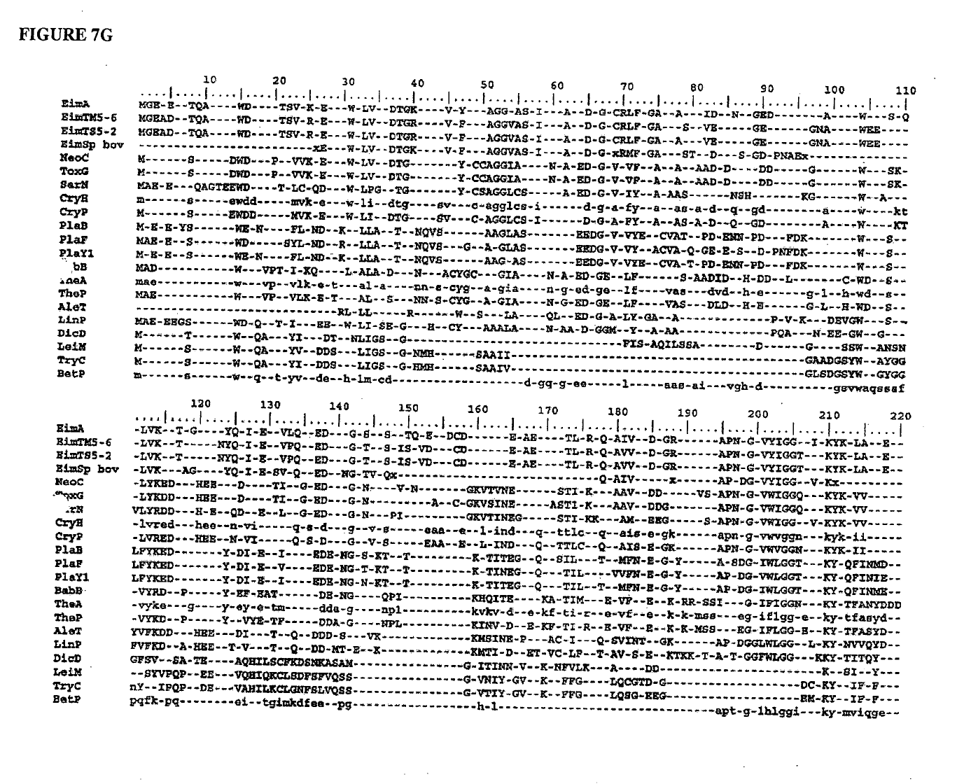

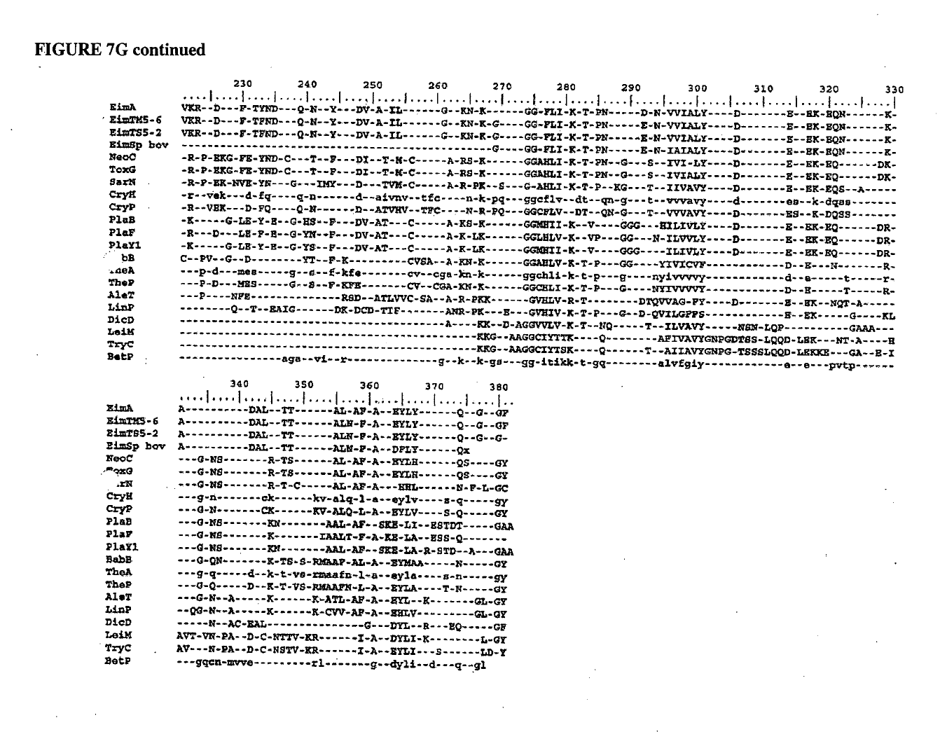

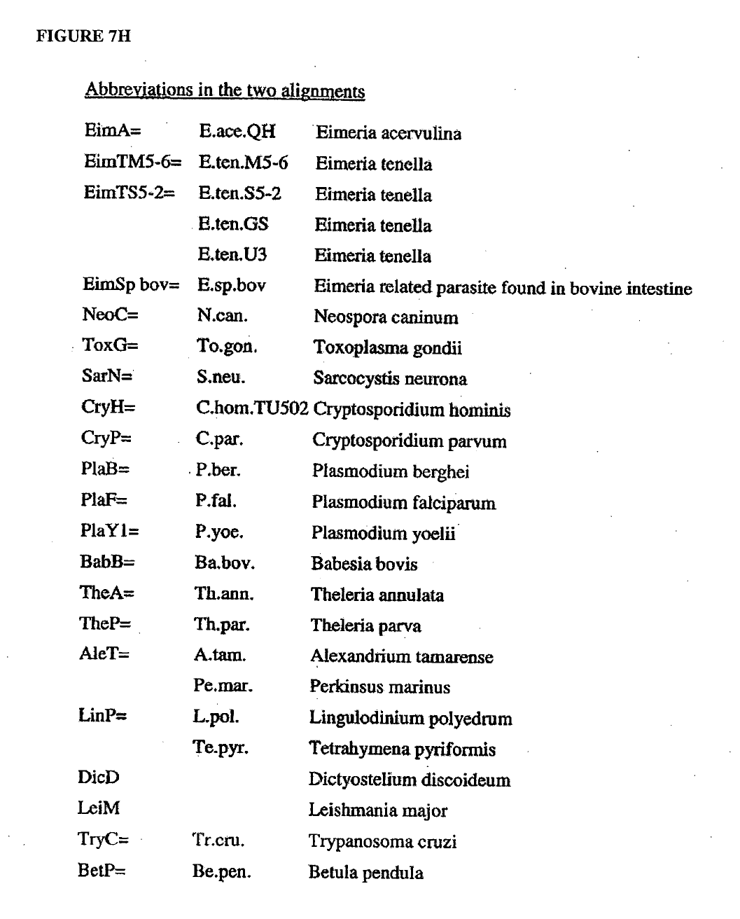

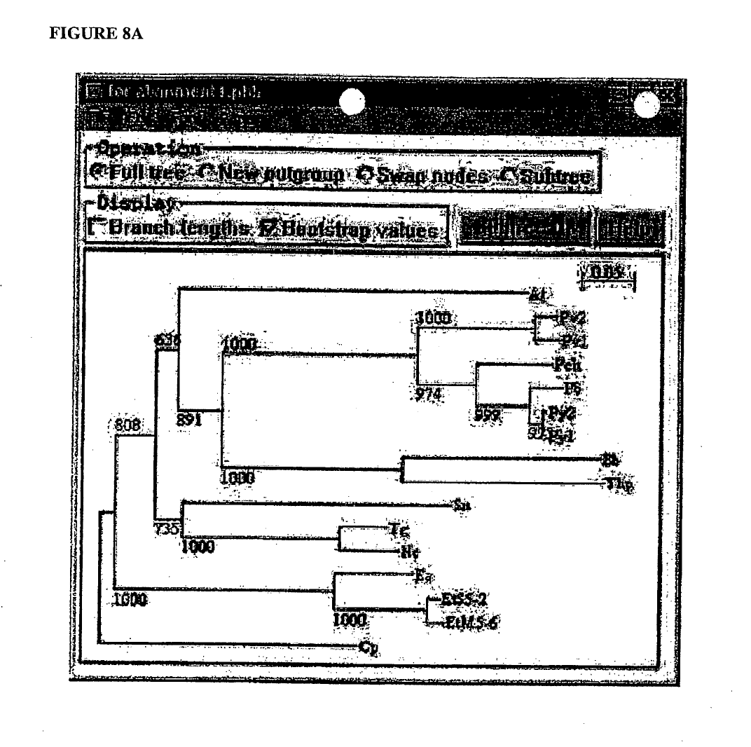

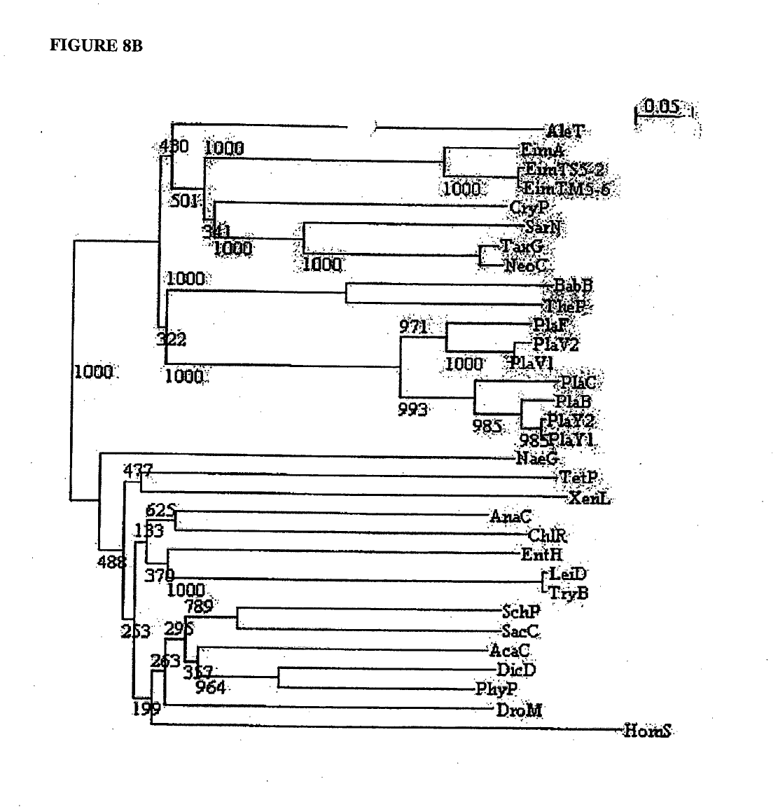

The profilin-related immunomodulatory polypeptides (PRIPs) of the invention include those featuring the conserved motif: LYXXDHEXDXXGEDGNXXGKVXXNEXSTIKXAXXXXSAPNGVWIGGXKYKVVRPEK (SEQ. ID. NO: ______). An alignment of novel protozoan polypeptides from N. caninum, S. neurona, and T. gondii (SEQ ID NOS: 1, 2, and 3, respectively) as compared to the E. tenella polypeptide sequence (SEQ ID NO: 6), shows that there is a novel conserved subsequence: LYXXDHEXDXXGEDGNXXGKVXXNEXSTIKXAXXXXSAPNGVWIGGXKYKVVRPEK (SEQ ID NO: 13), in which X can be any amino acid. The alignment is shown in FIG. 7A and consensus sequence is shown in FIG. 7B. An additional novel conserved subsequence, which is a subsequence of SEQ ID NO: 13, is: LYXXDHEXDXXGEDGNXXGKVXXNEXSTIK (SEQ ID NO: 14), in which X can be any amino acid. An alignment of novel protozoan polypeptides from N. caninum, S. neurona, T. gondii, and P. falciparum (SEQ ID NOS: 1, 2, 3, 4, respectively) as compared to the E. tenella polypeptide sequence (SEQ ID NO: 6) shows that there is a novel conserved subsequence: YXXDXXXXXXXEXGXXXXKXXXNEXXTIXXXXXXXXAPXGVWXGGXKY, in which X can be any amino acid or a gap. This alignment without the E. tenella sequence is shown in FIG. 7D and the consensus (SEQ ID NO: 6) is shown in FIG. 7E.

Accordingly, in one aspect, the invention provides profilin-like and profilin-related immunomodulatory polypeptides that are both structurally related to profilin and possess immunomodulatory activity. Further profilin-related and profilin-like, as well as PA19-like proteins, may be identified by their structural relatedness to profilin and immunomodulatory activities using standard analytical techniques.

The structure-function relationship of the immunomodulatory polypeptides of the invention have been further addressed by the analysis of mutants of PA19 protein. Removal of 5 or more amino acid residues from the C-terminus of the protein completely destroys the ability of the PA19 to activate dendritic cells. Removal of up to 20 amino acids from the N-terminus of PA19, as well as adding a FLAG-tag, or more than 30 total amino acids from the pre-ATG region of the gene joined to the N-terminal peptide of beta-galactosidase, showed no such drastic effect on activity. Mutations of cysteine residues in PA19 abolishes immunomodulatory activity when both of the cysteine residues are modified. Several other mutants with a significantly lower level of DCA activity were obtained, but all of them contained multiple mutations. The E. tenella PA19 (“PA19-ET”) gene has been re-cloned into mammalian expression vector p3xFLAG-CMV9, which is designed to secrete the expressed protein into the medium. This version of PA19 protein is more active in the DC activation assay than the molecule expressed by E. coli, suggesting that post-translational modifications of the protein may affect its activity.

The skilled artisan will appreciate that the profilin-related immunomodulatory polypeptides of the invention, while structurally related to profilin, are not necessarily highly homologous to human profilin or other animal profilins. For example, protozoan profilin-related immunomodulatory polypeptides of the invention, including the PA19-like protozoan polypeptides, do not appear to possess significant homology to mammalian profilin, although significant homology to other profilins can be identified. A BLAST comparison of Eimeria tenella PA19 protein showed that it is approximately 28% identical and 45% similar to a plant profilin. No significant similarity to a mammalian profilin was identified by this analysis.

Accordingly, the profilin-related polypeptides of the invention include those with little or no homology to mammalian or plant profilin, but which possess significant structural similarity to profilin and possess immunomodulatory activity.

Conserved structural domains of the profilin protein family include cd00148.2 and smart-00392.10 (see the conserved protein domain database at http://www.ncbi.nlm.nih.gov/entrez).

Structural predictions performed using various simulation programs known in the art provide further structural bases for identifying the immunomodulatory profilin-like and profilin-related polypeptides of the invention. For example, the sequences of PA19 from P. falciparum, S. neurona, E. tenella, T. gondii, and N. caninum were used for such calculations with publicly available programs, e.g.,

JNET (www.compbio.dundee.ac.uk/˜www-jpred/),

COILS v. 2.1 (www.ch.embnet.org/cgi-bin/coils_form_parser/),

PSA (bmerc-www.bu.edu/psa),

JUFO (www.tools.bakerlab.org/˜mj/jufo_results.php), and

TURNPRED (www.tools.bakerlab.org/˜mj/turnpred_result.php).

In performing the above described searches, each sequence (NNseq) was aligned with a summary of structural predictions for it (NNstr—above the sequence with the letters standing for: H-helix, S-strand, P-local hairpin, R-diverging turn) with a summary of predictions for possible exposition/burial of the particular amino acid in the secondary structure of the protein (NNexp—below the sequence with the letters there standing for: B-buried in the structure, X-exposed to solvent). Based on these calculations, the structural features of PA19 from these five organisms are similar in two regions: the N-terminal portion, which is a coiled structure extended by a long helix (approximately residues 1-20), and the C-terminal portion, which consists of three beta-sheet structures followed by a long helix (approximately residues 125-176). The middle part of the molecule (approximately residues 21-120) shows considerable variability.

Sites of post-translational modification of PA19 isolated from different protozoa are summarized in Table 2, which shows the probable sites of post-translational modifications in PA19 proteins from different protozoa. Sites with maximal probability to be modified are in bold, the least probable sites are in parentheses.

TABLE 2
Sites of Post-Translational Modification of PA19 Isolated From Different Protozoa

| | E. tenella | N. caninum | P. falciparum | S. neurona | T. gondii |
|---|---|---|---|---|---|
| | Position | Position | Position | Position | Position |
| phosphorylated- | S12, S30, | S71, S81, | S9, S28, S44, | S32, S45, | S66, S117, |
| S¹ | S70 | S117, S147, | S155 | S150 | S147, S150 |
| | | S150 | | | |
| phosphorylated - | T6, T11, | T54, T72, | T74, T80, | T13, T79, | T54, T72, |
| T | T20, T78, | T126 | T99 | T118, T133 | T126 |
| | T97, T132 | | | | |
| phosphorylated - | none | Y17 | Y10, Y35, | Y24 | Y17, (Y104) |
| Y | | | Y59, Y89, | | |
| | | | Y101 | | |
| sulfolated-S | none | none | Y55, Y59 | none | none |
| sumoylated K | K119, K145 | K100, K125 | K126, K133 | K125, K132 | K125 |
| N-glycosilation | none | N128 | N67 | none | N128 |
| O-glycosilation | (T6) | none | (S170) | (T8) | none |
| O-GlcNAc | T6, T11, S12, | S81, S161 | S22, S169, | T22 | S161 |
| | T67 | | S170 | | |
| acetylation | none | S2 | (A2) | (A2) | (S2) |
| Dicty-O-Glyc | (T132) | (T126) | none | (T133) | (T122) |

¹The one letter code for amino acid identification has been employed in Table 2: K = lysine; N = asparagine; S = serine; T = threonine; and Y = tyrosine

Phosphorylation and sumoylation are the two most probable modifications for PA19 proteins from all five organisms. The (potentially) most active protein out of these five (PA19 from E. tenella) is the only one that does not show any sites for Tyr phosphorylation.

Several sites on the surface of the molecule that are involved in interaction with ligands are known (Bjorkegren, C., et al., FEBS Lett., 1993 333, 123-6; Bjorkegren-Sjogren, C., et al., FEBS Lett, 1997, 418, 258-64; Hajkova, L. et al., Exp. Cell. Res., 1997, 234, 66-77; Chaudhary, A., et al., Chem. Biol., 1998, 5, 273-81; Skare and Karlsson, FEBS Lett., 2002, 522, 119-24).

Within the variable middle part of the molecule, there are some notable differences between highly-active and less-active protozoan PA19 polypeptides. Accordingly, useful polypeptides of the invention include those having one or more characteristic features of active immunomodulatory PA19 polypeptides as follows. At positions 21 and 119, all active PA19s (ET, TG, NC) have an exposed negatively-charged amino acid (D), while the inactive PA19's do not. At positions 33-35, the most active PA19 ET has only one exposed negative charge (D35), while the rest have at least two (ED), and PA19 PF has three (EED); the same situation applies for position 64-66. However, the opposite situation applies for positions 61-62 and 116 (exposed negative charges for all PA19 but from ET). At positions 24, 38, 94 and 112, PA19 ET is the only one which has a positive charge (R), and at position 77 it is the only one which does not have it (K for all the rest). There are some additional similar features, but in those cases the charged amino acids may be (partially) buried and thus may not contribute to the activity. A notable structural difference between PA19 ET and the others in the middle section of the molecule is the presence of a diverging turn structure at positions 58-65 (instead of a helix structure), of a helix at 70-76 (instead of a coiled structure), and a coiled structure at 80-90 (instead of a strong helix) (see FIG. 20).

Profilin-related immunomodulatory proteins of the invention can be further assessed by computer-aided analysis of tertiary and higher structures of profilins from different organisms (including yeast, plant, and animals) and by computer-aided modeling of profilin complexes with actin, PIP2, and/or poly-L-Pro.

Thus, the foregoing analysis of the primary, secondary and tertiary structural features of profilin, PA19 and UvrABC complexes and UvrB and UvrC polypeptides and polypeptide fragments, as well as other similar proteins, provides data necessary for guidance in identifying and/or designing novel ligands, including proteins and polypeptides, that display PRIP immunomodulatory activity. PA19 is an identified ligand for TLR11/TLR12, as well as certain protein(s) from some uropathogenic bacteria and bacterial flagellin. TLR11/TLR12 has regions with leucine-rich repeats, but PA19 does not have any (recognizable) regions for recognition of Leu-rich domains. Thus, while not wishing to be bound by theory, PA19 may bind with TLR11/TLR12 indirectly. For example, PA19 may interact with the receptor indirectly via an adaptor protein, such as the SH3/SH2-domain-containing protein. Accordingly, PA19 interacts with an SH3-domain called SH3P7, and/or interacts with a leucine/isoleucine-rich protein called APRIL. The presence of TLR11/TLR12 in a complex with PA19 can occur either by direct interaction with PA19 or by indirect interaction with a SH3P7/PA19 protein complex.

4.3.3 Profilin-Related Immunomodulatory Protein-Encoding Nucleic Acids

Another aspect of the invention pertains to isolated nucleic acids encoding PRIPs, variants, and/or equivalents of such nucleic acids.

Useful nucleic acids include coding sequences from the vertebrate profilin gene, especially a mammalian profilin gene. Regardless of the species, particularly useful PRIP nucleic acids encode polypeptides that are at least 70%, 75%, 80%, 90%, 95%, 97%, or 98% similar to an amino acid sequence of a vertebrate profilin protein. For example, the nucleic acid is a cDNA encoding a polypeptide having at least one bio-activity of the subject PRIP. The nucleic acid includes all or a portion of the nucleotide sequence corresponding to the nucleic acid of SEQ ID NOS:7-12.

Still other nucleic acids of the present invention encode a PRIP which is comprised of at least 2, 5, 10, 25, 50, 100, 150 or 200 contiguous amino acid residues. For example, nucleic acid molecules for use as probes/primer or antisense molecules (i.e., noncoding nucleic acid molecules) can comprise at least about 6, 12, 20, 30, 50, 60, 70, 80, 90 or 100 base pairs in length, whereas coding nucleic acid molecules can comprise about 50, 60, 70, 80, 90, or 100 base pairs.

Another aspect of the invention provides a nucleic acid which hybridizes under low, medium, or high stringency conditions to a nucleic acid sequences represented by SEQ ID NOS:7-12. As used herein, “stringent conditions” or “stringent hybridization conditions” are generally those that (1) employ low ionic strength and high temperature for washing, for example, 0.015 M NaCl/0.0015 M sodium citrate/0.1% SDS at 50° C.; or (2) employ, during hybridization, a denaturing agent such as formamide, for example, 50% (vol/vol) formamide with 0.1% bovine serum albumin/0.1% Ficoll/0.1% polyvinylpyrrolidone/50 mM sodium phosphate buffer at pH 6.5 with 750 mM NaCl, 75 mM sodium citrate at 42° C. Another example is use of 50% formamide, 5×SSC (750 mM NaCl, 75 mM sodium citrate), 50 mM sodium phosphate (pH 6.8), 0.1% sodium pyrophosphate, 5×Denhardt's solution, sonicated salmon sperm DNA (50 ug/ml), 0.1% SDS, and 10% dextran sulfate at 42° C., with washes at 42° C. in 0.2×SSC and 0.1% SDS. “Moderately stringent conditions” are described in Sambrook et al., Molecular Cloning: A Laboratory Manual (New York: Cold Spring Harbor Laboratory Press, 1989), and include the use of a washing solution and hybridization conditions (e.g., temperature, ionic strength, and % SDS) less stringent than described above. An example of moderately stringent conditions is a condition such as overnight incubation at 37° C. in a solution comprising: 20% formamide, 5×SSC (150 mM NaCl, 15 mM trisodium citrate), 50 mM sodium phosphate (pH 7.6), 5×Denhardt's solution, 10% dextran sulfate, and 20 mg/ml denatured sheared salmon sperm DNA, followed by washing the filters in 1×SSC at about 37° C.-50° C. The skilled artisan will recognize how to adjust the temperature, ionic strength, etc., as necessary to accommodate factors such as probe length and the like. Other examples of stringency conditions for given hybrid lengths are shown in Table 3 below: highly stringent conditions are those that are at least as stringent as, for example, conditions A-F; stringent conditions are at least as stringent as, for example, conditions G-L; and reduced stringency conditions are at least as stringent as, for example, conditions M-R.

TABLE 3

| | | | Hybridization | |
|---|---|---|---|---|
| Stringency | | Hybrid Length | Temp. (T) and | Wash Temp. (T) |
| Condition | Hybrid | (bp)¹ | Buffer² | and Buffer² |
| A | DNA:DNA | >50 | 65° C.; 1x SSC - | 65° C.; 0.3x SSC |
| | | | or −42° C.; 1x SSC, | |
| | | | 50% formamide | |
| B | DNA:DNA | <50 | T_{B}*; 1x SSC | T_{B}*; 1x SSC |
| C | DNA:RNA | >50 | 67° C.; 1x SSC - | 67° C.; 0.3x SSC |
| | | | or −45° C.; 1x SSC, | |
| | | | 50% formamide | |
| D | DNA:RNA | <50 | T_{D}*; 1x SSC | T_{D}*; 1x SSC |
| E | RNA:RNA | >50 | 70° C.; 1x SSC - | 70° C.; 0.3xSSC |
| | | | or −50° C.; 1x SSC, | |
| | | | 50% formamide | |
| F | RNA:RNA | <50 | T_{F}*; 1x SSC | T_{F}*; 1x SSC |
| G | DNA:DNA | >50 | 65° C.; 4x SSC - | 65° C.; 1x SSC |
| | | | or −42° C.; 4x SSC, | |
| | | | 50% formamide | |
| H | DNA:DNA | <50 | T_{H}*; 4x SSC | T_{H}*; 4x SSC |
| I | DNA:RNA | >50 | 67° C.; 4x SSC - | 67° C.; 1x SSC |
| | | | or −45° C.; 4x SSC, | |
| | | | 50% formamide | |
| J | DNA:RNA | <50 | T_{J}*; 4x SSC | T_{J}*; 4x SSC |
| K | RNA:RNA | >50 | 70° C.; 4x SSC - | 67° C.; 1x SSC |
| | | | or −50° C.; 4x SSC, | |
| | | | 50% formamide | |
| L | RNA:RNA | <50 | T_{L}*; 2x SSC | T_{L}*; 2x SSC |
| M | DNA:DNA | >50 | 50° C.; 4x SSC - | 50° C.; 2x SSC |
| | | | or −40° C.; 6x SSC, | |
| | | | 50% formamide | |
| N | DNA:DNA | <50 | T_{N}*; 6x SSC | T_{N}*; 6x SSC |
| O | DNA:RNA | >50 | 55° C.; 4x SSC - | 55° C.; 2x SSC |
| | | | or −42° C.; 6x SSC, | |
| | | | 50% formamide | |
| P | DNA:RNA | <50 | T_{P}*; 6X SSC | T_{P}*; 6X SSC |
| Q | RNA:RNA | >50 | 60° C.; 4x SSC - | 60° C.; 2x SSC |
| | | | or −45° C.; 6x SSC, | |
| | | | 50% formamide | |
| R | RNA:RNA | <50 | T_{R}*; 4x SSC | T_{R}*; 4x SSC |
| ¹The hybrid length which is that anticipated for the hybridized region(s) of the hybridizing polynucleotides. When hybridizing a polynucleotide to a target polynucleotide of unknown sequence, the hybrid length is assumed to be that of the hybridizing polynucleotide. When polynucleotides of known sequence are hybridized, the hybrid length can be determined by aligning the sequences of the polynucleotides and identifying the region or regions of optimal sequence complementarity. | | | | |
| ²SSPE (1x SSPE is 0.15 M NaCl, 10 mM NaH₂PO₄, and 1.25 mM EDTA, pH 7.4) can be substituted for SSC (1xSSC is 0.15 M NaCl and 15 mM sodium citrate) in the hybridization and wash buffers; washes are performed for 15 minutes after hybridization is complete. | | | | |
| T_{B}*-T_{R}*: This temperature refers to the hybridization temperature for hybrids anticipated to be less than 50 base pairs in length should be 5-10EC less than the melting temperature (Tₘ) of the hybrid, where Tₘ is determined according to the following equations. For hybrids less than 18 base pairs in length, Tₘ(EC) = 2(# of A + T bases) + 4(# of G + C bases). For hybrids between 18 and 49 base pairs in length, | | | | |
| # Tₘ(EC) = 81.5 + 16.6(log₁₀ Na+) + 0.41(% G + C) − (600/N), where N is the number of bases in the hybrid, and Na⁺is the concentration of sodium ions in the hybridization buffer (Na+ for 1xSSC = 0.165 M). | | | | |

Still other examples of stringency conditions for polynucleotide hybridization are also provided in Sambrook et al., and Ausubel et al.

For example, a PRIP nucleic acid of the present invention binds to a nucleic acid having one of the sequences of SEQ ID NOS:7-12 under moderately stringent conditions, (e.g., at about 2×SSC and about 40° C.). Alternatively, a PRIP nucleic acid of the present invention will bind a nucleic acid sequence of one of SEQ ID NOS:7-12 under high stringency conditions.

Useful nucleic acids have a sequence at least 70%, at least 80%, at least 90%, or at least 95% identical to a nucleic acid encoding an amino acid sequence of a profilin gene. Nucleic acids at least 90%, at least 95%, or at least about 98-99% identical with a nucleic sequence represented in one of SEQ ID NOS:7-12 are of course also within the scope of the invention. The nucleic acid may be mammalian, and further, may include all or a portion of the nucleotide sequence corresponding to the coding region of one of SEQ ID NOS:7-12.

Nucleic acids having a sequence that differ from the nucleotide sequences shown in one of SEQ ID NOS:7-12 due to degeneracy in the genetic code are also within the scope of the invention. Such nucleic acids encode functionally equivalent peptides (i.e., a peptide having a biological activity of a profilin) but differ in sequence from the sequence shown in the sequence listing due to degeneracy in the genetic code. For example, a number of amino acids are designated by more than one triplet. Codons that specify the same amino acid, or synonyms (for example, CAU and CAC each encode histidine) may result in “silent” mutations which do not affect the amino acid sequence of a PRIP. However, it is expected that DNA sequence polymorphisms that do lead to changes in the amino acid sequences of the subject PRIPs will exist among mammals. One skilled in the art will appreciate that these variations in one or more nucleotides (e.g., up to about 3-5% of the nucleotides) of the nucleic acids encoding polypeptides having an activity of a PRIP may exist among individuals of a given species due to natural allelic variation.

Also within the scope of the invention are nucleic acids encoding splicing variants of profilin proteins or natural homologs thereof. Such homologs can be cloned by hybridization or PCR, as further described herein.

The polynucleotide sequence may also encode a leader sequence, e.g., the natural leader sequence or a heterologous leader sequence. For example, the desired DNA sequence may be fused in the same reading frame to a DNA sequence which aids in expression and secretion of the polypeptide from the host cell, for example, a leader sequence which functions as a secretory sequence for controlling transport of the polypeptide from the cell. The protein having a leader sequence is a preprotein and may have the leader sequence cleaved by the host cell to form the mature form of the protein.

The polynucleotide of the present invention may also be fused in frame to a marker sequence, also referred to herein as “Tag sequence” encoding a “Tag peptide”, which allows for marking and/or purification of the polypeptide of the present invention. The marker sequence is a hexahistidine tag, e.g., supplied by a PQE-9 vector. Numerous other Tag peptides are available commercially. Other frequently used Tags include myc-epitopes (e.g., see Ellison et al., (1991) J. Biol. Chem. 266:21150-21157) which includes a 10-residue sequence from c-myc, the pFLAG system (International Biotechnologies, Inc., New Haven, Conn.), and the pEZZ-protein A system (Pharmacia, Peapack, N.J.). Furthermore, any polypeptide can be used as a Tag so long as a reagent, e.g., an antibody interacting specifically with the Tag polypeptide is available or can be prepared or identified.

Additional PA19-related polypeptides of the invention include those encoded by nucleic acid sequences that hybridize under stringent conditions to one or more or to all of the nucleic acids encoding the newly discovered PA19 sequences discussed above, such as, for example, SEQ ID NOS: 1-3.

Alternatively, the coding sequences for the polypeptide can be incorporated as a part of a fusion gene including a nucleotide sequence encoding a different polypeptide. This type of expression system can be useful under conditions where it is desirable to produce an immunogenic fragment of a profilin protein. For example, the VP6 capsid protein of rotavirus can be used as an immunologic carrier protein for portions of the PRIP, either in the monomeric form or in the form of a viral particle. The nucleic acid sequences corresponding to the portion of a profilin protein to which antibodies are to be raised can be incorporated into a fusion gene construct that includes coding sequences e.g., for a late vaccinia virus structural protein to produce a set of recombinant viruses expressing fusion proteins comprising profilin epitopes as part of the virion. Recombinant Hepatitis B virions including Hep B surface antigen fusion proteins can be utilized in this role as well. Similarly, chimeric constructs coding for fusion proteins containing a portion of a profilin protein and the poliovirus capsid protein can be created to enhance immunogenicity of the set of polypeptide antigens (see, e.g., EP Publication No: 0259149; Evans et al. (1989) Nature 339:385; Huang et al. (1988) J. Virol. 62:3855; and Schlienger et al. (1992) J. Virol. 66:2).

The multiple antigen peptide system for peptide-based immunization can also be utilized to generate an immunogen, wherein a desired portion of a PRIP is obtained directly from organo-chemical synthesis of the peptide onto an oligomeric branching lysine core (see, for example, Posnett et al. (1988) J. Biol. Chem. 263:1719; and Nardelli et al. (1992) J. Immunol. 148:914). Antigenic determinants of profilin proteins can also be expressed and presented by bacterial cells.

In addition to utilizing fusion proteins to enhance immunogenicity, it is widely appreciated that fusion proteins can also facilitate the expression of proteins, and accordingly, can be used in the expression of the PRIPs of the present invention. For example, PRIPs can be generated as glutathione-S-transferase (GST-fusion) proteins. Such GST-fusion proteins can enable easy purification of the PRIP, as for example by the use of glutathione-derivatized matrices (see, e.g., Current Protocols in Molecular Biology, eds. Ausubel et al. (N.Y.: John Wiley & Sons, 1991)).

A fusion gene coding for a purification leader sequence, (such as a poly-(His)/enterokinase cleavage site sequence) at the N-terminus of the desired portion of the recombinant protein, can allow purification of the expressed fusion protein by affinity chromatography, e.g., using a Ni²⁺ metal resin. The purification leader sequence can then be subsequently removed by treatment with enterokinase to provide the purified protein (e.g., see Hochuli et al. (1987) J. Chromatog. 411:177; and Janknecht et al. Proc. Nat. Acad. Sci. (USA) 88:8972). Techniques for making fusion genes are known to those skilled in the art. Essentially, the joining of various DNA fragments coding for different polypeptide sequences is performed in accordance with conventional techniques, employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. The fusion gene can be synthesized by conventional techniques including automated DNA synthesizers. Alternatively, PCR amplification of gene fragments can be carried out using anchor primers which give rise to complementary overhangs between two consecutive gene fragments which can subsequently be annealed to generate a chimeric gene sequence (see, e.g., Ausubel et al.).

The present invention further pertains to methods of expressing and isolating PRIPs. For example, a host cell transfected with a nucleic acid directing expression of a nucleotide sequence encoding the PRIPs can be cultured under appropriate conditions to allow expression of the peptide to occur within the cell. Suitable media for cell culture are well known in the art. The recombinant PRIP can be isolated from cell culture medium, host cells, or both using techniques well known in the art for purifying proteins including, but not limited to, ion-exchange chromatography, gel filtration chromatography, ultrafiltration, electrophoresis, and/or immunoaffinity purification with ligands, (e.g., antibodies) specific for such PRIP. As described above, the recombinant PRIP so isolated can be a fusion protein containing a domain which facilitates its purification, such as, but not limited to, GST fusion protein.

Moreover, it will be generally appreciated that, under certain circumstances, it may be advantageous to provide homologs of one of the PRIPs which function in a limited capacity as one of either a profilin agonist (mimetic) or a profilin antagonist, in order to promote or inhibit only a subset of the biological activities of the naturally-occurring form of the protein. Thus, specific biological effects can be elicited by treatment with a homolog of limited function, and with fewer side effects relative to treatment with agonists or antagonists which are directed to all of the biological activities of naturally occurring forms of profilin proteins.

Homologs of the PRIPs can be generated by mutagenesis, such as by discrete point mutation(s), or by truncation. Mutation can give rise to homologs which retain substantially the same, or merely a subset, of the biological activity of the PRIP from which it was derived. Alternatively, antagonistic forms of the PRIP can be generated which are able to inhibit the function of the naturally occurring form of the protein, such as by competitively binding to a profilin receptor.

The recombinant PRIPs of the present invention also include homologs of the wildtype profilin proteins, such as versions of those protein which are resistant to proteolytic cleavage, as for example, due to mutations which alter ubiquitination or other enzymatic targeting associated with the protein.

PRIPs may also be chemically modified to create profilin derivatives by forming covalent or aggregate conjugates with other chemical moieties, such as, but not limited to, glycosyl groups, lipids, phosphate, acetyl groups and the like. Covalent derivatives of PRIPs can be prepared, e.g., by linking the chemical moieties to functional groups on amino acid sidechains of the protein or at the N-terminus or at the C-terminus of the polypeptide.

Modification of the structure of the PRIPs can be for such purposes as enhancing therapeutic or prophylactic efficacy, stability (e.g., ex vivo shelf life and resistance to proteolytic degradation), or post-translational modifications (e.g., to alter phosphorylation pattern of protein). Such modified peptides, when designed to retain at least one activity of the naturally-occurring form of the protein, or to produce specific antagonists thereof, are considered functional equivalents of the PRIPs described in more detail herein. Such modified peptides can be produced, for instance, by amino acid substitution, deletion, or addition. Such chemical modifications are well known in the art. Alternatively, the substitutional variant may be a substituted conserved amino acid or a substituted non-conserved amino acid.

This invention further contemplates a method for generating sets of combinatorial mutants of the PRIPs as well as truncation mutants, and is especially useful for identifying potential variant sequences (e.g., homologs). The purpose of screening such combinatorial libraries is to generate, for example, novel profilin homologs which can act as either agonists or antagonist, or alternatively, possess novel activities all together. Thus, combinatorially-derived homologs can be provided which have an increased potency relative to a naturally occurring form of the protein.

A variegated library of profilin variants can be generated by combinatorial mutagenesis at the nucleic acid level. For instance, a mixture of synthetic oligonucleotides is enzymatically ligated into gene sequences such that the degenerate set of potential profilin sequences are expressible as individual polypeptides, or alternatively, as a set of larger fusion proteins (e.g., for phage display) containing the set of profilin sequences therein. There are many ways by which such libraries of potential profilin homologs can be generated from a degenerate oligonucleotide sequence. Chemical synthesis of a degenerate gene sequence can be carried out in an automatic DNA synthesizer, and the synthetic genes then ligated into an appropriate expression vector. The purpose of a degenerate set of genes is to provide, in one mixture, all of the sequences encoding the desired set of potential profilin sequences. The synthesis of degenerate oligonucleotides is well known in the art (see, e.g., Narang, (1983) Tetrahedron 39:3; Itakura et al. (1981) Recombinant DNA, Proc 3rd Cleveland Sympos. Macromolecules, ed. A G Walton, Amsterdam: Elsevier pp 273-289; Itakura et al. (1984) Ann. Rev. Biochem. 53:323, Itakura et al. (1984) Science 198:1056; Ike et al. (1983) Nucleic Acid Res. 11:477.

Likewise, a library of coding sequence fragments can be provided for a profilin clone in order to generate a variegated population of profilin fragments for screening and subsequent selection of bioactive fragments. A variety of techniques are known in the art for generating such libraries, including chemical synthesis. For instance, a library of coding sequence fragments can be generated by (i) treating a double stranded PCR fragment of a profilin coding sequence with a nuclease under conditions wherein nicking occurs only about once per molecule; (ii) denaturing the double stranded DNA; (iii) renaturing the DNA to form double stranded DNA which can include sense/antisense pairs from different nicked products; (iv) removing single stranded portions from reformed duplexes by treatment with S1 nuclease; and (v) ligating the resulting fragment library into an expression vector. By this exemplary method, an expression library can be derived which codes for N-terminal, C-terminal and internal fragments of various sizes.

A wide range of techniques are known in the art for screening gene products of combinatorial libraries made by point mutations or truncation, and for screening cDNA libraries for gene products having a certain property. Such techniques will be generally adaptable for rapid screening of the gene libraries generated by the combinatorial mutagenesis of PRIP homologs. The most widely used techniques for screening large gene libraries typically comprises cloning the gene library into replicable expression vectors, transforming appropriate cells with the resulting library of vectors, and expressing the combinatorial genes under conditions in which detection of a desired activity facilitates relatively easy isolation of the vector encoding the gene whose product was detected. Each of the nonlimiting, illustrative assays described below are amenable to high through-put analysis as necessary to screen large numbers of degenerate profilin sequences created by combinatorial mutagenesis techniques. Combinatorial mutagenesis has a potential to generate very large libraries of mutant proteins.

Such combinatorial libraries may be technically challenging to screen even with high throughput screening assays. To overcome this problem, a new technique has been developed recently, recrusive ensemble mutagenesis (REM), which allows one to avoid the very high proportion of non-functional proteins in a random library and simply enhances the frequency of functional proteins, thus decreasing the complexity required to achieve a useful sampling of sequence space. REM is an algorithm which enhances the frequency of functional mutants in a library when an appropriate selection or screening method is employed (see, e.g., Arkin, 1992, Proc. Nat. Acad. Sci. (USA) 89:7811-7815).

As indicated by the examples set out below, PRIP-encoding nucleic acids can be obtained from mRNA present in any of a number of eukaryotic cells, e.g., metazoan cells, vertebrate cells, and mammalian cells. Nucleic acids encoding PRIPs of the present invention can be obtained from genomic DNA from both adults and embryos. For example, a gene encoding a PRIP is cloned from either a cDNA or a genomic library in accordance with protocols described herein, as well as those generally known to persons skilled in the art. cDNA encoding a PRIP is obtained by isolating total mRNA from a cell. Double-stranded cDNAs is then be prepared from the total mRNA, and subsequently inserted into a suitable plasmid or bacteriophage vector using any one of a number of known techniques. The gene encoding a PRIP can also be cloned using established PCR techniques in accordance with the nucleotide sequence information provided by the invention. A useful nucleic acid is a cDNA represented by a sequence selected from the group consisting of SEQ ID NOS:7-12.

Some useful nucleic acids encode a vertebrate PRIP comprising an amino acid sequence at least 80%, at least 90%, and at least 95% identical with an amino acid sequence contained in any of SEQ ID NOS: 1-6. Nucleic acids which encode PRIP polypeptides having at least 90%, at least 95%, or at least 98-99% homology with an amino acid sequence represented in SEQ ID NOS:1-6 are also within the scope of the invention. A nonlimiting representative nucleic acid of the invention is a cDNA encoding a peptide having at least one activity of the subject vertebrate PRIP. The nucleic acid may include all or a portion of the nucleotide sequence corresponding to the coding region of SEQ ID NOS:7-12.

Some nucleic acids of the invention encode a bioactive fragment of a vertebrate PRIP comprising an amino acid sequence at least 80%, at least 90%, or at least 95% identical with an amino acid sequence selected from the group consisting of SEQ ID NOS: 1-6. Nucleic acids which encode polypeptides which are at least 90%, at least 95%, at least 98-99%, or 100% homologous, with an amino acid sequence represented in SEQ ID NOS: 1-6 are also within the scope of the invention.

Some bioactive fragments of PRIPs include polypeptides having one or more of the following biological activities: activity as a tumor suppressor, functions in cell cycle control of various developmental processes, apoptosis, gene expression, modulation of proliferation and differentiation, and tumorigenesis. Assays for determining whether given fragment or homolog of a profilin exhibits these or other biological activities are known in the art and are further described herein.

Some PRIP fragments include the core domain of profilin and comprise at least 5, at least 20, or at least 50 contiguous amino acid residues of SEQ ID NOS: 1-6.

The nucleotide sequences determined from the cloning of profilin genes from mammalian organisms further allows for the generation of probes and primers designed for identifying and/or cloning profilin homologs in other cell types, e.g., from other tissues, as well as profilin homologs from other mammalian organisms. For instance, the present invention provides a probe/primer comprising a substantially purified oligonucleotide comprising a nucleotide sequence that hybridizes under stringent conditions to at least 12, at least 25, at least 40, at least 50 or at least 75 consecutive nucleotides of sense or anti-sense sequence from a nucleic acid sequence such as any of SEQ ID NOS:7-12, or naturally occurring mutants thereof. Such primers based on the nucleic acid represented in SEQ ID NOS:7-12 can be used in PCR reactions to clone profilin homologs.

Other probes/primers are provided that comprise a substantially purified oligonucleotide comprising a nucleotide sequence that hybridizes under moderately stringent conditions to at least 12, at least 16, at least 25, at least 40, at least 50, or at least 75 consecutive nucleotides sense or antisense sequence having one of SEQ ID NOS:7-12, or naturally occurring mutants thereof. Nucleic acid probes which are complementary to the wild-type profilin and can form mismatches with mutant profilin genes are also provided, which allow for detection by enzymatic or chemical cleavage or by shifts in electrophonetic mobility. Likewise, probes based on profilin sequences can be used to detect transcripts or genomic sequences encoding the same or homologous proteins, for use, e.g., in prognostic or diagnostic assays. The probe may further comprises a label group attached thereto and able to be detected, e.g., the label group is selected from amongst radioisotopes, fluorescent compounds, enzymes, and enzyme co-factors.

Another aspect relates to the use of isolated nucleic acids according to the invention in “antisense” therapy. As used herein, “antisense” therapy refers to administration or in situ generation of oligonucleotide molecules or their derivatives which specifically hybridize (e.g., bind) under cellular conditions, with the cellular mRNA and/or genomic DNA encoding one or more of the subject PRIPs so as to inhibit expression of that protein, e.g., by inhibiting transcription and/or translation. The binding may be by conventional base pair complementarity, or, for example, in the case of binding to DNA duplexes, through specific interactions in the major groove of the double helix. In general, “antisense” therapy refers to the range of techniques generally employed in the art, and includes any therapy which relies on specific binding to oligonucleotide sequences.

An antisense construct of the present invention can be delivered, for example, as an expression plasmid which, when transcribed in the cell, produces RNA which is complementary to at least a unique portion of the cellular mRNA which encodes a PRIP. Alternatively, the antisense construct is all oligonucleotide probe which is generated ex vivo and which, when introduced into the cell causes inhibition of expression by hybridizing with the mRNA and/or genomic sequences of a profilin gene. Such oligonucleotide probes are usefully modified oligonucleotides which are resistant to endogenous nucleases, e.g., exonucleases and/or endonucleases, and are therefore stable in vivo. Exemplary nucleic acid molecules for use as antisense oligonucleotides are phosphoramidate, phosphothioate and methylphosphonate analogs of DNA (see also U.S. Pat. Nos. 5,176,996; 5,264,564; and 5,256,775). Additionally, general approaches to constructing oligomers useful in antisense therapy have been reviewed, for example, by Van der Krol et al. (1988) BioTechniques 6:958-976; and Stein et al. (1988) Cancer Res. 48:2659-2668. With respect to antisense DNA, oligodeoxyribonucleotides derived from the translation initiation site, e.g., between the −10 and +10 regions of the profilin nucleotide sequence of interest, are useful.

Antisense approaches involve the design of oligonucleotides (either DNA or RNA) that are complementary to profilin mRNA. The antisense oligonucleotides will bind to the profilin mRNA transcripts and prevent translation. Absolute complementarity, although useful, is not required. In the case of double-stranded antisense nucleic acids, a single strand of the duplex DNA may thus be tested, or triplex formation may be assayed. The ability to hybridize depends on both the degree of complementarity and the length of the antisense nucleic acid. Generally, the longer the hybridizing nucleic acid, the more base mismatches with an RNA it may contain and still form a stable duplex (or triplex, as the case may be). One skilled in the art can ascertain a tolerable degree of mismatch by use of standard procedures to determine the melting point of the hybridized complex.

Oligonucleotides that are complementary to the 5′ end of the mRNA, e.g., the 5′ untranslated sequence up to and including the AUG initiation codon, work most efficiently at inhibiting translation. However, sequences complementary to the 3′ untranslated sequences of mRNAs are also effective at inhibiting translation of mRNAs as well. Therefore, oligonucleotides complementary to either the 5′ or 3′ untranslated, non-coding regions of a profilin gene are useful to inhibit translation of endogenous profilin mRNA. Oligonucleotides complementary to the 5′ untranslated region of the mRNA may include the complement of the AUG start codon. Antisense oligonucleotides complementary to mRNA coding regions also be used in accordance with the invention. Whether designed to hybridize to the 5′, 3′ or coding region of profilin mRNA, antisense nucleic acids should be at least 6 to about 100 nucleotides in length, such as about and more usefully less than about 50, about 25, about 17 or about 10 nucleotides in length.

The antisense oligonucleotides can be DNA or RNA or hybrid or chimeric mixtures or derivatives or modified versions thereof, and can be single-stranded or double-stranded. The oligonucleotide can be modified at the base moiety, sugar moiety, or phosphate backbone, for example, to improve stability of the molecule, hybridization, etc. The oligonucleotide may include other appended groups such as peptides (e.g., for targeting host cell receptors), or agents facilitating transport across the cell membrane (see, e.g., Letsinger et al., 1989, Proc. Natl. Acad. Sci. (USA) 86:6553-6556; Lemaitre et al., 1987, Proc. Natl. Acad. Sci. (U.S.A.) 84:648-652; PCT Pub. No. WO88/09810) or the blood-brain barrier (see, e.g., PCT Pub. No. WO89/10134), hybridization-triggered cleavage agents (see, e.g., Krol et al., 1988, BioTechniques 6:958-976) or intercalating agents (see, e.g., Zon, 1988, Pharm. Res. 5:539-549). To this end, the oligonucleotide may be conjugated to another molecule, e.g., a peptide, hybridization triggered cross-linking agent, transport agent, hybridization-triggered cleavage agent, etc.

The antisense oligonucleotide may comprise at least one modified base moiety including, but not limited to, 5-fluorouracil, 5-bromouracil, 5-chlorouracil, 5-iodouracil, hypoxanthine, xanthine, 4-acetylcytosine, 5-(carboxyhydroxytiethyl)uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguaninc, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl)uracil, and 2,6-diaminopurine.

The antisense oligonucleotide may also comprise at least one modified sugar moiety selected from the group including, but not limited to, arabinose, 2-fluoroarabinose, xylulose, and hexose.

The antisense oligonucleotide can also contain a neutral peptide-like backbone. Such molecules are termed peptide nucleic acid (PNA)-oligomers and are described, e.g., in Perry-O'Keefe et al. (1996) Proc. Natl. Acad. Sci. (USA.) 93:14670 and in Eglom et al. (1993) Nature 365:566. One advantage of PNA oligomers is their capability to bind to complementary DNA essentially independently from the ionic strength of the medium due to the neutral backbone of the DNA. The antisense oligonucleotide comprises at least one modified phosphate backbone such as, but not limited to, a phosphorothioate, a phosphorodithioate, a phosphoramidothioate, a phosphoramidate, a phosphordiamidate, a methylphosphonate, an methyl phosphotriester, and a formacetal or analog thereof.

An antisense oligonucleotide according to the invention may be an α-anomeric oligonucleotide. An α-anomeric oligonucleotide forms specific double-stranded hybrids with complementary RNA in which, contrary to the usual β-units, the strands run parallel to each other (Gautier et al., 1987, Nucl. Acids Res. 15:6625-6641).

Antisense oligonucleotides of the invention, like the nucleic acids of the invention, may be synthesized by standard methods known in the art, e.g., by use of an automated DNA synthesizer (such as are commercially available from Biosearch, Applied Biosystems, etc.). As examples, phosphorothioate oligonucleotides may be synthesized by the method of Stein et al. (1988, Nucl. Acids Res. 16:3209), methylphosphonate oligonucleotides can be prepared by use of controlled pore glass polymer supports (Sarin et al., 1988, Proc. Natl. Acad. Sci. USA 85:7448-7451), etc.

The antisense molecules can be delivered to cells which express profilin in vivo. A number of methods have been developed for delivering antisense DNA or RNA to cells; e.g., antisense molecules can be injected directly into the tissue site, or modified antisense molecules, designed to target the desired cells (e.g., antisense linked to peptides or antibodies that specifically bind receptors or antigens expressed on the target cell surface) can be administered systematically.

An alternative delivery approach utilizes a recombinant DNA construct in which the antisense oligonucleotide is placed under the control of a strong pol III or pol II promoter. The use of such a construct to transfect target cells in the patient will result in the transcription of sufficient amounts of single-stranded RNAs that will form complementary base pairs with the endogenous profilin transcripts and thereby prevent translation of the profilin mRNA. For example, a vector can be introduced in vivo such that it is taken up by a cell and directs the transcription of an antisense RNA. Such a vector can remain episomal or become chromosomally integrated, as long as it can be transcribed to produce the desired antisense RNA. Such vectors can be constructed by recombinant DNA technology methods standard in the art as described above. Vectors can be plasmid, viral, or others known in the art, used for replication and expression in mammalian cells. Expression of the sequence encoding the antisense RNA can be by any promoter known in the art to act in mammalian, usefully human cells. Such promoters can be inducible or constitutive. Such promoters include but are not limited to: the SV40 early promoter region (Bemoist and Chambon, 1981, Nature 290:304-310), the promoter contained in the 3′ long terminal repeat of Rous sarcoma virus (Yamamoto et al., 1980, Cell 22:787-797), the herpes thymidine kinase promoter (Wagner et al., 1981, Proc. Natl. Acad. Sci. USA 78:1441-1445), the regulatory sequences of the metallothionein gene (Brinster et al, 1982, Nature 296:39-42), etc. Any type of plasmid, cosmid, YAC or viral vector can be used to prepare the recombinant DNA construct which can be introduced directly into the tissue site; e.g., the choroid plexus or hypothalamus. Alternatively, viral vectors can be used which selectively infect the desired tissue; (e.g., for brain, herpesvirus vectors may be used), in which case administration may be accomplished by another route (e.g., systematically).

Ribozyme molecules designed to catalytically cleave profilin mRNA transcripts can also be used to prevent translation of profilin mRNA and expression of profilin (see, e.g., PCT Pub. WO90/11364; Sarver et al., 1990, Science 247:1222-1225 and U.S. Pat. No. 5,093,246). While ribozymes that cleave mRNA at site specific recognition sequences can be used to destroy profilin mRNAs, the use of hammerhead ribozymes is useful. Hammerhead ribozymes cleave mRNAs at locations dictated by flanking regions that form complementary base pairs with the target mRNA. The sole requirement is that the target mRNA have the following sequence of two bases: 5′-UG-3′. The construction and production of hammerhead ribozymes is well known in the art (see, e.g., Haseloff et al., 1988, Nature, 334:585-591). There are a number of potential hammerhead ribozyme cleavage sites within the nucleotide sequence of human profilin cDNA (FIG. 1). The ribozyme can be engineered so that the cleavage recognition site is located near the 5′ end of the profilin mRNA; i.e., to increase efficiency and minimize the intracellular accumulation of non-functional mRNA transcripts.

The ribozymes of the present invention also include RNA endoribonucleases (hereinafter “Cech-type ribozymes”) such as the one which occurs naturally in Tetrahymena thermophila (known as the IVS, or L-19 IVS RNA) (see, e.g., Zaug, et al., 1984, Science, 224:574-578; PCT Pub. No. WO88/04300). The Cech-type ribozymes have an eight base pair active site which hybridizes to a target RNA sequence whereafter cleavage of the target RNA takes place. The invention encompasses those Cech-type ribozymes which target eight base-pair active site sequences that are present in a profilin gene.

As for antisense nucleic acids of the invention approach, the ribozymes can be composed of modified oligonucleotides (e.g., for improved stability, targeting, etc.) and can be delivered to cells which express the profilin gene in vivo. A useful method of delivery involves using a DNA construct “encoding” the ribozyme under the control of a strong constitutive pol III or pol II promoter, so that transfected cells will produce sufficient quantities of the ribozyme to destroy endogenous profilin messages and inhibit translation. Because ribozymes, unlike antisense molecules, are catalytic, a lower intracellular concentration is useful for efficiency.

Endogenous profilin gene expression can also be reduced by inactivating or “knocking out” the profilin gene or its promoter using targeted homologous recombination. For example, a mutant, non-functional profilin (or a completely unrelated DNA sequence) flanked by DNA homologous to the endogenous profilin gene (either the coding regions or regulatory regions of the profilin gene) can be used, with or without a selectable marker and/or a negative selectable marker, to transfect cells that express profilin in vivo. Insertion of the DNA construct, via targeted homologous recombination, results in inactivation of the profilin gene. Such approaches are particularly suited in the agricultural field where modifications to ES (embryonic stem) cells can be used to generate animal offspring with an inactive profilin (see, e.g., Smithies et al., 1985, Nature 317:230-234). However, this approach can be adapted for use in humans provided the recombinant DNA constructs are directly administered or targeted to the required site in vivo, e.g., using appropriate viral vectors, e.g., herpes virus vectors for delivery to brain tissue; e.g., the hypothalamus and/or choroid plexus.

Alternatively, endogenous profilin gene expression can be reduced by targeting DNA sequences complementary to the regulatory region of the profilin gene (i.e., the profilin promoter and/or enhancers) to form triple helical structures that prevent transcription of the profilin gene in target cells in the body (see e.g., Helene, C. 1991, Anticancer Drug Des., 6(6);569-84).

Nucleic acid molecules to be used in triple helix formation for the inhibition of transcription are usefully single-stranded and composed of deoxyribonucleotides. The base composition of these oligonucleotides promotes triple helix formation via Hoogsteen base pairing rules, which generally require sizable stretches of either purines or pyrimidines to be present on one strand of a duplex. Nucleotide sequences may be pyrimidine-based, which will result in TAT and CGC triplets across the three associated strands of the resulting triple helix. The pyrimidine-rich molecules provide base complementarity to a purine-rich region of a single strand of the duplex in a parallel orientation to that strand. In addition, nucleic acid molecules may be chosen that are purine-rich, for example, containing a stretch of G residues. These molecules form a triple helix with a DNA duplex that is rich in GC pairs, in which the majority of the purine residues are located on a single strand of the targeted duplex, resulting in CGC triplets across the three strands in the triplex.

Alternatively, the potential sequences that can be targeted for triple helix formation may be increased by creating a so called “switchback” nucleic acid molecule. Switchback molecules are synthesized in an alternating 5′-3′, 3′-5′ manner, such that they base pair with first one strand of a duplex and then the other, eliminating the necessity for a sizable stretch of either purines or pyrimidines to be present on one strand of a duplex.

Antisense, ribozyme, and triple helix nucleic acid molecules of the invention may be prepared by any method known in the art for the synthesis of DNA and RNA molecules. These include techniques for chemically synthesizing oligodeoxyribonucleotides and oligoribonucleotides well known in the art such as for example solid phase phosphoramidite chemical synthesis. Alternatively, RNA molecules may be generated by in vitro and in vivo transcription of DNA sequences encoding the antisense RNA molecule. Such DNA sequences may be incorporated into a wide variety of vectors which incorporate suitable RNA polymerase promoters such as the T7 or SP6 polymerase promoters. Alternatively, antisense cDNA constructs that synthesize antisense RNA constitutively or inducibly, depending on the promoter used, can be introduced stably into cell lines.

Moreover, various well-known modifications to nucleic acid molecules may be introduced as a means of increasing intracellular stability and half-life, as described above.

The invention further provides nucleic acid plasmids and vectors encoding a PRIP, which can be used to express a PRIP in a host cell. The host cell may be any prokaryotic or eukaryotic cell. Thus, a nucleotide sequence derived from the cloning of mammalia profilins, encoding all or a selected portion of the full-length protein, can be used to produce a recombinant form of a PRIP via microbial or eukaryotic cellular processes. Ligating the polynucleotide sequence into a gene construct, such as an expression vector, and transforming or transfecting into hosts, either eukaryotic (e.g., yeast, avian, insect or mammalian) or prokaryotic (bacterial cells), are standard procedures well known in the art.

Vectors that allow expression of a nucleic acid in a cell are referred to as expression vectors. As described above, expression vectors used for expressing a PRIP typically contain a nucleic acid encoding a PRIP, operably linked to at least one transcriptional regulatory sequence.

Regulatory sequences are art-recognized and are selected to direct expression of the subject PRIPs. Transcriptional regulatory sequences are described in Goeddel, Meth. Enzymol. 185, Academic Press, San Diego, Calif. (1990). The expression vector can include a recombinant gene encoding a peptide having an agonistic activity of a subject PRIP, or alternatively, encoding a peptide which is an antagonistic form of a PRIP.

Suitable vectors for the expression of a PRIP include plasmids of the types: pBR322-derived plasmids; pEMBL-derived plasmids; pEX-derived plasmids; pBTac-derived plasmids; and pUC-derived plasmids for expression in prokaryotic cells, such as E. coli.

A number of vectors exist for the expression of recombinant proteins in yeast. For instance, YEP24, YIP5, YEP51, YEP52, pYES2, and YRP17 are cloning and expression vehicles useful in the introduction of genetic constructs into S. cerevisiae (see, e.g., Broach et al. (1983) in Experimental Manipulation of Gene Expression, (ed. M. Inouye) Academic Press, p. 83,). These vectors can replicate in E. coli due the presence of the pBR322 ori, and in S. cerevisiae due to the replication determinant of the yeast 2 micron plasmid. In addition, drug resistance markers such as ampicillin can be used. A PRIP can be produced recombinantly utilizing an expression vector generated by sub-cloning the coding sequence of one of the profilin genes represented in SEQ ID NOS:7-12.

The useful mammalian expression vectors contain both prokaryotic sequences (to facilitate the propagation of the vector in bacteria), and one or more eukaryotic transcription units that are expressed in eukaryotic cells. The pcDNAI/amp, pcDNAI/neo, pRc/CMV, pSV2gpt, pSV2neo, pSV2-dhfr, pTk2, pRSVneo, pMSG, pSVT7, pko-neo and pHyg derived vectors are nonlimiting examples of mammalian expression vectors suitable for transfection of eukaryotic cells. Some of these vectors are modified with sequences from bacterial plasmids, such as pBR322, to facilitate replication and drug resistance selection in both prokaryotic and eukaryotic cells. Alternatively, derivatives of viruses such as the bovine papillomavirus (BPV-1), or Epstein-Barr virus (pHEBo, pREP-derived and p205) can be used for transient expression of proteins in eukaryotic cells. The various methods employed in the preparation of the plasmids and transformation of host organisms are well known in the art. For other suitable expression systems for both prokaryotic and eukaryotic cells, as well as general recombinant procedures, see Molecular Cloning: A Laboratory Manual, 2nd Ed., (ed. by Sambrook, Fritsch and Maniatis) Cold Spring Harbor Laboratory Press (1989) Chapters 16-17.

In some instances, it may be desirable to express the recombinant PRIP by the use of a baculovirus expression system. Nonlimiting examples of such baculovirus expression systems include pVL-derived vectors (such as pVL1392, pVL1393 and pVL941), pAcUW-derived vectors (such as pAcUW1), and pBlueBac-derived vectors (such as the β-gal containing pBlueBac III)

When it is desirable to express only a portion of a PRIP, such as a form lacking a portion of the N-terminus (i.e., a truncation mutant which lacks the signal peptide), it may be useful to add a start codon (ATG) to the oligonucleotide fragment containing the desired sequence to be expressed. It is well known in the art that a methionine at the N-terminal position can be enzymatically cleaved by the use of the enzyme methionine aminopeptidase (MAP), (Ben-Bassat et al. (1987) J. Bacteriol. 169:751-757); Miller et al. (1987) Proc. Nat. Acad. Sci. (USA) 84:2718-1722). Therefore, removal of an N-terminal methionine, if desired, can be achieved either in vivo by expressing profilin derived polypeptides in a host which produces MAP (e.g., E. coli, CM89 or S. cerevisiae), or in vitro by use of purified MAP (e.g., procedure of Miller et al., supra).

Moreover, the gene constructs of the present invention can also be used as part of a gene therapy protocol to deliver nucleic acids encoding either an agonistic or antagonistic form of one of the subject PRIPs. Thus, another aspect of the invention features expression vectors for in vivo or in vitro transfection and expression of a PRIP in particular cell types so as to reconstitute the function of, or alternatively, abrogate the function of, profilin in a tissue. This is useful, for example, when the naturally-occurring form of the protein is misexpressed or the natural protein is mutated and less active.

In addition to viral transfer methods, non-viral methods can also be employed to cause expression of a subject PRIP in the tissue of an animal. Most nonviral methods of gene transfer rely on normal mechanisms used by mammalian cells for the uptake and intracellular transport of macromolecules. Some non-viral targeting means of the present invention rely on endocytic pathways for the uptake of the subject PRIP gene by the targeted cell. Non-limiting exemplary targeting means of this type include liposomal derived systems, poly-lysine conjugates, and artificial viral envelopes.

4.3.4 Transgenic Animals

The invention further provides non-human transgenic animals useful for studying the function and/or activity of a PRIP and for identifying and/or evaluating modulators of profilin activity. As used herein, a “transgenic animal” is a non-human animal, such as a mammal, a rodent, or mouse, in which one or more of the cells of the animal includes a transgene. Other nonlimiting examples of useful transgenic animals include non-human primates, sheep, dogs, cows, goats, chickens, amphibians, and the like. A transgene is exogenous DNA or a rearrangement, e.g., a deletion of endogenous chromosomal DNA, which usefully is integrated into or occurs in the genome of the cells of a transgenic animal. A transgene can direct the expression of an encoded gene product in one or more cell types or tissues of the transgenic animal, other transgenes, e.g., a knockout, reduce expression. Thus, a transgenic animal can be one in which an endogenous profilin gene has been altered by, e.g., by homologous recombination between the endogenous gene and an exogenous DNA molecule introduced into a cell of the animal, e.g., an embryonic cell of the animal, prior to development of the animal.

Intronic sequences and polyadenylation signals can also be included in the transgene to increase the efficiency of expression of the transgene. A tissue-specific regulatory sequence(s) can be operably linked to a transgene of the invention to direct expression of a PRIP to particular cells. A transgenic founder animal can be identified based upon the presence of a profilin transgene in its genome and/or expression of profilin mRNA in tissues or cells of the animals. A transgenic founder animal can then be used to breed additional animals carrying the transgene. Moreover, transgenic animals carrying a transgene encoding a PRIP can further be bred to other transgenic animals carrying other transgenes.

PRIPs can be expressed in transgenic animals or plants, e.g., a nucleic acid encoding the PRIP or fragment thereof can be introduced into the genome of an animal. The nucleic acid can be placed under the control of a tissue specific promoter, e.g., a milk or egg specific promoter, and recovered from the milk or eggs produced by the animal. Suitable animals include, but are not limited to, mice, pigs, cows, goats, and sheep.

### 4.4 TLR11/TLR12- and/or TLR5-Targeted Therapeutics

4.4.1 TLR11/TLR12 and/or TLR5 Polypeptides and Nucleic Acids

While not wishing to be bound by any particular theory, the profilin immunomodulatory polypeptides of the invention interact, directly or indirectly, with the Toll-like Receptor TLR11/TLR12 and/or TLR5 to produce one or more of their immunomodulatory effects. The amino acid and nucleic acid sequences of TLR11/TLR12 are disclosed in WO 03/078573 and WO 03/089602, and methods and products for identification and assessment of TLR ligands are disclosed in WO 04/094671. The amino acid and nucleic acid sequences of TLR5 and methods and products for identification and assessment of TLR5 ligands are disclosed in US2005/0147627.

The invention also provides a nucleic acid encoding the amino acid sequence of SEQ ID NO: 40 or 42 of FIG. 25B or 26B, respectively, or a nucleic acid complementary to the nucleic acid sequences of SEQ ID NO: 39 or 41 of FIG. 25A or 26A, respectively. The encoded amino acid sequence can be at least 70%, at least 80%, at least 90%, at least 95%, or at least 97-98%, or greater than at least 99% identical to a sequence corresponding to at least 12, at least 15, at least 25, or at least 40, at least 100, at least 200, at least 300, at least 400 or at least 500 consecutive amino acid residues up to the full length of SEQ ID NO: 40 or 42.

Optionally, a TLR11 or a TLR12 nucleic acid (see FIGS. 14-17, 39, and 41) will genetically complement a partial or complete TLR11 or TLR12 loss of function phenotype in a cell. For example, a TLR11 or a TLR12 nucleic acid may be expressed in a cell in which endogenous TLR11 or TLR12 has been reduced by RNAi, and the introduced TLR11 or TLR12 nucleic acid will mitigate a phenotype resulting from the RNAi. The term “RNA interference” or “RNAi” refers to any method by which expression of a gene or gene product is decreased by introducing into a target cell one or more double-stranded RNAs which are homologous to the gene of interest (particularly to the messenger RNA of the gene of interest).

As used herein the term “toll-like receptor 5” or “TLR5” is intended to mean a toll-like receptor 5 of any species, such as the murine and human polypeptides containing an amino acid sequence set forth as SEQ ID NO: 44 or 46 of FIGS. 27B and 28B, respectively, encoded by a nucleic acid sequence identified as SEQ ID NO: 43 or 45 of FIGS. 27A and 28A, respectively. A TLR5 is activated upon binding to a PRIP or other TLR5 agonists. Upon activation, a TLR5 induces a cellular response by transducing an intracellular signal that is propagated through a series of signaling molecules from the cell surface to the nucleus. For example, the intracellular domain of TLR5 recruits an adaptor protein, MyD88, which recruits the serine kinase IRAK. IRAK forms a complex with TRAF6, which then interacts with various molecules that participate in transducing the TLR signal. These molecules and other TRL5 signal transduction pathway components stimulate the activity of transcription factors, such as fos, jun and NF-κB, and the corresponding induction of gene products of fos-, jun- and NF-κB-regulated genes, such as, for example, TNF-α, IL-1 and IL-6. The activities of signaling molecules that mediate the TLR5 signal, as well as molecules produced as a result of TLR5 activation are TLR5 activities that can be observed or measured. Therefore, a TLR5 activity includes binding to a PRIP, recruitment of intracellular signaling molecules, as well as downstream events resulting from TLR5 activation, such as transcription factor activation and production of immunomodulatory molecules. A TLR5 cellular response mediates an innate immune system response in an animal because cytokines released by TLR5-expressing cells regulate other immune system cells to promote an immune response in an animal. Therefore, as used herein the term “TLR5-mediated response” is intended to mean the ability of a PRIP to induce a TLR5-mediated cellular response. Exemplary TLR5-mediated cellular responses include activation of transcription factors such as fos, jun and NF-κB, production of cytokines such as IL-1, IL-6 and TNF-α, and the stimulation of an immune response in an animal.

A TLR5 also encompasses polypeptides containing minor modifications of a native TLR5, and fragments of a full-length native TLR5, so long as the modified polypeptide or fragment retains one or more biological activities of a native TLR5, such as the abilities to stimulate NF-κB activity, stimulate the production of cytokines such as TNF-α, IL-1, and IL-6 and stimulate an immune response in response to TLR5 binding to a known TLR5 activating ligand such as flagellin polypeptide, immunomodulatory peptide or modifications thereof. A modification of a TLR5 can include additions, deletions, or substitutions of amino acids, so long as a biological activity of a native TLR5 is retained. For example, a modification can serve to alter the stability or activity the polypeptide, or to facilitate its purification. Modifications of polypeptides as described above in reference to flagellin polypeptides and peptides are applicable to TLR5 polypeptides of the invention. A “fragment” of a TLR5 is intended to mean a portion of a TLR5 that retains at least about the same activity as a native TLR5.

Nucleic acids encoding for TLR5 further include nucleic acids that comprise variants of SEQ ID NO: 43 or 45. Variants will also include sequences that will hybridize under highly stringent conditions to a nucleotide sequence of a coding sequence designated in SEQ ID NO: 43 or 45.

In general, toll-like receptors (TLRs) are structurally related. All contain a TIR domain at their C-terminal end, and an extensive membrane-bound part preceding the TIR (Wittenmayer, N., et al., Mol. Biol. Cell, 2004, 15, 1600-1608). The TLRs are involved in the innate immune defense by recognizing specific molecular patterns of pathological microorganisms. Each TLR recognizes different ligands, though a single TLR can recognize many different patterns of ligands. The recognition is believed to occur through an exposed part of the molecule (N-terminal and adjacent part). In some cases (such as TLR4) the recognition is mediated through adaptor molecules (MD-2 in the case of TLR4 and LPS (Kennedy, et al., J. Biol. Chem., 2004, 279: 34698-704) and involves several other proteins in the formation of the active complex (such as CD14, LPS-binding protein, etc. in the case of TLR4 (Kennedy, et al.). TLR11/TLR12 and TLR5 are proteins from the TLR family which includes TLRs11-13, and 21-23. These receptors are abundant in fish, birds, and rodents (Stafford, et al., 2003, Dev Comp Immunol, 27: 685-98), but were not yet shown to be present in active form in large animals, including humans. In humans, TLR11/TLR12 has been found to be polymorphic and it is likely that TLR11/TLR12 in humans is either a pseudogene or a shorter version of the gene. In addition, the identity between chimpanzee and human TLR11/TLR12 is one order higher.

A RPS-BLAST search with highest expectation parameter (Dimopoulos, et al., Proc. Nat. Acad. Sci. (USA), 2002, 99, 8814-9) reveals a TIR domain (smart00255, position 761-902), leucine-rich repeat (LRP, COG4886, position 201-523), which is present in many protein-protein interacting systems, and several conserved domains with rather low similarity (0.26-0.87). A search with the program Phyre (http://www.sbg.bio.ic.ac.uk/phyre) revealed some motifs similar to clk5dC (Ran-GAP1 GTPase activating protein, 286-424), c1ww1A (monocyte differentiation antigen cd14, 90-280), clt3gA (membrane x-linked interleukin-1 receptor accessory, 762-902). It also predicted with very high probability some strong helical structures for the regions 85-89, 108-115, 245-265, 659-668, 715-746, 771-783, 808-818, 873-879, and 896-903; short coiled regions at 20-24, 29-35, 67-70, 87-89, 123-126, 150-152, 231-234, 339-342, 350-354, 398-400, 502-510, 571-573, 589-592, 600-608, 613-616, 649-651, 786-790, 801-805, 850-853, and 887-890; and beta-sheet like structures at 71-75, 119-122, 272-274, 300-302, 345-349, 394-396, 567-569, 597-599, 619-623, 761-769, 821-827, 855-860, and 884-887. The same program predicted that several regions of the protein are disordered: 1-7, 125-140, 191-198, 237-241, 249-259, 288-292, 753-759, 784-789, and 904-905.

The locus of the mammalian TLR11/TLR12 was well conserved in mammals: the gene is flanked at the 5′ side by polyhomeotic-like protein 2 (PHC2, HomoloGene #75090), and at the 3′ side by zinc finger protein 31 (ZNF31, HomoloGene #51463) in mouse, rat, human, chimpanzee, and dogs. (These genes may serve as good markers for locating the TLR12 gene on a chromosome for a new organism or in a patient. The murine TLR12 gene (protein NP 991392, AAS37673, AAS83531, BAE23434) is located on chromosome 4. Rat TLR12 gene is located on chromosome 5 (protein XP_{—}342923, has 87% identities, and 92% positives on protein level with mTLR12). Both human and chimpanzee analogs of TLR12 (pseudo)-gene are located on chromosome 1 (locus LOC441882 for human) and have a number of internal stop codons as well as frame shifts; comparison of these regions in human and chimp genomes showed unusually high level of similarity (99.3% on nucleotide level), while the average level of similarity between human and chimpanzee is only 96%, arguing that the region has to be functionally important in these organisms. The dog analog of TLR12 is located on chromosome 2; the exact chromosome location is yet unknown for the cow analogue, but the (pseudo)-gene is flanked by the same PHC2 and ZNF31 genes. Reconstruction of the theoretical gene/protein sequence for both dog and cow TLR12 is being prepared (see FIG. 17). For example, the sequences of TLR11/TLR12 for mouse, rat, and chicken are shown in FIG. 13. A database search of partially known genomes from other organisms may reveal similar genes.

Conservation of human TLR11 cDNA from placenta has been reported Klaffenbach, D., et al., (Am. J. Reprod. Immunol. 2005, 53, 77-84). The gene is interrupted by several premature stop codons, which would make the projected expressed protein non-functional. The first stop codon at position 167 (in contrast to the previous data on a stop codon at position 119), which suggests that the human TLR11/TLR12 and/or TLR5 gene is polymorphic. Analysis of sequences from genomes of dog, and chimpanzee were similar to the sequence from the human genome: several STOP codons in the middle of the sequence plus a couple of frame-shifts (which can be attributed either to errors in the sequences from the database or to the presence of short introns, not recognized by standard programs).

Both the secondary and higher structures of TLR11/TLR12 protein are unknown; some predictions of the structures are summarized in FIGS. 18A-18D. FIG. 18A shows the possible topology of mTLR11/TLR12 by hydrophobicity. FIG. 18B shows the possible topology of mTLR11/TLR12 and/or TLR5 by exposure on cell surface (inwards or outwards). FIGS. 18C and 18D show signalP-NN prediction and signalP-HMM prediction (respectively) eukaryote models for mTLR11/TLR12. Since the first 22 amino-acids of the N-terminus most probably represent a leading peptide responsible for transport of the TLR12 protein through the membrane, the topology of the adjacent region would be highly unpredictable. The algorithm predicts that the region spanning amino acids 22-77 is not located in cytoplasm (as it is represented in FIG. 18B), but instead is extra-cellularly exposed; that a short region spanning amino acids 78-94 is a transmembrane, and that the region spanning amino acids 95-446 is extracellular too.

Comparison of the TLR11/TLR12 protein sequences from different organisms revealed that the chromosomal region which would encode for the TLR11/TLR12 protein is interrupted by several stop codons and frame shifts in human and chimp genomes (7 out of 10 such sites are in the TIR domain region; the sequences are virtually identical for both human and chimpanzee. The presence of STOP codons in simian TLR11/TLR12 genes is an interesting distinction from other mammals. While not wishing to be limited by any particular theory, two possibilities exist: 1) the gene is highly polymorphic and is present in its normal form only in a small number of (human) individuals, or 2) the gene evolved this way is similar and is not polymorphic. The latter seems more probable because the region of the genome in dog and cow also appears to be interrupted by several stop-codons and frame-shifts as well).

Accordingly, the TLR11/TLR12 gene in humans and chimps may function to encode for a TLR11/TLR12 polypeptide of reduced size. The shortest version of the protein would be about 170 amino-acids long; if the frame-shift at position close to 260 AA is a misread or polymorphic, the next stop codon is at position of about 680 AA. Thus, if the first stop codon is polymorphic, the 680 AA-long protein can exist too. Indeed, the 170 AA-long polypeptide should be expressed. Furthermore, the above-mentioned topological model predicts that with the possible exception of a short middle section of the polypeptide, it should be exposed extracellularly. The size of the polypeptide is long enough to carry several binding sites (for example, a shorter protein—profilin (120 AA) is known to have at least three different binding sites). One of these sites can be a binding site for PA19 or for an adapter binding PA19. Toll-like receptors are known to function as homo- or hetero-dimers. TLR11/TLR12 could also work through formation of a hetero-dimer. The second binding site on the 170-AA long polypeptide could contain the binding site participating in such dimerization.

Accordingly, the short polypeptide, which is expressed from the gene for TLR11/TLR12 in humans, functions as an adaptor so as to bind PA19 and bring it to an unspecified toll-like receptor, which would lead to activation of that receptor (for example, conformational changes in the cytoplasmic part of it, TIR-domain, followed by binding to it MyD88 adaptor and activation of the NF-κB pathway by multiple phosphorylations) and, as the result, to release cytokines by the cell. Therefore, PA19 is expected to be active in most humans.

Further, it is possible that several regions inside the TLR11/TLR12 gene in the human genome evolved to become introns or are otherwise recognized for splicing them out of the final mRNA. The final mRNA would serve for expression of a slightly shorter version of the protein (compared to murine TLR11/TLR12), still containing the same major domains and functioning the same way as the mTLR11/TLR12 does. The result of this would be the same as above: most human patients would be susceptible to treatment with PA19 alone, and in combination with gene therapy.

The E. coli UvrABC system may mimic the interaction of PA19 (possibly dimerized) with an adapter molecule. In this case, UvrA may mimic the component of the system that interacts with TLR11/TLR12 and/or TLR5. Indeed, experiments with the UvrABC system have shown that UvrA may enhance the effect of PA19 on the activation of dendritic cells, while UvrBC complex may decrease the effect. Therefore, some unknown proteins with or without Leucine-binding domains, may physically interact with the TLR11/TLR12 and/or TLR5 receptor, and may activate it.

Where direct interaction between PA19 and TLR11/TLR12 and/or TLR5 exists, an antibody to a certain site on TLR11/TLR12 and/or TLR5 mimics the effects of PA19. Accordingly, such an antibody exhibits the same anti-cancer properties as PA19 does. Such antibodies to TLR11/TLR12 and/or TLR5 (generated to a synthetic peptide derived from the TLR11/TLR12 and/or TLR5 sequence) can be prepared and screened for TLR11/TLR12 and/or TLR5 agonist activity as is known in the art. There are also commercially available antibodies. eBioscience offers a polyclonal antibody to a 16 amino acid long peptide in the middle of the molecule (www.ebioscience.com). Psi-ProSci (www.prosci-inc.com) offers two different polyclonal antibodies to a 16 amino acid long peptide near the middle of TLR11 and a 15 amino acid long peptide near its the C-terminus (which is a TIR domain). Imgenex (www.imgenex.com) offers two polyclonal and one monoclonal antibody (the latter to a TIR domain (residues 750-850), the former to the TIR domain (residues 700-800) and to a portion closer to N-terminus (peptide 147-159). They also offer a polyclonal antibody to the TIR domain (residues 900-950) of murine TLR12 (which is the same protein as TLR11). USBiological (www.usbio.net) offers two polyclonal antibodies: one against a 15 amino acid long peptide near the C-terminus (TIR domain), and the other against a 16-amino acid long peptide from the middle of the TLR11 sequence. Serotec (www.serotec.com) also offers non-specified antibody to murine TLR11.

As used herein, a “biologically active portion” of a TLR11/TLR12 or TLR5 protein includes a fragment of a TLR11/TLR12 or TLR5 protein which participates in an interaction between a TLR11/TLR12 or TLR5 molecule and a non-TLR11/TLR12 or TLR5 molecule. Biologically active portions of a TLR11/TLR12 or TLR5 protein include peptides comprising amino acid sequences sufficiently homologous to or derived from the amino acid sequence of the TLR11/TLR12 or TLR5 protein, e.g., the amino acid sequence shown in SEQ ID NOS: 40 or 42, which include fewer amino acids than the full length TLR11/TLR12 or TLR5 protein, and exhibit at least one activity of a TLR11/TLR12 or TLR5 protein, e.g., amino acids comprising a LIM domain (about amino acids 126 to 188 (“LIM domain 1”), 191 to 248, (“LIM domain 2”) and 251 to 311 (“LIM domain 3”) of SEQ ID NOS: 40 or 42).

A biologically active portion of a TLR11/TLR12 or TLR5 protein can be a polypeptide which is, for example, 10, 15, 25, 30, 35, 40, 45, 50, 55, 60, 65, 70, 75, 80, 85, 90, 95, 100, 110, 120, 130, 140, 150, 160, 170, 180, 190, 200, 210, 220, 230, 240, 250, 260, 270, 280, 300, 310, 320, 330, 340, 350, 360, 370, 380, 390, 400, 410, 420, 430, 440, 450, 460, 470, 480, 490, 500, or more amino acids in length. Biologically active portions of a TLR11/TLR12 or TLR5 protein can be used as targets for developing agents which modulate a TLR11/TLR12 and/or TLR5 mediated activity.

Particular TLR11/TLR12 polypeptides have an amino acid sequence substantially identical to the amino acid sequence of SEQ ID NO:40 or 42, and particular TLR5 polypeptides of the present invention have an amino acid sequence substantially identical to the amino acid sequence of SEQ ID NO: 44, or 46. In the context of an amino acid sequence, the term “substantially identical” is used herein to refer to a first amino acid that contains a sufficient or minimum number of amino acid residues that are i) identical to, or ii) conservative substitutions of aligned amino acid residues in a second amino acid sequence such that the first and second amino acid sequences can have a common structural domain and/or common functional activity.

The invention further encompasses nucleic acid molecules that differ from the nucleotide sequence shown in SEQ ID NO:39, 41, 43, or 45. Such differences can be due to degeneracy of the genetic code (and result in a nucleic acid which encodes the same TLR11/TLR12 or TLR5 proteins as those encoded by the nucleotide sequence disclosed herein. For example, an isolated nucleic acid molecule of the invention can have a nucleotide sequence encoding a protein having an amino acid sequence which differs, by at least 1, but less than 5, 10, 20, 50, or 100 amino acid residues that shown in SEQ ID NO:40, 42, 44, or 46. If alignment is needed for this comparison the sequences should be aligned for maximum homology. “Looped” out sequences from deletions or insertions, or mismatches, are considered differences.

Nucleic acids of the invention can be chosen for having codons, which are useful, or non-useful, for a particular expression system. For example, the nucleic acid can be one in which at least one codon, at usefully at least 10%, or at least 20% of the codons has been altered such that the sequence is optimized for expression in E. coli, yeast, human, insect, or CHO cells.

The nucleic acid may differ from that of SEQ ID NO:39, 41, 43, or 45, e.g., as follows: by at least one but less than 10, 20, 30, or 40 nucleotides; at least one but less than 1%, 5%, 10% or 20% of the nucleotides in the subject nucleic acid. If necessary for this analysis the sequences are aligned for maximum homology. “Looped” out sequences from, deletions or insertions, or mismatches, are considered differences.

Allelic variants of TLR11/TLR12 and TLR5, e.g., human TLR11/TLR12 and TLR5, include both functional and non-functional proteins. Functional allelic variants will typically contain only conservative substitution of one or more amino acids of SEQ ID NO:40, 42, 44, or 46 or substitution, deletion or insertion of non-critical residues in non-critical regions of the protein. Non-functional allelic variants are naturally-occurring amino acid sequence variants of the TLR11/TLR12 or of TLR5.

Non-functional allelic variants typically contain a non-conservative substitution, a deletion, or insertion, or premature truncation of the amino acid sequence of SEQ ID NO:40, 42, 44, or 46 or a substitution, insertion, or deletion in critical residues or critical regions of the protein.

Moreover, nucleic acid molecules encoding other TLR11/TLR12 and/or TLR5 family members and, thus, which have a nucleotide sequence which differs from the TLR11/TLR12 and/or TLR5 sequences of SEQ ID NO:39, 41, 43, or 45 are intended to be within the scope of the invention.

4.4.2 TLR11/TLR12 and TLR5 Activities and Assays

Aspects of the present invention provides assays for identifying therapeutic agents which either interfere with or promote TLR11 and/or TLR12 function. For example, agents of the invention specifically modulate TLR11 activity, TLR12 activity, activity of TLR11 and/or TLR12, and are used to treat certain diseases and disorders e.g., such as those related to an inflammatory disorder, an autoimmune disease, a cardiovascular disorder, or a systemic infection that is responsive to Toll-like receptor modulation.

The assays of the invention are useful to identify, optimize or otherwise assess agents that increase or decrease the activity of a TLR11 polypeptide, a TLR12 polypeptide or both a TLR11 and a TLR12 polypeptide.

In particular, one assay comprises screening for activation of NF-κB. For example, mammalian cells such as 293T cells transfected with an NF-κB luciferase reporter construct and expressing a constitutively active TLR11 or TLR12 polypeptide or TLR11 or TLR12 fusion protein (e.g., the cytoplasmic domain of TLR11 or TLR12 fused to the extracellular domain of a CD4 receptor) are assayed for NF-κB activation. Activation of NF-κB by constitutively active TLR11 or TLR12 can be determined, for example, by NF-κB induced luciferase activity which is measured by means of a luminometer.

Another assay of the invention comprises screening for activation of NF-κB by TLR11 or TLR12 polypeptides activated by means of an agent such as an endogenous ligand or a therapeutic compound. For example, mammalian cells such as 293T cells are transfected with an NF-κB luciferase rcporter construct and express a TLR11 or a TLR12 polypeptide. The TLR11 or TLR12 polypeptide is contacted with an agent which activates TLR11 or TLR12.

TLR11 or TLR12 activation by the agent is measured by the activation of NF-κB, which activity is measured by luciferase activity by means of a luminometer.

Yet another assay of the invention comprises detecting the production of cytokines. For example, mammalian cells such as RAW 264.7 macrophages expressing a constitutively active TLR11 or TLR12 polypeptide or TLR11 or TLR12 fusion protein (e.g., the cytoplasmic domain of TLR11 or TLR12 fused to the extracellular domain of a CD4 receptor) are tested for production of a cytokine at the cell surface of the cells by immunostaining for TNF-α followed by flow cytometry.

An assay as described above may be used in a screening assay to identify agents that modulate an immunomodulatory activity of a TLR11 and/or TLR12 polypeptide. Such a screening assay will generally involve adding a test agent to one of the above assays, or any other assay designed to assess an immunomodulatory-related activity of a TLR11 or a TLR12 polypeptide. The parameters detected in a screening assay may be compared to a suitable reference. A suitable reference may be an assay run previously, in parallel or later that omits the test agent. A suitable reference may also be an average of previous measurements in the absence of the test agent. In general the components of a screening assay mixture may be added in any order consistent with the overall activity to be assessed, but certain variations may be useful.

Assays of the invention are useful for identifying agents that bind to a TLR11 or a TLR12 polypeptide, optionally a particular domain of TLR11 or TLR12 such as an extracellular domain (e.g., a leucine rich repeat domain) or an intracellular domain such as a TIR domain. For example, an assay of the invention may be useful for identifying agents that bind to both a TLR11 and a TLR12 polypeptide. A wide variety of assays are useful for this purpose, including, but not limited to, labeled in vitro protein-protein binding assays, electrophoretic mobility shift assays, and immunoassays for protein binding. The purified protein may also be used for determination of three-dimensional crystal structure, which can be used for modeling intermolecular interactions and design of test agents. The assays detect agents which inhibit or modulate the intrinsic biological activity of a TLR11 and/or a TLR12 polypeptide, such as activation of NF-κB or stimulation of the production of cytokines.

Some assays formats include those which approximate such conditions as formation of protein complexes, and TLR11 or TLR12 immunomodulatory activity, e.g., purified proteins or cell lysates, as well as cell-based assays which utilize intact cells. Simple binding assays can also be used to detect agents which bind to TLR11 and/or TLR12. Such binding assays may also identify agents that act by disrupting the interaction between a TLR1 or a TLR12 polypeptide and a TLR11 or a TLR12 interacting protein, respectively or the binding of a TLR11 or a TLR12 polypeptide or complex to a substrate. Agents to be tested can be obtained by any means available. For example, agents to be produced, by bacteria, yeast or other organisms (e.g., natural products), produced chemically (e.g., small molecules, including peptidomimetics), or produced recombinantly. In a specific example, the test agent is a small organic molecule having a molecular weight of less than about 2 kD.

The invention also provides an assay for identifying a test compound that inhibits or potentiates the activation of a TLR11 and/or TLR12 polypeptide. In this assay a reaction mixture including TLR11 or TLR12 polypeptides and a test compound is formed. Then, the activation of the TLR11 or TLR12 polypeptides is detected. A change in the activation of the TLR1 or TLR12 polypeptide in the presence of the test compound, relative to activation in the absence of the test compound, indicates that the test compound potentiates or inhibits activation of said TLR11 and/or TLR12 polypeptide.

The involvement of MyD88 and NFkB has been demonstrated in the TLR11/TLR12 signaling pathway (Yarovinsky, F., et al., Science, 308, 1626-1629, 2005). The proposed pathway for TLR11/TLR12 signaling is depictured in FIG. 19. Several specific inhibitors affecting the pathway to different stages are indicated in FIG. 19, most of which can be purchased through Sigma Chemical. In addition, MyD88-, AP-1-, and NF-κB-defective mice are available (Jackson Lab, Bar Harbor, Me.). All the proteins involved in the pathway are affected by PA19. These include, without limitation, MyD88, IRAK, TRAF-6, NIK, IKK, IkB, NF-κB and products of activation by NF-κB (IL-12, IL-6, etc.) and possibly Erk, p38, AP-1, Akt, PI(3,4,5)P3, PI3-kinase, p85, p100 as well as other proteins that have not yet been identified using antibodies to one or more of these proteins. An assay (in the format of ELISA or Western Blot) can be performed to monitor involvement of a specific protein in the activation of TLR12 by PA19 protein. Lysis of the cells is performed to monitor the level of these intracellular proteins.

PA19 protein from E. tenella activates dendritic cells and directs NK cells to kill murine sarcoma S-180 cells in vitro as well as cure mice of that particular cancer in vivo, and efficiency increases significantly when additional specific agonists are present (Rosenberg, et al., Int. J. Cancer, 2005, 114, 756-65). While not wishing to be bound by a single theory of operability, the mechanism of anti-cancer effect of PA19 protein (Rosenberg, B., et al.,) may be through a specific receptor(s) on dendritic cells, namely TLR11/TLR12). Binding and activation of TLR11/TLR12 stimulates secretion of several inflammatory cytokines, including, without limitation, IL-12, by dendritic cells and, indirectly, by NK cells. Local release of these cytokines triggers rejection of cancer cells. The effect resembles the effect of Coley's bacterial extract, or bacilli Calmette-Guérin (BCG). However, PA19 is not toxic and works at extremely low concentrations (0.1-10 ng/mouse), which are three-six orders of magnitude lower than for the Coley bacterial extract.

Methods of determining TLR5 functional activities in response to a PRIP include methods described herein, in Examples 5.11, as well as methods known in the art. A variety of methods well known in the art can be used for determining transcription factor activities. For example, fos, jun, and NF-κB activation in response to TLR5 binding to a PRIP can be detected, for example, by electrophoretic mobility shift assays well known in the art that detect NF-κB binding to specific polynucleic acid sequences. Promoter-reporter nucleic acid constructs can be used for detecting transcription factor activation. In such a construct, a reporter is expressed e.g., β-lactamase, luciferase, green fluorescent protein or β-galactosidase, in response to contacting a TLR5 with a PRIP. For example, a luciferase reporter plasmid in which luciferase protein expression is driven by one or more NF-κB binding sites can be transfected into a cell, as described in US2005/0147627. Activation of NF-κB results in activation of luciferase reporter expression, resulting in production of luciferase enzyme able to catalyze the generation of a molecule that can be detected, e.g., by colorimetric, fluorescence, chemilluminescence or radiometric assay.

An amount or activity of a polypeptide, including a cytokine such as TNF-α, IL-1 or IL-6, can be assayed for activation of a TLR5 in response to binding a PRIP. A variety of methods well known in the art can be used to measure cytokine amounts, such as, e.g., flow cytometry methods, immunoassays such as ELISA and RIA, and cytokine RNA protection assays. Commercially available cytokine assay kits, such as ELISA assay formats, can be conveniently used to determine the amount of a variety of cytokines in a sample. Those skilled in the art will determine the particular cytokines to be measured when assessing an immune response in a cell or animal. For example, to determine whether a particular response is characterized as a TH1 or TH2 immune response, those skilled in the art will be able to select appropriate cytokines within the TH1 and TH2 categories, which are well known in the art.

IL-12 Induction

One of the major effects of an PA19 both in vitro and in vivo is the induction of interleukin-12 (IL-12) release from dendritic cells (WO 2005/010040, U.S. 2005/0169935). IL-12 has been proposed for a variety of uses, for example, without limitation, in immune regulation. However, such uses have been limited by severe toxicity associated with administration of IL-12. PA19 provides an alternative to systemic IL-12 administration and that could provide the benefits of IL-12 administration without the associated toxicity.

Moreover, WO 2005/010040 further provides methods for assessing the plasmocological induction of serum IL-12 in selective patients in the phase I clinical trial.

Further, tests known to those of skill in the art can be used to determine whether one or more polypeptides is achieving IL-12 induction results and is thereby a candidate novel PRIP of the invention. A description follows.

Dendritic Cell Activation (DCA) Assay

Another assay which can be used to show activation of TLR11/TLR12 and/or TLR5 is the Dendritic Cells Activation (DCA) assay. To follow the activity in semi-purified preparations of BEX, an assay which follows IL-12 release from freshly isolated dendritic cells (DCs) as an index of DC activation was used. This activity is highly correlated with both NK-CMC in vitro and anti-tumor activity in vivo. This is described in detail in Example 5.2.

Transfected Cell Assay

The immunomodulatory activity of the PRIP compositions of the invention, as well as the TLR11/TLR12 and TLR5 agonists of the invention, can further be detected using a “robust cell culture-based” assay. This assay uses a murine cell line (such as S-180 sarcoma), which is transfected with the mTLR11/TLR12 or TLR5 gene. The recombinant cells express the TLR11/TLR12 or TLR5 protein and assemble it on their surface. Addition of PA19 to such cells leads to activation of the receptor and initiates the signaling cascade, which results in activation of NF-κB transcriptional factor and expression of mIL-12, mIL-6, and other cytokines. The level of expression of these cytokines can be estimated on the basis of ELISA (similar to the part 2 of the DCA-assay). This assay can be used with PA19, deletion mutants, or other modifications to the PA19 protein.

In certain instances, the assay includes transfecting the immortal murine sarcoma cell line S-180 with a plasmid containing mTLR11/TLR12 and/or TLR5 gene under a strong promoter, and using the resulting cell line as a substitute for dendritic cells. The cell line expresses TLR11/TLR12 and/or TLR5 protein in significant amounts, which will be assembled on the surface of the cells. Activation of the TLR11/TLR12 and/or TLR5 by PA19 starts the MyD88/NF-κB pathway and results in activation of expression of several cytokines (including, without limitation, mIL-6, and mIL-12). These cytokines are secreted outside the cells and the accumulation of one of the cytokines can be monitored by an ELISA kit. (The ELISA test can be based on the complete mIL-12 molecule, or a mIL-6 ELISA kit, as well as mIL-12 p35, or mIL-12 p40 kits). These assays can initially be performed in parallel to the DCA-assay to show that both produce similar results. The new assay shows that PA19 indeed works through the activation of the TLR11/TLR12 and/or TLR5.

Knock-Out Tests

Negative controls testing can be done by using TLR11/TLR12 or TLR5 knock-out mice. Other experiments can be performed with MyD88-knock-out mice (available through Jackson Lab, Bar Harbor, Me.). MyD88 is the adapter interacting with the intra-cellular part of the activated form of the TLR11/TLR12 or TLR5 molecule and starts the pathway leading to activation of NF-κB. In both cases, athymic mice are bred with these knock-out mice to produce athymic knock-out mice, which can then be used for experiments with human cancer cell lines. To demonstrate that TLR11/TLR12 or TLR5 is involved in the anti-cancer activity of the PA19, the TLR12 (or MyD-88) or TLR5 knock-out mice and murine sarcoma S-180 can be injected with PA19. The results will demonstrate that TLR11/12 and/or TLR5 mediate the anti-cancer and anti-infectious disease immunomodulatory effects of PA19 and other PRIPs.

Binding to TLR11/12 and/or TLR5

Binding of TLR11/12 and/or TLR5 to candidate PRIPs or TLR agonists can be assessed using any of the methods known in the art by detecting or measuring potential protein-protein interaction. Nonlimiting examples include co-immunoprecipitation, BIACOR, GST-pull-down assays and the like. For example, physical interaction between PA19 and TLR11/TLR12 can be detected using conventional in vivo physical chemical studies such as BIA core binding assays, or in vivo methods such as the yeast two-hybrid system. The hybrid method is well-developed, and consists of creating a “bait” (TLR11/TLR12 fused with a DNA-binding domain (like GAL4 BD) at its N-terminus), and a “prey” (library of genes fused to activation domain (GAL4 AD) in an expression vector). Transfection of the “bait” and “prey” into yeast cells containing LacZ gene attached to GAL4 promoter will result by selection the cells containing both of the targets (by antibiotics) and for the cells producing LacZ (visible by the blue color of the colony). The methods are reviewed at the following web sites: http://www.uib.no/aasland/two-hybrid.html and http://www.bioteach.ubc.ca/MolecularBiology/AYeastTwoHybridAssay/.

A useful method of screening for a TLR5 ligand, agonist or antagonist, involves, (a) contacting a TLR5 with a candidate compound in the presence of a PRIP under conditions wherein binding of the PRIP to the TLR5 produces a predetermined signal; (b) determining the production of the predetermined signal in the presence of the candidate compound; and (c) comparing the predetermined signal in the presence of the candidate compound with a predetermined signal in the absence of the candidate compound, wherein a difference between the predetermined signals in the presence and absence of the candidate compound indicates that the compound is a TLR5 ligand, agonist or antagonist (U.S. Patent Pub. No. US2005/0147627).

4.4.3 TLR11/TLR12 and TLR5 Agonists

Aspects of the invention include synthetic and other novel TLR11/TLR12 and TLR5 agonists that activate this toll-like receptor and induce an immunomodulatory response. Exemplary synthetic TLR11/TLR12 and TLR5 agonists of the invention include antibodies, particularly monoclonal antibodies that have been screened for their ability to bind to and activate the TLR11/TLR12 and TLR5 receptor. Other agonists include aptamers, particularly nucleic acid aptamers that have been selected for their affinity to the TLR11/TLR12 or TLR5 and screened for a cognate TLR11/TLR12 or TLR5 agonist function. Still other TLR11/TLR12 and TLR5 agonists of the invention include synthetic polypeptides, such as circular polypeptides and peptidomimetics, and small molecules, including those available as members of chemical libraries.

The TLR11/TLR12 and TLR5 agonists of the invention are most readily identified and isolated using either a TLR11/TLR12 or TLR5 receptor target polypeptide. Various full-length and extracellular receptor domain TLR11/TLR12 and TLR5 polypeptides known in the art may be utilized for this purpose.

Antibody Agonists

Antibody agonists recognize and induce TLR11/TLR12 activity and or TLR5 activity. Novel monoclonal antibodies or fragments thereof refer in principle, to all immunoglobulin classes such as IgM, IgG, IgD, IgE, IgA or their subclasses such as the IgG subclasses or mixtures thereof. IgG and its subclasses are useful, such as IgG₁, IgG₂, IgG₂ₐ, IgG_{2b}, IgG₃ or IgG_{M}. The IgG subtypes IgG_{1/kappa} and IgG_{2b/kappa} are also useful. Fragments which may be mentioned are all truncated or modified antibody fragments with one or more antigen-complementary binding sites with high binding and neutralizing activity toward mammalian TLR11/TLR12 and/or TLR5, such as parts of antibodies having a binding site which corresponds to the antibody and is formed by light and heavy chains, such as Fv, Fab or F(ab′)₂ fragments, or single-stranded fragments. Truncated double-stranded fragments such as Fv, Fab or F(ab′)₂ are useful. These fragments can be obtained, for example, by enzymatic means by eliminating the Fc part of the antibody with enzymes such as papain or pepsin, by chemical oxidation or by genetic manipulation of the antibody genes. It is also possible and advantageous to use genetically manipulated, non-truncated fragments. The TLR11/TLR12 and/or TLR5 antibodies, or fragments thereof, can be used alone or in mixtures. For example, the invention provides assays for screening antibodies to a TLR11/TLR12 or TLR5 protein or polypeptide or a biologically active portion thereof. The invention also provides assays for screening antibodies which bind to or modulate the activity of a TLR11/TLR12 or TLR5 protein, or polypeptide, or a biologically active portion thereof.

The novel antibodies or antibody fragments or mixtures or derivatives thereof, advantageously have a binding affinity for TLR11/TLR12 or TLR5 with a dissociation constant value within a log-range of from about 1×10⁻¹¹ M (0.01 nM) to about 1×10⁻⁸ M (10 nM), or about 1×10⁻¹⁰ M (0.1 nM) to about 3×10⁻⁹ M (3 nM).

The antibody genes for the genetic manipulations can be isolated, for example from hybridoma cells, in a manner known to the skilled worker. For this purpose, antibody-producing cells are cultured and, when the optical density of the cells is sufficient, the mRNA is isolated from the cells in a known manner by lysing the cells with guanidinium thiocyanate, acidifying with sodium acetate, extracting with phenol, chloroform/isoamyl alcohol, precipitating with isopropanol and washing with ethanol. cDNA is then synthesized from the mRNA using reverse transcriptase. The synthesized cDNA can be inserted, directly or after genetic manipulation, for example by site-directed mutagenesis, introduction of insertions, inversions, deletions or base exchanges, into suitable animal, fungal, bacterial or viral vectors and be expressed in appropriate host organisms. Some useful bacterial or yeast vectors include, but are not limited to, pBR322, pUC18/19, pACYC184, lambda or yeast mu vectors for the cloning of the genes and expression in bacteria such as E. coli or in yeasts such as Saccharomyces cerevisiae.

The invention further relates to cells that synthesize TLR11/TLR12 or TLR5 antibodies. These include animal, fungal, bacterial cells or yeast cells after transformation as mentioned above. They are advantageously hybridoma cells or trioma cells. Hybridoma cells can be produced, in a manner well known in the art (see, e.g., Koehler et al., (1975) Nature 256: 496) or may be made by recombinant DNA methods (U.S. Pat. No. 4,816,567). The “monoclonal antibodies” may also be isolated from phage libraries generated using the techniques described in McCafferty et al., Nature 348:552-554 (1990). The mAb antibodies of the invention, bind with high affinity and activate the immunomodulatory activity of TLR11/TLR12 or TLR5.

The invention further includes derivates of these anti-TLR11/TLR12 or TLR5 antibodies, which retain their TLR11/TLR12 or TLR5-activating activity while altering one or more other properties related to their use as a pharmaceutical agent, e.g., serum stability or efficiency of production. Examples of such anti-TLR11/TLR12 or TLR5 antibody derivatives include, but are not limited to, peptides, peptidomimetics derived from the antigen-binding regions of the antibodies, and antibodies, fragments or peptides bound to solid or liquid carriers such as polyethylene glycol, glass, synthetic polymers such as polyacrylamide, polystyrene, polypropylene, polyethylene or natural polymers such as cellulose, Sepharose or agarose, or conjugates with enzymes, toxins or radioactive or nonradioactive markers such as ³H, ¹²³I, ¹²⁵I, ¹³¹I, ³²P, ³⁵S, ¹⁴C, ⁵¹Cr, ³⁶Cl, ⁵⁷Co, ⁵⁵Fe, ⁵⁹Fe, ⁹⁰Y, ^{99m}Tc (metastable isomer of Technetium 99), ⁷⁵Se, or antibodies, fragments or peptides covalently bonded to fluorescent/chemiluminescent labels such as rhodamine, fluorescein, isothiocyanate, phycoerythrin, phycocyanin, fluorescamine, metal chelates, avidin, streptavidin or biotin.

The novel antibodies and antibody fragments, mixtures and derivatives thereof, can be used directly, after drying, for example freeze drying, after attachment to the abovementioned carriers or after formulation with other pharmaceutical active and ancillary substances for producing pharmaceutical preparations. Examples of active and ancillary substances which may be mentioned are other antibodies, antimicrobial active substances with a microbiocidal or microbiostatic action such as antibiotics in general or sulfonamides, antitumor agents, water, buffers, salines, alcohols, fats, waxes, inert vehicles or other substances customary for parenteral products, such as amino acids, thickeners or sugars. These pharmaceutical preparations are used to control diseases, usefully to control arthritic disturbances, advantageously disturbances of joint cartilage.

The anti-TLR11/TLR12 or TLR5 antibodies of the invention can be administered orally, parenterally, subcutaneously, intramuscularly, intravenously or interperitoneally. Furthermore, direct administration to affected joints, e.g., through intramuscular or intravenous administration, is useful.

The human TLR11/TLR12 or TLR5 monoclonal antibody of the present invention may be obtained as follows. Those of skill in the art will recognize that other equivalent procedures for obtaining TLR11/TLR12 or TLR5 antibodies are also available and are included in the invention.

First, a mammal is immunized with human TLR11/TLR12 or TLR5. Purified human TLR11/TLR12 or TLR5 is available by the procedures described herein. The mammal used for raising anti-human TLR11/TLR12 or TLR5 antibody is not restricted and may be a primate, a rodent such as mouse, rat or rabbit, bovine, sheep, goat or dog.

Next, antibody-producing cells such as spleen cells are removed from the immunized animal and are fused with myeloma cells. The myeloma cells are well-known in the art (e.g., p3x63-Ag8-653, NS-0, NS-1 or P3UI cells may be used). The cell fusion operation may be carried out by a well-known conventional method.

The cells, after being subjected to the cell fusion operation, are then cultured in HAT selection medium so as to select hybridomas. Hybridomas, which produce antihuman monoclonal antibodies, are then screened. This screening may be carried out by, for example, sandwich ELISA (enzyme-linked immunosorbent assay) or the like in which the produced monoclonal antibodies are bound to the wells to which human profilin is immobilized. In this case, as the secondary antibody, an antibody specific to the immunoglobulin of the immunized animal, which is labeled with an enzyme such as peroxidase, alkaline phosphatase, glucose oxidase, beta-D-galactosidase or the like, may be employed. The label may be detected by reacting the labeling enzyme with its substrate and measuring the generated color. As the substrate, 3,3-diaminobenzidine, 2,2-diaminobis-o-dianisidine, 4-chloronaphthol, 4-aminoantipyrine, o-phenylenediamine or the like may be produced.

By the above-described operation, hybridomas, which produce anti-human TLR11/TLR12 or TLR5 antibodies, can be selected. The selected hybridomas are then cloned by the conventional limiting dilution method or soft agar method. If desired, the cloned hybridomas may be cultured on a large scale using a serum-containing or a serum free medium, or may be inoculated into the abdominal cavity of mice and recovered from ascites, thereby a large number of the cloned hybridomas may be obtained. Human myeloma and mouse-human heteromyeloma cell lines for the production of human monoclonal antibodies have been described, for example, by Kozbor (1984) J. Immunol., 133, 3001; Brodeur, et al., Monoclonal Antibody Production Techniques and Applications, pp. 51-63 (Marcel Dekker, Inc., New York, 1987); and Boerner et al., (1991) J. Immunol., 147:86-95. Specific methods for the generation of such human antibodies using, for example, phage display, transgenic mouse technologies and/or in vitro display technologies, such as ribosome display or covalent display, have been described (see Osbourn et al. (2003) Drug Discov. Today 8: 845-51; Maynard et al., (2000) Ann. Rev. Biomed. Eng. 2: 339-76; and U.S. Pat. Nos. 4,833,077; 5,811,524; 5,958,765; 6,413,771; and 6,537,809.

From among the selected anti-human TLR11/TLR12 or TLR5 monoclonal antibodies, those that have an ability to activate the TLR11/TLR12 or TLR5 immunomodulatory activity are then chosen for further analysis and manipulation. That is, the monoclonal antibody specifically recognizes and activates TLR11/TLR12 or TLR5.

The monoclonal antibodies herein further include hybrid and recombinant antibodies produced by splicing a variable (including hypervariable) domain of an anti-profilin antibody with a constant domain (e.g., “humanized” antibodies), or a light chain with a heavy chain, or a chain from one species with a chain from another species, or fusions with heterologous proteins, regardless of species of origin or immunoglobulin class or subclass designation, as well as antibody fragments as described above as long as they exhibit the desired biological activity. (See, e.g., U.S. Pat. No. 4,816,567 and Mage & Lamoyi, in Monoclonal Antibody Production Techniques and Applications, pp. 79-97 (Marcel Dekker, Inc.), New York (1987)).

“Humanized” forms of non-human (e.g., murine) antibodies are specific chimeric immunoglobulins, immunoglobulin chains or fragments thereof which contain minimal sequence derived from non-human immunoglobulin. For the most part, humanized antibodies are human immunoglobulins (recipient antibody) in which residues from the complementary determining regions (CDRs) of the recipient antibody are replaced by residues from the CDRs of a non-human species (donor antibody) such as mouse, rat or rabbit having the desired specificity, affinity and capacity. In some instances, Fv framework region (FR) residues of the human immunoglobulin are replaced by corresponding non-human FR residues. Furthermore, the humanized antibody may comprise residues that are found neither in the recipient antibody nor in the imported CDR or FR sequences. These modifications are made to further refine and optimize antibody performance. In general, the humanized antibody will comprise substantially all of at least one, and typically two, variable domains, in which all or substantially all of the CDR regions correspond to those of a non-human immunoglobulin and all or substantially all of the FR residues are those of a human immunoglobulin consensus sequence. The humanized antibody optimally also will comprise at least a portion of an immunoglobulin constant region (Fc), typically that of a human immunoglobulin.

Methods for humanizing non-human antibodies are well known in the art (see, e.g., Jones et. al., (1986) Nature 321: 522-525; Riechmann et al., (1988) Nature, 332: 323-327; and Verhoeyen et al., (1988) Science 239: 1534-1536).

The choice of human variable domains, both light and heavy, to be used in making the humanized antibodies is very important to reduce antigenicity. The human sequence which is closest to that of the rodent is usually accepted as the human framework (FR) for the humanized antibody (Sims et al., (1993) J. Immunol., 151:2296; and Chothia and Lesk (1987) J. Mol. Biol., 196:901). Alternatively, a particular framework is used that is derived from the consensus sequence of all human antibodies of a particular subgroup of light or heavy chains. The same framework may be used for several different humanized antibodies (Carter et al., (1992) Proc. Natl. Acad. Sci, (USA), 89: 4285; and Presta et al., (1993) J. Immunol., 151:2623).

Antibodies are humanized with retention of high affinity for the antigen and other favorable biological properties.

It is now possible to produce transgenic animals (e.g., mice) that are capable, upon immunization, of producing a full repertoire of human antibodies in the absence of endogenous immunoglobulin production. For example, it has been described that the homozygous deletion of the antibody heavy-chain joining region (J_{H}) gene in chimeric and germ-line mutant mice results in complete inhibition of endogenous antibody production. Transfer of the human germ-line immunoglobulin gene array in such gem-line mutant mice results in the production of human antibodies upon antigen challenge (see, e.g., Jakobovits et al., (1993) Proc. Natl. Acad. Sci. (USA), 90: 2551; Jakobovits et al., (1993) Nature, 362:255-258; and Bruggermann et al., (1993) Year in Immuno., 7:33).

Alternatively, phage display technology (McCafferty et al., (1990) Nature, 348: 552-553) can be used to produce human antibodies and antibody fragments in vitro, from immunoglobulin variable (V) domain gene repertoires from unimmunized donors.

In a natural immune response, antibody genes accumulate mutations at a high rate (somatic hypermutation). Some of the changes introduced will confer higher affinity, and B cells displaying high-affinity surface immunoglobulin are preferentially replicated and differentiated during subsequent antigen challenge. This natural process can be mimicked by employing the technique known as “chain shuffling” (see Marks et al., (1992) Bio/Technol., 10:779-783). In this method, the affinity of “primary” human antibodies obtained by phage display can be improved by sequentially replacing the heavy and light chain V region genes with repertoires of naturally occurring variants (repertoires) of V domain genes obtained from unimmunized donors. This technique allows the production of antibodies and antibody fragments with affinities in the nM range. A strategy for making very large phage antibody repertoires has been described by Waterhouse et al., (1993) Nucl, Acids Res., 21:2265-2266).

Gene shuffling can also be used to derive human antibodies from rodent antibodies, where the human antibody has similar affinities and specificities to the starting rodent antibody. According to this method, which is also referred to as “epitope imprinting”, the heavy or light chain V domain gene of rodent antibodies obtained by phage display technique is replaced with a repertoire of human V domain genes, creating rodent-human chimeras. Selection on antigen results in isolation of human variable capable of restoring a functional antigen-binding site, i.e., the epitope governs (imprints) the choice of partner. When the process is repeated in order to replace the remaining rodent V domain, a human antibody is obtained (see PCT WO 93/06213, published 1 Apr. 1993). Unlike traditional humanization of rodent antibodies by CDR grafting, this technique provides completely human antibodies, which have no framework or CDR residues of rodent origin.

By using the above-described antibodies of the present invention, human profilin in a sample can be detected or quantified. The detection or quantification of the human TLR11/TLR12 and/or TLR5 in a sample can be carried out by an immunoassay utilizing the specific binding reaction between the antibody and human TLR11/TLR12 and/or TLR5. Various immunoassays are well-known in the art and any of them can be employed. Examples of the immunoassays include, but are not limited to, sandwich method employing a monoclonal antibody to a PRIP and another monoclonal antibody as primary and secondary antibodies, respectively; sandwich methods employing the monoclonal antibody and a polyclonal antibody as primary and secondary antibodies; staining methods employing gold colloid; agglutination method; latex method; and chemical luminescence.

Aptamer Agonists

Other agonists useful in the invention are aptamers. Aptamers are chemically synthesized short strands of nucleic acid that adopt specific three-dimensional conformations and are selected for their affinity to a particular target through a process of in vitro selection referred to as systematic evolution of ligands by exponential enrichment (SELEX). SELEX is a combinatorial chemistry methodology in which vast numbers of oligonucleotides are screened rapidly for specific sequences that have appropriate binding affinities and specificities toward any target. Using this process, novel aptamer nucleic acid ligands that are specific for a particular target may be created. Aptamers can be prepared that bind to a wide variety of target molecules. The aptamer nucleic acid sequences of the invention can be comprised entirely of RNA or partially of RNA, or entirely or partially of DNA and/or other nucleotide analogs. Methods of making aptamers are described in, e.g., Ellington et al., (1990) Nature 346:818; U.S. Pat. Nos. 5,582,981, 5,270,163; 5,756,291 and Huizenga et al., (1995) Biochem. 34:656-665; PCT Publication Nos. WO 00/20040, WO 99/54506, WO 99/27133, and WO 97/42317.

The aptamer nucleic acid sequences may also be modified. For example, certain modified nucleotides can confer improved characteristic on high-affinity nucleic acid ligands containing them, such as improved in vivo stability or improved delivery characteristics. Representative examples of such modifications are described in U.S. Pat. No. 5,660,98.

The invention provides aptamers that function to inhibit the binding of any of various biological targets to one or more binding partners. The aptamer thereby functions as an antagonist of the biological target (TLR11/TLR12 or TLR5). In most instances, the disruption of the target/binding partner interaction functions to inhibit one or more biological functions of the target protein.

Polypeptides and Peptidomimetic Agonists

Other useful agonists of the invention are peptidomimetics, e.g., peptide or non-peptide agents, such as small molecules, which are able to bind to, modulate and/or activate either TLR11/TLR12 or TLR5. Thus, the mutagenic techniques as described above for the PRIPs are also useful to map the determinants of the TLR11/TLR12 and TLR5 proteins which participate in protein-protein interactions involved in, for example, binding of the subject profilin to a TLR11/TLR12 or TLR5 polypeptide.

A “peptide mimetic” is a molecule that mimics the biological activity of a peptide but is no longer peptidic in chemical nature. By strict definition, a “peptidomimetic” is a molecule that no longer contains any peptide bonds (that is, amide bonds between amino acids). However, the term “peptide mimetic” is sometimes used to describe molecules that are no longer completely peptidic in nature, such as pseudo-peptides, semi-peptides and peptoids. Whether completely or partially non-peptide, peptidomimetics according to this invention provide a spatial arrangement of reactive chemical moieties that closely resembles the three-dimensional arrangement of active groups in the peptide on which the peptidomimetic is based. As a result of this similar active-site geometry, the peptidomimetic has effects on biological systems which are similar to the biological activity of the peptide.

The present invention encompasses peptidomimetic compositions which are analogs that mimic the activity of biologically active peptides according to the invention, i.e., the peptidomimetics are capable of modulating and/or activating the immunomodulatory activity of TLR11/TLR12 or TLR5. The peptidomimetics of this invention can be usefully substantially similar in both three-dimensional shape and biological activity to the profilin peptides set forth above. “Substantial similarity” means that the geometric relationship of groups in the profilin peptide that react with TLR11/TLR12 or TLR5 is preserved and at the same time, that the peptidomimetic modulates and/or activates TLR11/TLR12 or TLR5 activity.

Peptide bonds can be replaced by non-peptide bonds that allow the peptidomimetic to adopt a similar structure, and therefore biological activity, to the original peptide. Further modifications can also be made by replacing chemical groups of the amino acids with other chemical groups of similar structure. The development of peptidomimetics can be aided by determining the tertiary structure of the original profilin peptide, either free or bound to TLR11/TLR12 or TLR5, by NMR spectroscopy, crystallography and/or computer-aided molecular modeling. These techniques aid in the development of novel compositions of higher potency and/or greater bioavailability and/or greater stability than the original peptide (see, e.g., Dean (1994), BioEssays, 16: 683-687; Cohen and Shatzmiller (1993), J. Mol. Graph., 11: 166-173; Wiley and Rich (1993), Med. Res. Rev., 13: 327-384; Moore (1994), Trends Pharmacol. Sci., 15: 124-129; Hruby (1993), Biopolymers, 33: 1073-1082; Bugg et al. (1993), Sci. Am., 269: 92-98). Once a potential peptidomimetic compound is identified, it may be synthesized and assayed using the TLR11/TLR12 and/or TLR5 assays described herein to assess its activity.

The peptidomimetic compounds obtained by the above methods, having the biological activity of the above named peptides and similar three dimensional structure, are encompassed by this invention. It will be readily apparent to one skilled in the art that a peptidomimetic can be generated from any of the modified peptides described in the previous section or from a peptide bearing more than one of the modifications described from the previous section. It will furthermore be apparent that the peptidomimetics of this invention can be further used for the development of even more potent non-peptidic compounds, in addition to their utility as therapeutic compounds.

To illustrate, the critical residues of a subject PRIP which are involved in molecular recognition of its receptor are used to generate profilin-derived peptidomimetics or small molecules which competitively bind to the authentic TLR11/TLR12 or TLR5 protein with that moiety. Scanning mutagenesis can be employed to map the amino acid residues of the subject PRIPs which are involved in binding TLR11/TLR12 or TLR5. Peptidomimetic compounds are then generated which mimic those residues of the PRIP which facilitate the interaction. Such mimetics may then be used to mimic the normal function of a PRIP. For instance, non-hydrolyzable peptide analogs of such residues can be generated using benzodiazepine (e.g., see Freidinger et al. in Peptides: Chemistry and Biology, (G. R. Marshall ed.), ESCOM: Leiden, Netherlands, 1988), azepine (see e.g., Huffman et al., ibid), substituted gamma lactam rings (Garvey et al., ibid), keto-methylene pseudopeptides (Ewenson et al. (1986) J. Med. Chem. 29:295; and Ewenson et al. in Peptides: Structure and Function (Proceedings of the 9th American Peptide Symposium) Pierce Chemical Co. Rockland, Ill., 1985), ρ-turn dipeptide cores (Nagai et al. (1985) Tet. Lett. 26:647; Sato et al. (1986) J. Chem. Soc. Perkin Trans. 1:1231); and β-aminoalcohols (Gordon et al. (1985) Biochem. Biophys. Res. Commun. 126:419; and Dann et al. (1986) Biochem. Biophys. Res. Commun. 134:71).

Small Molecule Agonists

The invention also provides methods or screening assays for identifying modulators, i.e., candidate or test compounds or agents which bind to TLR11/TLR12 or TLR5 proteins, have a stimulatory or inhibitory effect on, for example, TLR11/TLR12 or TLR5 expression or TLR11/TLR12 or TLR5 activity, or have a stimulatory or inhibitory effect on, e.g., the expression or activity of a TLR11/TLR12 or TLR5 substrate. Exemplary small molecules include, but are not limited to, peptides, peptidomimetics (e.g., peptoids), amino acids, amino acid analogs, nucleotides, nucleotide analogs, organic or inorganic compounds (i.e., including heterorganic and organometallic compounds) having a molecular weight less than about 10 kD, organic or inorganic compounds having a molecular weight less than about 5 kD, organic or inorganic compounds having a molecular weight less than about 1 kD, organic or inorganic compounds having a molecular weight less than about 0.5 kD, and salts, esters, and other pharmaceutically acceptable forms of such compounds.

Compounds thus identified can be used to modulate the activity of target gene products (e.g., TLR11/TLR12 and/or TLR5 genes) in a therapeutic protocol, to elaborate the biological function of the target gene product, or to identify compounds that disrupt normal target gene interactions.

For example, the invention provides assays for screening candidate or test compounds which bind to or modulate the activity of a TLR11/TLR12 or TLR5 protein or polypeptide or a biologically active portion thereof.

The test compounds of the present invention can be obtained using any of the numerous approaches in combinatorial library methods known in the art, including: biological libraries; peptide libraries (libraries of molecules having the functionalities of peptides, but with a novel, non-peptide backbone which are resistant to enzymatic degradation but which nevertheless remain bioactive; (see, e.g., Zuckermann et al. (1994) J. Med. Chem. 37:2678-85); spatially addressable parallel solid phase or solution phase libraries; synthetic library methods requiring deconvolution; and the “one-bead one-compound” library method; and synthetic library methods using affinity chromatography selection. The biological library and peptide library approaches are limited to peptide libraries, while the other four approaches are applicable to peptide, non-peptide oligomer or small molecule libraries of compounds (Lam (1997) Anticancer Drug Des. 12:145).

Methods for the synthesis of molecular libraries are well known in the art, (see e.g., DeWitt et al. (1993) Proc. Natl. Acad. Sci. U.S.A. 90:6909-13 and Gallop et al. (1994) J. Med. Chem. 37:1233-51.

Libraries of compounds can be presented in solution (e.g., Houghten (1992) Biotech. 13:412-421), or on beads (Lam (1991) Nature 354:82-84), chips (Fodor (1993) Nature 364:555-556), bacteria (Ladner, U.S. Pat. No. 5,223,409), spores (Ladner U.S. Pat. No. '409), plasmids (Cull et al. (1992) Proc. Nat. Acad. Sci. (USA) 89:1865-1869) or on phage (see e.g., Felici (1991) J. Mol. Biol. 222:301-310).

The assay may be a cell-based assay in which a cell which expresses a TLR11/TLR12 or TLR5 protein or biologically active portion thereof is contacted with a test compound, and the ability of the test compound to modulate TLR11/TLR12 or TLR5 activity is determined.

The ability of the test compound to modulate TLR11/TLR12 or TLR5 binding to a compound, e.g., a TLR11/TLR12 or TLR5 substrate, or to bind to TLR11/TLR12 and/or TLR5 can also be evaluated. This can be accomplished, for example, by coupling the compound, e.g., the substrate, with a radioisotope or enzymatic label such that binding of the compound, e.g., the substrate, to TLR11/TLR12 or TLR5 can be determined by detecting the labeled compound, e.g., substrate, in a complex. Alternatively, TLR11/TLR12 or TLR5 could be coupled with a label (e.g., radioisotope or enzymatic label) to monitor the ability of a test compound to modulate TLR11/TLR12 or TLR5 binding to a TLR11/TLR12 or TLR5 substrate in a complex. For example, compounds (e.g., TLR11/TLR12 and/or TLR5 substrates) can be labeled with e.g., ¹²⁵I, ¹⁴C, ³⁵S or ³H, either directly or indirectly, and the radioisotope detected by direct counting of radioemmission or by scintillation counting. Alternatively, compounds can be enzymatically labeled with, e.g., horseradish peroxidase, alkaline phosphatase, or luciferase, and the enzymatic label detected by determination of conversion of an appropriate substrate to product.

The ability of a compound to interact with TLR11/TLR12 or TLR5 with or without the labeling of any of the interactants can be evaluated. For example, a microphysiometer can be used to detect the interaction of a compound with TLR11/TLR12 or TLR5 without the labeling of either the compound or the TLR11/TLR12 or TLR5. McConnell et al. (1992) Science 257:1906-1912. As used herein, a “microphysiometer” (e.g., Cytosensor) is an analytical instrument that measures the rate at which a cell acidifies its environment using a light-addressable potentiometric sensor (LAPS). Changes in this acidification rate can be used as an indicator of the interaction between a compound and TLR11/TLR12 or TLR5.

The invention also provides a cell-free assay in which a TLR11/TLR12 or TLR5 protein, or biologically active portion thereof, is contacted with a test compound, and the ability of the test compound to bind to the TLR11/TLR12 or TLR5 protein, or biologically active portion thereof, is evaluated. Useful biologically active portions of the TLR11/TLR12 or TLR5 proteins to be used in assays of the present invention include fragments which participate in interactions with non-TLR11/TLR12 or TLR5 molecules, e.g., fragments with high surface probability scores.

Soluble and/or membrane-bound forms of isolated proteins (e.g., TLR11/TLR12 or TLR5 proteins or biologically active portions thereof) can be used in the cell-free assays of the invention. When membrane-bound forms of the protein are used, it may be desirable to utilize a solubilizing agent. Nonlimiting examples of such solubilizing agents include non-ionic detergents such as n-octylglucoside, n-dodecylglucoside, n-dodecylmaltoside, octanoyl-N-methylglucamide, decanoyl-N-methylglucamide, Triton®X-100, Triton®X-114, Thesit®, Isotridecypoly(ethylene glycol ether)ₙ, 3-[(3-cholamidopropyl)dimethylamminio]-1-propane sulfonate (CHAPS), 3-[(3-cholamidopropyl)dimethylamminio]-2-hydroxy-1-propane sulfonate (CHAPSO), and N-dodecyl-N,N-dimethyl-3-ammonio-1-propane sulfonate.

Cell-free assays involve preparing a reaction mixture of the target gene protein and the test compound under conditions and for a time sufficient to allow the two components to interact and bind, thus forming a complex that can be removed and/or detected.

The interaction between two molecules can be detected, e.g., using fluorescence energy transfer (FET) (see U.S. Pat. Nos. 5,631,169; and 4,868,103). A fluorophore label on the first, “donor” molecule is selected such that its emitted fluorescent energy will be absorbed by a fluorescent label on a second, “acceptor” n molecule, which in turn is able to fluoresce due to the absorbed energy. Alternately, the “donor” protein molecule can simply utilize the natural fluorescent energy of tryptophan residues. Labels are chosen that emit different wavelengths of light, such that the “acceptor” molecule label can be differentiated from that of the “donor”. Since the efficiency of energy transfer between the labels is related to the distance separating the molecules, the spatial relationship between the molecules can be assessed. In a situation in which binding occurs between the molecules, the fluorescent emission of the “acceptor” molecule label in the assay should be maximal. An FET binding event can be conveniently measured through standard fluorometric detection means well known in the art (e.g., using a fluorimeter).

Alternatively the ability of the TLR11/TLR12 or TLR5 protein to bind to a target molecule can be accomplished using real-time Biomolecular Interaction Analysis (BIA) (see, e.g., Sjolander et al. (1991) Anal. Chem. 63:2338-2345 and Szabo et al. (1995) Curr. Opin. Struct. Biol. 5:699-705). “Surface plasmon resonance” or “BIA” detects biospecific interactions in real time, without labeling any of the interactants (e.g., BIAcore). Changes in the mass at the binding surface (indicative of a binding event) result in alterations of the refractive index of light near the surface (the optical phenomenon of surface plasmon resonance (SPR)), resulting in a detectable signal which can be used as an indication of real-time reactions between biological molecules.

The target gene product or the test substance can be anchored onto a solid phase and can be detected at the end of the reaction. The target gene product can be anchored onto a solid surface, and the test compound, (which is not anchored), can be labeled, either directly or indirectly, with detectable labels discussed herein.

It may be desirable to immobilize either TLR11/TLR12 or an anti-TLR11/TLR12 antibody, or the TLR5 or an anti-TLR5 antibody or its target molecule to facilitate separation of complexed from uncomplexed forms of one or both of the proteins, as well as to accommodate automation of the assay. Binding of a test compound to a TLR11/TLR12 or TLR5 protein, or interaction of a TLR11/TLR12 or TLR5 protein with a target molecule in the presence and absence of a candidate compound, can be accomplished in any vessel suitable for containing the reactants. Nonlimiting examples of such vessels include microtiter plates, test tubes, and micro-centrifuge tubes. In some instances a fusion protein can be provided which adds a domain that allows one or both of the proteins to be bound to a matrix. For example, glutathione-S-transferase/TLR11/TLR12 or TLR5 fusion proteins or glutathione-S-transferase/target fusion proteins can be adsorbed onto glutathione sepharose beads (Sigma Chemical, St. Louis, Mo.) or glutathione derivatized microtiter plates, which are then combined with the test compound or the test compound and either the non-adsorbed target protein or TLR11/TLR12 or TLR5 protein, and the mixture incubated under conditions conducive to complex formation (e.g., at physiological conditions for salt and pH). Following incubation, the beads or microtiter plate wells are washed to remove any unbound components, the matrix immobilized in the case of beads, complex determined either directly or indirectly, for example, as described above. Alternatively, the complexes can be dissociated from the matrix, and the level of TLR11/TLR12 and/or TLR5 binding or activity determined using standard techniques.

Other techniques for immobilizing either a TLR11/TLR12 or TLR5 protein or a target molecule on matrices include using conjugation of biotin and streptavidin. Biotinylated TLR11/TLR12 or TLR5 protein or target molecules can be prepared from biotin-NHS (N-hydroxy-succinimide) using techniques known in the art (e.g., biotinylation kit, Pierce Chemicals, Rockford, Ill.), and immobilized in the wells of streptavidin-coated 96 well plates (Pierce Chemical).

In order to conduct the assay, the non-immobilized component is added to the coated surface containing the anchored component. After the reaction is complete, unreacted components are removed (e.g., by washing) under conditions such that any complexes formed will remain immobilized on the solid surface. The detection of complexes anchored on the solid surface can be accomplished in a number of ways. Where the previously non-immobilized component is pre-labeled, the detection of label immobilized on the surface indicates that complexes were formed. Where the previously non-immobilized component is not pre-labeled, an indirect label can be used to detect complexes anchored on the surface; e.g., using a labeled antibody specific or selective for the immobilized component (the antibody, in turn, can be directly labeled or indirectly labeled with, e.g., a labeled anti-Ig antibody).

This assay can be performed utilizing antibodies reactive with TLR11/TLR12 or TLR5 protein or target molecules but which do not interfere with binding of the TLR11/TLR12 or TLR5 protein to its target molecule. Such antibodies can be derivatized to the wells of the plate, and unbound target or TLR11/TLR12 or TLR5 protein trapped in the wells by antibody conjugation. Methods for detecting such complexes, in addition to those described above for the GST-immobilized complexes, include immunodetection of complexes using antibodies reactive with the TLR11/TLR12 and/or TLR5 protein or target molecule, as well as enzyme-linked assays which rely on detecting an enzymatic activity associated with the TLR11/TLR12 and/or TLR5 protein or target molecule.

Alternatively, cell free assays can be conducted in a liquid phase. In such an assay, the reaction products are separated from unreacted components, by any of a number of standard techniques, including but not limited to: differential centrifugation (see, for example, Rivas and Minton (1993) Trends Biochem Sci 18:284-7); chromatography (gel filtration chromatography, ion-exchange chromatography); electrophoresis (see, e.g., Ausubel et al., (1999) Current Protocols in Molecular Biology, J. Wiley, New York.); and immunoprecipitation (see, Ausubel et al., (1999) Current Protocols in Molecular Biology, J. Wiley, New York). Such resins and chromatographic techniques are known to one skilled in the art (see, e.g., Heegaard (1998) J. Mol. Recognit. 11:141-8; Hage et al. (1997) J. Chromatogr. B. Biomed. Sci. Appl. 699:499-525). Further, fluorescence energy transfer can also be conveniently utilized, as described herein, to detect binding without further purification of the complex from solution.

The assay includes contacting the TLR11/TLR12 or TLR5 protein or biologically active portion thereof with a known compound which binds TLR11/TLR12 or TLR5 to form an assay mixture, contacting the assay mixture with a test compound, and determining the ability of the test compound to interact with a TLR11/TLR12 or TLR5 protein, wherein determining the ability of the test compound to interact with a TLR11/TLR12 or TLR5 protein includes determining the ability of the test compound to preferentially bind to TLR11/TLR12 or TLR5 or biologically active portion thereof, or to modulate the activity of a target molecule, as compared to the known compound.

The target gene products of the invention can, in vivo, interact with one or more cellular or extracellular macromolecules, such as proteins. For the purposes of this discussion, such cellular and extracellular macromolecules are referred to herein as “binding partners.” Compounds that disrupt such interactions can be useful in regulating the activity of the target gene product. Such compounds can include, but are not limited to molecules such as antibodies, peptides, and small molecules. The useful target genes/products are the TLR11/TLR12 or TLR5 genes herein identified. The invention also provides methods for determining the ability of the test compound to modulate the activity of a TLR11/TLR12 or TLR5 protein through modulation of the activity of a downstream effector of a TLR11/TLR12 or TLR5 target molecule. For example, the activity of the effector molecule on an appropriate target can be determined, or the binding of the effector to an appropriate target can be determined, as previously described.

To identify compounds that interfere with the interaction between the target gene product and its cellular or extracellular binding partner(s), a reaction mixture containing the target gene product and the binding partner is prepared, under conditions and for a time sufficient, to allow the two products to form complex. In order to test an inhibitory agent, the reaction mixture is provided in the presence and absence of the test compound. The test compound can be initially included in the reaction mixture, or can be added at a time subsequent to the addition of the target gene and its cellular or extracellular binding partner. Control reaction mixtures are incubated without the test compound or with a placebo. The formation of any complexes between the target gene product and the cellular or extracellular binding partner is then detected. The formation of a complex in the control reaction, but not in the reaction mixture containing the test compound, indicates that the compound interferes with the interaction of the target gene product and the interactive binding partner. Additionally, complex formation within reaction mixtures containing the test compound and normal target gene product can also be compared to complex formation within reaction mixtures containing the test compound and mutant target gene product. This comparison can be important in those cases wherein it is desirable to identify compounds that disrupt interactions of mutant but not normal target gene products.

These assays can be conducted in a heterogeneous or homogeneous format. Heterogeneous assays involve anchoring either the target gene product or the binding partner onto a solid phase, and detecting complexes anchored on the solid phase at the end of the reaction. In homogeneous assays, the entire reaction is carried out in a liquid phase. In either approach, the order of addition of reactants can be varied to obtain different information about the compounds being tested. For example, test compounds that interfere with the interaction between the target gene products and the binding partners, e.g., by competition, can be identified by conducting the reaction in the presence of the test substance. Alternatively, test compounds that disrupt preformed complexes, e.g., compounds with higher binding constants that displace one of the components from the complex, can be tested by adding the test compound to the reaction mixture after complexes have been formed. The various formats are briefly described below.

In a heterogeneous assay system, either the target gene product or the interactive cellular or extracellular binding partner, is anchored onto a solid surface (e.g., a microtiter plate), while the non-anchored species is labeled, either directly or indirectly. The anchored species can be immobilized by non-covalent or covalent attachments. Alternatively, an immobilized antibody specific or selective for the species to be anchored can be used to anchor the species to the solid surface.

In order to conduct the assay, the partner of the immobilized species is exposed to the coated surface with or without the test compound. After the reaction is complete, unreacted components are removed (e.g., by washing) and any complexes formed will remain immobilized on the solid surface. Where the non-immobilized species is pre-labeled, the detection of label immobilized on the surface indicates that complexes were formed. Where the non-immobilized species is not pre-labeled, an indirect label can be used to detect complexes anchored on the surface; e.g., using a labeled antibody specific or selective for the initially non-immobilized species (the antibody, in turn, can be directly labeled or indirectly labeled with, e.g., a labeled anti-Ig antibody). Depending upon the order of addition of reaction components, test compounds that inhibit complex formation or that disrupt preformed complexes can be detected.

Alternatively, the reaction can be conducted in a liquid phase in the presence or absence of the test compound, the reaction products separated from unreacted components, and complexes detected; e.g., using an immobilized antibody specific or selective for one of the binding components to anchor any complexes formed in solution, and a labeled antibody specific or selective for the other partner to detect anchored complexes. Again, depending upon the order of addition of reactants to the liquid phase, test compounds that inhibit complex or that disrupt preformed complexes can be identified.

Alternatively, a homogeneous assay can be used. For example, a preformed complex of the target gene product and the interactive cellular or extracellular binding partner product is prepared in that either the target gene products or their binding partners are labeled, but the signal generated by the label is quenched due to complex formation (see, e.g., U.S. Pat. No. 4,109,496 that utilizes this approach for immunoassays). The addition of a test substance that competes with and displaces one of the species from the preformed complex will result in the generation of a signal above background. In this way, test substances that disrupt target gene product-binding partner interaction can be identified.

In yet another aspect, the TLR11/TLR12 or TLR5 proteins can be used as “bait proteins” in a two-hybrid assay or three-hybrid assay (see, e.g., U.S. Pat. No. 5,283,317; Zervos et al. (1993) Cell 72:223-232; Madura et al. (1993) J. Biol. Chem. 268:12046-12054; Bartel et al. (1993) Biotechniques 14:920-924; Iwabuchi et al. (1993) Oncogene 8:1693-1696; and Brent WO94/10300), to identify other proteins, which bind to or interact with TLR11/TLR12 and/or TLR5 (“TLR11/TLR12 and/or TLR5-binding proteins” or “TLR11/TLR12 and/or TLR5-bp”) and are involved in TLR11/TLR12 and/or TLR5 activity. Such TLR11/TLR12 and/or TLR5-bps can be activators or inhibitors of signals by the TLR11/TLR12 and/or TLR5 proteins or TLR11/TLR12 and/or TLR5 targets as, for example, downstream elements of a TLR11/TLR12 and/or TLR5-mediated signaling pathway.

The two-hybrid system is based on the modular nature of most transcription factors, which consist of separable DNA-binding and activation domains. Briefly, the assay utilizes two different DNA constructs. In one construct, the gene that codes for a TLR11/TLR12 and/or TLR5 protein is fused to a gene encoding the DNA binding domain of a known transcription factor (e.g., GAL-4). In the other construct, a DNA sequence, from a library of DNA sequences, that encodes an unidentified protein (“prey” or “sample”) is fused to a gene that codes for the activation domain of the known transcription factor. (Alternatively the: TLR11/TLR12 and/or TLR5 protein can be the fused to the activator domain.) If the “bait” and the “prey” proteins are able to interact, in vivo, forming a TLR11/TLR12 and/or TLR5-dependent complex, the DNA-binding and activation domains of the transcription factor are brought into close proximity. This proximity allows transcription of a reporter gene (e.g., lacZ) which is operably linked to a transcriptional regulatory site responsive to the transcription factor. Expression of the reporter gene can be detected and cell colonies containing the functional transcription factor can be isolated and used to obtain the cloned gene which encodes the protein which interacts with the TLR11/TLR12 and/or TLR5 protein.

### 4.5 Pharmaceutical Formulations

In yet another aspect, the present invention provides pharmaceutical formulations that include one or more of the polypeptides or immunomodulatory and/or immunostimulatory compounds, including, without limitation, immunostimulatory agonists, as discussed above and/or PRIPs, in combination with a pharmaceutically acceptable carrier.

The polypeptides or immunomodulatory and/or immunostimulatory compounds (also referred to herein as “active compounds”) of the invention can be incorporated into pharmaceutical compositions. Such compositions typically include the polypeptide or immunomodulatory and/or immunostimulatory compound and a pharmaceutically acceptable carrier. As used herein the language “pharmaceutically acceptable carrier” includes solvents, dispersion media, coatings, antibacterial and antifungal agents, isotonic and absorption delaying agents, and the like, compatible with pharmaceutical administration. Supplementary active compounds can also be incorporated into the compositions. The pharmaceutical compositions may be formulated according to conventional pharmaceutical practice (see, e.g., Remington: The Science and Practice of Pharmacy (20th ed.), ed. A. R. Gennaro, Lippincott Williams & Wilkins, 2000 and Encyclopedia of Pharmaceutical Technology, (eds. J. Swarbrick and J. C. Boylan), 1988-1999, Marcel Dekker, New York).

A pharmaceutical composition is formulated to be compatible with its intended route of administration. Examples of routes of administration include parenteral, e.g., intravenous, intradermal, subcutaneous, inhalation, transdermal (topical), transmucosal, and rectal administration, or oral. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringability exists. It should be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the selected particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, isotonic agents, for example, sugars, polyalcohols such as mannitol, sorbitol, and sodium chloride are included in the composition. Prolonged absorption of the injectable compositions can be brought about by including in the composition an agent which delays absorption, for example, aluminum monostearate and gelatin.

Sterile injectable solutions can be prepared by incorporating the active compound in the specified amount in an appropriate solvent with one or a combination of ingredients enumerated above, as needed, followed by filtered sterilization. Generally, dispersions are prepared by incorporating the active compound into a sterile vehicle, which contains a basic dispersion medium and other ingredients selected from those enumerated above or others known in the art. In the case of sterile powders for the preparation of sterile injectable solutions, the methods of preparation can be vacuum drying and freeze-drying, which yields a powder of the active ingredient plus any additional desired ingredient from a previously sterile-filtered solution thereof.

Oral compositions generally include an inert diluent or an edible carrier. For the purpose of oral therapeutic administration, the active compound can be incorporated with excipients and used in the form of tablets, troches, or capsules, e.g., gelatin capsules. Oral compositions can also be prepared using a fluid carrier for use as a mouthwash. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose; a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from a pressured container or dispenser, which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art. The compounds can also be prepared in the form of suppositories (e.g., with conventional suppository bases such as cocoa butter and other glycerides) or retention enemas for rectal delivery.

For example, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially, for example, from Alza Corporation (Mountain View, Calif.). Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is often advantageous to formulate oral or parenteral compositions in dosage unit form for ease of administration and uniformity of dosage. “Dosage unit form” as used herein refers to physically discrete units suited as unitary dosages for the subject to be treated; each unit containing a predetermined quantity of active compound calculated to produce the desired therapeutic effect in association with the selected pharmaceutical carrier.

Toxicity and therapeutic efficacy of such compounds can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index, and it can be expressed as the ratio LD₅₀/ED₅₀. In some instances, the compounds used exhibit high therapeutic indices. While compounds that exhibit toxic side effects may be used, care should be taken to design a delivery system that targets such compounds to the site of affected tissue to minimize potential damage to uninfected cells and, thereby, reduce side effects.

The pharmaceutical compositions can be included in a container, pack, or dispenser together with instructions for administration.

### 4.6 Methods of Treatment

The invention further includes methods of treating or preventing diseases that are subject to PRIP immunotherapy, including cancers and infection diseases. Subjects amenable to these methods of treatment and prevention include mammals as well as non-mammalian animals. Mammalian subjects treated by the method of the invention include, but are not limited to, humans, as well as non-human mammals such as dogs, cats, cows, monkeys, mice, and rats. The subject treated can also be an avian species, such as a chicken or other fowl.

Cancers subject to treatment and invention include lymphomas, sarcomas, and carcinomas as well as cancers affecting various tissues including breast cancer, bladder cancer, prostate cancer, ovarian cancer, pancreatic cancer, rectal cancer, lung cancer, bowl cancer, colorectal cancer, leukemia, lung cancer, skin cancer, stomach cancer and uterine, endometrial and cervical cancer. Further examples of cellular proliferative and/or differentiative disorders include metastatic disorders or hematopoietic neoplastic disorders. A metastatic tumor can arise from a multitude of primary tumor types, including but not limited to those of prostate, colon, lung, breast and liver origin, and metastasize to other organs or tissues.

The term “carcinoma” is art recognized and refers to malignancies of epithelial or endocrine tissues including respiratory system carcinomas, gastrointestinal system carcinomas, genitourinary system carcinomas, testicular carcinomas, breast carcinomas, prostatic carcinomas, endocrine system carcinomas, and melanomas. Exemplary carcinomas include those forming from tissue of the cervix, lung, prostate, breast, head and neck, colon and ovary. The term also includes carcinosarcomas, e.g., which include malignant tumors composed of carcinomatous and sarcomatous tissues. An “adenocarcinoma” refers to a carcinoma derived from glandular tissue or in which the tumor cells form recognizable glandular structures. The term “sarcoma” is art recognized and refers to malignant tumors of mesenchymal tissue.

As used herein, the terms “cancer”, “hyperproliferative” and “neoplastic” refer to cells having the capacity for autonomous growth, i.e., an abnormal state or condition characterized by rapidly proliferating cell growth. Hyperproliferative and neoplastic disease states may be categorized as pathologic, i.e., characterizing or constituting a disease state, or may be categorized as non-pathologic, i.e., a deviation from normal but not associated with a disease state. The term is meant to include all types of cancerous growths or oncogenic processes, metastatic tissues or malignantly transformed cells, tissues, or organs, irrespective of histopathologic type or stage of invasiveness. Examples include malignancies of the various organ systems, such as those affecting lung, breast, thyroid, lymphoid, gastrointestinal, and genito-urinary tract, as well as adenocarcinomas which include malignancies such as most colon cancers, renal-cell carcinoma, prostate cancer and/or testicular tumors, non-small cell carcinoma of the lung, cancer of the small intestine and cancer of the esophagus. “Pathologic hyperproliferative” cells occur in disease states characterized by malignant tumor growth. Examples of non-pathologic hyperproliferative cells include proliferation of cells associated with wound repair.

The invention further provides methods for treating infectious disease. Infectious diseases that can be treated using this invention include those caused by pathogens such as bacteria, viruses, protozoa, helminths, and the like. These diseases include such chronic diseases such as acute respiratory infections, diarrheal diseases, tuberculosis, malaria, hepatitis (hepatitis A, B C, D, E, F virus), measles, mononucleosis (Epstein-Barr virus), whooping cough (pertussis), AIDS (human immunodeficiency virus I & 2), rabies, yellow fever, and the like. Other diseases caused by human papilloma virus or various strains of virus are treatable by this method.

The methods of the invention allow the treatment of a subject for infection by both gram-positive and gram-negative bacteria. Bacterial pathogens, often found extracellularly on mucosal surfaces, which may be targets for the PRIPS and TLR agonists of the invention include, but are not limited to, Streptococcus pneumonia, Streptococcus pyogenes, Group B Streptococci, Gardnerella vaginalis, Klebsiella pneumoniae, Acinetobacter spp., Haemophilus aegyptius, Haemophilus influenzae, S. epidermis, Propionibacterium acnes, and oral pathogens such as Actinomyces spp., Porphyromonas spp., and Prevotella melaminogenicus. Both gram-positive and gram-negative bacterial targets of treatment are included in the methods of the invention. These include, but are not limited to, gram-positive bacteria such as Listeria monocytogenes, Bacillus subtilis, Enterococcus faecalis, Enterococcus faecium, Staphylococcus aureus, Staphylococcus epidermidis, Staphylococcus salivarius, Corynebacterium minutissium, Corynebacterium pseudodiphtheriae, Corynebacterium stratium, Corynebacterium group G1, Corynebacterium group G2, Streptococcus pneumonia, Streptococcus mitis and Streptococcus sanguis; as well as gram-negative bacteria including Escherichia coli, Klebsiella pneumoniae, Pseudomonas aeruginosa, Burkholderia cepacia, Serratia marcescens, Haemophilus influenzae, Moraxella sp., Neisseria meningitidis, Neisseria gonorrhoeae, Salmonella typhimurium, Actinomyces spp., Porphyromonas spp., Prevotella melaminogenicus, Helicobacter pylori, Helicobacter felis, and Campylobacter jejuni, as well as antibiotic-resistant forms of each of these gram-positive and gram-negative bacteria. Other microbial pathogens may also be targets for these PRIPS and TLR agonists of the invention, as would be understood to those skilled in the art.

The invention further provides methods for treating other non-bacterial microbial infections such as mycoplasma infections. Mycoplasma belongs to the class Mollicutes, eubacteria that appear to have evolved regressibly by genome reduction from gram-positive ancestors. Unlike classic bacteria, they have no cell wall but instead are bounded by a single triple-layered membrane, and may be susceptible to therapeutic formulations of certain peptides of the present invention. Representative mycoplasma human pathogens include Mycoplasma pneumoniae (a respiratory pathogen), Mycoplasma hominis (a urogenital pathogen) and Ureaplasma urealyticum (a urogenital pathogen).

Fungi also may be susceptible to the PRIPs and TLR agonists of the invention Specific fungal pathogens which may be targets for the methods of the invention include, but are not limited to, Microsporum spp., Epidermophyton spp., Candida albicans, Cryptococcus neoformans, Trichophyton spp., Sporothrix schenkii and Aspergillus fumigatus, as well as other known fungal pathogens.

The invention provides for both prophylactic and therapeutic methods of treating a subject at risk of (or susceptible to) a cancer or infectious disease. As used herein, the term “treatment” is defined as the application or administration of a therapeutic agent to a subject, or application or administration of a therapeutic agent to an isolated tissue or cell line from a subject who has a disease, a symptom of disease, or a predisposition toward a disease, with the purpose to cure, heal, alleviate, relieve, alter, remedy, ameliorate, improve or affect the disease, the symptoms of the disease or the predisposition toward the disease. A therapeutic agent includes, but is not limited to, a PRIP, TLR11/TLR12 and/or TLR5 agonist immunomodulatory compounds, small molecules, peptides, antibodies, or any other compounds or compositions of the invention.

The invention also provides methods of preventing infectious disease. In some instances, the mammal, in particular human, can be treated prophylactically, such as when there may be a risk of developing disease. An individual traveling to or living in an area of endemic infectious disease may be considered to be at risk and a candidate for prophylactic vaccination against the particular infectious agent. For example, therapeutic formulations certainly PRIPs can be administered to a human expecting to enter a malarial area and/or while in the malarial area to lower the risk of developing malaria. Preventative treatment can also be applied to any number of diseases including those listed above, where there is a known relationship between the particular disease and a particular risk factor, such as geographical location or work environment.

In some instance, these treatments can be used in combination with other known therapies or pharmaceutical formulations useful for treating cancer or infectious diseases. Such treatments can be administered simultaneously or sequentially.

With regard to both prophylactic and therapeutic methods of treatment, such treatments may be specifically tailored or modified, based on knowledge obtained from the field of pharmacogenomics. “Pharmacogenomics”, as used herein, refers to the application of genomics technologies such as gene sequencing, statistical genetics, and gene expression analysis to drugs in clinical development and on the market. More specifically, the term refers to the study of how a patient's genes determine his or her response to a drug (e.g., a patient's “drug response phenotype” or “drug response genotype”). Thus, another aspect of the invention provides methods for tailoring an individual's prophylactic or therapeutic treatment with either the PRIP, TLR11/TLR12 and/or TLR5 agonist immunomodulatory compounds, small molecules, peptides, antibodies, or any other compounds or compositions of the various embodiments of the invention according to that individual's drug response genotype. Pharmacogenomics allows a clinician or physician to target prophylactic or therapeutic treatments to patients who will most benefit from the treatment and to avoid treatment of patients who will experience toxic drug-related side effects. This technology also allows a clinician or physician in this instance to distinguish between patients who have an active or functional TLR11/TLR12 and/or TLR5 and those who may need gene therapy in order to respond to treatment. The clinician or physician can thereby tailor the type of treatment that may be necessary to the specific patient.

In some cases, therapeutic formulations including nucleic acid molecules that encode and express TLR11/TLR12 and/or TLR5 exhibiting normal activity can be introduced into cells via gene therapy method. Alternatively, in some instances, normal TLR11/TLR12 and/or TLR5 can be co-administered into the cell or tissue to maintain or introduce the requisite level of cellular or tissue TLR11/TLR12 and/or TLR5 activity.

The phrase “therapeutically-effective amount,” as used herein, means that amount of a compound, material, or composition comprising a PRIP or TLR agonist of the invention which is effective for producing some desired therapeutic effect when administered to an animal, at a reasonable benefit/risk ratio applicable to any medical treatment.

The data obtained from in vitro and animal studies can be used in formulating a range of dosage for use in humans. In some instances, the dosage of such compounds lies within a range of circulating concentrations that include the ED₅₀ with little or no toxicity. The dosage can vary within this range depending upon the dosage form employed and the route of administration utilized. For any compound used in the method of the invention, the therapeutically effective dose can be estimated initially from cell culture assays. A dose can be formulated in animal models to achieve a circulating plasma concentration range that includes the IC₅₀ (i.e., the concentration of the test compound that achieves a half-maximal inhibition of symptoms) as determined in cell culture. Such information can be used to more accurately determine useful doses in humans. Levels in plasma can be measured, for example, by high performance liquid chromatography.

The therapeutic methods of the present invention encompass the use of agents that modulate expression or activity. An agent may, for example, be a small molecule. Exemplary doses include, without limitation, milligram (mg) or microgram (μg) amounts of the small molecule per kg of subject or sample weight (e.g., about 1 microgram per kilogram to about 500 mg/kg, about 100 μg/kg to about 50 mg/kg, or about 1 μg/kg to about 5 mg/kg). It is furthermore understood that appropriate doses of a small molecule depend upon the potency of the small molecule with respect to the expression or activity to be modulated. When one or more of these small molecules is to be administered to an animal (e.g., a human or a non-human mammal or other animal), a physician, veterinarian, or researcher may, for example, prescribe a relatively low dose at first, subsequently increasing the dose until an appropriate response is obtained. In addition, it is understood that the specific dose level for any particular animal subject will depend upon a variety of factors including the activity of the specific compound employed, the age, body weight, general health, gender, and diet of the subject, the time of administration, the route of administration, the rate of excretion, any drug combination, and the degree of expression or activity to be modulated (see, e.g., Remington: The Science and Practice of Pharmacy (20th ed.), ed. A. R. Gennaro, Lippincott Williams & Wilkins, 2000 and Encyclopedia of Pharmaceutical Technology, (eds. J. Swarbrick and J. C. Boylan), 1988-1999, Marcel Dekker, New York).

For example, a therapeutically effective amount of protein or polypeptide (i.e., an effective dosage) ranges from about 0.001 to 30 mg/kg body weight, in some instances from about 0.01 to 25 mg/kg body weight, in other instances from about 0.1 to 20 mg/kg body weight, and in additional instances from about 1 to 10 mg/kg, 2 to 9 mg/kg, 3 to 8 mg/kg, 4 to 7 mg/kg, or 5 to 6 mg/kg body weight. The protein can be administered one or more times per week for between about 1 to about 10 weeks. It can be administered between about 2 to about 8 weeks, between about 3 to about 7 weeks, or for about 4, about 5, or about 6 weeks. The skilled artisan will appreciate that certain factors may influence the dosage and timing to effectively treat a subject, including but not limited to the severity of the disease or disorder, previous treatments, the general health and/or age of the subject, and other diseases present. Moreover, treatment of a subject with a therapeutically effective amount of a protein, polypeptide, or anti-TLR agonist antibody according to the invention can include a single treatment or, can include a series of treatments.

For anti-TLR agonist antibodies the dosage can be about 0.1 mg/kg of body weight (generally about 10 mg/kg to about 20 mg/kg). If the antibody is to act in the brain, a dosage of about 50 mg/kg to about 100 mg/kg is usually appropriate. Generally, partially human antibodies and fully human antibodies have a longer half-life within the human body than other antibodies. Accordingly, lower dosages and less frequent administration are often possible with such antibodies. Modifications such as lipidation can be used to stabilize antibodies and to enhance uptake and tissue penetration (e.g., into the brain). (See, Cruikshank et al. 1997, J. Acquired Imm. Defic. Syndromes Hum. Retrovirol. 14:193).

The compounds of the invention may be administered intravenously, intramuscularly, intraperitoneally, subcutaneously, topically, orally, or by other acceptable means. In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution, which, in turn, may depend upon crystal size and crystalline form. Alternatively, delayed absorption of a parenterally-administered drug form is accomplished by dissolving or suspending the drug in an oil vehicle. One strategy for depot injections includes the use of polyethylene oxide-polypropylene oxide copolymers wherein the vehicle is fluid at room temperature and solidifies at body temperature.

Injectable depot forms are made by forming microencapsule matrices of the subject compounds in biodegradable polymers such as polylactide-polyglycolide. Depending on the ratio of drug to polymer, and the nature of the particular polymer employed, the rate of drug release can be controlled. Examples of other biodegradable polymers include poly (orthoesters) and poly (anhydrides). Depot injectable formulations are also prepared by entrapping the drug in liposomes or microemulsions, which are compatible with body tissue.

When the compounds of the present invention are administered as pharmaceuticals, to humans and animals, they can be given per se or as a pharmaceutical composition containing, for example, 0.1% to 99.5% or 0.5% to 90% of active ingredient in combination with a pharmaceutically acceptable carrier.

The invention also provides pharmaceutical packs or kits comprising one or more containers filled with one or more of the ingredients of the pharmaceutical compositions of the invention. Optionally associated with such container(s) can be a notice in the form prescribed by a governmental agency regulating the manufacture, use or sale of pharmaceuticals or biological products, which notice reflects approval by the agency of manufacture, use or sale for human administration. Such container(s) further may include instructions for use of the supplied pharmaceutical compositions of the invention.

### 5. EXAMPLES

This invention is further illustrated by the following examples, which should not be construed as limiting.

### 5.1 Example 1

### NK Immunomodulatory Activating Assay

Several assays have been developed to determine when PA19 has been activated. Many of these assays are provided and described in WO 2005/010040, the contents of which are incorporated herein by reference in their entirety (see Rosenberg et al., Int. J. Cancer 2005 114: 756-765).

Reagents and Media

All the salts and glucose (#1916-01) were from Mallinckrodt Backer Inc. if not specially noticed; Basal Medium Eagle (Gibco #08202334DJ), F-12 nutrient medium (Gibco #11765-088), MEM Non-Essential Amino Acids Solution (Gibco #1806), bovine calf serum (BCS) (HyClone, #SH30072.03), 0.9% sodium chloride for injection, USP (Abbott Labs #NDC0074-7983-03), IPTG (#12481C) & X-Gal (#4281C, both Gold Biotechnology), trypsin (Gibco#840-7250), TRIS (Invitrogen #15504-020), Dodium Dodecyl Sulphate (SDS, Sigma L3771), all the primers used were synthesized at the MSU core facility, mIFNγ (Calbiochem. #N-407303), mIL-4 (R&D #404-ML-005), mGM-CSF (#G0282-5UG), Bovine Serum Albumin (BSA fraction V, #A-9418), Human Albumin (HSA, #A1653) and Phenol Red (P4758) (all from Sigma), DetoxiGel Endotoxin Removal Gel (Pierce #20339); DEAE-Sepharose CL-6B (Sigma #DCL-6B-100).

Antibiotics

Kanamycin (K4000), ampicillin (A6140), chloramphenicol (C0378), streptomycin (S6501), penicillin (P3032), puromycin (P7255) (all from Sigma), geneticin (Gibco #10131-027).

Antibodies

Anti-mCD40 (clone1c10, R&D #MAB440); anti-FLAG-M2, rabbit anti-goat IgG-HRP, and goat anti-rabbit IgG-HRP (all from Sigma, numbers: A8592, A5420, and A0545 respectively); Anti-actin (Santa Cruz I-19); anti-mTLR12 (two polyclonal antibodies from Imgenex: IMG-5034 and IMX-5088, against mTLR12 peptides 743-756, and 147-159 respectively).

Enzymes

Alkaline protease (Promega #A144A), RNaseA (Boeringer #109142), DNaseI (Roche #776785), Taq-DNA polymerase (Applied Biosystems #58002040), native Pfu-polymerase (Stratagene #600135-81), Platinum Pfx (Invitrogen #11708013), Therminator polymerase (NEB #M0261S); Collagenase D (Roche #1088874), Alkaline phosphatase from calf intestine (Roche #713023); Restriction enzymes: EcoRI (Invitrogen #15202-013), EcoRV (NEB #R0195S), HindIII (NEB #Ro104S), NotI (NEB #R0189S), EagI (NEB #R505S).

Transfection Reagents

Lipofectamine (LFA), lipofectamine-2000 (LF2K), optifect (OPTI) (all from Invitrogen, #18324, #11668 and #12579 respectively).

Chemically Competent E. coli Strains

GC5 (GeneChoice#62-7000-22, or Sigma#G2669), DH5a (#18265) and TOP10 (#440301) (both Invitrogen), Rosetta2 (DE3) pLacI (genotype: F⁻ ompT hsdS_{B}(r_{B}⁻m_{B}⁻) gal dcm(DE3) pLacIRARE2 (Cam^{R}) (BD#71404-3).

Vectors

pCR2.1 TOPO (Invitrogen #450641), pIRESpuro3 (BD #6986-1), p3xFLAG-CMV-9 (Sigma #E-4276), pET Blue1 AccepTor vector (BD #N70599-3).

Cell Culture, and Animal Use

Murine sarcoma S-180 (ATCC #CCL-8), human ovarian carcinoma ES-2 (ATCC #CRL-7394), human fibrosarcoma HT1080 (ATCC #CCL-121) was transfected with pCMV vector containing DsRedX gene and a desired red fluorescence positive clone was selected among the clones resistant to geneticin. All the mammalian cell lines above were grown in the culture growth medium (Eagle medium with 10% BCS, 100 U/ml penicillin and 100 μg/ml streptomycin). BALB/c mice, both regular, and athymic strains, were bread in house. For mouse experiments, 10⁶ cells of HT 080 derived lines, or 10⁵ cells of ES-2 (in 0.1 ml of culture growth medium without BSC), were injected either i.p. or s.c. All procedures involving the use of animals and their care have been approved by MSU's Institutional Animal Care & Use Committee and are in accordance with State and Federal Guidelines.

NK (LGL) Cell Isolation

Spleens (10-15 per experiment) are aseptically removed from male Balb/C mice 6-10 weeks of age. Splenocytes are “squeezed” out of spleens using 2 sterile glass microscope slides. Cells are collected in approximately 10 ml of DMEM/F12 containing 10% fetal calf serum (FCS) and gentamicin (50 μg/ml). Single cell suspensions are generated by passing collected cells through a 70 μm nylon mesh screen. After washing the cell pellet once with PBS (centrifuged at 675×g for 5 min), red blood cells are lysed by brief hypotonic shock (i.e., exposure to sterile distilled, deionized water, followed immediately with appropriate volumes of 10×PBS to return to isotonicity). Remaining cells are centrifuged and resuspended in DMEM/F12+10% FCS and transferred to 75 cm² flasks each containing 25 ml DMEM/F12+10% FCS (cells from 5 spleens per flask). The cells are incubated for 60 min. at 37° C. to selectively remove readily adherent cells (mainly fibroblasts and macrophages). After the incubation, the flasks are gently shaken and the non-adherent cells are removed, pelleted by centrifugation, and resuspended in DMEM/F12 (no supplements) (1.0 ml/10⁸ cells). A 1.0 ml volume of these cell suspensions are carefully layered onto a 70%/60%/40% (2 ml/4 ml/4 ml) Percoll™ gradients in 15 ml centrifuge tubes and centrifuged for 30 min at 675×G. The cells which sediment at the 40/60 interface represent primarily large granular lymphocytes (LGLs), enriched with NK cells. These cells are carefully removed with a pasteur pipet, centrifuged, washed once with PBS and resuspended in approximately 5 ml DMEM/F12 supplemented with gentamicin (50 μg/ml) and 10% fetal calf serum. Cell counts and appropriate dilutions of LGL cells are made with these cells, as described below. For convenience this LGL cell preparation is interchangeably referred to as NK cells.

NK Cell Mediated Cytotoxicity (NK-CMC) Assay

Mouse sarcoma 180 cells are seeded into 96 well plates at a density of 5×10³ cells per well in 100 μl DMEM/F12, supplemented with 10% FCS and gentamicin (50 μg/ml). After several hours to ensure proper attachment, test samples (e.g., samples containing an immunomodulatory profilin, profilin-related and profilin-like polypeptide or protein, positive or negative controls) are added to each well in volumes of 10-25 μl. NK cells are added in 100 μl of supplemented DMEM/F12 at designated densities: 0 NK cells/well; 5×10⁴ NK cells/well (1:10 target/effector ratio); 1.25×10⁵ NK cells/well (1:25 target/effector ratio); 2.5×10⁵ NK cells/well (1:50 target/effector ratio); and, 5×10⁵ NK cells/well (1:100 target/effector ratio). IL2 is added as a media supplement with final concentration of 125 U/m L.). Co-cultures are incubated for 4 days at 37° C., 5% CO₂, and terminated and vitally stained using a MTT cell viability quantification assay (see Mossman, T. J. Immunol. Meth. 65:55 (1983); and Sigma Chemical MTT (M5655) product application note). Culture media is carefully aspirated from each well and the remaining cells are washed twice and replaced with 100 μl DMEM/F12+10% FCS, containing MTT (50 μg/ml). The plates are further incubated for 4-5 hours at 37° C. Absorbance is measured with the aid of a plate reader (600 nm filter). Decreased absorbance indicates a decrease in the number of viable cells per well (i.e., cytotoxicity). Absorbance is measured again after the MTT is solubilized by replacement of the medium with 200 μl of 2-propanol containing 0.04 M HCl. This gives a uniform color throughout the well and minimizes discrepancies in absorbance readings due to uneven cell distribution. NK-inducing activity is calculated relative to negative (PBS/BSA) and positive (internal standard) controls.

### 5.2 Example 2

### Dendritic Cell (DC) Immunomodulatory Activating Assay

Special Reagents

Mouse recombinant GM-CSF, IL4 and IFNγ and anti-mouse CD40 were obtained from R&D Systems (Minneapolis, Minn.). Collagenase D was obtained from Boehringer Mannheim (Indianapolis, Ind.). MiniMACS magnetic cell isolation system was obtained from Miltenyi Biotech (Auburn, Calif.).

Isolation and Culture of DCs

Isolation of DCs and DCA assay was performed as specified in [Int. J. Cancer, 2005, 114:756] in accordance to the manufacturer's recommendations (MicroBeads mCD11c (N418) Miltenyi Biotec#130-052-001; mIL-12 p70 DuoSet R&DCat.#DY419). Briefly, spleens were aseptically removed from 6-12 weeks old male mice, placed into a 60 mm sterile Petri dish with 5 ml of Collagenase D solution (1 mg/ml in 10 mM HEPES, 150 mM NaCl, 5 mM MgCl₂, 1.8 mM CaCl₂, pH 7.4) and injected with 0.5 ml of the same solution. After 2-3 min incubation at room temperature, the spleens were cut into several pieces and incubated at 37 for 1 hr. Collagenase processed pieces of spleens were further reduces in size by two glass slides and passed through 100 μm nylon mesh cell strainer. The strainer was washed with 20 ml of MACS buffer and the splenocytes were counted (after. 200-fold dilution in 20 ml vial (Coulter)) in Coulter Z1 particle counter, centrifuged for 10 min at level 15 (about 1000 RPM, IEC model PR-2) and re-suspended in MACS buffer to final density about 2×10⁸/ml. The resulting suspension (1.0 ml) was incubated with 200 μl of paramagnetic anti-CD11c-coated microbeads for 15 min at +4 C, washed with 10 ml, and re-suspended in 1 ml of MACS buffer. CD11c+ cells were then isolated and eluted into 1-2 ml of MACS buffer and diluted in supplemented culture medium (culture growth medium with 1 ng/ml of both mGM-CSF and mIL-4, 3 ng/ml mIFNγ and 0.5 μg/ml anti-mCD40) to final density 5×10⁵ cells/ml. The cells were distributed to wells of 96-well plate ( ) (100 μl/well or 5×10⁴ cells) containing test samples in 100 μl of culture medium and cultured overnight at 37 C in 5% CO₂.

Determination of Mouse IL-12 (p70) Release from DCs

Mouse IL-12 release from CD11c⁺ splenocytes was measured using an ELISA assay. Briefly, CD11c⁺ cell culture supernatants were sampled following an overnight incubation (usually 15-18 hrs). Media samples (100 μl) were added to ELISA plates coated with anti mouse IL-12 (p70) capture antibody (R&D #MAB419; 250 ng in 100 μl per well) and incubated either at 37° C. or room temperature for 2 hrs. The ELISA plates were washed extensively with wash buffer after which 100 μl per well of detection antibody/detection reagent (biotinylated anti-mouse IL-12; R&D #BAF419 at 50 ng/ml and streptavidin-HRP) was added. The ELISA plates were again incubated for 2 hours, washed and exposed to TMP substrate solution (Pierce #34021) for 20 min. The substrate reaction was stopped by adding 100 μl/well 2 M H₂SO₄. ELISA plates were read at 450 nm (corrected at 540 nm) and mIL-12(p70) levels were calculated using an intra-ELISA standard curve.

Preparation of PRIPs

PA19 genes from five different protozoan parasites (Eimeria tenella (ET), T. gondii (TG), N. caninum (NC), P. falciparum (PF), and Sarcosistis neurona (SN)) were cloned from corresponding EST clones kindly provided by Dr. David Sibley (Washington University, St. Louis, Mo., special thanks to Mr. Robert Cole from that lab). All the EST clones were completely sequenced with either T7/T3, or M13 Reverse/M13 Forward, pair of primers at MSU core facility to verify the identity of the clone and determine if it represents the complete gene for PA19. About 40% of the clones analyzed appeared to be either non-related to PA19 gene, or containing an incomplete gene. The EST clones with correct and complete PA19 gene (one for each of the protozoan parasite) were used as the source of the corresponding PA19 gene. These were: EtESTee7602.y1 (E. tenella M5-6), NcEST3d63 g10.y1 (N. caninum Nc-LIV), PfESToac16g04.y1 (P. falciparum 3D7), SnEST4a01g09.y1 (S. neurona cSn1), TgESTzyc77f02.y1 (T. gondii RH type1). The genes were then re-cloned into pET Blue-1 vector to set up the gene expression under a very strong IPTG-dependent T7 promoter. The bacterial cells were collected and disintegrated by sonication, and the resulting mixture was assayed by DCA-assay. Specific activities were not measured, but for a rough estimation of the relative activities of the proteins it can be assumed that expression of the proteins in E. coli was at a close level for each case. Thus, the amount of PA19 protein in the individual bacterial cell lysates added to DCA-assay were comparable, and thus, the relative activity in the assay adequately reflects the specific activity of these proteins. The activities of the PA19 protein from the organisms tested were: ET>TG>NC>PF-SN (See FIG. 21). FIG. 21 shows the activities of different PA19 proteins measured by DCA assay.

In further detail, the recombinant PA19 protein was preparatively isolated from bacteria grown in 1 L of LB medium substituted with 0.4% of glucose and contained ampicillin and chloramphenicol at concentrations 100 and 34 μg/ml respectively. The medium was inoculated with bacteria grown overnight in 10 ml of the same type of medium, separated from conditioned medium and washed once with sterile PBS. The bacterial growth after inoculation was performed at 37 C in a shacking incubator at 200 RPM and monitored by measuring turbidity at 600 nm and when the bacterial cell density reached about 0.8 optical units, IPTG (final concentration of 1 mM) was added to the suspension. The bacterial suspension was shaken at the same conditions as above for another 4 hr. The bacteria were isolated from the suspension by centrifugation at 10,000 RPM for 15 min, washed with 200 ml sterile PBS, and weighed (the yield was about 3.6 g of wet bacteria from 1 L of the suspension). Bacterial cells were re-suspended in 10 ml of PBS with 2 mM of PMSF and broken by repeated sonication (20×10 sec. pulse with 1 min intervals) while on ice. The lysate was cleared by centrifugation (12,000 RPM, 20 min) and the supernatant was fractionated by ammonium sulfate. The fraction of 40-80% saturation of ammonium sulfate was collected, re-dissolved in PBS diluted 1:1 with water, and after clearing by centrifugation as above, applied onto the DEAE-Sepharose column. The column was washed with PBS, and the fraction containing the PA19 protein was eluted from the column by PBS containing 0.5 M NaCl. This fraction contained the vast majority of PA19 as confirmed by gel-electrophoresis and DCA assay. Nucleic acids co-eluting with PA19 were removed by application of 1 U of each of protease-free RNaseA and DNase1 (incubation at 4 C for 16 hr). The latter sample was diluted 3-fold with phosphate buffer and re-applied onto DEAE-Sepharose column for additional separation and concentration. The fractions eluted by 0.5M NaCl/PBS were analyzed by SDS-gel electrophoresis and those containing more that 90% pure PA19 were combined and passed several times through a DetoxiGel column to reduce the level of LPS (bacterial endotoxin) in the prep to below 50 U/ml.

For mice experiments, the prep was diluted with 0.1% HSA in 0.9% saline (until final concentrations 1, 10, or 100 ng/ml of PA19 (at least, 1000-fold) and filter-sterilized. Injection (i.p.) schedule was: 30 min after injection of the human cancer cells, followed by repeated injections at days 2, 4 and 7.

Effect of Profilin Binding Proteins

PA19 protein has a low level of homology to actin-binding protein profilin, which also has been shown to form complexes with PIP2, and many cellular proteins having oligo-Pro stretches. (Fetterer, R. H., et al., J. Parasitol. 2004. 90(6): 1321-8) Accordingly, several profilin binding proteins, including bovine actin, poly-L-Pro and PIP2, were tested for their effect on PA19 activity or DCA assay. None of the compounds used (bovine actin, poly-L-Pro, or PIP2) were found to interfere with the release of IL-12, and therefore either PA19 binds none of these molecules, or the site on the PA19 protein responsible for interaction with dendritic cells is not overlapping with the sites for the above-mentioned ligands.

### 5.3 Example 3

### IFN-γ Immunomodulatory Activating Assay

The production and release of IFNγ by large granular lymphocytes (LGLs) into the culture media was assessed as follows: LGLs, enriched with NK cells, were isolated (a method for doing so has been described above) and seeded into 96 well plates at densities of 2.5 or 5.0×10⁵ cells/well, in 200 uL of DMEM/F12 supplemented with 10% fetal calf serum, gentamycin (50 ug/ml), and IL2 (125 U/ml). Test samples (e.g., samples containing an immunomodulatory profilin, profilin-related and profilin-like polypeptide or protein, positive or negative controls) were added and the cells were cultured overnight, following which the condition media (CM) was removed and centrifuged to remove any aspirated cells. Aliquots of the CM were then measured for IFNγ using an ELISA kit purchased from various sources (BD PharMingen Inc., Genzyme Corp., and R&D Systems Inc.). Briefly, in the ELISA assay, CM or serum samples (and IFNγ standard) are incubated in ELISA plate wells, previously coated with a capture antibody (hamster monoclonal anti-mouse IFNγ) for 1 hr at 37° C. After extensive washing the wells are exposed to a biotinylated second antibody (polyclonal anti-mouse IFNγ) for 1 hr at 37° C. After washing, the wells are then exposed to a detection reagent (streptavidin conjugated with horseradish peroxidase) for 15-20 min. at 37° C. Once again, after extensive washing, 100 μl TMB substrate is added to the wells and incubated for 5-7 min. at room temperature. The reaction is stopped by adding 100 μl 2 M H₂SO₄. Absorbance at 450 nm is read using a plate reader and IFNγ concentrations are calculated from the standard curve.

### 5.4 Example 4

### Transfected Cell Assay

The principle of the transfected cell assay is to use a murine cell line (such as S-180 sarcoma), which is transfected with the mTLR11/TLR12 and/or TLR5 gene. The cells are able to express the TLR11/TLR12 and/or TLR5 protein and assemble it on their surface. Addition of PA19 to such cells leads to activation of the receptor and start the signaling cascade, which will result in activation of NF-κB transcriptional factor and expression of mIL-12, mIL-6, and some other cytokines. The level of expression of these cytokines is estimated on the basis of ELISA (similar to the part 2 of the DCA-assay). This assay is used with PA19, deletion mutants, or other modifications to the PA19 protein.

The TLR12 gene was re-cloned from corresponding chromosomal (BAC) clones. The use of chromosomal clones instead of cDNA-derived EST clones was justified on the basis that the rodent' gene for TLR12 has no introns. The BAC clones containing the part of chromosomal DNA surrounding the mTLR12 gene, or hTLR12 pseudo-gene where chosen by the BLAST and public availability. The chosen clones were purchased from BACPAC Resources (CHORI Research Center at Oakland, Calif.) (murine BAC's), or obtained as a courtesy from The Wellcome Trust Sanger Institute, Cambridge, UK) (human clone RP1-149P10) and analyzed by amplification of the DNA by PCR with the primers specific to N- and C-termini of the corresponding genes. The fragments of about 3 kB obtained after PCR amplification from DNA of three murine BAC clones (RP23-200P22, RP23-392K10, RP23-305015) with the primers above were mixed together and sub-cloned into pCR2.1 TOPO vector. As the result, several clones containing the plasmid with the mTLR12 gene inserted inside the multiple cloning region of the vector were obtained. One of these plasmids was obtained in large amounts and the DNA was used to cut out the mTLR12 gene by EcoRI enzyme and re-clone the fragment into EcoRI-cut vector pIRESpuro3. The clones containing an insert after this step were selected and confirmed for the presence of mTLR12 gene in the desired orientation by both PCR, and complete sequence analysis. Bacterial clones with mTLR12 gene in opposite orientations were used for isolation of the corresponding plasmids which were used for transfection of the mammalian cell lines (murine sarcoma S-180, human fibrosarcoma HT1080, or hamster ovary cell line CHO-9). Transfection was done by using one of the available transfection reagents (see above) according to the manufacturer's recommendations. The transfected clones further were selected by applying selective pressure (antibiotic puromycin at concentrations: 1 μg/ml (HT1080), 5 μg/ml (S-180), 10 μg/ml (CHO-9)).

The TLR11/TLR12 and/or TLR5 gene is constructed based on clones of pieces of the gene and reintroduced into a plasmid. The plasmid is used to create murine cells that express the TLR11/TLR12 and/or TLR5 gene. Those cell lines that overexpress TLR11/TLR12 and/or TLR5 are used as a substitute for dendritic cells in the dendritic cell assay. The assay may include transfecting the immortal murine sarcoma cell line S-180 with a plasmid containing mTLR11/TLR12 and/or TLR5 gene under a strong promoter, and the resulting cell line is used as a substitute for Dendritic Cells. The murine TLR11/TLR12 and/or TLR5 gene are cloned from a chromosomal BAC clone into a mammalian expression vector (pIRESpuro3), and a murine sarcoma S-180 cell line is transfected with the plasmid. The cell line expresses TLR11/TLR12 and/or TLR5 protein in significant amounts, which are assembled on the surface of the cells. Activation of the TLR12 by PA19 starts the MyD88-NF-κB pathway and results in activation of expression of several cytokines (including mIL-6, and mIL-12). These cytokines are secreted outside the cells and the accumulation of one or more of these cytokines is monitored by an ELISA assay. (The ELISA test may also be based on the complete mIL-12 molecule, or a mIL-6 ELISA kit, as well as mIL-12 p35, or mIL-12 p40 kits). These assays are performed in parallel with the DCA-assay to show that both produce similar results. Results show that PA19 indeed works through the activation of the TLR11/TLR12 and/or TLR5. The assay takes only about a day to complete and can be used for checking activities of various PA19 proteins, including mutants, as well as TLR11/TLR12 and/or TLR5 agonist compounds including antibodies, aptainers, small mole cells and peptides or peptide mimetics.

In addition, the tumorigenicity of these transfected S-180 cells over-expressing mTLR11/TLR12 and/or TLR5 is compared against the tumorigenicity of the original S-180 cells (as well as S-180 cells transfected with vector alone as a negative control) in mice. These experiments provide an understanding of the importance of TLR11/TLR12 and/or TLR5 in carcinogenesis and promoting the anti-cancer effect of PRIPs.

### 5.5 Example 5

### Verification of Binding to TLR11/TLR12 and/or TLR5

Physical interaction between PA19 and TLR11/TLR12 and/or TLR5 is verified by 1) BIA core measuring, and 2) the yeast two-hybrid system.

The yeast two-hybrid method is well-developed, and consists of creating a “bait” (like a GAL4 DNA binding domain fusion protein) and a “prey” (like a GAL4 activation domain fusion protein). The methods are well known to tone of skill in the art. Briefly, the GAL4-lacZ reporter is activated only if the GAL4 DNA binding domain is fused to a polypeptide that binds to the polypeptide to which the GAL4 DNA actiating domain has been fused. Accordingly, a fusion of the GAL4 DNA binding domain to the TLR11/12 (or TLR5) receptor extracellular domain activates expression of the GAL4 promotor-lacZ reporter when a GAL4 activation domain-PRIP fusion is co-expressed in the same yeast cell. The assay provides a facile means for measuring PRIP TLR receptor binding activity, as well as for screening for TLR receptor binding on TLR11/12 (or 5) receptor agonist candidates.

### 5.6 Example 6

### Analysis of Structure-Function Relationship in PA19 Protein

The structure-function relationship in PA19 protein has been studied by mutagenesis. It has been shown that removal of 5 or more amino acid residues from the C-terminus of the protein completely destroys the ability of the PA19 to activate dendritic cells. Indeed, the results of experiments with C-terminal deletions of PA19 showed that activity of the truncated molecules is lost when the length of the peptide deleted from C-terminus and that a 10 amino acid shorter version (C-10) was completely inactive (while C-3 retained some activity).

In contrast, removal of up to 14 amino acids from the N-terminus of PA19, as well as adding a FLAG-tag, or more than 30 total amino acids from the pre-ATG region of the gene joined to the N-terminal peptide of beta-galactosidase, showed no such drastic effect on activity. The significant role of Cysteine residues in PA19 has been shown by directed point mutations. These mutations become lethal for activity when both of the Cys residues are modified. Several other mutants with a significantly lower level of DCA activity were obtained, but all of them contained multiple mutations.

A different series of truncated PA19 molecules have been generated, including those truncated from both ends, which will be used to find out whether any of those retain DCA-activity. In addition, some of these molecules can be used for mouse experiments to confirm that DCA-activity is actually related to anti-tumor activity. Additional experiments investigate how removing of a certain region from the middle of the molecule affects the DCA- and anti-cancer activity of the protein.

The above analyses demonstrate that a peptide of about 23 amino-acids (AA) shorter than the original (20-AA from N-terminal plus 3-AA from C-terminal) still may retain DCA-activity.

Several mutant forms of PA19 (E. tenella) have been expressed in E. coli: (N-1)PA19, (N-20)PA19, (C-20)PA19, (N-1)/(C-20)PA19, and (N-20)/(C-20)PA19. The activity of these mutants has been examined by DCA-assay. The only active form was the (N-1)PA19 (I amino acid deleted from the N-terminus). All the rest (truncated molecules (N-20)PA19, (C-20)PA19, (N-1)/(C-20)PA19, and (N-20)/(C-20)PA19) showed no activity in the assay. The presence of the recombinant protein in the analyzed sample was confirmed by gel-electrophoresis. The expressed protein (N-1)/(C-20)PA19 has been purified (it was obtained in enriched form after two steps of purification). Because the bacterial cell lysates of the E. coli expressing the truncated forms of PA19 did not show any activity in most cases, actual measurement of specific activity for these truncated forms are not necessary.

The N-terminal portion of PA19 does not participate in manifestation of DCA-activity because removal of this part (up to 14 amino acids) does not reduce the ability of PA19 ET to activate DCs. In some cases it appears that these truncated molecules (which have lost 4-5 negative charges) are more active than normal. In support of the hypothesis that the presence of negative charges on the N-terminus reduces the ability of PA19 to activate DCs, the N-termini of PA19 species having lower activity (i.e., those from P. falciparum and sarcosystis neuroma PF, SN) appear highly negatively charged, and they do not have a positively charged amino acid (R/K) in position 16. It is likely that up to almost the entire length of the E. tenella, and related profilin-like immunomodulatory proteins is required to elicit an immune response. This is because experiments with different proteases show that PA19 loses activity very quickly, and that one cut is sufficient to inactivate it. Also, in experiments to isolate an active peptide from the mixture of trypsin-digested PA19, none of the peptides have shown any activity on DCA-assay.

Point Mutations

The following PA19 point mutations were also prepared (the C-terminal, N-terminal, and primer directed Cys→Ser mutants as well as terminal C-20, N-1, N-20, C-20/N-1, C-20/N-20 mutants) by using specific terminal primers; also a couple of spontaneous mutants have been generated as a result of PCR amplification: 43E→K for PA19 of E. tenella, as well as (99E→K 156E→D), (20A→P, 140K→E, 143D→Q, 144K→G), and (155A→G, 159H→S) of N. caninum. These mutants are also tested to compare their DCA-activity. All the mutants are tested on the basis of their DCA-activity without complete purification of the mutant proteins. Based on the gel-electrophoresis pattern, the concentrations of the mutant proteins in the mixture are comparable to concentration of the native PA19 in the DCA reaction mixture). Additional experiments are performed to test their activities in other assays described herein as well as in an athymic mouse system using a human cancer cell line.

Creation of Random Mutants of PA19 Protein

To create random mutants of PA19, a plasmid carrying the PA19 gene of E. tenella attached to Shine-Dalgarno region (SDR) 8 nucleotides before the ATG start codon in the pCR2.1 vector to create a plasmid, pEt2.7 is subject to PCR amplifications with thermophilic 9oN A485L DNA polymerase (“Therminator,” NEB, Ipswich, Mass.), primers M13 F, and M13 R, and dNTP mixture containing 1 mM rITP. This creates random mutations in the region flanked by the primers. The mutated fragment is purified by agarose gel electrophoresis, is cleaned with Wizard PCR kit (Promega, Madison, Wis.), and is subjected to secondary PCR with Taq-polymerase and primers complementary to the C-terminal end and specific to the N-terminal end (with SDR attached 8 nucleotides before the ATG start codon) of the PA19 gene. The product of amplification is TA-ligated into the pCR2.1 TOPO vector (Invitrogen, Carlsbad, Calif.) and is blue-white selected on agar plates containing LB supplemented with X-gal and Amp. Plasmids isolated from the white colonies are then sequenced to identify sites of mutation. About 90% of the white colonies contain PA19 gene mutations, with the average number of mutations per gene being 3. The selected colonies with proven mutations will be checked by in vitro DCA or the assay. The information on mutations causing partial, or full reduction in activity, will be used to engineer site-specific mutations in the gene.

Creation of Specific Mutant Forms of PA19 Protein

To further investigate the structure/function relationship of PA19, site-specific mutagenesis is used. Plasmid pET2.7 is subject to PCR amplifications with proofreading DNA-polymerase (Pfu, of Pfx) and one of two sets of primers: a) a long (50-mer) primer containing a single nucleotide exchange to the first half of the PA19 sequence, flanked by non-mutated regions, and a primer complementary to the vector at the C-terminal end of PA19 (M13R); or b) a primer complimentary to the second half of the PA19 sequence (a long primer with a single mutation similar to that described above, can be used as well) and a primer specific to a vector at the N-terminal part of PA19 (M13 F).

The products of the PCR amplification are separated by agarose gel electrophoresis, cut out of the gel and cleaned with Wizard PCR mini-columns (Promega, Madison, Wis.). By design, the two fragments overlap by at least 40 nucleotides. The purified fragments then are mixed together in equimolar proportion, melted down and annealed to form some amount of hybrid molecules at the overlapping region. The hybrids are filled up to form double stranded copies by DNA-polymerase I (Klenow fragments), and are used for another round of PCR amplification with Taq-polymerase and primers M13 F and M13R. The amplification product is cut with restriction enzymes Hind III and Xho I, purified by gel electrophoresis and the Wizard cleaning procedure, and inserted into the pCR2.1 vector cut with Hind III and Xho I restriction enzymes. The vector is dephosphorylated by calf intestinal alkaline phosphatase (CIAP) and is cleaned as above. Both fragments are ligated together and cloned into chemically competent cefls GC5. Plasmids are isolated from white clones selected on agarized LB plates containing X-gal and Ampicillin, and are sequenced to determine whether they contain the desired point mutation. It is expected that about 80% of the white clones are mutants. The clones with confirmed mutations are also analyzed by in vitro DCA assay and by in vivo mouse test after partial purification.

### 5.7 Example 7

### PA19 Domains and Fragments

As discussed above, the C-terminal region of PA19 ET appears to affect activation of dendritic cells (i.e., DCA-activity declines roughly proportionally to the number of amino acids removed from the C-terminus). At the C-terminus, there is a stretch of 12 amino-acids virtually identical in five organisms: XXXAXYDEEKEQ (SEQ ID NO. ______) (where X=I/L/V). Accordingly, while not wishing to be bound to any particular theory, it appears that the DEEKEQ is most probably exposed to solvent or situated on the surface of the molecule (see FIG. 20, which is a comparative analysis of primary and probable secondary structures of PA19 protein from different protozoan parasites). Accordingly, it is likely a part of the active center of the PA19 protein.

A small stretch of amino acids immediately following DEEKEQ is unique for PA19 ET (ADAL) while identical in all the rest of PA19's (GNS(K/R)); thus, negative charge (D) in this position may enhance the DCA-activity. Another stretch 10 amino acids long is very similar for PA19 from all three organisms with significant DCA-activity (ET, TG, NC): FAEYL(H/Y)Q(S/G)GY. From this comparison, it may be hypothesized that exposed unbalanced negative charge (E) is important for DCA-activity.

The structure-function relationship of the immunomodulatory polypeptides of the invention have been further addressed by the analysis of mutants of PA19 protein. Removal of 5 or more amino acid residues from the C-terminus of the protein completely destroys the ability of the PA19 to activate dendritic cells. Removal of up to 20 amino acids from the N-terminus of PA19, as well as adding a FLAG-tag, or more than 30 total amino acids from the pre-ATG region of the gene joined to the N-terminal peptide of beta-galactosidase, showed no such drastic effect on activity. The significant role of cysteine residues in PA19 has been shown by directed point mutations. These mutations abolish immunomodulatory activity when both of the Cys residues were modified. Several other mutants with a significantly lower level of DCA activity were obtained, but all of them contained multiple mutations. The E. tenella PA19 (“PA19-ET”) gene has been re-cloned into mammalian expression vector p3xFLAG-CMV9, which is designed to secrete the expressed protein into the medium.

3D structure analysis of different profilins shows that the C-terminal part of profilin is involved in one alpha-helix structure (the region equivalent to that in PA19 with homology to UvrB), and several beta-layers (the one equivalent to PA19 homologous to UvrC). A 3D structure for individual subunits UvrB and UvrC were published (Hsu et al. (1995) J. Biol. Chem. 270: 8319-27; Theis et al. (1999) EMBO J. 18: 6899-907; Singh et al. (2002) EMBO J. 21: 6257-66) and a model for UvrBC complex has been constructed (Sohi et al. (2000) FEBS Lett. 465: 161-4). The B subunit of the complex is a helicase, while the C subunit is a nuclease.

### 5.8 Example 8

### Screening PRIPs

PA19 homologs from several Apicomplexan parasites, including E. tenella, T. gondii, N. caninum, S. neurona, and P. falciparum have been identified by linking previously uncharacterized EST sequences from GenBank and other sources to contain at least partial cDNA for the PA19 gene. Accordingly, at least one cDNA clone for each of these protozoan profilin-related PA19 series was obtained. The corresponding cDNA and protein sequences for these parasites had not been submitted to GenBank. Using a BLAST EST search with the PA19 protein sequence as a reference, sets of EST sequences for this PA19 protein from some other protozoan parasites, including B. bovis, T. parva, C. parvum, and several P. species: P. vivax, P. yoelii, P. berghei, P. chabaudi, and marine isolates A. tamarense, P. marinus, and L. polyedrum were obtained. A comparative study of these EST sequences lead to generation of complete, or near complete cDNA sequences for all of these organisms. Translation of these cDNA sequences by ExPASy Translate Tool (http://au.expasy.org/tools/dna.htm) generated a set of protein sequences, which were further aligned by the BioEdit Sequence Alignment Editor and ClustalX programs. The alignments were modified manually to highlight the conservative regions, and to group the sequences in the less conservative regions at each position on the basis of amino acid similarity. Some known sequences of profilin, a protein with limited similarity to PA19, were added to the alignment reflecting one-two examples for most classes of organisms (plants, animals, insects, etc.).

### 5.9 Example 9

### Generation of Human Fibrosarcoma Cell Lines Expressing PRIP

By using a dendritic cell activation assay, it was shown that PA19 from T. gondii and N. caninum possess similar properties in enhancing activation of dendritic cells in vitro. Indeed, the PRIP protein from both N. caninum, and T. gondii were shown to be quite active in the in vitro assay.

To investigate the immunomodulatory activity of a PRIP further, the PA19 gene from E. tenella was re-cloned into mammalian bicistronic expression vector pIRES-puro3 and the construction was successfully introduced into human cancer cell lines HT1080 modified with red-fluorescent protein. Several clonal populations of HT1080 cells with the PA19 gene in their genome were obtained and shown to express the PA19 protein (as judged by the in vitro dendritic cell activation assay). Some independent clones were used for injection S.C. into athymic (nude) mice. HT1080 cell lines transfected with the empty vector served as negative controls. About 20 (at least seven independent) clones of HT1080 cell lines expressing PA19 protein in its native (non-secreted) form have been obtained. Several clones were shown by DCA assay to express a higher amount of PA19. These clones will be tested first in mouse experiments. The cell lines transfected with the empty vector will be used as negative controls. Currently, at least ten cell lines of HT1080 transfected with the empty vector pIRES-puro3 have been prepared.

For construction of a plasmid expressing a secreted form of PA19, the gene for PA19 protein of E. tenella was sub-cloned into MCS of the vector p3xFLAG-CMV-9 in-frame to pre-pro-trypsin leading peptide, and 3×FLAG peptide. Because the HT1080 cell line was previously transfected with a neomycin-resistance-gene-containing plasmid encoding for Red-Fluorescence protein, the above construction for the PA19 gene in p3xFLAG-CMV-9 vector (containing the same gene for neomycin resistance) could not be used as it is. Therefore, for purpose of further re-cloning of the PA19 gene, two different constructions had been made, the first being insertion at the HindIII/EcoRV site of the vector, and the second being insertion at the NotI/EcoRV site of the vector. The first construction extends the native PA19 protein of E. tenella from the N-terminus by 23 amino acids (3×FLAG and extra Leu); the second one differs from the first one by extra LeuAlaAla-peptide after the 3×FLAG. (Note that the N-terminal Met is absent in both of these constructions in order to minimize expression of a non-tagged and non-secreted version of PA19 protein.) The first construction has been shown to secrete an active form of PA19 protein from transfected CHO or S-180 cell lines. Human fibrosarcoma cell line HT1080 has been used for stable transfection with the native (non-secreted) form of the PA19 gene in pIRESpuro3 vector. For comparative purpose, the same vector (pIRSpuro3) was chosen for re-cloning of the gene for expression of an artificially created secreted form of the PA19 protein. The insertion of the latter gene was done the way the start codon for the both native, and artificially created gene for PA19 is situated at the constant distance from the vector-supplied CMV promoter. In both cases, the stably transfected clones were selected by addition 1 μg/ml of puromycin to the culture medium, the resulted clones were confirmed to be human fibrosarcoma HT1080 by morphology and red fluorescence of the cells. The expression of PA19 was confirmed by DCA assay of both conditioned media, and cell lysates.

### 5.10 Example 10

### Screening for Agonists

To screen for agonists of TLR11/TLR12 or TLR5, a candidate substance is first obtained. Examples of agonist compounds to be screened include antibodies, aptamers, small molecules, and circular polypeptides. The candidate substance is used in the DC assay in order to determine activation of TLR11/TLR12 or TLR5 by the substance (note that, in addition to the DC assay, any of the above other assays, including the NK assay, the IFN-γ assay, the transfected cell assay, or any subsequently developed assay may also be used, may also be used). Activation of TLR11/TLR12 or TLR5 as indicated by such an assay indicates that the candidate substance is an agonist of TLR11/TLR12 or TLR5.

The level of these cytokines, such as for example, IL-12, IL-6, TNF-α, and interferon, can be measured after treatment of the mDCs, or TLR11/TLR12 and/or TLR5-transfected cells with PA19 by ELISA procedures specific to the cytokine as is known in the art. ELISA kits are available for each of these cytokines. The procedures for ELISA are standard and independent of the cytokine of interest.

### 5.11 Example 11

### Preparation of TLR11/TLR12 and/or TLR5Agonist Antibodies

Immunization of Mice

Purified human TLR11/TLR12 or TLR5 protein, or peptide fragments thereof, is mixed with an equivolume of Freund's complete adjuvant to form an emulsion. This emulsion is intraperitoneally administered to a mouse (e.g., a BALB/c, female, 8 weeks old). Several weeks later, additional immunization are carried out with an emulsion of an equivolume mixture of human TLR11/TLR12 or TLR5 and Freund's incomplete adjuvant. Three or four days before the cell fusion described below, the antigen alone is administered to the mouse.

Cell Fusion

Three to four days after the final immunization, spleen is taken out from the immunized mouse. The spleen is disrupted using a mesh and spleen cells are suspended in PBS. The spleen cells are mixed with myeloma cells at a ratio of 10:1 and the resulting mixture is left to stand for 3 minutes in the presence of 50% polyethylene glycol. The resulting mixture is centrifuged at 1200 rpm for 8 minutes and the supernatant is removed. The cells are then suspended in HAT RPMI-1640 medium containing 10% FCS at a population density of 3.5×10⁶ cells/ml, and the resulting suspension is divided into wells of a 96-well microtiter plate in 0.1 ml/well aliquots. The 96-well microtiter plate is incubated at 37° C. under an atmosphere of 5% CO2. After 2-3 days from the beginning of the incubation, 0.1 ml of HAT RPMI-1640 medium containing 10% FCS is added to each well and then half of the medium was replaced every 3-4 days. After 7-10 days from the beginning of the incubation, colony formation is observed, and sufficient amount of antibody specific to the immunogen is produced in at least one well. The culture supernatants of the antibody-producing wells were subjected to screening.

Screening

The screening of the antibodies is carried out by ELISA (Immunochem., 8:871-874, 1971). That is, to the wells of a 96-well microtiter plate to which 50 μl of an antigen solution in PBS was preliminarily adsorbed, 50 ul of the culture supernatant was placed in each well, and the microtiter plate is incubated at 30° C. for 2 hours. A solution of peroxidase-labeled anti-mouse immunoglobulin antibody is placed in each well and the microtiter plate is incubated at 30° C. for 1 hour. Finally, o-phenylenediamine as a substrate is added. The presence or absence of the anti-human TLR11/TLR12 and/or TLR5 antibody is evaluated by the generated color.

Cloning

Cells are taken out from the antigen-specific antibody producing wells and subjected to cloning by the soft agar method. That is, a suspension of hybridomas (10×10⁶ cells/ml) in HT-RPMI 1640 medium containing 10% FCS is mixed with soft agar and the mixture is divided into petri dishes in an amount of 5 ml/dish. After incubation at 37° C. for 7-10 days, colonies are picked up and the positive colonies are evaluated to be hybridomas producing anti-human TLR11/TLR12 and/or TLR5 monoclonal antibody. The above-described cloning procedure is repeated twice to obtain three hybridomas producing anti-human TLR11/TLR12 and/or TLR5 monoclonal antibodies.

Preparation of Monoclonal Antibodies

The hybridomas are transplanted to abdominal cavities of pristane-treated mice. Two to three weeks later, ascites fluid is recovered from the mice.

TLR11/TLR12 or TLR5-Agonist Ability

A purified preparation of human TLR11/TLR12 or TLR5 and each ascites fluid containing an antibody are mixed and the mixture. Thereafter, the TLR11/TLR12 or TLR5 activities of the formed antigen-antibody complexes are measured. Monoclonal antibodies corresponding to ascites fluid having TLR11/TLR12 and/or TLR5 agonist activity are selected.

### 5.12 Example 12

### Expression of PA19 Protein by HT1080 Human Sarcoma Cells in Athymic Mice Leads to Increased Life Span of the Animals

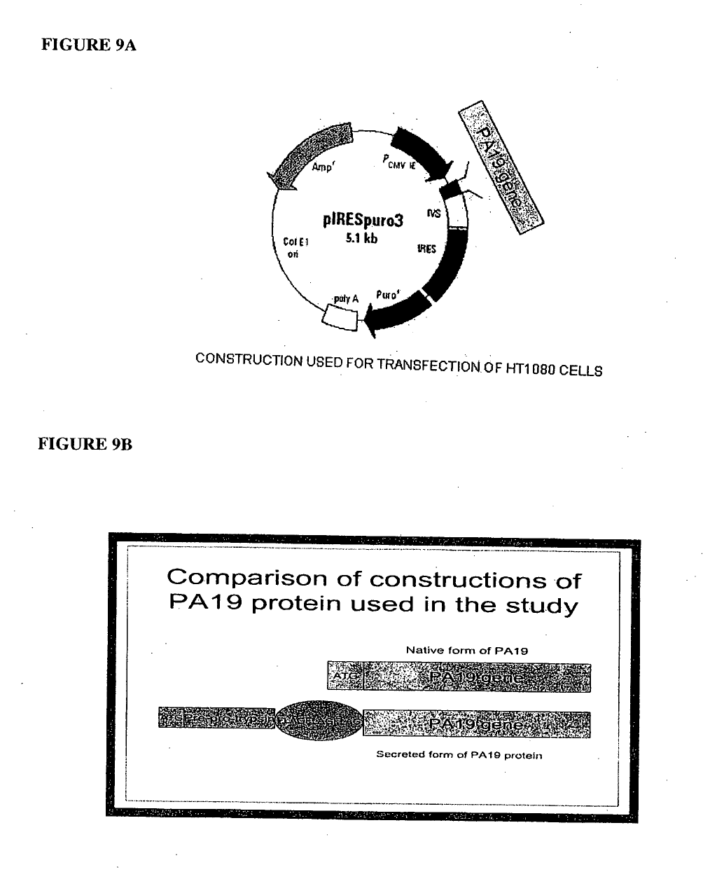

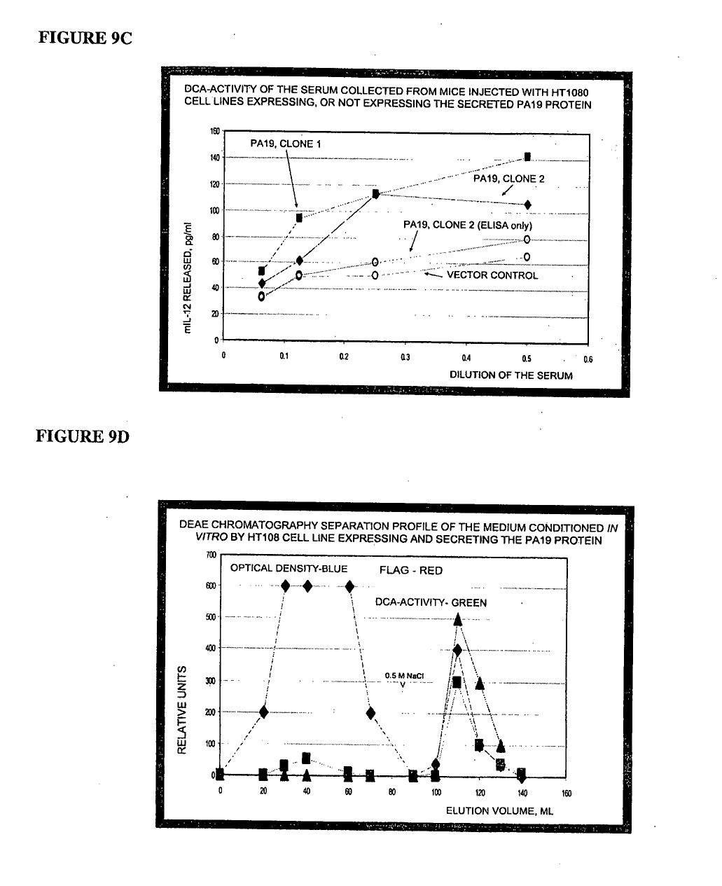

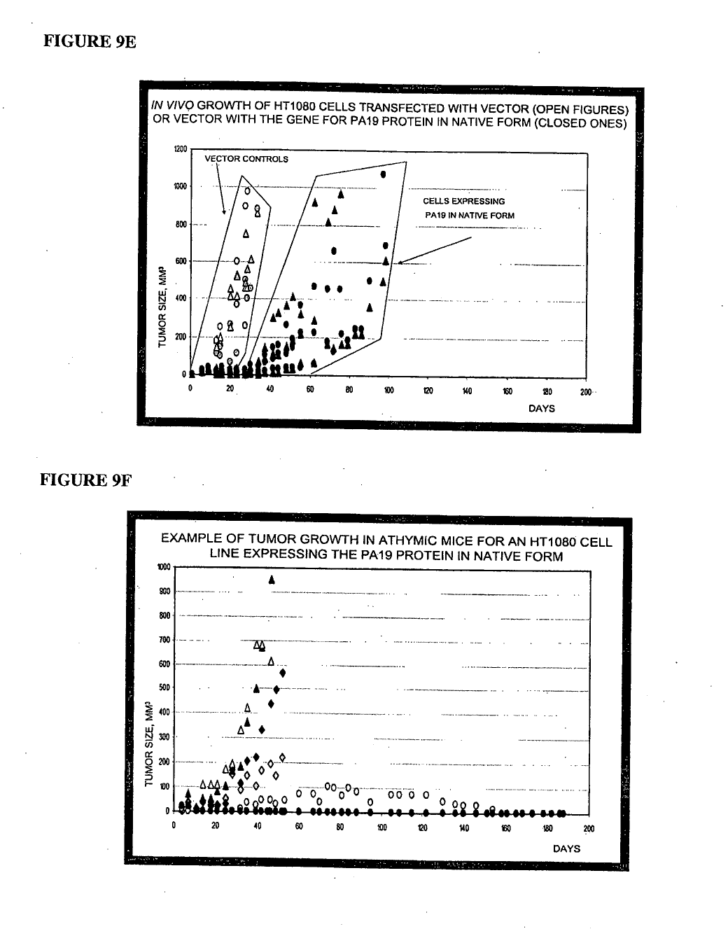

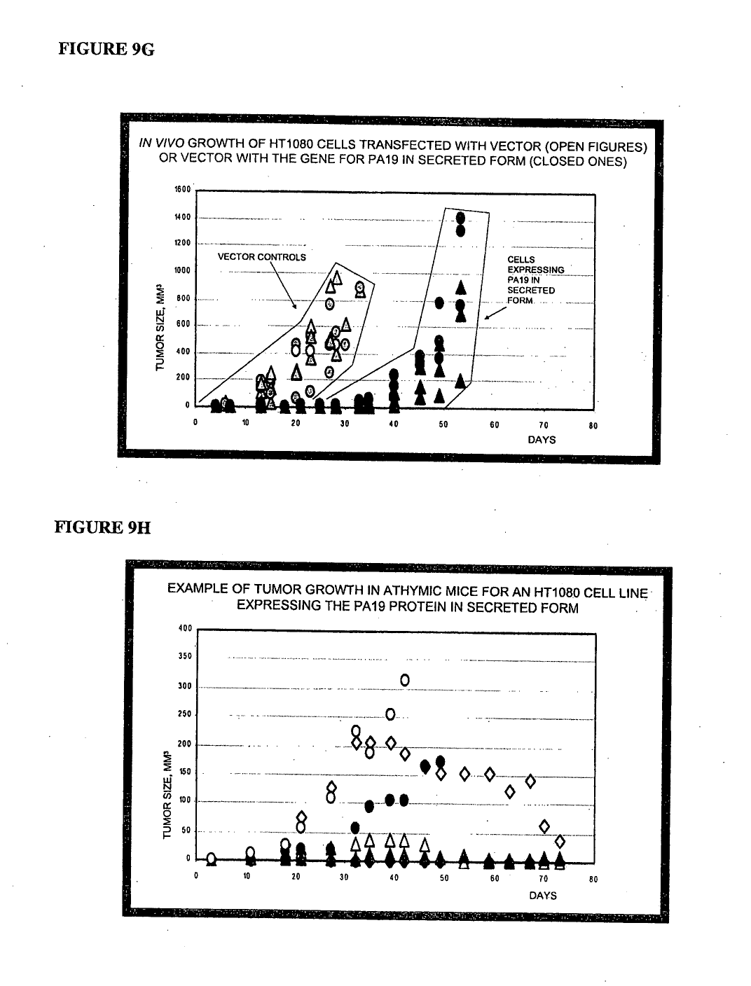

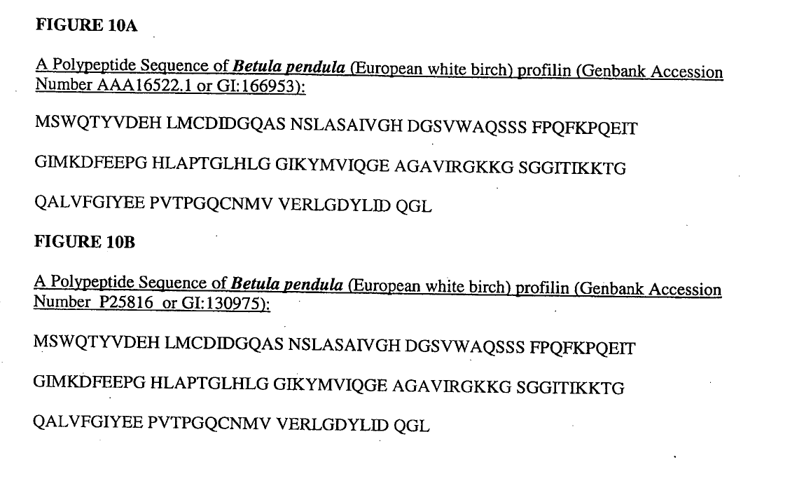

For cloning purposes, EST clones showing some similarity to the DNA sequence of the PA19 gene from Eimeria tenella were obtained. The inserts in each clone were completely sequenced from both ends, and the clone containing the full copy of the gene was used to re-clone into TA-cloning vector pCR2.1 TOPO (Invitrogen, Carlsbad, Calif.). The gene in pCR2.1 was used for all further procedures of cloning into expression vectors. EcoRI sites were used for cloning of both the native PA19 gene, and the secreted form of the gene, into mammalian expression vector, pIRESpuro3 (BD) (shown in FIG. 9A) to insure the equal distance for the ATG-start codon of the construction from the CMV promoter site. The final clone with the gene in direct orientation was selected in each case by PCR, and confirmed by complete sequencing. Cloning of the secreted form of the PA19 gene was done through intermediate cloning of the gene lacking the ATG-start codon into vector p3xFLAG-CMV9 (Sigma). A comparison of these constructions is shown in FIG. 9B.

PA19 protein activates dendritic cells (DCs) as well as natural killer (NK) cells in vitro. (See Rosenberg et al., Int. J. Cancer 2005 114: 756-765.) When Balb/C mice are injected intraperitoneally (i.p.) with S-180 murine sarcoma cells followed by an i.p. injection of PA19 protein, tumor formation was completely blocked. To determine whether PA19 would also be effective in inhibiting human tumor formation, several HT1080 human sarcoma cell lines were established that permanently express the PA19 protein (in secreted or native, non-secreted form). Because the gene for resistance to puromycin was bicistronically linked to the PA19 gene in the plasmid (pIRES puro3), this antibiotic was used for selection of the cells potentially expressing the PA19 protein. Expression of the PA19 protein was confirmed in all the analyzed clones and quantified on the basis of the degree the cell line lysates, or conditioned media, were able to activate DCs in vitro. Fourteen clonally independent cell lines expressing the PA19 protein at different levels, as well as five independent vector control cell lines and one parent cell line were subcutaneously (s.c.) injected into the rear flanks of athymic mice (10⁶ cells per flank, three mice per cell line). Tumor growth was measured biweekly and when a tumor reached a volume of 0.5 cm³, the mouse was euthanized. Tumors were removed, fixed, stained and analyzed histologically. For each strain, two randomly chosen tumor masses from two out of three different mice were removed aseptically, and the cells were cultured in selective complete medium. The level of PA19 expressed by these tumor-derived cell lines was calculated from the ability of the conditioned medium to activate DCs.

DCA assay was used as described in Rosenberg et al. (Int. J. Cancer 2005 114: 756-765) with slight modifications. Dendritic cells were isolated from 7-10 weeks old hairy males Balb/C mice. (DCs from male mice younger than 5 weeks have been shown to be able to be activated by PA19 to much lesser extent, while inclusion of female mice into the pool leads to less reliable results). Dendritic cells were positively selected by usage of MACS mCD11c magnetic beads (Miltenyi Biotech, Auburn, Calif.). Since cell fractions with stronger affinity to the column (only eluted from the column after applying additional pressure) are as active in the DCA-assay as the standard fraction of cells removed from the column by free-flow of the buffer, the combination of these two fractions of DC-enriched cells was used in most assays. The evaluation of the level of activation of the DCs was performed on the basis of analysis by ELISA (R&D kit) of the level of mIL-12 released. FIG. 9C shows the DCA activity of the serum collected from mice injected with HT1080 cell lines expressing, or not expressing the secreted PA19 protein. The level of mIL-12 released was generally higher for the cell lines expressing the secreted PA19 protein. FIG. 9D is a DEAE chromatography separation profile of the medium conditioned in vitro by HT108 cell line expressing and secreting the PA19 protein.

The athymic mice injected s.c. with human sarcoma HT1080 cell lines expressing PA19 protein exhibited a statistically significant increase in tumor latency compared to the control cell lines as shown in Tables 5 and 6 below. Table 5 shows comparative data of the in vivo tumorigenicity of the fibrosarcoma malignant human HT1080 cells expressing, or not expressing, the PA19 protein in native form. FIG. 9E shows the in vivo growth of HT1080 cells transfected with vector (open figures) or vector with the gene for PA19 protein in native form (closed figures). FIG. 9F shows an example of tumor growth in athymic mice for an HT1080 cell line expressing the PA19 protein in native form. FIG. 9G shows the in vivo growth of HT1080 cells transfected with vector (open figures) or vector with the gene for PA19 in secreted form (closed figures). FIG. 9H shows an example of tumor growth in athymic mice for an HT1080 cell line expressing the PA19 protein in secreted form. Histology analysis showed that the tumors formed by the PA19-expressing cells are fibrosarcomas. However, they are atypically soft and contain a central necrotic area. Because the athymic mice are highly deficient in T-cells, the necrosis observed is most probably caused by NK cells recruited by murine DCs activated by PA19 protein. About 15% of the sites injected with HT1080 cells expressing the native form of PA19 remained tumor free for more than 150 days. (The tumor free period of mice injected with HT1080 cells transfected with the empty vector was 10 days, with the average time for the tumor mass to reach the size of 1 cm³ being 35 days). When athymic mice were injected with HT1080 cells expressing the secreted form of PA19, about 40% of the sites remained tumor free for more than 70 days.

TABLE 5

| Type of HT1080 | | | |
|---|---|---|---|
| cell line injected | No. of sites with | Size of tumors (cm³) | No. of days for tumors to reach 1 |
| into athymic mice | tumor/total sites injected¹ | (minimal size/maximal size)¹ | cm³ (Average ± standard deviation) |
| Transfected with | 6/6 | 0.9 −> 1.2 | 33 ± 3 |
| the vector control, | | | |
| typical clone | | | |
| Clone 1A | 4/6 | 0-0.092 | 83 ± 18 |
| Clone 2A | 5/6 | 0-0.34 | 71 ± 45 |
| Clone 3A | 2/6 | 0-0.478 | 119 ± 63 |

¹By day 35

Evidence that PA19 interferes with the formation of fibrosarcomas by malignant human HT1080 cells injected into athymic mice is shown in Table 6.

TABLE 6

| | | | | Average | | |
|---|---|---|---|---|---|---|
| Type of | Sites with a | | | number of days | No. of sites | |
| HT1080 cells | tumor/sites | | Average size of | for tumors to | with tumors per | |
| injected | injected¹ | | tumors (cm³)¹ | reach 1 cm³ | sites injected² | |
| Parental | 6/6 | (100%) | 1.16 | 35 | 6/6 | (100%)* |
| Transfected | 28/30 | (93%) | >1.2 | 35 | 30/30 | (100%)* |
| with vector | | | | | | |
| control | | | | | | |
| Expressing the | 32/46 | (69.6%) | 0.38 | 68 | 40/46 | (87) |
| native form of | | | | | | |
| PA19 | | | | | | |
| Expressing the | 5/30 | (16.7%) | 0.096 | 76 | 20/30 | (66.7%) |
| secreted form of | | | | | | |
| PA19 | | | | | | |

¹By day 35
²By day 70

### 5.13 Example 13

### Methods of Treatment

Treatment of Human Cancer

In an early study also described in WO 2005/010040 (and U.S. 2005/0169935) of PA19, two human phase I trials were approved by the FDA for use of a partially purified (still containing hundreds of proteins) extract of bovine small intestine containing PA19 (called BBX-01/01c). While the trial was designed to evaluate toxicity of the extracts, vigilance for signs of tumor regression was maintained throughout both trials.

In the trial using BBX-01, no consistent clinical response was observed. However, in the second trial using the more highly purified form, BBX-01c, a single patient with a germ cell ovarian carcinoma (see Table 4 below showing CT scan summaries for patient with a germ ovarian carcinoma who received multiple doses of BBX-01c drug) demonstrated elevated serum level of IL-12 and dramatic reduction in a 8 cm pelvic tumor mass after a single 5-day course of BBX-01c. This was followed by complete elimination of the tumor and all peritoneal ascites. Other metastatic masses in her liver and spleen were refractory, but they remained stable for almost two years. The patient went from having severe pain and being bed-ridden, to a pain-free status allowing her to return to work.

This reaction would be expected from a patient containing an active form of TLR12. Thus, there is reason to believe that TLR11/TLR12 and/or TLR5 in humans is indeed polymorphic, and that some potential patients would have TLR11/TLR12 and/or TLR5 in active form. This fact also provides a good tool for selecting patients for treatment with PA19 by analyzing the pattern of TLR11/TLR12 and/or TLR5 in their genome. (In mice, the gene for TLR11/TLR12 and/or TLR5 is intron-free, so analysis of this gene is straightforward.)

TABLE 4

| Date | Pelvic | | | |
|---|---|---|---|---|
| m-d-yy | Mass | Liver & Spleen Masses | Ascites | Comments |
| 12-4-00 | 8 × 8 cm | 8 × 9 cm liver mass, | yes | Prior to BBX-01c therapy |
| | | 6 × 6 cm spleen mass | | |
| 2-1-01 | 2 × 3 cm | unchanged | no | 2 wks after the therapy |
| 3-6-01 | 2 × 3 cm | unchanged | Yes | 7 wks after the therapy |
| | | | (minimal) | |
| 5-9-01 | ND* | unchanged | no | 6 wks after the therapy** |
| 11-9-01 | ND* | unchanged | no | 32 wks after the therapy |
| 6-30-02 | ND* | unchanged | no | 66 wks after the therapy |

*ND = not detectable
**the patient has received second course therapy with higher doses of the BBX-01c.

Since no full-length, intact gene for TLR11/TLR12 has been consistently seen in humans, the possibility of artificially “repairing” the gene arises. The process involves cloning of the TLR11/TLR12 gene from a human cell line/tissue, complete sequencing of the gene in order to deduce where the premature stop codons are located, and changing the stop codons one by one by point mutagenesis to an amino acid most common at the position in murine, rat, etc genes. The resulting mutated “repaired” gene is used for expression of the hTLR11 protein which it is expected, will be totally active. The gene could be delivered to a patient via viral vector, or the patient-derived cell line expressing the gene. This opens the possibility of using the gene in conjunction with PRIP treatment for human patients with cancer, infectious disease, or any other illness proven to be treatable through TLR11/TLR12. It is even possible that delivering a murine copy of the TLR11/TLR12 gene into human patients would work, since it has been shown that the mTLR11/TLR12 gene expresses in 293 Human embryonic kidney cell lines and activates the NF-κB pathway. The same scheme can be used for veterinary purpose.

Analysis of the human (pseudo)-gene for TLR11/TLR12 (see alignment in FIG. 14) shows that it contains about 10 sites that need to be repaired (premature stop-codons and frame-shifts, which are highlighted in the alignment). This can be accomplished in the course of 5-10 consecutive repairs by site-directed mutagenesis. As the result, the gene will return to a form usable for expression of the whole protein (see FIG. 15) for the predicted form of hTLR11/TLR12 gene for a functional protein; the prediction was guided by comparative analysis of mouse, rat, and human sequences). This strategy would cover all possible situations in human patients, and allow all human patients to have treatment with PA19 available to them. To prove that such a scheme works, experiments could be performed on dog pets with tumors (it appears that dogs do not have the full version of the TLR11/TLR12 protein expressed, although the genome of dogs is at much lower level of confidence than the mouse genome at this point). This apparently straight-forward experiment would require the mTLR11/TLR12 gene to be expressible in a virus that could be used in dogs and other veterinary applications. Several BAC clones containing the region of the mTLR12 gene, as well as a BAC clone with the region of hTLR11/TLR12 (pseudo)-gene have been described.

Assay for Genotyping the TLR11/TLR12 and/or TLR5 Locus in Humans

In order to determine the genotype of the TLR11/TLR12 and/or TLR5 locus in humans, an assay will be developed that is similar to standard SNP assays used for mapping polymorphic proteins in human patients. In general, sufficient information in regard to the polymorphic loci in the TLR11/TLR12 or TLR5 gene would need to be generated and the primers to each locus would need to be constructed separately. The rules for construction of SNP-related primers are well established, for example, at http://www.ncbi.nlm.nih.gov/About/primer/snps.html (general review) or at http://snp.wustl.edu/snp-and-fp-tdi-resources/genotyping-primers/assay-design.html (more in-depth information on designing the primers and PCR regimes).

Evidence for Anti-Viral Activity of Protozoan PA19 Profilin-Related Protein in Humans

As described in WO 2005/101140, during the Phase I human trial of BBX-01, a terminal lung cancer patient reported the complete disappearance of long-term warts, likely to be of papillomavirus origin, from two body regions. The first along the left and central region of the back (along the line of the spine), and the second along the upper region of the left arm. The report stated that the warts suddenly dried up and disappeared. This occurred after the patient received three progressively larger single doses of BBX-01 spaced at approximately two-week intervals, but before receiving a multiple-dose course. The patient was under no other therapy during this period.

Evidence for Anti-Viral Activity of Protozoan PA19 Profilin-Related Protein in Mice

As described in WO 2005/101140, specific pathogen-free female BALB/c mice were infected intranasally with an LD90 dose of influenza virus A/NWS/33 (H1N1). The mice were then treated with a protozoan PA19 profilin-related protein E1 by one of two treatment protocols. In the first protocol mice received 100 ng of protozoan PA19 profilin-related protein E1 given intraperitoneally 48 hours before viral exposure, 4 hours after viral exposure (day 0) and on days 3 and 6 after viral exposure. In the second protocol mice received 100, 1,000, or 10,000 ng of protozoan PA19 profilin-related protein E1 intraperitoneally 4 hours after viral exposure (day 0) and on days 3 and 6 after viral exposure. Placebo treated mice received bovine serum albumin in phosphate-buffered saline. Mice were observed daily for death. The survival of the mice exposed to influenza is shown in Table 8.

TABLE 8

| | | Treatment | | Mean Day to |
|---|---|---|---|---|
| Compound | Dose (ng/day) | Schedule | Survive/Total | Death^{a} ± SD |
| E1 | 100 | −2, 0, 3, 6 | 0/10 | 12.3 ± 1.2* |
| E1 | 100 | 0, 3, 6 | 2/10 | 12.1 ± 1.6 |
| E1 | 1,000 | 0, 3, 6 | 3/10 | 12.7 ± 2.7 |
| E1 | 10,000 | 0, 3, 6 | 5/10* | 12.0 ± 1.4* |
| Placebo | | 0, 3, 6 | 2/20 | 11.3 ± 1.2 |

^{a}Mean day to death of mice dying before day 21
*P < 0.05

There was a significant increase in the number of survivors and time to death in the mice treated with 10,000 ng of protozoan PA19 profilin-related protein E1. Arterial oxygen saturation was also measured in these mice on days 3-11. There was a statistically significant reduction of the decline in oxygen saturation in the mice treated with 1,000 ng and 10,000 ng of protozoan PA19 profilin-related protein E1.

### 5.14 Example 14

### Detection of PRIP TLR5-Stimulating Activity

CHO cells expressing human TLR5 and a luciferase-linked reporter are used to screen for PRIPs recognized by the receptor. CHO cells are transiently transfected with TLR5, or empty expression vectors together with a NF-kB luciferase reporter. The cells are treated with 100 ng/ml LPS, 100 ng/ml lipopeptide, 10⁷ yeast particles/ml, or untreated (control), and luciferase activity was measured. The cells are treated with the PRIP, or LB alone (control), and the luciferase activity is measured.

Human TLR5 are generated by PCR from cDNA derived from human peripheral blood mononuclear cells and is cloned into pEF6-TOPO (Invitrogen, Carlsbad, Calif.) (pEF6-hTLR5). Murine TLR5 is generated by PCR using cDNA derived from RAW-TTIO cells and cloned into pEF6 (pEF6-mTLR5).

For luciferase assays, CHO cells are transfected by electroporation as described above, with 1 mg of the indicated TLR expression vector, 1 mg of ELAM-firefly luciferase, 0.1 mg of TK-renilla luciferase (Promega, Madison, Wis.). The medium is replaced with medium containing the stimuli at the indicated concentration/dilution. Bacterial lipopeptide can be obtained from Roche (Nutley, N.J.), LPS (Salmonella minnesota R595) was from List, and yeast particles (zymosan) were from Molecular Probes (Eugene, Oreg.). Cells are stimulated for 5 hours at 37° C., and firefly and Renilla luciferase activities are measured using the Dual Luciferase Assay System (Promega, Madison, Wis.).

For preparation of bacterial supernatants, bacteria ware grown either in Luria broth (LB) (E. coli TOP 10 (Invitrogen, Carlsbad, Calif.), Salmonella minnesota (ATCC#49284), mutant Salmonella typhimurium (TH4778fliB− fliC+), TH2795 (fliB− fliC−), (Dr. Kelly Hughes, University of Washington), or grown in trypticase soy broth (TSB) (Listeria monocytogenes, Listeria innocua (ATCC#33090), Bacillus subtilis and Pseudomonas aeruginosa. Bacteria are grown to saturation (about 16 hours, 37° C. with vigorous aeration). The bacterial culture supernatants are centrifuged for 30 min at 2000×g, are filtered (0.2 mM), and stored at 4° C. prior to use. For flaA transfections, E. coli TOP10 containing pTrcHis2-flaA or pTrcHis2-flaArev are selected from bacterial plates and grown to OD₆₀₀ of 0.6 in LB with 100 ug/ml ampicillin and 1% w/v glucose. The bacteria are centrifuged for 30 minutes at 2000×g, and split into two LB cultures, one containing 100 mg/ml ampicillin and 1% w/v glucose (to repress flaA) and the other containing 100 mg/ml ampicillin and 1 mM IPTG (to induce flaA). Samples are taken at 4 hours after induction, centrifuged 5 min at 10,000×g, and the supernatants stored at 4° C. before use.

### 5.15 Example 15

### In Vitro Treatment of Human Fibrosarcoma Cells with PA19

In this study, the responsiveness of a human cell line to PA19 was confirmed. A human fibrosarcoma cell line (HT1080/pCMV-DsRed-X/pIRESpuro3 clone B5) was used for this experiment. The cells (approximately 80% confluent at the time of harvesting) were seeded into 24-wells plate at cell density 3×10⁴, or 7.5×10⁴ cells/well. The cells in complete medium were allowed to attach to the surface and incubated overnight at 37° C. in a CO₂ incubator. The conditioned medium was then replaced with complete Eagle's medium containing either 0.1 mg/ml human serum albumin (HSA), or 0.1 mg/ml HSA and 1 ng/ml of recombinant PA19 from Eimeria tenella. Conditioned medium was sampled from each well at 8.5 hrs after treatment and the level of the hIL-6 secreted into the medium was determined using the ELISA Duo-kit (R&D) as recommended by the manufacturer.

The results are shown in FIG. 31. The coded bar graph values represent the average from three independent wells (three readings from each well). The standard deviation for each set of data is shown by the error bar. The results demonstrate that human cells are responsive to an immunomodulatory profilin-related polypeptide.

### 5.16 Example 16

### In Vivo Treatment of Human Cancers with PA19

In this study, the in vivo responsiveness of human cancers to PA19 was confirmed.

In the first study, protective effect of purified recombinant PA19 on survival of mice injected intraperoneously with a human fibrosarcoma was analyzed. Mice were injected intraperitoneously with 10⁶ cells of the HT1080 human fibrosarcoma cell line. Thirty minutes after injection of the cells the mice were injected i.p. with recombinant PA19 (more than 95% purity by gel electrophoresis). Treatment groups were as follows:

1. Cells only

2. 0.1% HSA (human serum albumin)

3. 0.1 ng PA19 in 0.1% HSA

4. 1.0 ng PA19 in 0.1% HSA

5. 10 ng PA19in 0.1% HSA

These doses were administered again to the mice on days 2, 4 and 7. The mice were weighed and their abdominal circumference measured twice a week. Mice that showed signs of ill health were euthanized. At the time of euthanasia each mouse was photographed, had blood drawn and was necropsed for histopathological examination. All procedures were carried out with approval from the Institutional Animal Care and Use Committee (IACUC) at MSU.

The results are shown in FIG. 32A, which demonstrates the protective effect of purified recombinant PA19 on survival of mice injected intraperoneously with the human fibrosarcoma.

In the second study, protective effect of purified recombinant PA19 on survival of mice injected intraperoneously with a human ovarian carcinoma was analyzed. Mice were injected intraperitoneously with 10⁵ cells of the ES-2 human ovarian carcinoma cell line. Thirty minutes after injection of the cells the mice were injected i.p. with recombinant PA19 (more than 95% purity by gel electrophoresis). Treatment groups were as follows:

1. Cells only

2. 0.1% HSA (human serum albumin)

3. 0.1 ng PA19 in 0.1% HSA

4. 1.0 ng PA19 in 0.1% HSA

5. 10 ng PA19 in 0.1% HSA

These doses were administered again to the mice on days 2, 4 and 7. The mice were weighed and their abdominal circumference measured twice a week. Mice that showed signs of ill health were euthanized. At the time of euthanasia each mouse was photographed, had blood drawn and was necropsed for histopathological examination. All procedures were carried out with approval from the Institutional Animal Care and Use Committee (IACUC) at MSU.

The results are shown in FIG. 32B, which demonstrates the protective effect of purified recombinant PA19 on survival of mice injected intraperoneously with the human ovarian carcinoma. These results demonstrate that multiple types of human cancers, including sarcomas and carcinomas, are responsive to an immunomodulatory profilin-related polypeptide.

### EQUIVALENTS

Those skilled in the art will recognize, or be able to ascertain, using no more than routine experimentation, numerous equivalents to the specific substances and procedures described herein. Such equivalents are considered to be within the scope of this invention, and are covered by the following claims.

We claim:

1. An isolated immunomodulatory polypeptide encoded by a nucleic acid that hybridizes under stringent conditions to a nucleic acid selected from the group consisting of (SEQ ID NO:7) [Neospora caninum], (SEQ ID NO:8) [Sarcocystis neurona], (SEQ ID NO:9) [Toxoplasma gondii] and (SEQ ID NO:10) [Plasmodium falciparum].

2. The polypeptide of claim 1 having a toll-like receptor agonist activity.

3. The polypeptide of claim 2, wherein the toll-like receptor is selected from the group consisting of TLR11, TLR12, and TLR5.

4. The polypeptide of claim 1, wherein the immunomodulatory polypeptide causes an increase in the level of IL-12 when administered to a subject.

5. The polypeptide of claim 4, wherein the subject is a mammal.

6. The polypeptide of claim 5, wherein the mammal is a human.

7. The isolated polypeptide of claim 4, wherein the immunomodulatory polypeptide stimulates Interleukin-12 (IL-12) synthesis in dendritic cells (DCs).

8. The isolated polypeptide of claim 1, wherein the stringent hybridization conditions comprise hybridization at 65° C. in 4×SSC.

9. The isolated polypeptide of claim 8, wherein the stringent hybridization conditions further comprise washing at 65° C. in 1×SSC.

10. An isolated profilin-related immunomodulatory polypeptide encoded by a nucleic acid that hybridizes under stringent conditions to a nucleic acid encoding a polypeptide having an amino acid sequence selected from the group consisting of (SEQ ID NOS:1-4).

11. The isolated polypeptide of claim 10, wherein the stringent hybridization conditions comprise hybridization at 65° C. in 4×SSC.

12. The isolated polypeptide of claim 11, wherein the stringent hybridization conditions further comprise washing at 65° C. in 1×SSC.

13. An isolated immunomodulatory polypeptide encoded by a nucleic acid selected from the group consisting of (SEQ ID NOS:1-4).

14. The isolated immunomodulatory polypeptide of claim 1 which, when transgenically expressed from the hybridizing nucleic acid in a human HT1080 fibrosarcoma cell line, causes a delay and/or reduced tumor growth in an implanted athymic mouse.

15. An isolated profilin-related immunomodulatory polypeptide encoded by a nucleic acid that hybridizes under stringent conditions to a nucleic acid selected from the group consisting of (SEQ ID NO: 127) [Betula verrucosa nucleic acid] (FIG. 30B), and (SEQ ID NO: 125) [Pinus pinaster nucleic acid] (FIG. 29B).

16. The isolated polypeptide of claim 15, wherein the stringent hybridization conditions comprise hybridization at 65° C. in 4×SSC.

17. The isolated polypeptide of claim 16, wherein the stringent hybridization conditions further comprise washing at 65° C. in 1×SSC.

18. An isolated immunomodulatory polypeptide selected from the group consisting of (SEQ ID NO: 126) [Betula verrucosa polypeptide] (FIG. 30A), and (SEQ ID NO: 124) [Pinus pinaster polypeptide] (FIG. 29A).

19. An isolated profilin-related immunomodulatory UvrBC polypeptide complex comprising a UvrB polypeptide and a UvrC polypeptide.

20. The isolated profilin-related immunomodulatory UvrBC polypeptide complex of claim 19 comprising a UvrB polypeptide having the contiguous sequence MVLAPNKTLAAQLYGEM-KEFFPENAVEYFVSYYDY (SEQ ID NO: 47) and a UvrC polypeptide having the contiguous sequence KAIDD-SKIPDVILIDGGKGQLAQAKNVFAELDVSWDKNHPLLLGVAKGA (SEQ ID NO: 48).

21. The isolated profilin-related immunomodulatory UvrBC polypeptide complex of claim 19, wherein the UvrB polypeptide has the sequence of (SEQ ID NO: 30) (E. coli UvrB subunit in FIG. 12) and the UvrC polypeptide has the sequence of (SEQ ID NO: 32) (E. coli UvrC subunit in FIG. 12).

22. A polypeptide comprising an immunomodulatory polypeptide sequence of claim 1 fused to an heterologous polypeptide sequence.

23. The polypeptide of claim 22, wherein the heterologous polypeptide sequence comprises pre-pro-trypsin.

24. The polypeptide of claim 22, wherein the heterologous polypeptide sequence comprises an affinity tag.

25. The polypeptide of claim 24, wherein the affinity tag is a FLAG tag.

26. An immunostimulatory TLR11/12 agonist selected from the group consisting of antibodies, aptamers, small molecules, and circular polypeptides.

27. The immunostimulatory TLR11/12 agonist of claim 26, which is a high affinity ligand of TLR11/12.

28. The immunostimulatory TLR11/12 agonist of claim 26, which causes an increase in the level of IL-12 when administered to a subject.

29. The polypeptide of claim 28, wherein the subject is a mammal.

30. The polypeptide of claim 29, wherein the mammal is a mouse.

31. The immunostimulatory TLR11/12 agonist of claim 26, wherein the agonist stimulates Interleukin-12 (IL-12) synthesis in dendritic cells (DCs).

32. The immunostimulatory TLR11/12 agonist of claim 26, wherein the agonist is an antibody.

33. The antibody of claim 32 which is a monoclonal antibody.

34. The antibody of claim 32, wherein the antibody causes an increase in the level of IL-12 when administered to a subject.

35. The immunostimulatory TLR11/12 agonist of claim 26, which is an aptamer.

36. The immunostimulatory TLR11/12 agonist of claim 26, which is a small molecule.

37. The immunostimulatory TLR11/12 agonist of claim 26, which is an aptamer.

38. The immunostimulatory TLR11/12 agonist of claim 26, which is a circular polypeptide.

39. A pharmaceutical formulation comprising the immunomodulatory polypeptide of claim 1 and a pharmaceutically acceptable carrier.

40. A pharmaceutical formulation, comprising an immunomodulatory polypeptide sequence of claim 1 and a pharmaceutically acceptable carrier.

41. A pharmaceutical formulation, comprising an immunomodulatory polypeptide sequence of claim 10 and a pharmaceutically acceptable carrier.

42. A pharmaceutical formulation, comprising an immunomodulatory polypeptide sequence of claim 15 and a pharmaceutically acceptable carrier.

43. A pharmaceutical formulation, comprising an immunomodulatory polypeptide sequence of claim 18 and a pharmaceutically acceptable carrier.

44. A pharmaceutical formulation comprising the immunostimulatory TLR11/12 agonist of claim 26 and a pharmaceutically acceptable carrier.

45. A method of activating TLR11/12 and/or increasing the level of IL-12 in a subject, comprising administering to the subject an effective amount of a composition comprising an amino acid sequence selected from the group consisting of (SEQ ID NO:1) [Neospora caninum], (SEQ ID NO:2) [Sarcocystis neurona], (SEQ ID NO:3) [Toxoplasma gondii], and (SEQ ID NO:4) [Plasmodium falciparum].

46. A method of activating TLR11/12 and/or increasing the level of IL-12 in a subject, comprising administering to the subject an effective amount of a composition comprising an amino acid sequence selected from the group consisting of (SEQ ID NO: 126) [Betula verrucosa polypeptide] (FIG. 30A), and (SEQ ID NO: 124) [Pinus pinaster polypeptide] (FIG. 29A).

47. A method of activating TLR11/12 and/or increasing the level of IL-12 in a subject, comprising administering to the subject an effective amount of a composition comprising a profilin-related immunostimulatory polypeptide, wherein the profilin-related immunostimulatory polypeptide is encoded by a nucleic acid that hybridizes under stringent conditions to a nucleic acid selected from the group consisting of (SEQ ID NOS:7-10).

48. A method of activating TLR11/12 and/or increasing the level of IL-12 in a subject, comprising administering to the subject an effective amount of a composition comprising a profilin-related immunostimulatory polypeptide, wherein the profilin-related immunostimulatory polypeptide is encoded by a nucleic acid that hybridizes under stringent conditions to a nucleic acid selected from the group consisting of (SEQ ID NO: 127) [Betula verrucosa nucleic acid] (FIG. 30B), and (SEQ ID NO: 125) [Pinus pinaster nucleic acid] (FIG. 29B).

49. The method of claim 45, wherein the subject is a non-human animal.

50. The method of claim 49, wherein the subject is a mammal.

51. The method of claim 45, wherein the subject is a human.

52. A method of activating TLR11/12 and/or increasing the level of IL-12 in a subject, comprising administering to the subject an effective amount of a composition comprising an immunostimulatory TLR11/12 agonist selected from the group consisting of antibodies, aptamers, small molecules, and circular polypeptides.

53. The method of claim 45, wherein the subject is in need of treatment for a cancer.

54. The method of claim 45, wherein the subject is in need of treatment for an infectious disease.

55. A method of treating an infectious disease in a subject, comprising administering to the subject an effective amount of a pharmaceutical formulation comprising an amino acid sequence selected from the group consisting of (SEQ ID NO:1) [Neospora caninum], (SEQ ID NO:2) [Sarcocystis neurona], and (SEQ ID NO: 3) [Toxoplasma gondii].

56. A method of treating an infectious disease in a subject, comprising administering to the subject an effective amount of a composition comprising an amino acid sequence selected from the group consisting of (SEQ ID NO: 126) [Betula verrucosa polypeptide] (FIG. 30A), and (SEQ ID NO: 124) [Pinus pinaster polypeptide] (FIG. 29A).

57. A method of treating an infectious disease in a subject, comprising administering to the subject an effective amount of a profilin-related immunostimulatory fragment, wherein the profilin-related immunostimulatory polypeptide is encoded by a nucleic acid that hybridizes under stringent conditions to a nucleic acid selected from the group consisting of (SEQ ID NOS:7-10).

58. A method of treating an infectious disease in a subject, comprising administering to the subject an effective amount of a profilin-related immunostimulatory fragment, wherein the profilin-related immunostimulatory polypeptide is encoded by a nucleic acid that hybridizes under stringent conditions to a nucleic acid selected from the group consisting of (SEQ ID NO: 127) [Betula verrucosa nucleic acid] (FIG. 30B), and (SEQ ID NO: 125) [Pinus pinaster nucleic acid] (FIG. 29B).

59. The method of claim 55, wherein the infectious disease is caused by a virus.

60. The method of claim 55, wherein the infectious disease is caused by a bacteria.

61. The method of claim 55, wherein the infectious disease is caused by a protozoa.

62. The method of claim 55, wherein the subject is a non-human animal.

63. The method of claim 55, wherein the subject is a mammal.

64. The method of claim 55, wherein the subject is a human.

65. A method of treating a cancer in a subject, comprising administering to the subject an effective amount of a pharmaceutical formulation comprising an amino acid sequence selected from the group consisting of (SEQ ID NO:1) [Neospora caninum], (SEQ ID NO:2) [Sarcocystis neurona], and (SEQ ID NO: 3) [Toxoplasma gondii].

66. A method of treating a cancer in a subject, comprising administering to the subject an effective amount of a composition comprising an amino acid sequence selected from the group consisting of (SEQ ID NO: 127) [Betula verrucosa polypeptide] (FIG. 30B), and (SEQ ID NO: 125) [Pinus pinaster polypeptide] (FIG. 29A).

67. A method of treating a cancer in a subject, comprising administering to the subject an effective amount of a profilin-related immunostimulatory fragment, wherein the profilin-related immunostimulatory polypeptide is encoded by a nucleic acid that hybridizes under stringent conditions to a nucleic acid selected from the group consisting of (SEQ ID NOS:7-10).

68. A method of treating a cancer in a subject, comprising administering to the subject an effective amount of a profilin-related immunostimulatory fragment, wherein the profilin-related immunostimulatory polypeptide is encoded by a nucleic acid that hybridizes under stringent conditions to a nucleic acid selected from the group consisting of (SEQ ID NO: 127) [Betula verrucosa nucleic acid] (FIG. 30B), and (SEQ ID NO: 125) [Pinus pinaster nucleic acid] (FIG. 29B).

69. The method of claim 65, wherein the cancer is a sarcoma.

70. The method of claim 65, wherein the cancer is a fibrosarcoma.

71. The method of claim 65, wherein the cancer is a carcinoma.

72. The method of claim 65, wherein the subject is a non-human animal.

73. The method of claim 65, wherein the subject is a mammal.

74. The method of claim 65, wherein the subject is a human.

75. A method of identifying a candidate subject for treatment with a profilin-related immunomodulatory polypeptide comprising:
obtaining a cellular sample from the subject; and
detecting the presence of a TLR11/TLR12 polypeptide or a TLR11/TLR12-encoding nucleic acid sequence in the subject sample,
wherein the presence of the TLR11/TLR12 polypeptide or TLR11/TLR12-encoding nucleic acid sequence in the subject sample indicates that the subject is a candidate for treatment with a profilin-related immunomodulatory polypeptide.

76. The method of claim 75, comprising determining a TLR12 polymorphism present in the subject.

77. The method of claim 75, wherein the subject is a mammal.

78. The method of claim 75, wherein the subject is a human.
